(19) United States
(12) Patent Application Publication
(10) Pub. No.: US 2024/0190860 A1
(43) Pub. Date: Jun. 13, 2024

(54) 8-CYCLO-SUBSTITUTED QUINAZOLINE DERIVATIVES AS LPA RECEPTOR 2 INHIBITORS

(71) Applicant: CHIESI FARMACEUTICI S.p.A., Parma (IT)

(72) Inventors: Gabriele AMARI, Parma (IT); Elisabetta ARMANI, Parma (IT); Mafalda PAGANO, Parma (IT); Marta GIULIANI, Parma (IT); Luca RAVEGLIA, Parma (IT); Claudia BEATO, Parma (IT)

(21) Appl. No.: 18/551,942

(22) PCT Filed: Mar. 24, 2021
(86) PCT No.: PCT/EP2021/057609

§ 371 Date: Sep. 22, 2023

(51) Int. Cl. C07D 417/14; A61K 31/517; C07D 401/04; C07D 413/14

(52) U.S. Cl. CPC C07D 417/14; A61K 31/517; C07D 401/04; C07D 413/14

(57) ABSTRACT

The present invention relates to compounds of general formula (I) inhibiting lysophosphatidic acid receptor 2 (LPA2), particularly the invention relates to compounds that are 8-cyclo-substituted quinazoline derivatives, methods of preparing such compounds, pharmaceutical compositions containing them and therapeutic use thereof. The compounds of the invention may be useful in the treatment of diseases or conditions associated with a dysregulation of LPA receptors, in particular fibrosis.

### FIELD OF THE INVENTION

The present invention generally relates to compounds inhibiting lysophosphatidic acid receptors (hereinafter LPA inhibitors); the invention relates to compounds that are 8-cyclo-substituted quinazoline derivatives, methods of preparing such compounds, pharmaceutical compositions containing them and therapeutic use thereof.

The compounds of the invention may be useful for instance in the treatment of many disorders associated with LPA receptors mechanisms.

### BACKGROUND OF THE INVENTION

Lysophosphatidic acid (LPA) is a phospholipid mediator concentrated in serum that acts as a potent extracellular signalling molecule through at least six cognate G protein-coupled receptors (GPCRs) in numerous developmental and adult processes including cell survival, proliferation, migration, differentiation, vascular regulation, and cytokine release.

These LPA-mediated processes involve nervous system function, vascular development, immune system function, cancer, reproduction, fibrosis, and obesity (see e.g. Yung et al., J Lipid Res. 2014 July; 55(7):1192-214). The formation of an LPA species depends on its precursor phospholipid, which can vary typically by acyl chain length and degree of saturation. The term LPA generally refers to 18:1 oleoyl-LPA (1-acyl-2-hydroxy-sn-glycero3-phosphate), that is the most quantitatively abundant forms of LPA in human plasma with 16:0-, 18:2-, and 18:1-LPA (see e.g. Sano et al., J Biol Chem. 2002 Dec. 13; 277(50):21197-206). All LPA species are produced from membrane phospholipids via two major metabolic routes. Depending upon the site of synthesis, membrane phospholipids get converted to the corresponding lysophospholipids by the action of phospholipase A1 (PLA1), phospholipase A2 (PLA2), or PLA1 and lecithin-cholesterol acyltransferase (LCAT). Autotaxin (ATX) then acts on the lysophospholipids and converts them into LPA species. The second pathway first converts the phospholipids into phosphatidic acid by the action of phospholipase D. Then PLA1 or PLA2 metabolize phosphatidic acid to the lysophosphatidic acids (see e.g. Riaz et al., Int J Mol Sci. 2016 February; 17(2): 215).

ATX activity is the major source of plasma extracellular LPA but the source of tissue LPA that contributes to signalling pools likely involves not only ATX but other enzymes as well. The biological functions of LPA are mediated by at least six recognized cell-surface receptors. All LPA receptors are rhodopsin-like 7-TM proteins that signal through at least two of the four Ga subunit families (Gα12/13, Gαq/11, Gαi/o and GαS). LPA receptors usually trigger response from multiple heterotrimeric G-proteins, resulting in diverse outcomes in a context and cell type dependent manner. Gα12/13-mediated LPA signalling regulates cell migration, invasion and cytoskeletal re-adjustments through activation of RHO pathway proteins. RAC activation downstream of Gαi/o-PI3K also regulates similar processes, but the most notable function of LPA-induced Gαi/o is mitogenic signalling through the RAF-MEK-MAPK cascade and survival signalling through the PI3K-AKT pathway. The LPA-coupled Gαq/11 protein primarily regulates Ca2+ homeostasis through PLC and the second messengers IP3 and DAG. Lastly, GαS can activate adenylyl cyclase and increase cAMP concentration upon LPA stimulation (see e.g. Riaz et al., Int J Mol Sci. 2016 February; 17(2): 215).

LPA, especially LPA1, LPA2 and LPA3, have been implicated in migration, invasion, metastasis, proliferation and survival and differ in their tissue distribution and downstream signalling pathways.

LPA1 is a 41-kD protein that is widely expressed, albeit at different levels, in all human adult tissues examined and the importance of LPA1 signalling during development and adult life has been demonstrated through numerous approaches (see e.g. Ye at al., 2002, Neuroreport. December 3; 13(17):2169-75). Wide expression of LPA1 is observed in adult mice, with clear presence in at least brain, uterus, testis, lung, small intestine, heart, stomach, kidney, spleen, thymus, placenta, and skeletal muscle. LPA1 is also widely expressed in humans where the expression is more spatially restricted during embryonic development. LPA1 couples with and activates three types of G proteins: Gαi/o, Gαq/11, and Gα12/13. LPA1 activation induces a range of cellular responses: cell proliferation and survival, cell migration, cytoskeletal changes, Ca2+ mobilization, adenylyl cyclase inhibition and activation of mitogen-activated protein kinase, phospholipase C, Akt, and Rho pathways (see e.g. Choi et al., Annu Rev Pharmacol Toxicol. 2010; 50:157-86).

LPA2 in humans is a 39-kD protein and shares ˜55% amino acid sequence homology with LPA1 (see e.g. Yung et al., J Lipid Res. 2014 July; 55(7):1192-214). In mouse, LPA2 is highly expressed in kidney, uterus, and testis and moderately expressed in lung; in human tissues, high expression of LPA2 is detected in testis and leukocytes, with moderate expression found in prostate, spleen, thymus, and pancreas. In terms of signalling activity, LPA2 mostly activates the same pathways as triggered by LPA1 with some exceptions that regards its unique cross-talk behaviour. For example, LPA2 promotes cell migration through interactions with focal adhesion molecule TRIP6 (see e.g. Lai Y J, 2005, Mol. Cell. Biol. 25:5859-68), and several PDZ proteins and zinc finger proteins are also reported to interact directly with the carboxyl-terminal tail of LPA2 (see e.g. Lin F T, 2008, Biochim. Biophys. Acta 1781:558-62).

Human LPA3 is a 40-kD protein and shares sequence homology with LPA1 (˜54%) and LPA2 (˜49%). In adult humans LPA3 is highly expressed in heart, pancreas, prostate and testis. Moderate levels of expression are also found in brain, lungs and ovary. Like LPA1 and LPA2 the signalling activity of LPA3 results from its coupling to Gαi/o and Gαq/11 (see e.g Ishii et al., Mol Pharmacol 58:895-902, 2000). Each LPA has multiple important regulatory functions throughout the body.

As LPA signalling has been strongly implicated in many disease states, great interest has been expressed in developing specific LPA inhibitors (see e.g. Stoddard et el., Biomol Ther (Seoul) 2015 January; 23(1):1-11). Different studies have demonstrated a positive role for LPA in the pathogenesis of pulmonary fibrosis (PF), a devastating disease characterized by alveolar epithelial cell injury, accumulation of myofibroblasts and deposition of extracellular matrix proteins leading to a loss of lung function and death (see e.g. Wilson M S, Wynn T A (2009), Mucosal Immunol 2: 103-121).

Evidences showed that lysophosphatidic acid levels dramatically increase in bronchoalveolar lavage fluid of PF patients where it mediates fibroblast migration in the injured lung acting through LPA1 (see e.g. Tager et al., Nat Med. 2008 January; 14(1):45-54). In addition, mice lacking LPA1 or LPA2 are markedly protected from fibrosis and mortality in a mouse model of the bleomycin induced pulmonary fibrosis (see e.g. Huang et al., Am J Respir Cell Mol Biol. 2013 December; 49(6): 912-922 and Tager et al., Nat Med. 2008 January; 14(1):45-54).

In vitro, LPA1 is known to induce the proliferation and differentiation of lung fibroblasts (see e.g. Shiomi et al., Wound Repair Regen. 2011 March-April; 19(2): 229-240), and to augment the fibroblast-mediated contraction of released collagen gels (see e.g. Mio et al., Journal of Laboratory and Clinical Medicine, Volume 139, Issue 1, January 2002, Pages 20-27). In human lung fibroblasts, the knockdown of LPA2 attenuated the LPA-induced expression of TGF-β1 and the differentiation of lung fibroblasts to myofibroblasts, resulting in the decreased expression of different profibrotic markers such as FN, α-SMA, and collagen, as well as decreased activation of extracellular regulated kinase 1/2, Akt, Smad3, and p38 mitogen-activated protein kinase (see e.g. Huang et al., Am J Respir Cell Mol Biol. 2013 December; 49(6): 912-922). Moreover Xu et al., confirmed that the expression of LPA2 was also up-regulated in lungs from bleomycin-challenged mice where it is able to induce the activation of TGF-β pathway, a key cytokine that play an essential role during the development of the disease, via a RhoA and Rho kinase pathway (see e.g. Xu et al., Am J Pathol. 2009 April; 174(4):1264-79). In in vivo preclinical model, the oral administration of an LPA1 antagonist significantly reduced bleomycin-induced pulmonary fibrosis in mice (Tager et al., Nat Med. 2008 January; 14(1):45-54; Swaney et al., Br J Pharmacol. 2010 August; 160(7): 1699-1713), and the intraperitoneal injection of an LPA1/3 antagonist ameliorated irradiation-induced lung fibrosis (see e.g. Gan et al., 2011, Biochem Biophys Res Commun 409: 7-13). In a renal fibrosis model, LPA1 administration of an LPA1 antagonist suppressed renal interstitial fibrosis (see e.g Pradere et al., J Am Soc Nephrol 2007; 18:3110-3118).

Various compounds have been described in the literature as LPA1 or LPA2 antagonist.

WO2019126086 and WO2019126087 (Bristol-Myers Squibb) disclose cyclohexyl acid isoxazole azines as LPA1 antagonist, useful for the treatment of disorder or condition associated with dysregulation of lysophosphatidic acid receptor 1.

WO2019126099 (Bristol-Myers Squibb) discloses isoxazole N-linked carbamoyl cyclohexyl acid as LPA1 antagonist for the treatment of disorder or condition associated with dysregulation of lysophosphatidic acid receptor 1.

WO2019126090 (Bristol-Myers Squibb) discloses triazole N-linked carbamoyl cyclohexyl acids as LPA1 antagonists. The compounds are selective LPA1 receptor inhibitors and are useful for the treatment of disorder or condition associated with dysregulation of lysophosphatidic acid receptor 1.

WO2017223016 (Bristol-Myers Squibb) discloses carbamoyloxymethyl triazole cyclohexyl acids as LPA1 antagonist for the treatment of fibrosis including idiopathic pulmonary fibrosis.

WO2012028243 (Merck) discloses pyrazolopyridinone derivatives according to formula (I) and a process of manufacturing thereof as LPA2 receptor antagonists for the treatment of various diseases.

Amgen Inc. discloses in “Discovery of potent LPA2 (EDG4) antagonists as potential anticancer agents” Bioorg Med Chem Lett. 2008 Feb. 1; 18(3):1037-41, LPA2 antagonists. Key compounds were evaluated in vitro for inhibition of LPA2 mediated Erk activation and proliferation of HCT-116 cells. These compounds could be used as tool compounds to evaluate the anticancer effects of blocking LPA2 signalling.

Of note, antagonizing the LPA receptors may be useful for the treatment of fibrosis and diseases, disorders and conditions that result from fibrosis, and even more antagonizing receptor LPA2 may be particularly efficacious in the treatment of the above-mentioned diseases, disorders and conditions.

Several efforts have been done in the past years to develop novel LPA1 receptor antagonist useful for the treatment of several diseases and some of those compounds have shown efficacy also in humans.

Thus, there remains a potential for developing inhibitors of receptors LPA2 useful for the treatment of diseases or conditions associated with a dysregulation of LPA receptors, in particular fibrosis.

In this respect, the state of the art does not describe or suggest 8-cyclo-substituted quinazoline derivatives of general formula (I) of the present invention having an antagonist activity on receptor LPA2 which represents a solution to the aforementioned need.

### SUMMARY OF THE INVENTION

In a first aspect the invention refers to a compound of formula (I)

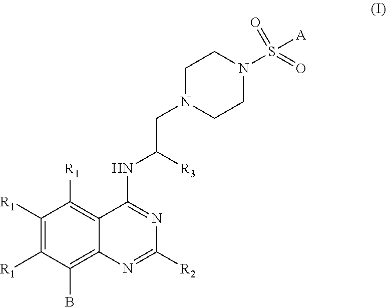

wherein
B is selected from the group consisting of (C₄-C₈) heterocycloalkyl, heteroaryl, (C₃-C₈)cycloalkyl, and aryl wherein each of said cycloalkyl, heterocycloalkyl, heteroaryl and aryl may be optionally substituted by one or more group selected from (C₁-C₄)alkyl, halo, (C₁-C₄)haloalkyl, —(C₁-C₄)alkylene-NR_{A}R_{B}, —NR_{A}R_{B}, —NR_{A}C(O)R₁, —C(O)R₁, —CN and (C₃-C₈)cycloalkyl, or
each of said aryl may be fused to second saturated or unsaturated ring optionally containing one or more heteroatoms selected from N, O and S to form a bicyclic ring system optionally substituted by one or more group selected from —C(O)R₁, (C₁-C₄)alkyl and oxo;
R₁ is H or (C₁-C₄)alkyl;
R₂ is H or selected from the group consisting of (C₁-C₄)alkyl, (C₁-C₄)haloalkyl, —(C₁-C₄)alkylene-OR₁ and (C₃-C₈)cycloalkyl;
R₃ is H or (C₁-C₄)alkyl;
A is selected from the group consisting of 5-6 membered heteroaryl and aryl wherein each of said heteroaryl and aryl may be optionally substituted by one or more group selected from (C₁-C₄)alkyl, —C(O)R₁, —C(O)ORI, —C(O)R₁, (C₁-C₄)haloalkyl, halo, —NR_{A}C(O)R₁, —NR_{A}C(O)OR₁, —NR_{A}C(O)—(C₁-C₄)alkylene-OR₁, —NR_{A}C(O)R_{C}, —NR_{A}C(O)NR_{A}R_{B}, —NR_{A}C(O)—(C₁-C₄)alkylene-NR_{A}R_{B}, —N(C₁-C₄)alkylene-NR_{A}R_{B}, aryl and heteroaryl optionally substituted by one or more (C₁-C₄)alkyl and (C₁-C₄)haloalkyl, or when A is aryl it may be fused to a second saturated or unsaturated ring optionally containing one or more heteroatoms selected from N, O and S to form a bicyclic ring system optionally substituted by one or more group selected from —C(O)R₁, (C₁-C₄)alkyl and oxo;
R_{C} is selected from the group consisting of heteroaryl, aryl, (C₃-C₈) cycloalkyl and (C₄-C₈) heterocycloalkyl wherein said heteroaryl, aryl, heterocycloalkyl and cycloalkyl may be optionally substituted by one or more (C₁-C₄)alkyl and —C(O)OR₁;
R_{A} and R_{B} are at each occurrence independently H or selected from the group consisting of (C₁-C₄)alkyl, (C₃-C₈)cycloalkyl, (C₁-C₆)haloalkyl and halo, or
R_{A} and R_{B} may form together with the nitrogen atom to which they are attached a 4-6 membered saturated heterocyclic ring system optionally containing a further heteroatom selected from N, S and O, said heterocyclic ring system may be optionally substituted by one or more groups selected from (C₁-C₄)alkyl, (C₁-C₄) haloalkyl and halo.

In a second aspect, the invention refers to pharmaceutical composition comprising a compound of formula (I) in admixture with one or more pharmaceutically acceptable carrier or excipient.

In a third aspect, the invention refers to a compound of formula (I) for use as a medicament.

In a further aspect, the invention refers to a compound of formula (I) for use in treating diseases, disorders, or conditions associated with dysregulation of lysophosphatidic acid receptor 2 (LPA2).

In a further aspect, the invention refers to a compound of formula (I) for use in the prevention and/or treatment of fibrosis and/or diseases, disorders, or conditions that involve fibrosis.

In a further aspect, the invention refers to a compound of formula (I) for use in the prevention and/or treatment idiopathic pulmonary fibrosis (IPF).

### DETAILED DESCRIPTION OF THE INVENTION

Unless otherwise specified, the compound of formula (I) of the present invention is intended to include also stereoisomer, tautomer or pharmaceutically acceptable salt or solvate thereof.

The term “pharmaceutically acceptable salts”, as used herein, refers to derivatives of compounds of formula (I) wherein the parent compound is suitably modified by converting any of the free acid or basic group, if present, into the corresponding addition salt with any base or acid conventionally intended as being pharmaceutically acceptable.

Suitable examples of said salts may thus include mineral or organic acid addition salts of basic residues such as amino groups, as well as mineral or organic basic addition salts of acid residues such as carboxylic groups.

Cations of inorganic bases which can be suitably used to prepare salts comprise ions of alkali or alkaline earth metals such as potassium, sodium, calcium or magnesium.

Those obtained by reacting the main compound, functioning as a base, with an inorganic or organic acid to form a salt comprise, for example, salts of hydrochloric acid, hydrobromic acid, sulfuric acid, phosphoric acid, methane sulfonic acid, camphor sulfonic acid, acetic acid, oxalic acid, maleic acid, fumaric acid, succinic acid and citric acid.

The term “solvate” means a physical association of a compound of this invention with one or more solvent molecules, whether organic or inorganic. This physical association includes hydrogen bonding. In certain instances, the solvate will be capable of
isolation, for example, when one or more solvent molecules are incorporated in the crystal lattice of the crystalline solid. The solvate may comprise either a stoichiometric or nonstoichiometric amount of the solvent molecules.

The term “stereoisomer” refers to isomers of identical constitution that differ in the arrangement of their atoms in space. Enantiomers and diastereomers are examples of stereoisomers.

The term “enantiomer” refers to one of a pair of molecular species that are mirror images of each other and are not superimposable.

The term “diastereomer” refers to stereoisomers that are not mirror images.

The term “racemate” or “racemic mixture” refers to a composition composed of equimolar quantities of two enantiomeric species, wherein the composition is devoid of optical activity.

The symbols “R” and “S” represent the configuration of substituents around a chiral carbon atom(s). The isomeric descriptors “R” and “S” are used as described herein for indicating atom configuration(s) relative to a core molecule and are intended to be used as defined in the literature (IUP AC Recommendations 1996, Pure and Applied Chemistry, 68:2193-2222 (1996)).

The term “tautomer” refers to each of two or more isomers of a compound that exist together in equilibrium and are readily interchanged by migration of an atom or group within the molecule.

The term “halogen” or “halogen atoms” or “halo” as used herein includes fluorine, chlorine, bromine, and iodine atom.

The term “5-membered heterocyclyl” refers to a mono satured or unsatured group containing one or more heteroatoms selected from N and O.

The term “(Cₓ-C_{y}) alkyl” wherein x and y are integers, refers to a straight or branched chain alkyl group having from x to y carbon atoms. Thus, when x is 1 and y is 6, for example, the term includes methyl, ethyl, n-propyl, isopropyl, n-butyl, isobutyl, sec-butyl, t-butyl, n-pentyl and n-hexyl.

The term “(Cₓ-C_{y})alkylene” wherein x and y are integers, refers to a Cₓ-C_{y}alkyl radical having in total two unsatisfied valencies, such as a divalent methylene radical.

The expressions “(Cₓ-C_{y}) haloalkyl” wherein x and y are integers, refer to the above defined “Cₓ-C_{y}alkyl” groups wherein one or more hydrogen atoms are replaced by one or more halogen atoms, which can be the same or different.

Examples of said “(Cₓ-C_{y}) haloalkyl” groups may thus include halogenated, poly-halogenated and fully halogenated alkyl groups wherein all hydrogen atoms are replaced by halogen atoms, e.g. trifluoromethyl.

The term “(Cₓ-C_{y}) cycloalkyl” wherein x and y are integers, refers to saturated cyclic hydrocarbon groups containing the indicated number of ring carbon atoms. Examples include cyclopropyl, cyclobutyl, cyclopentyl, cyclohexyl, cycloheptyl.

The term “aryl” refers to mono cyclic carbon ring systems which have 6 ring atoms wherein the ring is aromatic. Examples of suitable aryl monocyclic ring systems include, for instance, phenyl.

The term “heteroaryl” refers to a mono- or bi-cyclic aromatic group containing one or more heteroatoms selected from S, N and O, and includes groups having two such monocyclic rings, or one such monocyclic ring and one monocyclic aryl ring, which are fused through a common bond.

The term “(Cₓ-C_{y}) heterocycloalkyl” wherein x and y are integers, refers to saturated or partially unsaturated monocyclic (Cₓ-C_{y}) cycloalkyl groups in which at least one ring carbon atom is replaced by at least one heteroatom (e.g. N, S or O) or may bear an -oxo (═O) substituent group. Said heterocycloalkyl may be further optionally substituted on the available positions in the ring, namely on a carbon atom, or on an heteroatom available for substitution. Substitution on a carbon atom includes spiro disubstitution as well as substitution on two adjacent carbon atoms, in both cases thus form additional condensed 5 to 6 membered heterocyclic ring.

The term “(Cₓ-C_{y}) aminoalkyl” wherein x and y are integers, refers to the above defined “(C₁-C₆) alkyl” groups wherein one or more hydrogen atoms are replaced by one or more amino group.

The term “(Cₓ-C_{y}) hydroxyalkyl” wherein x and y are integers, refers to the above defined “(C₁-C₆) alkyl” groups wherein one or more hydrogen atoms are replaced by one or more hydroxy (OH) group.

The term “(Cₓ-C_{y}) alkoxy” or “(Cₓ-C_{y}) alkoxyl” wherein x and y are integers, refer to a straight or branched hydrocarbon of the indicated number of carbons, attached to the rest of the molecule through an oxygen bridge.

A dash (“-”) that is not between two letters or symbols is meant to represent the point of attachment for a substituent.

The carbonyl group is herein preferably represented as —C(O)— as an alternative to the other common representations such as —CO—, —(CO)— or —C(═O)—.

In general, the bracketed group is a lateral group, not included into the chain, and brackets are used, when deemed useful, to help disambiguating linear chemical formulas; e.g. the sulfonyl group —SO₂— might be also represented as —S(O)₂— to disambiguate e.g. with respect to the sulfinic group —S(O)O—.

In the presence of acidic groups such as COOH groups, corresponding physiological cation salts may be present as well, for instance including alkaline or alkaline earth metal ions.

As above indicated, the present invention refers to a series of compounds represented by the general formula (I) as herein below described in details, which are endowed with an inhibitory activity on receptor LPA2.

Advantageously, the antagonist action receptor LPA2 can be effective in the treatment of those diseases where the LPA receptors play a relevant role in the pathogenesis such as fibrosis and disease, disorder and condition from fibrosis.

Differently from similar compounds of the prior art, such as compounds disclosed for example in Merck WO2012028243 and Amgen compounds, the compounds of formula (I) of the present invention are much more active on the LPA2 receptor.

The Merck and Amgen compounds show a maximum potency expressed as half maximal inhibitory concentration (IC₅₀) on LPA2 around 500 nm.

As indicated in the experimental part, in particular in Table 2, the compounds of formula (I) of the present invention show a notable potency with respect to their inhibitory activity on receptor LPA2 below about 500 nm, confirming that they are able to antagonize the isoform of LPA2 receptor involved in fibrosis and diseases that result from fibrosis with a greater potency respect to the compounds of the prior art.

Advantageously, the compounds of the present invention characterized by a very high potency, could be administered in human at a lower dosage in comparison to the compounds of the prior art, thus reducing the adverse events that typically occur administering higher dosages of drug.

Therefore, the compounds of the present invention are particularly appreciated by the skilled person when looking at a suitable and efficacious compounds useful for the treatment of fibrosis, in particular idiopathic pulmonary fibrosis.

Thus, in one aspect the present invention relates to a compound of general formula (I) as LPA2 antagonist

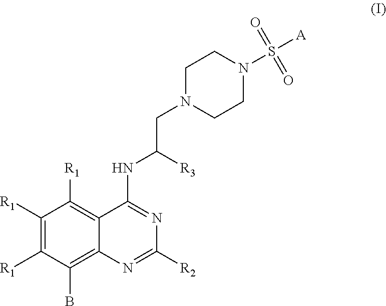

wherein
B is selected from the group consisting of (C₄-C₈) heterocycloalkyl, heteroaryl, (C₃-C₈)cycloalkyl, and aryl wherein each of said cycloalkyl, heterocycloalkyl, heteroaryl and aryl may be optionally substituted by one or more group selected from (C₁-C₄)alkyl, halo, (C₁-C₄)haloalkyl, —(C₁-C₄)alkylene-NR_{A}R_{B}, —NR_{A}R_{B}, —NR_{A}C(O)R₁, —C(O)R₁, —CN, (C₃-C₈)cycloalkyl, or
each of said aryl may be fused to a second saturated or unsaturated ring optionally containing one or more heteroatoms selected from N, O and S to form a bicyclic ring system optionally substituted by one or more group selected from —C(O)R₁, (C₁-C₄)alkyl and oxo;
R₁ is H or (C₁-C₄)alkyl;
R₂ is H or selected from the group consisting of (C₁-C₄)alkyl, (C₁-C₄)haloalkyl, —(C₁-C₄)alkylene-OR₁ and (C₃-C₈)cycloalkyl;
R₃ is H or (C₁-C₄)alkyl;
A is selected from the group consisting of 5-6 membered heteroaryl and aryl wherein each of said heteroaryl and aryl may be optionally substituted by one or more group selected from (C₁-C₄)alkyl, —C(O)R₁, —C(O)ORI, —C(O)R₁, (C₁-C₄)haloalkyl, halo, —NR_{A}C(O)R₁, —NR_{A}C(O)OR₁, —NR_{A}C(O)—(C₁-C₄)alkylene-OR₁, —NR_{A}C(O)R_{C}, —NR_{A}C(O)NR_{A}R_{B}, —NR_{A}C(O)—(C₁-C₄)alkylene-NR_{A}R_{B}, —N(C₁-C₄)alkylene-NR_{A}R_{B}, aryl and heteroaryl optionally substituted by one or more (C₁-C₄)alkyl and (C₁-C₄)haloalkyl, or when A is aryl it may be fused to a second saturated or unsaturated ring optionally containing one or more heteroatoms selected from N, O and S to form a bicyclic ring system optionally substituted by one or more group selected from —C(O)R₁, (C₁-C₄)alkyl and oxo;
R_{C} is selected from the group consisting of from heteroaryl, aryl, (C₃-C₈) cycloalkyl and (C₄-C₈) heterocycloalkyl wherein said heteroaryl, aryl, heterocycloalkyl and cycloalkyl may be optionally substituted by one or more (C₁-C₄)alkyl and —C(O)OR₁;
R_{A} and R_{B} are at each occurrence independently H or selected from the group consisting of (C₁-C₄)alkyl, (C₃-C₈)cycloalkyl, (C₁-C₆)haloalkyl and halo, or R_{A} and R_{B} may form together with the nitrogen atom to which they are attached a 4-6 membered saturated heterocyclic ring system optionally containing a further heteroatom selected from N, S and O, said heterocyclic ring system may be optionally substituted by one or more groups selected from (C₁-C₄)alkyl, (C₁-C₄) haloalkyl and halo.

In a preferred embodiment the invention refers to a compound of formula (I)
wherein
B is selected from the group consisting of (C₄-C₈) heterocycloalkyl, heteroaryl, (C₃-C₈)cycloalkyl, and aryl wherein each of said heterocycloalkyl, heteroaryl and aryl may be optionally substituted by one or more group selected from (C₁-C₄)alkyl, halo, (C₁-C₄)haloalkyl, —(C₁-C₄)alkylene-NR_{A}R_{B}, —NR_{A}R_{B}, —NR_{A}C(O)R₁, —C(O)R₁, —CN and (C₃-C₈)cycloalkyl, or
each of said aryl may be fused to saturated ring containing N to form a bicyclic ring system, optionally substituted by one or more (C₁-C₄)alkyl;
R₁ is H or (C₁-C₄)alkyl;
R₂ is H or selected from the group consisting of (C₁-C₄)alkyl, (C₁-C₄)haloalkyl, —(C₁-C₄)alkylene-OR₁ and (C₃-C₈)cycloalkyl;
R₃ is (C₁-C₄)alkyl;
A is selected from the group consisting of 5-6 membered heteroaryl and aryl wherein each of said heteroaryl and aryl may be optionally substituted by one or more group selected from (C₁-C₄)alkyl, halo, —NR_{A}C(O)R₁, —NR_{A}C(O)OR₁, —NR_{A}C(O)—(C₁-C₄)alkylene-OR₁, —NR_{A}C(O)R_{C}, —N(C₁-C₄)alkylene-NR_{A}R_{B}, heteroaryl optionally substituted by one or more (C₁-C₄)alkyl; or when A is aryl it may be fused to a second saturated or partially saturated ring optionally containing one or more heteroatoms selected from N, S and O, to form a bicyclic ring system optionally substituted by one or more group selected from oxo, —C(O)R₁, and (C₁-C₄)alkyl;
R_{C} is heteroaryl optionally substituted by one or more (C₁-C₄)alkyl;
R_{A} and R_{B} are at each occurrence independently H or selected from the group consisting of (C₁-C₄)alkyl, (C₃-C₈)cycloalkyl, (C₁-C₆)haloalkyl and halo, or R_{A} and R_{B} may form together with the nitrogen atom to which they are attached a 4-6 membered saturated heterocyclic ring system optionally containing a further heteroatom selected from N, S and O, said heterocyclic ring system may be optionally substituted by one or more groups selected from (C₁-C₄)alkyl, (C₁-C₄) haloalkyl and halo.

In one preferred embodiment, when B is heteroaryl said heteroaryl is selected from the group consisting of thiazole, pyrazine, isoxazole, pyrazole, pyridine and pyrimidine.

In one preferred embodiment A is selected from the group consisting of 5-6 membered heteroaryl 5 and aryl wherein each of said heteroaryl and aryl may be optionally substituted by one or more group selected from (C₁-C₄)alkyl, halo, —NR_{A}C(O)R₁, —NR_{A}C(O)OR₁, —NR_{A}C(O)—(C₁-C₄)alkylene-OR₁, —NR_{A}C(O)R_{C}, —N(C₁-C₄)alkylene-NR_{A}R_{B} and isoxazole optionally substituted by one or more (C₁-C₄)alkyl; or
when A is aryl it may be fused to a second saturated or unsaturated ring optionally containing one or more heteroatoms selected from N, O and S to form a bicyclic ring system optionally substituted by one or more group selected from —C(O)R₁, (C₁-C₄)alkyl and oxo;

In one preferred embodiment when A is 5-6 membered heteroaryl said 5-6 membered heteroaryl is selected from the group consisting of thiazole, thiophene and pyridine.

In one preferred embodiment when R_{C} is heteroaryl said heteroaryl is isoxazole optionally substituted by one or more (C₁-C₄)alkyl and —C(O)OR₁.

According to the preferred embodiment, the invention refers to at least one of the compounds listed in the Table 1 below; those compounds are active on LPA2, as shown in Table 2.

TABLE 1
List of preferred compounds of Formula (I)

| Ex. No | Structure | Chemical Name |
|---|---|---|
| 1 | 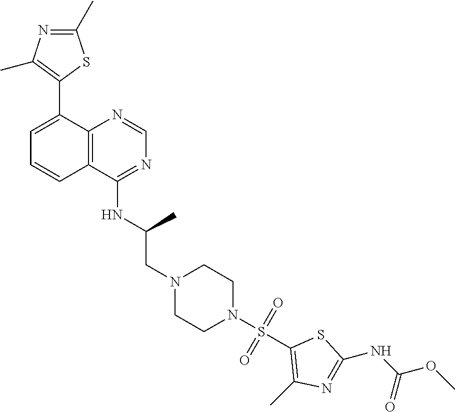 | methyl N-[5-({4-[(2S)-2-{[8-(2,4-dimethyl- 1,3-thiazol-5-yl)quinazolin-4- yl]amino}propyl]piperazin-1-yl}sulfonyl)-4- methyl-1,3-thiazol-2-yl]carbamate |
| 2 | 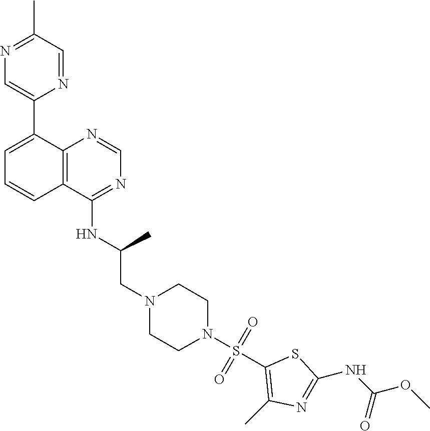 [SMILES: COC(=O)Nc1nc(C)c(S(=O)(=O)N2CCN(C[C@H](C)Nc3ncnc4c(-c5cnc(C)cn5)cccc34)CC2)s1] | methyl N-[4-methyl-5-({4-[(2S)-2-{[8-(5- methylpyrazin-2-yl)quinazolin-4- yl]amino}propyl]piperazin-1-yl}sulfonyl)- 1,3-thiazol-2-yl]carbamate |
| 3 | 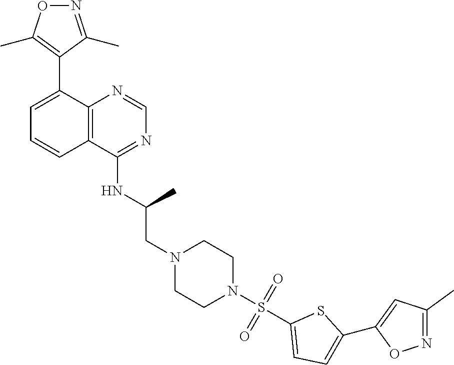 | 8-(3,5-dimethyl-1,2-oxazol-4-yl)-N-[(2S)-1- (4-{[5-(3-methyl-1,2-oxazol-5-yl)thiophen- 2-yl]sulfonyl}piperazin-1-yl)propan-2- yl]quinazolin-4-amine |
| 4 | 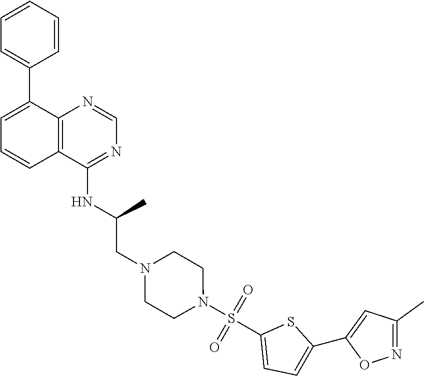 [SMILES: Cc1cc(-c2ccc(S(=O)(=O)N3CCN(C[C@H](C)Nc4ncnc5c(-c6ccccc6)cccc45)CC3)s2)on1] | N-[(2S)-1-(4-{[5-(3-methyl-1,2-oxazol-5- yl)thiophen-2-yl]sulfonyl}piperazin-1- yl)propan-2-yl]-8-phenylquinazolin-4-amine |
| 5 | 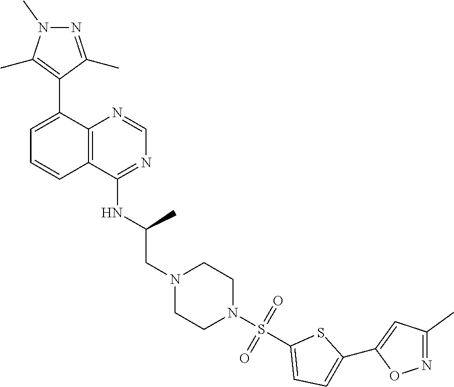 | N-[(2S)-1-(4-{[5-(3-methyl-1,2-oxazol-5- yl)thiophen-2-yl]sulfonyl}piperazin-1- yl)propan-2-yl]-8-(1,3,5-trimethyl-1H- pyrazol-4-yl)quinazolin-4-amine |
| 6 | 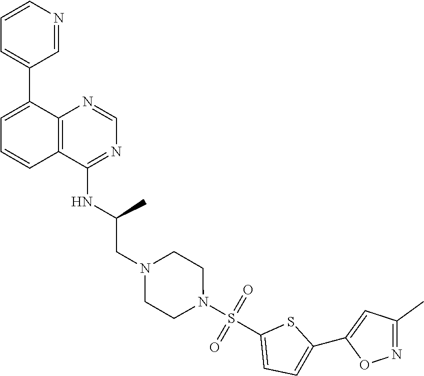 [SMILES: Cc1cc(-c2ccc(S(=O)(=O)N3CCN(C[C@H](C)Nc4ncnc5c(-c6cccnc6)cccc45)CC3)s2)on1] | N-[(2S)-1-(4-{[5-(3-methyl-1,2-oxazol-5- yl)thiophen-2-yl]sulfonyl}piperazin-1- yl)propan-2-yl]-8-(pyridin-3-yl)quinazolin- 4-amine |
| 7 | 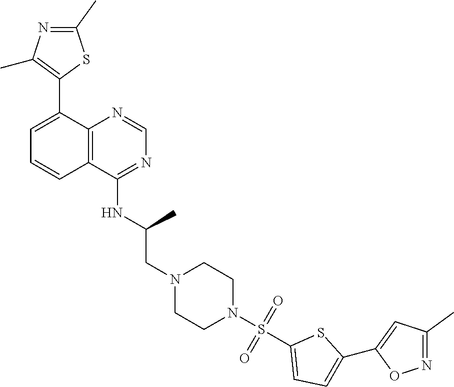 | 8-(2,4-dimethyl-1,3-thiazol-5-yl)-N-[(2S)-1- (4-{[5-(3-methyl-1,2-oxazol-5-yl)thiophen- 2-yl]sulfonyl}piperazin-1-yl)propan-2- yl]quinazolin-4-amine |
| 8 | 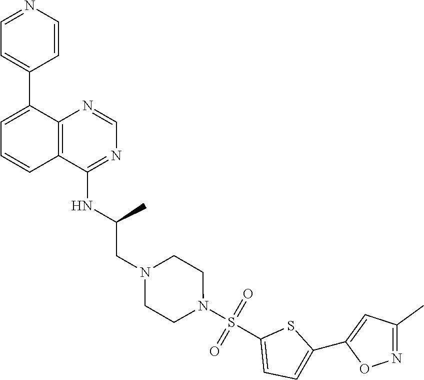 [SMILES: Cc1cc(-c2ccc(S(=O)(=O)N3CCN(C[C@H](C)Nc4ncnc5c(-c6ccncc6)cccc45)CC3)s2)on1] | N-[(2S)-1-(4-{[5-(3-methyl-1,2-oxazol-5- yl)thiophen-2-yl]sulfonyl}piperazin-1- yl)propan-2-yl]-8-(pyridin-4-yl)quinazolin- 4-amine |
| 9 | 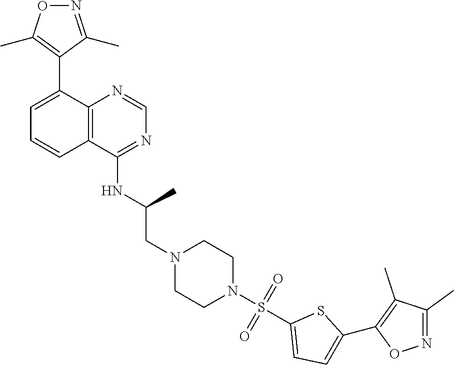 | 8-(3,5-dimethyl-1,2-oxazol-4-yl)-N-[(2S)-1- (4-{[5-(3,4-dimethyl-1,2-oxazol-5- yl)thiophen-2-yl]sulfonyl}piperazin-1- yl)propan-2-yl]quinazolin-4-amine |
| 10 | 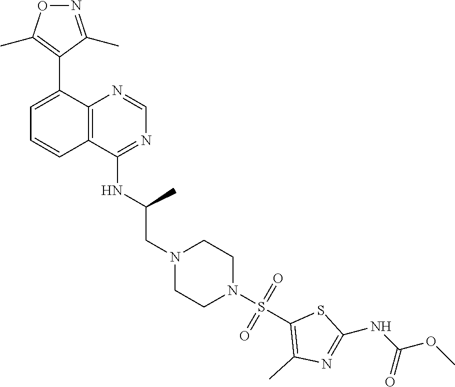 | methyl N-[5-({4-[(2S)-2-{[8-(3,5-dimethyl- 1,2-oxazol-4-yl)quinazolin-4- yl]amino}propyl]piperazin-1-yl}sulfonyl)-4- methyl-1,3-thiazol-2-yl]carbamate |
| 11 | 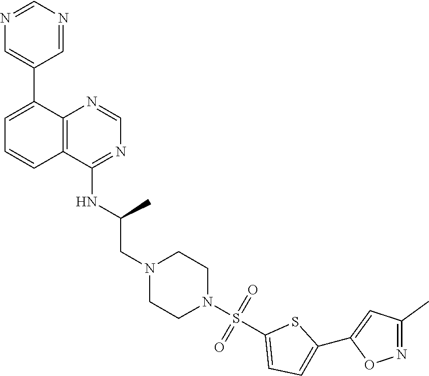 [SMILES: Cc1cc(-c2ccc(S(=O)(=O)N3CCN(C[C@H](C)Nc4ncnc5c(-c6cncnc6)cccc45)CC3)s2)on1] | N-[(2S)-1-(4-{[5-(3-methyl-1,2-oxazol-5- yl)thiophen-2-yl]sulfonyl}piperazin-1- yl)propan-2-yl]-8-(pyrimidin-5- yl)quinazolin-4-amine |
| 12 | 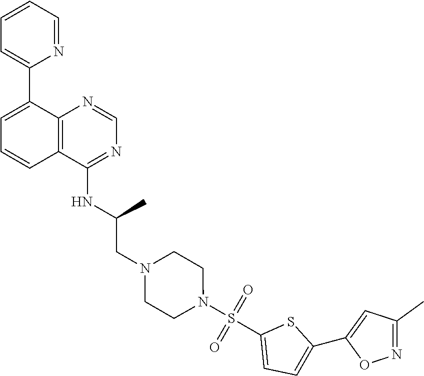 [SMILES: Cc1cc(-c2ccc(S(=O)(=O)N3CCN(C[C@H](C)Nc4ncnc5c(-c6ccccn6)cccc45)CC3)s2)on1] | N-[(2S)-1-(4-{[5-(3-methyl-1,2-oxazol-5- yl)thiophen-2-yl]sulfonyl}piperazin-1- yl)propan-2-yl]-8-(pyridin-2-yl)quinazolin- 4-amine |
| 13 | 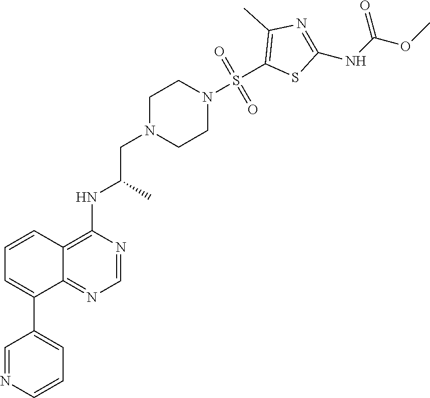 [SMILES: COC(=O)Nc1nc(C)c(S(=O)(=O)N2CCN(C[C@H](C)Nc3ncnc4c(-c5cccnc5)cccc34)CC2)s1] | methyl N-[4-methyl-5-({4-[(2S)-2-{[8- (pyridin-3-yl)quinazolin-4- yl]amino}propyl]piperazin-1-yl}sulfonyl)- 1,3-thiazol-2-yl]carbamate |
| 14 | 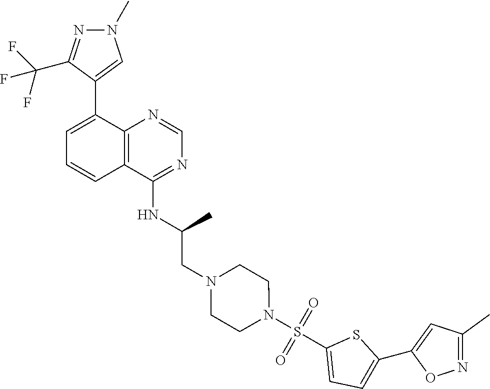 [SMILES: Cc1cc(-c2ccc(S(=O)(=O)N3CCN(C[C@H](C)Nc4ncnc5c(-c6cn(C)nc6C(F)(F)F)cccc45)CC3)s2)on1] | N-[(2S)-1-(4-{[5-(3-methyl-1,2-oxazol-5- yl)thiophen-2-yl]sulfonyl}piperazin-1- yl)propan-2-yl]-8-[1-methyl-3- (trifluoromethyl)-1H-pyrazol-4- yl]quinazolin-4-amine |
| 15 | 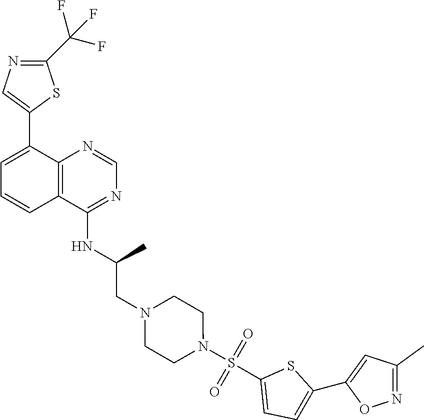 [SMILES: Cc1cc(-c2ccc(S(=O)(=O)N3CCN(C[C@H](C)Nc4ncnc5c(-c6cnc(C(F)(F)F)s6)cccc45)CC3)s2)on1] | N-[(2S)-1-(4-{[5-(3-methyl-1,2-oxazol-5- yl)thiophen-2-yl]sulfonyl}piperazin-1- yl)propan-2-yl]-8-[2-(trifluoromethyl)-1,3- thiazol-5-yl]quinazolin-4-amine |
| 16 | 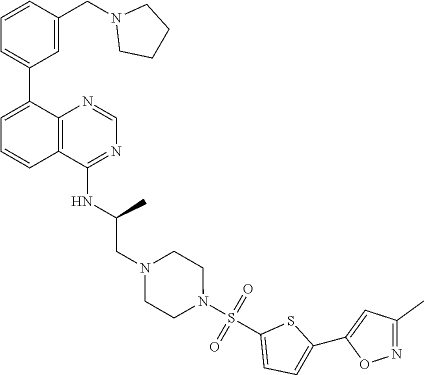 [SMILES: Cc1cc(-c2ccc(S(=O)(=O)N3CCN(C[C@H](C)Nc4ncnc5c(-c6cccc(CN7CCCC7)c6)cccc45)CC3)s2)on1] | N-[(2S)-1-(4-{[5-(3-methyl-1,2-oxazol-5- yl)thiophen-2-yl]sulfonyl}piperazin-1- yl)propan-2-yl]-8-{3-[(pyrrolidin-1- yl)methyl]phenyl}quinazolin-4-amine |
| 17 | 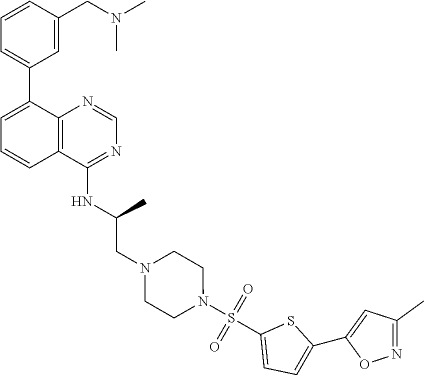 [SMILES: Cc1cc(-c2ccc(S(=O)(=O)N3CCN(C[C@H](C)Nc4ncnc5c(-c6cccc(CN(C)C)c6)cccc45)CC3)s2)on1] | 8-{3-[(dimethylamino)methyl]phenyl}-N- [(2S)-1-(4-{[5-(3-methyl-1,2-oxazol-5- yl)thiophen-2-yl]sulfonyl}piperazin-1- yl)propan-2-yl]quinazolin-4-amine |
| 18 | 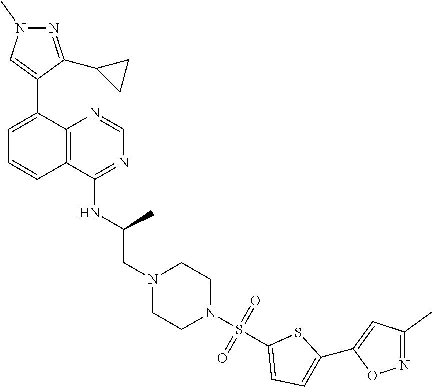 | 8-(3-cyclopropyl-1-methyl-1H-pyrazol-4- yl)-N-[(2S)-1-(4-{[5-(3-methyl-1,2-oxazol- 5-yl)thiophen-2-yl]sulfonyl}piperazin-1- yl)propan-2-yl]quinazolin-4-amine |
| 19 | 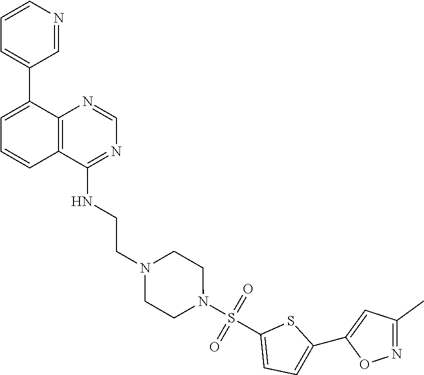 [SMILES: Cc1cc(-c2ccc(S(=O)(=O)N3CCN(CCNc4ncnc5c(-c6cccnc6)cccc45)CC3)s2)on1] | N-[2-(4-{[5-(3-methyl-1,2-oxazol-5- yl)thiophen-2-yl]sulfonyl}piperazin-1- yl)ethyl]-8-(pyridin-3-yl)quinazolin-4-amine |
| 20 | 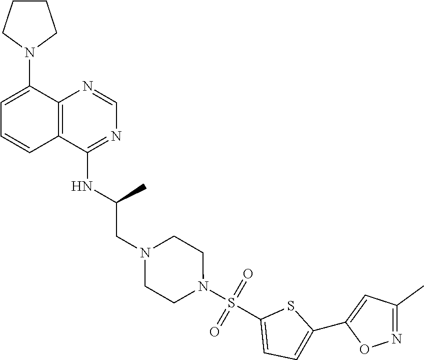 [SMILES: Cc1cc(-c2ccc(S(=O)(=O)N3CCN(C[C@H](C)Nc4ncnc5c(N6CCCC6)cccc45)CC3)s2)on1] | N-[(2S)-1-(4-{[5-(3-methyl-1,2-oxazol-5- yl)thiophen-2-yl]sulfonyl}piperazin-1- yl)propan-2-yl]-8-(pyrrolidin-1- yl)quinazolin-4-amine |
| 21 | 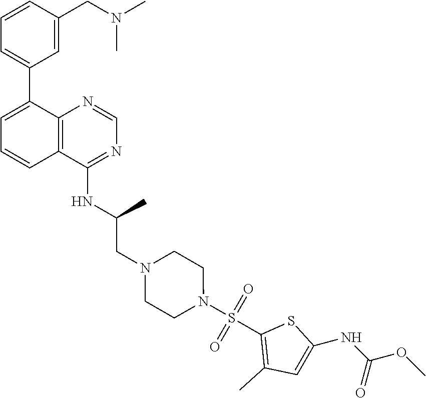 [SMILES: COC(=O)Nc1cc(C)c(S(=O)(=O)N2CCN(C[C@H](C)Nc3ncnc4c(-c5cccc(CN(C)C)c5)cccc34)CC2)s1] | methyl N-[5-({4-[(2S)-2-[(8-{3- [(dimethylamino)methyl]phenyl}quinazolin- 4-yl)amino]propyl]piperazin-1-yl}sulfonyl)- 4-methyl-1,3-thiazol-2-yl]carbamate |
| 22 | 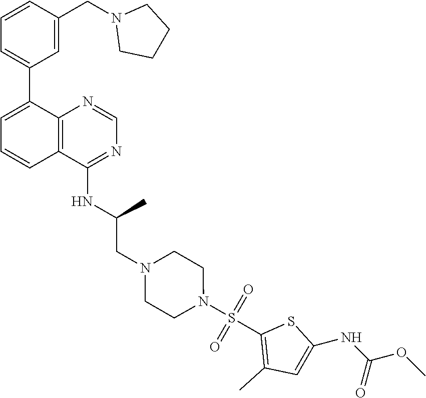 [SMILES: COC(=O)Nc1cc(C)c(S(=O)(=O)N2CCN(C[C@H](C)Nc3ncnc4c(-c5cccc(CN6CCCC6)c5)cccc34)CC2)s1] | methyl N-[4-methyl-5-({4-[(2S)-2-[(8-{3- [(pyrrolidin-1-yl)methyl]phenyl}quinazolin- 4-yl)amino]propyl]piperazin-1-yl}sulfonyl)- 1,3-thiazol-2-yl]carbamate |
| 23 | 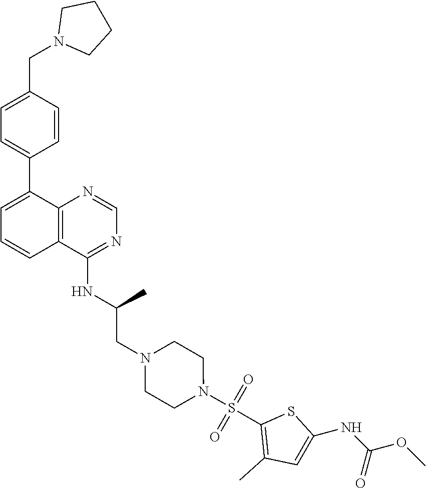 [SMILES: COC(=O)Nc1cc(C)c(S(=O)(=O)N2CCN(C[C@H](C)Nc3ncnc4c(-c5ccc(CN6CCCC6)cc5)cccc34)CC2)s1] | methyl N-[4-methyl-5-({4-[(2S)-2-[(8-{4- [(pyrrolidin-1-yl)methyl]phenyl}quinazolin- 4-yl)amino]propyl]piperazin-1-yl}sulfonyl)- 1,3-thiazol-2-yl]carbamate |
| 24 | 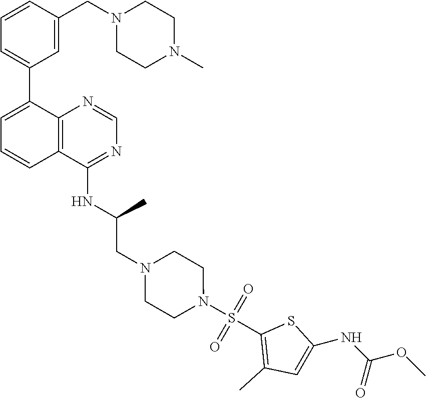 [SMILES: COC(=O)Nc1cc(C)c(S(=O)(=O)N2CCN(C[C@H](C)Nc3ncnc4c(-c5cccc(CN6CCN(C)CC6)c5)cccc34)CC2)s1] | methyl N-[4-methyl-5-({4-[(2S)-2-[(8-{3- [(4-methylpiperazin-1- yl)methyl]phenyl}quinazolin-4- yl)amino]propyl]piperazin-1-yl}sulfonyl)- 1,3-thiazol-2-yl]carbamate |
| 25 | 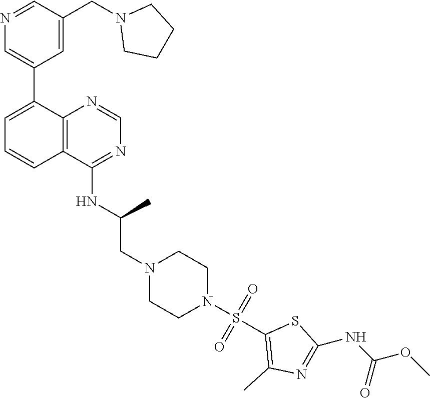 [SMILES: COC(=O)Nc1nc(C)c(S(=O)(=O)N2CCN(C[C@H](C)Nc3ncnc4c(-c5cncc(CN6CCCC6)c5)cccc34)CC2)s1] | methyl N-[4-methyl-5-({4-[(2S)-2-[(8-{5- [(pyrrolidin-1-yl)methyl]pyridin-3- yl}quinazolin-4-yl)amino]propyl]piperazin- 1-yl}sulfonyl)-1,3-thiazol-2-yl]carbamate |
| 26 | 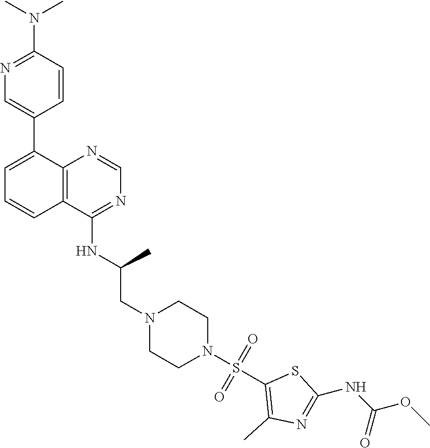 [SMILES: COC(=O)Nc1nc(C)c(S(=O)(=O)N2CCN(C[C@H](C)Nc3ncnc4c(-c5ccc(N(C)C)nc5)cccc34)CC2)s1] | methyl N-[5-({4-[(2S)-2-({8-[6- (dimethylamino)pyridin-3-yl]quinazolin-4- yl}amino)propyl]piperazin-1-yl}sulfonyl)-4- methyl-1,3-thiazol-2-yl]carbamate |
| 27 | 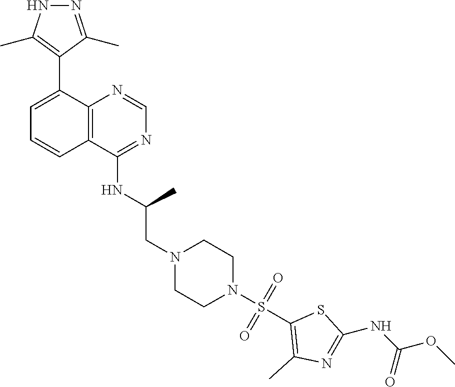 | methyl N-[5-({4-[(2S)-2-{[8-(3,5-dimethyl- 1H-pyrazol-4-yl)quinazolin-4- yl]amino}propyl]piperazin-1-yl}sulfonyl)-4- methyl-1,3-thiazol-2-yl]carbamate |
| 28 | 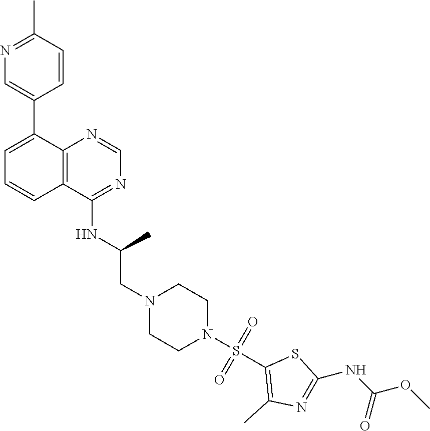 [SMILES: COC(=O)Nc1nc(C)c(S(=O)(=O)N2CCN(C[C@H](C)Nc3ncnc4c(-c5ccc(C)nc5)cccc34)CC2)s1] | methyl N-[4-methyl-5-({4-[(2S)-2-{[8-(6- methylpyridin-3-yl)quinazolin-4- yl]amino}propyl]piperazin-1-yl}sulfonyl)- 1,3-thiazol-2-yl]carbamate |
| 29 | 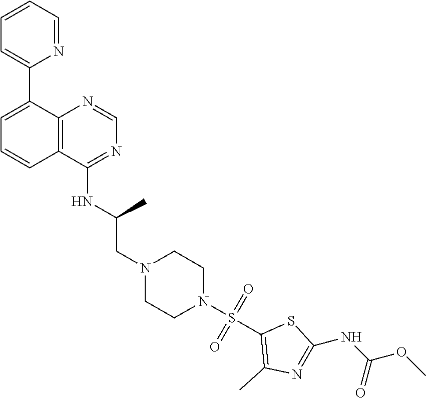 [SMILES: COC(=O)Nc1nc(C)c(S(=O)(=O)N2CCN(C[C@H](C)Nc3ncnc4c(-c5ccccn5)cccc34)CC2)s1] | methyl N-[4-methyl-5-({4-[(2S)-2-{[8- (pyridin-2-yl)quinazolin-4- yl]amino}propyl]piperazin-1-yl}sulfonyl)- 1,3-thiazol-2-yl]carbamate |
| 30 | 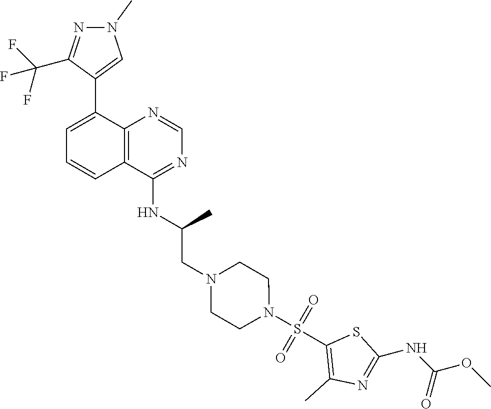 [SMILES: COC(=O)Nc1nc(C)c(S(=O)(=O)N2CCN(C[C@H](C)Nc3ncnc4c(-c5cn(C)nc5C(F)(F)F)cccc34)CC2)s1] | methyl N-[4-methyl-5-({4-[(2S)-2-({8-[1- methyl-3-(trifluoromethyl)-1H-pyrazol-4- yl]quinazolin-4-yl}amino)propyl]piperazin- 1-yl}sulfonyl)-1,3-thiazol-2-yl]carbamate |
| 31 | 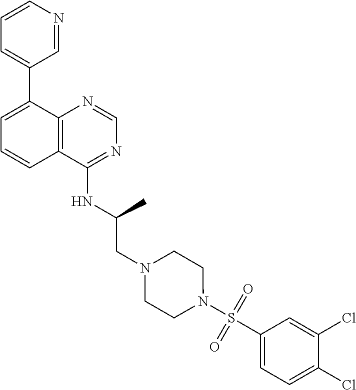 [SMILES: C[C@@H](CN1CCN(S(=O)(=O)c2ccc(Cl)c(Cl)c2)CC1)Nc1ncnc2c(-c3cccnc3)cccc12] | N-[(2S)-1-[4-(3,4- dichlorobenzenesulfonyl)piperazin-1- yl]propan-2-yl]-8-(pyridin-3-yl)quinazolin- 4-amine |
| 32 | 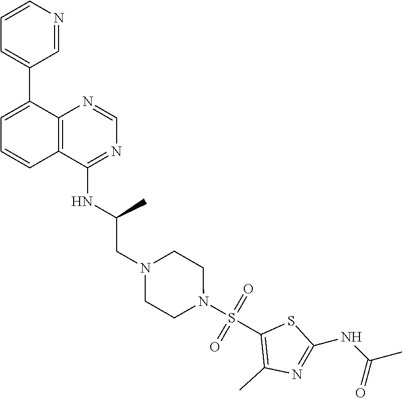 [SMILES: CC(=O)Nc1nc(C)c(S(=O)(=O)N2CCN(C[C@H](C)Nc3ncnc4c(-c5cccnc5)cccc34)CC2)s1] | N-[4-methyl-5-({4-[(2S)-2-{[8-(pyridin-3- yl)quinazolin-4-yl]amino}propyl]piperazin- 1-yl}sulfonyl)-1,3-thiazol-2-yl]acetamide |
| 33 | 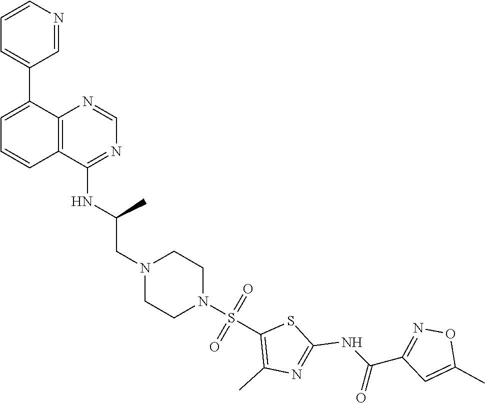 [SMILES: Cc1cc(C(=O)Nc2nc(C)c(S(=O)(=O)N3CCN(C[C@H](C)Nc4ncnc5c(-c6cccnc6)cccc45)CC3)s2)no1] | 5-methyl-N-[4-methyl-5-({4-[(2S)-2-{[8- (pyridin-3-yl)quinazolin-4- yl]amino}propyl]piperazin-1-yl}sulfonyl)- 1,3-thiazol-2-yl]-1,2-oxazole-3-carboxamide |
| 34 | 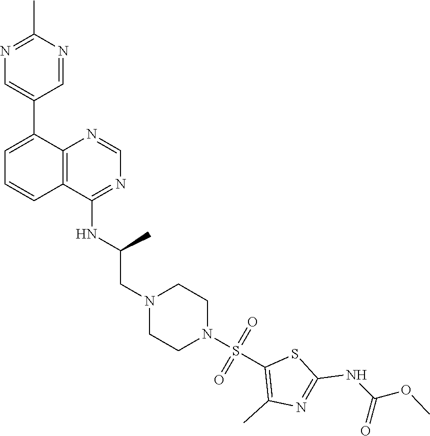 [SMILES: COC(=O)Nc1nc(C)c(S(=O)(=O)N2CCN(C[C@H](C)Nc3ncnc4c(-c5cnc(C)nc5)cccc34)CC2)s1] | methyl N-[4-methyl-5-({4-[(2S)-2-{[8-(2- methylpyrimidin-5-yl)quinazolin-4- yl]amino}propyl]piperazin-1-yl}sulfonyl)- 1,3-thiazol-2-yl]carbamate |
| 35 | 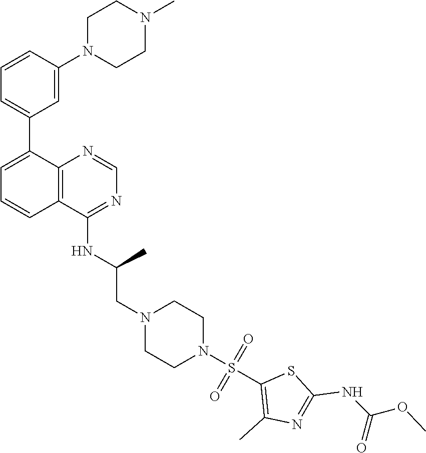 [SMILES: COC(=O)Nc1nc(C)c(S(=O)(=O)N2CCN(C[C@H](C)Nc3ncnc4c(-c5cccc(N6CCN(C)CC6)c5)cccc34)CC2)s1] | methyl N-[4-methyl-5-({4-[(2S)-2-({8-[3- (4-methylpiperazin-1-yl)phenyl]quinazolin- 4-yl}amino)propyl]piperazin-1-yl}sulfonyl)- 1,3-thiazol-2-yl]carbamate |
| 36 | 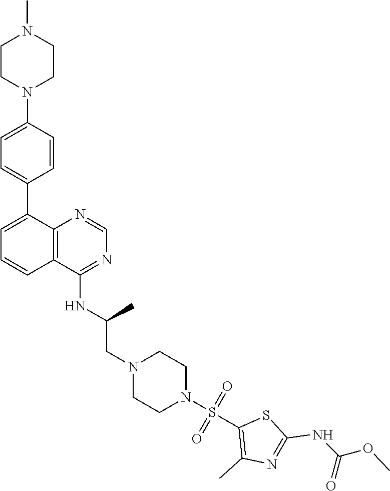 [SMILES: COC(=O)Nc1nc(C)c(S(=O)(=O)N2CCN(C[C@H](C)Nc3ncnc4c(-c5ccc(N6CCN(C)CC6)cc5)cccc34)CC2)s1] | methyl N-[4-methyl-5-({4-[(2S)-2-({8-[4- (4-methylpiperazin-1-yl)phenyl]quinazolin- 4-yl}amino)propyl]piperazin-1- yl}sulfonyl)-1,3-thiazol-2-yl]carbamate |
| 37 | 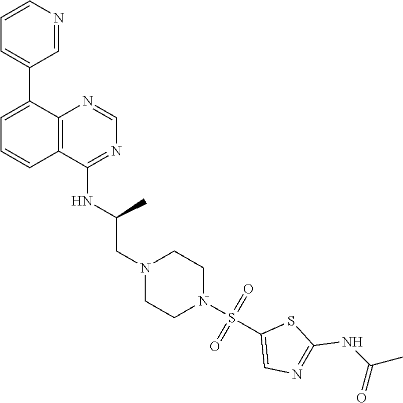 [SMILES: CC(=O)Nc1ncc(S(=O)(=O)N2CCN(C[C@H](C)Nc3ncnc4c(-c5cccnc5)cccc34)CC2)s1] | N-[5-({4-[(2S)-2-{[8-(pyridin-3- yl)quinazolin-4-yl]amino}propyl]piperazin- 1-yl}sulfonyl)-1,3-thiazol-2-yl]acetamide |
| 38 | 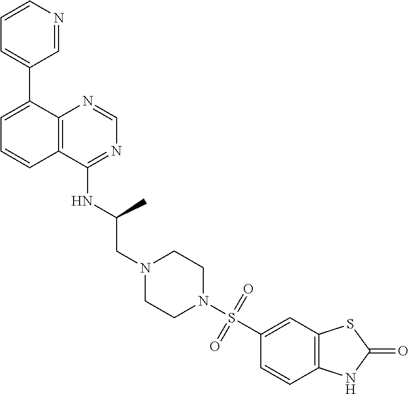 [SMILES: C[C@@H](CN1CCN(S(=O)(=O)c2ccc3[nH]c(=O)sc3c2)CC1)Nc1ncnc2c(-c3cccnc3)cccc12] | 6-({4-[(2S)-2-{[8-(pyridin-3-yl)quinazolin- 4-yl]amino}propyl]piperazin-1-yl}sulfonyl)- 2,3-dihydro-1,3-benzothiazol-2-one |
| 39 | 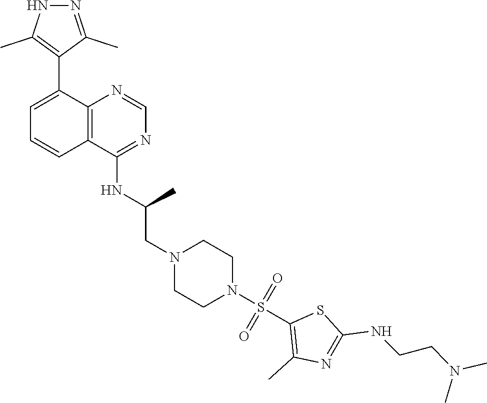 | 8-(3,5-dimethyl-1H-pyrazol-4-yl)-N-[(2S)- 1-{4-[(2-{[2-(dimethylamino)ethyl]amino}- 4-methyl-1,3-thiazol-5- yl)sulfonyl]piperazin-1-yl}propan-2- yl]quinazolin-4-amine |
| 40 | 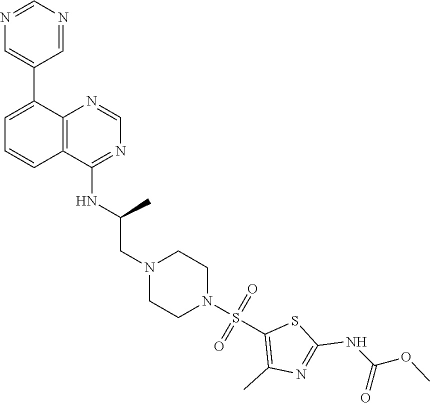 [SMILES: COC(=O)Nc1nc(C)c(S(=O)(=O)N2CCN(C[C@H](C)Nc3ncnc4c(-c5cncnc5)cccc34)CC2)s1] | methyl N-[4-methyl-5-({4-[(2S)-2-{[8- (pyrimidin-5-yl)quinazolin-4- yl]amino}propyl]piperazin-1-yl}sulfonyl)- 1,3-thiazol-2-yl]carbamate |
| 41 | 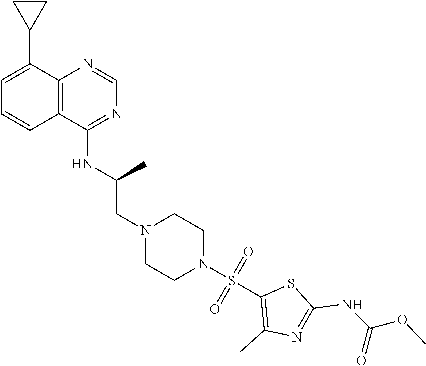 [SMILES: COC(=O)Nc1nc(C)c(S(=O)(=O)N2CCN(C[C@H](C)Nc3ncnc4c(C5CC5)cccc34)CC2)s1] | methyl N-[5-({4-[(2S)-2-[(8- cyclopropylquinazolin-4- yl)amino]propyl]piperazin-1-yl}sulfonyl)-4- methyl-1,3-thiazol-2-yl]carbamate |
| 42 | 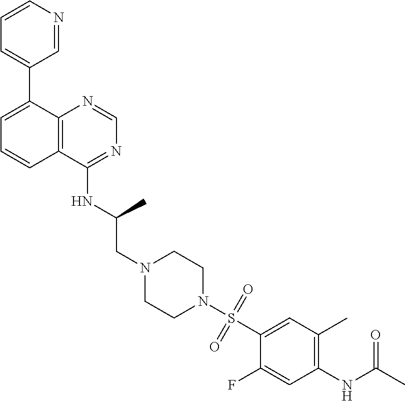 [SMILES: CC(=O)Nc1cc(F)c(S(=O)(=O)N2CCN(C[C@H](C)Nc3ncnc4c(-c5cccnc5)cccc34)CC2)cc1C] | N-[5-fluoro-2-methyl-4-({4-[(2S)-2-{[8- (pyridin-3-yl)quinazolin-4- yl]amino}propyl]piperazin-1- yl}sulfonyl)phenyl]acetamide |
| 43 | 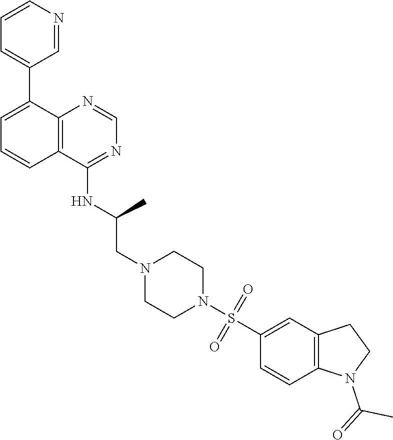 [SMILES: CC(=O)N1CCc2cc(S(=O)(=O)N3CCN(C[C@H](C)Nc4ncnc5c(-c6cccnc6)cccc45)CC3)ccc21] | 1-[5-({4-[(2S)-2-{[8-(pyridin-3- yl)quinazolin-4-yl]amino}propyl]piperazin- 1-yl}sulfonyl)-2,3-dihydro-1H-indol-1- yl]ethan-1-one |
| 44 | 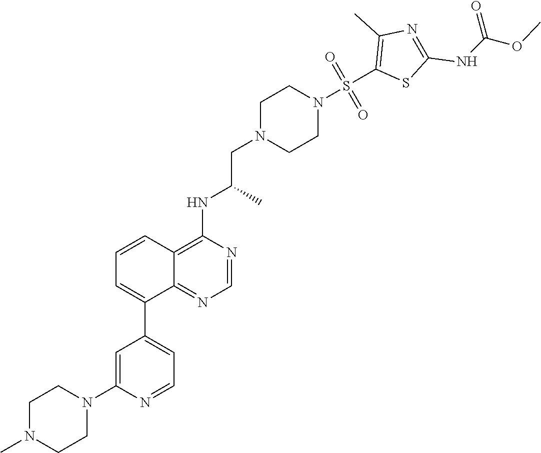 | methyl N-[4-methyl-5-({4-[(2S)-2-({8-[2- (4-methylpiperazin-1-yl)pyridin-4- yl]quinazolin-4-yl}amino)propyl]piperazin- 1-yl}sulfonyl)-1,3-thiazol-2-yl]carbamate |
| 45 | 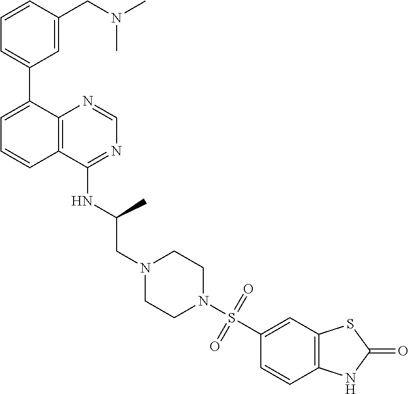 [SMILES: C[C@@H](CN1CCN(S(=O)(=O)c2ccc3[nH]c(=O)sc3c2)CC1)Nc1ncnc2c(-c3cccc(CN(C)C)c3)cccc12] | 6-({4-[(2S)-2-[(8-{3- [(dimethylamino)methyl]phenyl}quinazolin- 4-yl)amino]propyl]piperazin-1-yl}sulfonyl)- 2,3-dihydro-1,3-benzothiazol-2-one |
| 46 | 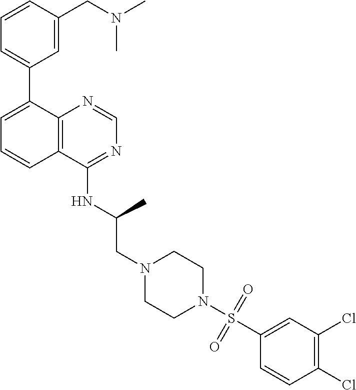 [SMILES: C[C@@H](CN1CCN(S(=O)(=O)c2ccc(Cl)c(Cl)c2)CC1)Nc1ncnc2c(-c3cccc(CN(C)C)c3)cccc12] | N-[(2S)-1-[4-(3,4- dichlorobenzenesulfonyl)piperazin-1- yl]propan-2-yl]-8-{3- [(dimethylamino)methyl]phenyl}quinazolin- 4-amine |
| 47 | 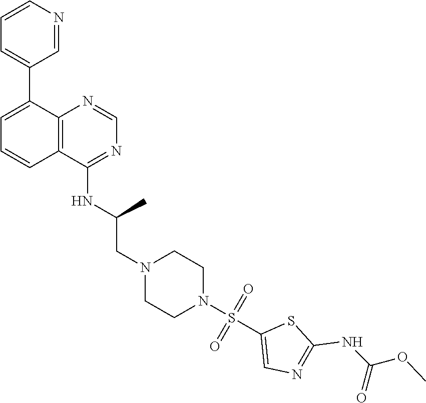 [SMILES: COC(=O)Nc1ncc(S(=O)(=O)N2CCN(C[C@H](C)Nc3ncnc4c(-c5cccnc5)cccc34)CC2)s1] | methyl N-[5-({4-[(2S)-2-{[8-(pyridin-3- yl)quinazolin-4-yl]amino}propyl]piperazin- 1-yl}sulfonyl)-1,3-thiazol-2-yl]carbamate |
| 48 | 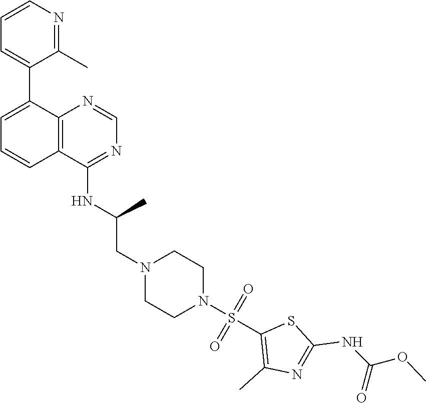 [SMILES: COC(=O)Nc1nc(C)c(S(=O)(=O)N2CCN(C[C@H](C)Nc3ncnc4c(-c5cccnc5C)cccc34)CC2)s1] | methyl N-[4-methyl-5-({4-[(2S)-2-{[8-(2- methylpyridin-3-yl)quinazolin-4- yl]amino}propyl]piperazin-1-yl}sulfonyl)- 1,3-thiazol-2-yl]carbamate |
| 49 | 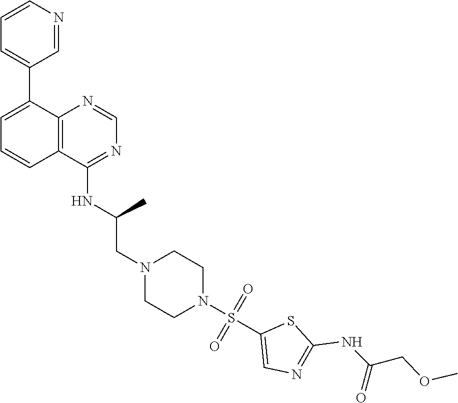 [SMILES: COCC(=O)Nc1ncc(S(=O)(=O)N2CCN(C[C@H](C)Nc3ncnc4c(-c5cccnc5)cccc34)CC2)s1] | 2-methoxy-N-[5-({4-[(2S)-2-{[8-(pyridin-3- yl)quinazolin-4-yl]amino}propyl]piperazin- 1-yl}sulfonyl)-1,3-thiazol-2-yl]acetamide |
| 50 | 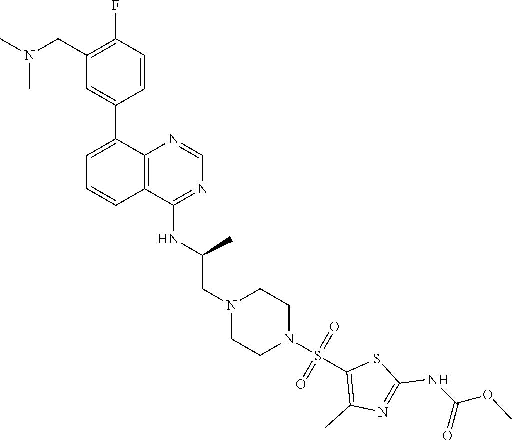 | methyl N-[5-({4-[(2S)-2-[(8-{3- [(dimethylamino)methyl]-4- fluorophenyl}quinazolin-4- yl)amino]propyl]piperazin-1-yl}sulfonyl)-4- methyl-1,3-thiazol-2-yl]carbamate |
| 51 | 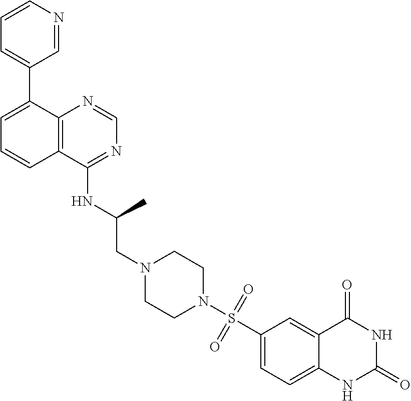 [SMILES: C[C@@H](CN1CCN(S(=O)(=O)c2ccc3[nH]c(=O)[nH]c(=O)c3c2)CC1)Nc1ncnc2c(-c3cccnc3)cccc12] | 6-({4-[(2S)-2-{[8-(pyridin-3-yl)quinazolin- 4-yl]amino}propyl]piperazin-1-yl}sulfonyl)- 1,2,3,4-tetrahydroquinazoline-2,4-dione |
| 52 | 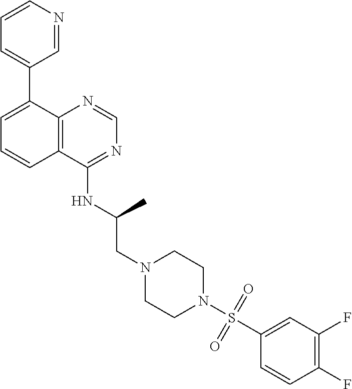 [SMILES: C[C@@H](CN1CCN(S(=O)(=O)c2ccc(F)c(F)c2)CC1)Nc1ncnc2c(-c3cccnc3)cccc12] | N-[(2S)-1-[4-(3,4- difluorobenzenesulfonyl)piperazin-1- yl]propan-2-yl]-8-(pyridin-3-yl)quinazolin- 4-amine |
| 53 | 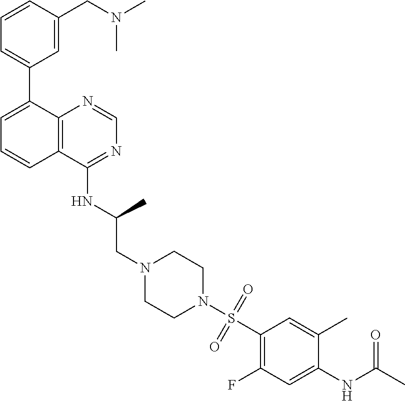 [SMILES: CC(=O)Nc1cc(F)c(S(=O)(=O)N2CCN(C[C@H](C)Nc3ncnc4c(-c5cccc(CN(C)C)c5)cccc34)CC2)cc1C] | N-[4-({4-[(2S)-2-[(8-{3- [(dimethylamino)methyl]phenyl}quinazolin- 4-yl)amino]propyl]piperazin-1-yl}sulfonyl)- 5-fluoro-2-methylphenyl]acetamide |
| 54 | 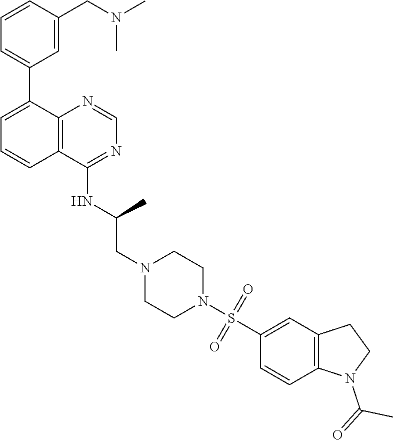 [SMILES: CC(=O)N1CCc2cc(S(=O)(=O)N3CCN(C[C@H](C)Nc4ncnc5c(-c6cccc(CN(C)C)c6)cccc45)CC3)ccc21] | 1-[5-({4-[(2S)-2-[(8-{3- [(dimethylamino)methyl]phenyl}quinazolin- 4-yl)amino]propyl]piperazin-1-yl}sulfonyl)- 2,3-dihydro-1H-indol-1-yl]ethan-1-one |
| 55 | 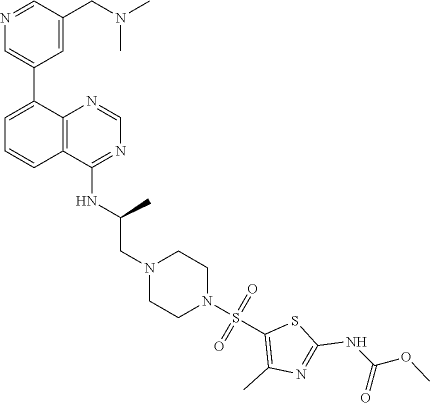 [SMILES: COC(=O)Nc1nc(C)c(S(=O)(=O)N2CCN(C[C@H](C)Nc3ncnc4c(-c5cncc(CN(C)C)c5)cccc34)CC2)s1] | methyl N-[5-({4-[(2S)-2-[(8-{5- [(dimethylamino)methyl]pyridin-3- yl}quinazolin-4-yl)amino]propyl]piperazin- 1-yl}sulfonyl)-4-methyl-1,3-thiazol-2- yl]carbamate |
| 56 | 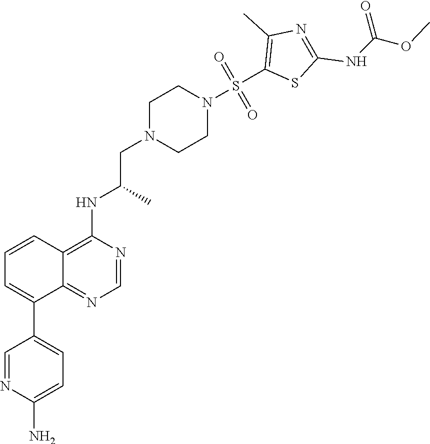 [SMILES: COC(=O)Nc1nc(C)c(S(=O)(=O)N2CCN(C[C@H](C)Nc3ncnc4c(-c5ccc(N)nc5)cccc34)CC2)s1] | methyl N-[5-({4-[(2S)-2-{[8-(6- aminopyridin-3-yl)quinazolin-4- yl]amino}propyl]piperazin-1-yl}sulfonyl)-4- methyl-1,3-thiazol-2-yl]carbamate |
| 57 | 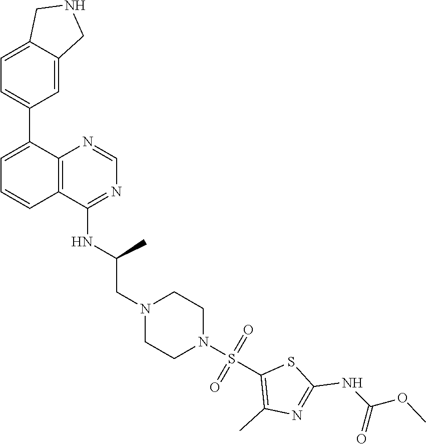 [SMILES: COC(=O)Nc1nc(C)c(S(=O)(=O)N2CCN(C[C@H](C)Nc3ncnc4c(-c5ccc6c(c5)CNC6)cccc34)CC2)s1] | methyl N-[5-({4-[(2S)-2-{[8-(2,3-dihydro- 1H-isoindol-5-yl)quinazolin-4- yl]amino}propyl]piperazin-1-yl}sulfonyl)-4- methyl-1,3-thiazol-2-yl]carbamate |
| 58 | 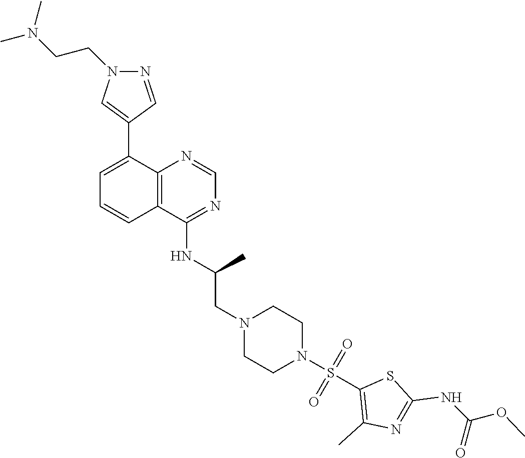 | methyl N-[5-({4-[(2S)-2-[(8-{1-[2- (dimethylamino)ethyl]-1H-pyrazol-4- yl}quinazolin-4-yl)amino]propyl]piperazin- 1-yl}sulfonyl)-4-methyl-1,3-thiazol-2- yl]carbamate |
| 59 | 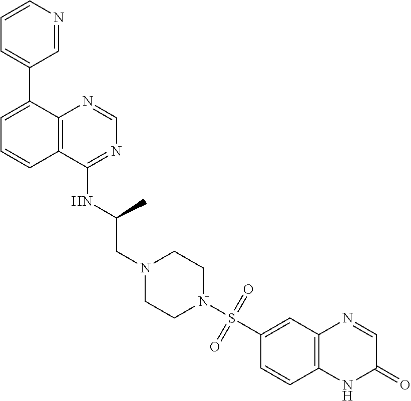 [SMILES: C[C@@H](CN1CCN(S(=O)(=O)c2ccc3[nH]c(=O)cnc3c2)CC1)Nc1ncnc2c(-c3cccnc3)cccc12] | 6-({4-[(2S)-2-{[8-(pyridin-3-yl)quinazolin- 4-yl]amino}propyl]piperazin-1-yl}sulfonyl)- 1,2-dihydroquinoxalin-2-one |
| 60 | 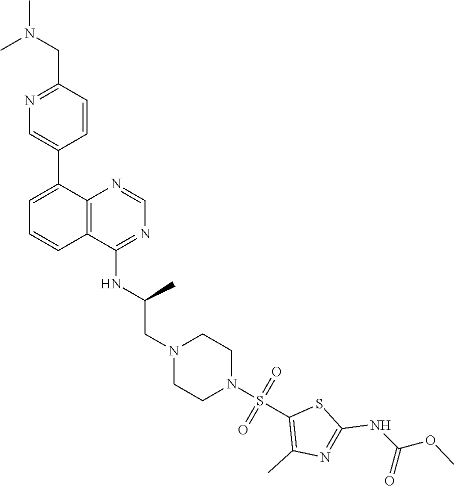 | methyl N-[5-({4-[(2S)-2-[(8-{6- [(dimethylamino)methyl]pyridin-3- yl}quinazolin-4-yl)amino]propyl]piperazin- 1-yl}sulfonyl)-4-methyl-1,3-thiazol-2- yl]carbamate |
| 61 | 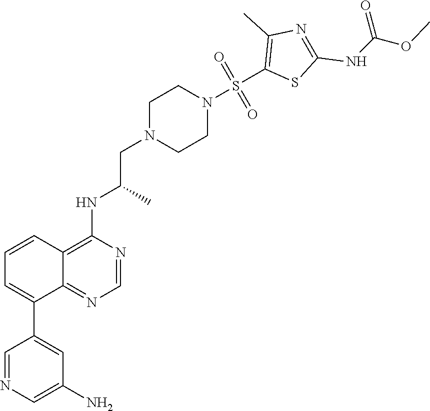 [SMILES: COC(=O)Nc1nc(C)c(S(=O)(=O)N2CCN(C[C@H](C)Nc3ncnc4c(-c5cncc(N)c5)cccc34)CC2)s1] | methyl N-[5-({4-[(2S)-2-{[8-(5- aminopyridin-3-yl)quinazolin-4- yl]amino}propyl]piperazin-1-yl}sulfonyl)-4- methyl-1,3-thiazol-2-yl]carbamate |
| 63 | 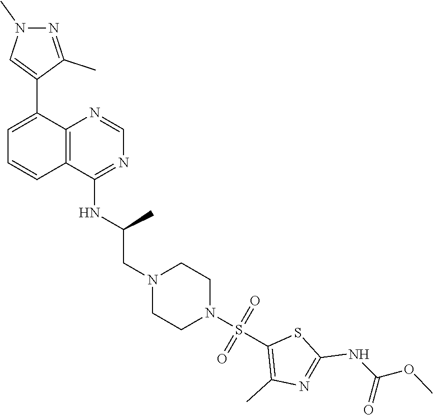 | methyl N-[5-({4-[(2S)-2-{[8-(1,3-dimethyl- 1H-pyrazol-4-yl)quinazolin-4- yl]amino}propyl]piperazin-1-yl}sulfonyl)-4- methyl-1,3-thiazol-2-yl]carbamate |
| 64 | 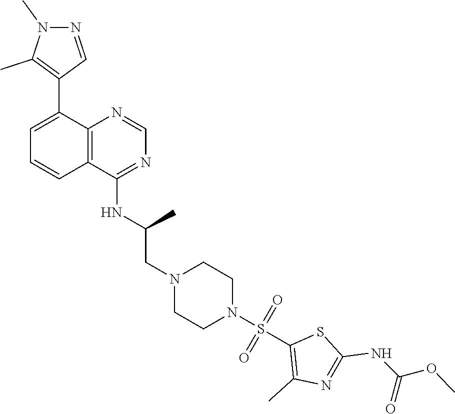 | methyl N-[5-({4-[(2S)-2-{[8-(1,5-dimethyl- 1H-pyrazol-4-yl)quinazolin-4- yl]amino}propyl]piperazin-1-yl}sulfonyl)-4- methyl-1,3-thiazol-2-yl]carbamate |
| 65 | 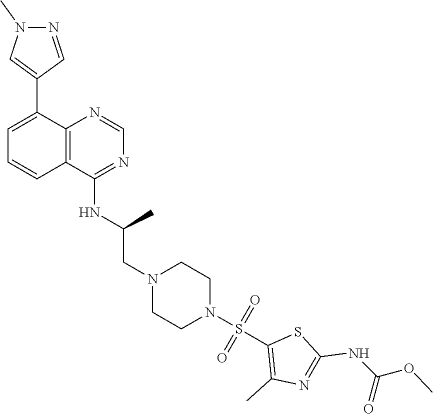 | methyl N-[4-methyl-5-({4-[(2S)-2-{[8-(1- methyl-1H-pyrazol-4-yl)quinazolin-4- yl]amino}propyl]piperazin-1-yl}sulfonyl)- 1,3-thiazol-2-yl]carbamate |
| 66 | 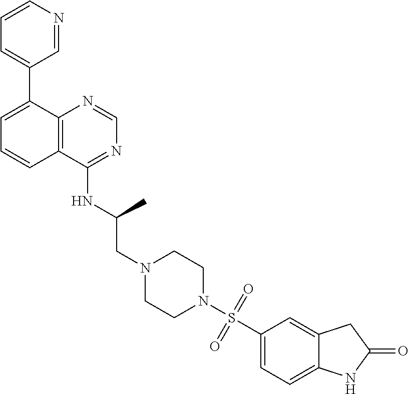 [SMILES: C[C@@H](CN1CCN(S(=O)(=O)c2ccc3c(c2)CC(=O)N3)CC1)Nc1ncnc2c(-c3cccnc3)cccc12] | 5-({4-[(2S)-2-{[8-(pyridin-3-yl)quinazolin- 4-yl]amino}propyl]piperazin-1-yl}sulfonyl)- 2,3-dihydro-1H-indol-2-one |
| 67 | 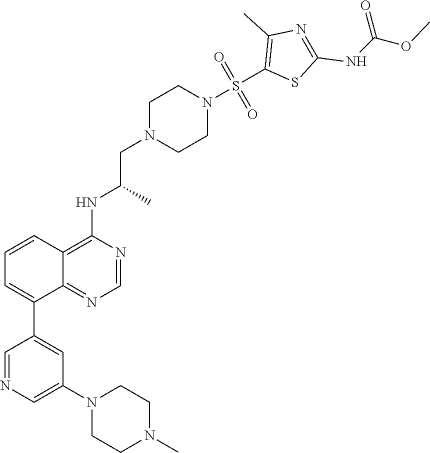 [SMILES: COC(=O)Nc1nc(C)c(S(=O)(=O)N2CCN(C[C@H](C)Nc3ncnc4c(-c5cncc(N6CCN(C)CC6)c5)cccc34)CC2)s1] | methyl N-[4-methyl-5-({4-[(2S)-2-({8-[5- (4-methylpiperazin-1-yl)pyridin-3- yl]quinazolin-4-yl}amino)propyl]piperazin- 1-yl}sulfonyl)-1,3-thiazol-2-yl]carbamate |
| 68 | 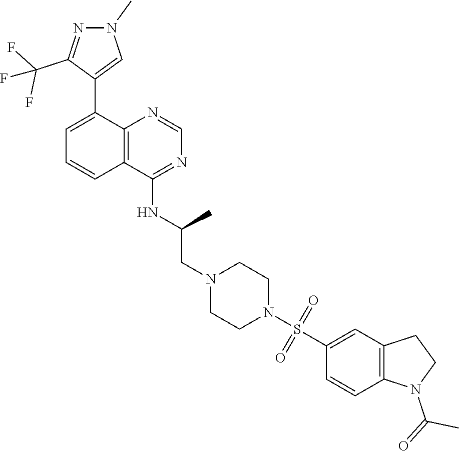 [SMILES: CC(=O)N1CCc2cc(S(=O)(=O)N3CCN(C[C@H](C)Nc4ncnc5c(-c6cn(C)nc6C(F)(F)F)cccc45)CC3)ccc21] | 1-[5-({4-[(2S)-2-({8-[1-methyl-3- (trifluoromethyl)-1H-pyrazol-4- yl]quinazolin-4-yl}amino)propyl]piperazin- 1-yl}sulfonyl)-2,3-dihydro-1H-indol-1- yl]ethan-1-one |
| 69 | 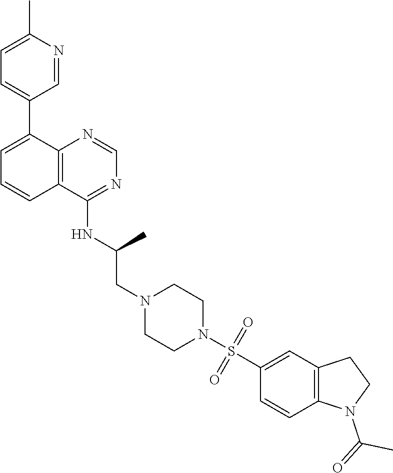 [SMILES: CC(=O)N1CCc2cc(S(=O)(=O)N3CCN(C[C@H](C)Nc4ncnc5c(-c6ccc(C)nc6)cccc45)CC3)ccc21] | 1-[5-({4-[(2S)-2-{[8-(6-methylpyridin-3- yl)quinazolin-4-yl]amino}propyl]piperazin- 1-yl}sulfonyl)-2,3-dihydro-1H-indol-1- yl}ethan-1-one |
| 71 | 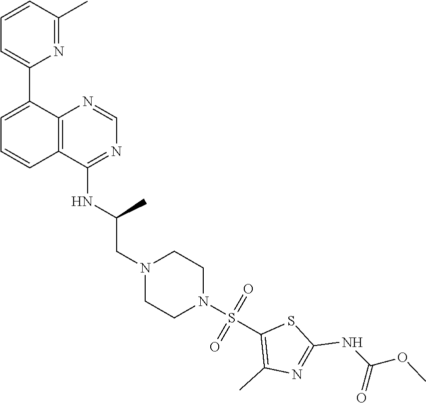 [SMILES: COC(=O)Nc1nc(C)c(S(=O)(=O)N2CCN(C[C@H](C)Nc3ncnc4c(-c5cccc(C)n5)cccc34)CC2)s1] | methyl N-[4-methyl-5-({4-[(2S)-2-{[8-(6- methylpyridin-2-yl)quinazolin-4- yl]amino}propyl]piperazin-1-yl}sulfonyl)- 1,3-thiazol-2-yl]carbamate |
| 72 | 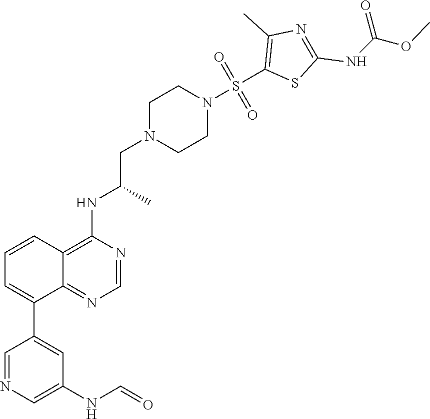 [SMILES: COC(=O)Nc1nc(C)c(S(=O)(=O)N2CCN(C[C@H](C)Nc3ncnc4c(-c5cncc(NC=O)c5)cccc34)CC2)s1] | methyl N-[5-({4-[(2S)-2-{[8-(5- formamidopyridin-3-yl)quinazolin-4- yl]amino}propyl]piperazin-1-yl}sulfonyl)-4- methyl-1,3-thiazol-2-yl]carbamate |
| 73 | 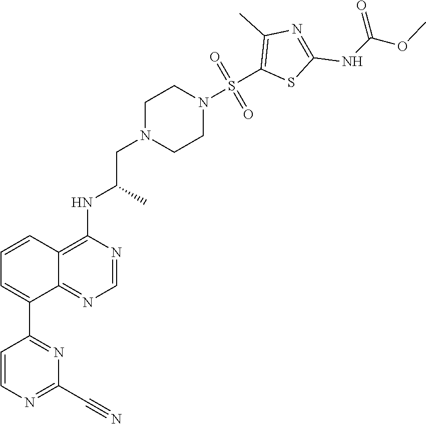 [SMILES: COC(=O)Nc1nc(C)c(S(=O)(=O)N2CCN(C[C@H](C)Nc3ncnc4c(-c5ccnc(C#N)n5)cccc34)CC2)s1] | methyl N-[5-({4-[(2S)-2-{[8-(2- cyanopyrimidin-4-yl)quinazolin-4- yl]amino}propyl]piperazin-1-yl}sulfonyl)- 4-methyl-1,3-thiazol-2-yl]carbamate |
| 74 | 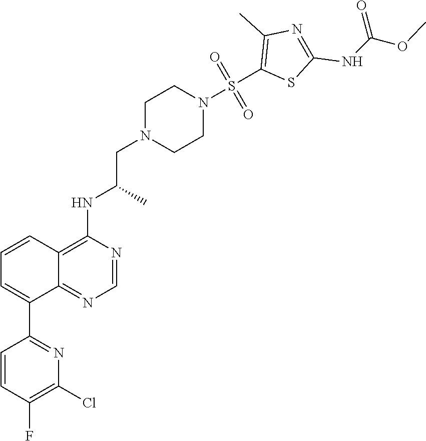 [SMILES: COC(=O)Nc1nc(C)c(S(=O)(=O)N2CCN(C[C@H](C)Nc3ncnc4c(-c5ccc(F)c(Cl)n5)cccc34)CC2)s1] | methyl N-[5-({4-[(2S)-2-{[8-(6-chloro-5- fluoropyridin-2-yl)quinazolin-4- yl]amino}propyl]piperazin-1-yl}sulfonyl)-4- methyl-1,3-thiazol-2-yl]carbamate |
| 75 | 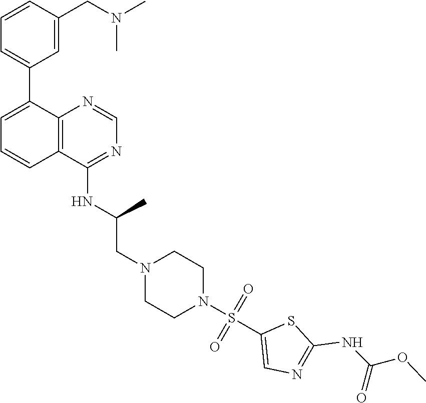 [SMILES: COC(=O)Nc1ncc(S(=O)(=O)N2CCN(C[C@H](C)Nc3ncnc4c(-c5cccc(CN(C)C)c5)cccc34)CC2)s1] | methyl N-[5-({4-[(2S)-2-[(8-{3- [(dimethylamino)methyl]phenyl}quinazolin- 4-yl)amino]propyl]piperazin-1-yl}sulfonyl)- 1,3-thiazol-2-yl]carbamate |
| 76 | 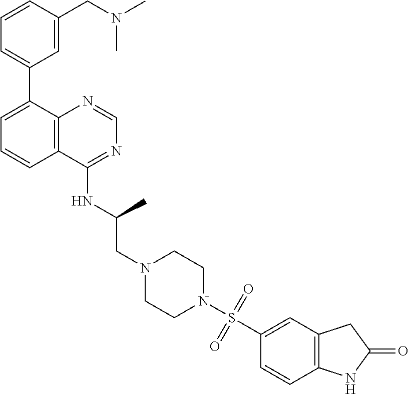 [SMILES: C[C@@H](CN1CCN(S(=O)(=O)c2ccc3c(c2)CC(=O)N3)CC1)Nc1ncnc2c(-c3cccc(CN(C)C)c3)cccc12] | 5-({4-[(2S)-2-[(8-{3- [(dimethylamino)methyl]phenyl}quinazolin- 4-yl)amino]propyl]piperazin-1-yl}sulfonyl)- 2,3-dihydro-1H-indol-2-one |
| 77 | 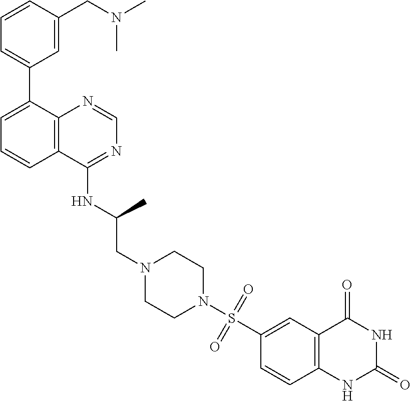 [SMILES: C[C@@H](CN1CCN(S(=O)(=O)c2ccc3[nH]c(=O)[nH]c(=O)c3c2)CC1)Nc1ncnc2c(-c3cccc(CN(C)C)c3)cccc12] | 6-({4-[(2S)-2-[(8-{3- [(dimethylamino)methyl]phenyl}quinazolin- 4-yl)amino]propyl]piperazin-1-yl}sulfonyl)- 1,2,3,4-tetrahydroquinazoline-2,4-dione |
| 78 | 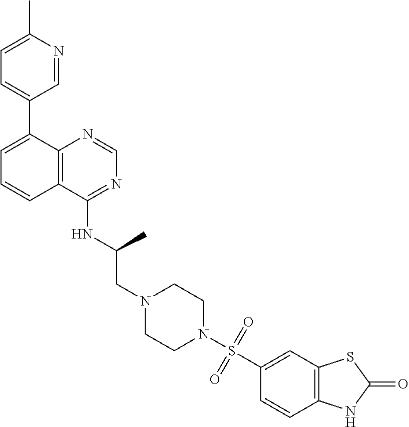 [SMILES: Cc1ccc(-c2cccc3c(N[C@@H](C)CN4CCN(S(=O)(=O)c5ccc6[nH]c(=O)sc6c5)CC4)ncnc23)cn1] | 6-({4-[(2S)-2-{[8-(6-methylpyridin-3- yl)quinazolin-4-yl]amino}propyl]piperazin- 1-yl}sulfonyl)-2,3-dihydro-1,3- benzothiazol-2-one |
| 79 | 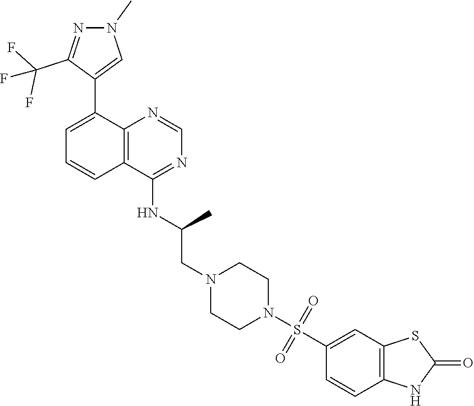 [SMILES: C[C@@H](CN1CCN(S(=O)(=O)c2ccc3[nH]c(=O)sc3c2)CC1)Nc1ncnc2c(-c3cn(C)nc3C(F)(F)F)cccc12] | 6-({4-[(2S)-2-({8-[1-methyl-3- (trifluoromethyl)-1H-pyrazol-4- yl]quinazolin-4-yl}amino)propyl]piperazin- 1-yl}sulfonyl)-2,3-dihydro-1,3- benzothiazol-2-one |
| 80 | 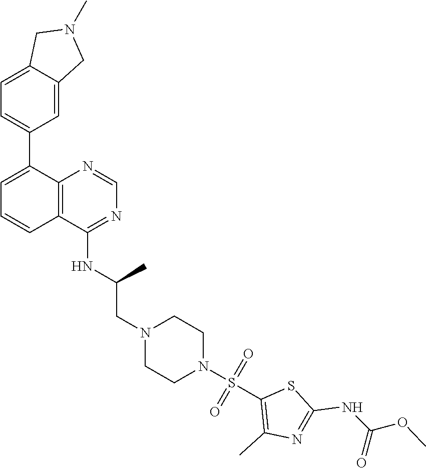 [SMILES: COC(=O)Nc1nc(C)c(S(=O)(=O)N2CCN(C[C@H](C)Nc3ncnc4c(-c5ccc6c(c5)CN(C)C6)cccc34)CC2)s1] | methyl N-[4-methyl-5-({4-[(2S)-2-{[8-(2- methyl-2,3-dihydro-1H-isoindol-5- yl)quinazolin-4-yl]amino}propyl]piperazin- 1-yl}sulfonyl)-1,3-thiazol-2-yl]carbamate |
| 81 | 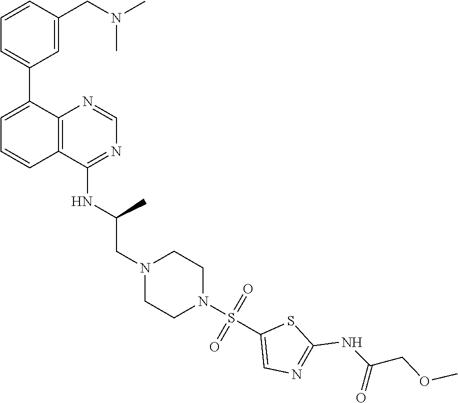 [SMILES: COCC(=O)Nc1ncc(S(=O)(=O)N2CCN(C[C@H](C)Nc3ncnc4c(-c5cccc(CN(C)C)c5)cccc34)CC2)s1] | N-[5-({4-[(2S)-2-[(8-{3- [(dimethylamino)methyl]phenyl}quinazolin- 4-yl)amino]propyl]piperazin-1-yl}sulfonyl)- 1,3-thiazol-2-yl]-2-methoxyacetamide |
| 82 | 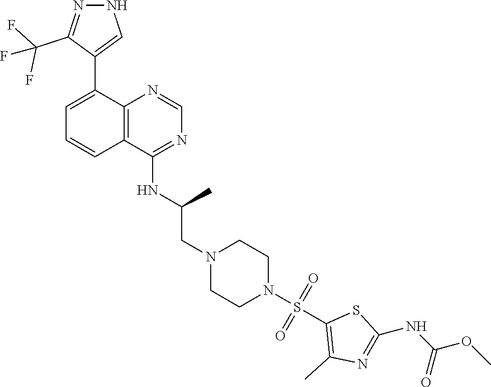 [SMILES: COC(=O)Nc1nc(C)c(S(=O)(=O)N2CCN(C[C@H](C)Nc3ncnc4c(-c5c[nH]nc5C(F)(F)F)cccc34)CC2)s1] | methyl N-[4-methyl-5-({4-[(2S)-2-({8-[3- (trifluoromethyl)-1H-pyrazol-4- yl]quinazolin-4-yl}amino)propyl]piperazin- 1-yl}sulfonyl)-1,3-thiazol-2-yl]carbamate |
| 83 | 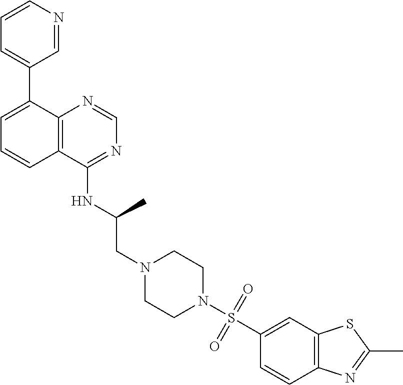 [SMILES: Cc1nc2ccc(S(=O)(=O)N3CCN(C[C@H](C)Nc4ncnc5c(-c6cccnc6)cccc45)CC3)cc2s1] | N-[(2S)-1-{4-[(2-methyl-1,3-benzothiazol- 6-yl)sulfonyl]piperazin-1-yl}propan-2-yl]- 8-(pyridin-3-yl)quinazolin-4-amine |
| 84 | 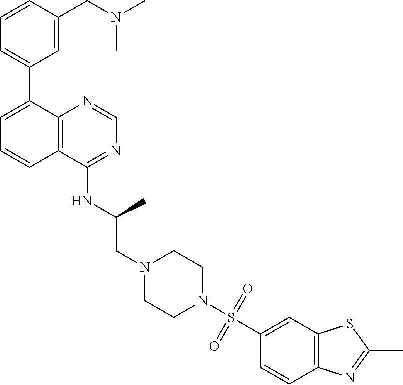 [SMILES: Cc1nc2ccc(S(=O)(=O)N3CCN(C[C@H](C)Nc4ncnc5c(-c6cccc(CN(C)C)c6)cccc45)CC3)cc2s1] | 8-{3-[(dimethylamino)methyl]phenyl}-N- [(2S)-1-{4-[(2-methyl-1,3-benzothiazol-6- yl)sulfonyl]piperazin-1-yl}propan-2- yl]quinazolin-4-amine |
| 85 | 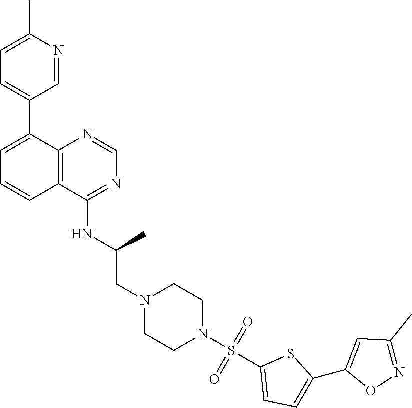 [SMILES: Cc1ccc(-c2cccc3c(N[C@@H](C)CN4CCN(S(=O)(=O)c5ccc(-c6cc(C)no6)s5)CC4)ncnc23)cn1] | N-[(2S)-1-(4-{[5-(3-methyl-1,2-oxazol-5- yl)thiophen-2-yl]sulfonyl}piperazin-1- yl)propan-2-yl]-8-(6-methylpyridin-3- yl)quinazolin-4-amine |
| 86 | 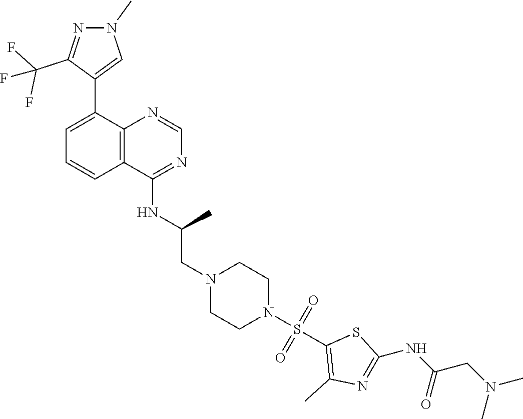 [SMILES: Cc1nc(NC(=O)CN(C)C)sc1S(=O)(=O)N1CCN(C[C@H](C)Nc2ncnc3c(-c4cn(C)nc4C(F)(F)F)cccc23)CC1] | 2-(dimethylamino)-N-[4-methyl-5-({4- [(2S)-2-({8-[1-methyl-3-(trifluoromethyl)- 1H-pyrazol-4-yl]quinazolin-4- yl}amino)propyl]piperazin-1-yl}sulfonyl)- 1,3-thiazol-2-yl]acetamide |
| 87 | 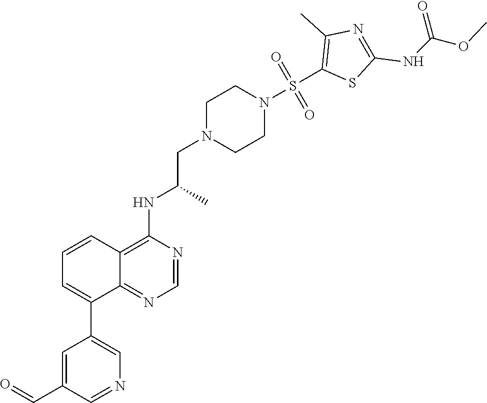 [SMILES: COC(=O)Nc1nc(C)c(S(=O)(=O)N2CCN(C[C@H](C)Nc3ncnc4c(-c5cncc(C=O)c5)cccc34)CC2)s1] | methyl N-[5-({4-[(2S)-2-{[8-(5- formylpyridin-3-yl)quinazolin-4- yl]amino}propyl]piperazin-1-yl}sulfonyl)-4- methyl-1,3-thiazol-2-yl]carbamate |
| 88 | 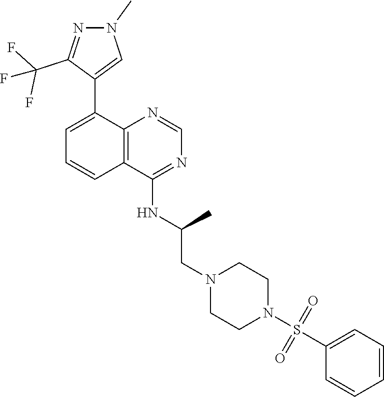 [SMILES: C[C@@H](CN1CCN(S(=O)(=O)c2ccccc2)CC1)Nc1ncnc2c(-c3cn(C)nc3C(F)(F)F)cccc12] | N-[(2S)-1-[4-(benzenesulfonyl)piperazin-1- yl]propan-2-yl]-8-[1-methyl-3- (trifluoromethyl)-1H-pyrazol-4- yl]quinazolin-4-amine |
| 89 | 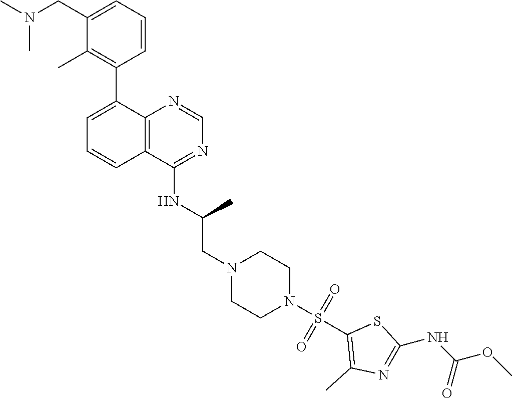 | methyl N-[5-({4-[(2S)-2-[(8-{3- [(dimethylamino)methyl]-2- methylphenyl}quinazolin-4- yl)amino]propyl]piperazin-1-yl}sulfonyl)-4- methyl-1,3-thiazol-2-yl]carbamate |
| 90 | 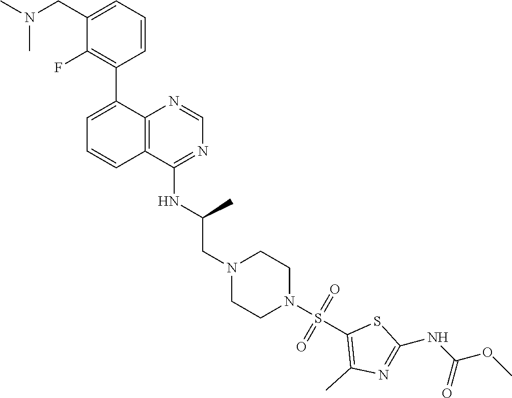 | methyl N-[5-({4-[(2S)-2-[(8-{3- [(dimethylamino)methyl]-2- fluorophenyl}quinazolin-4- yl)amino]propyl]piperazin-1-yl}sulfonyl)-4- methyl-1,3-thiazol-2-yl]carbamate |
| 91 | 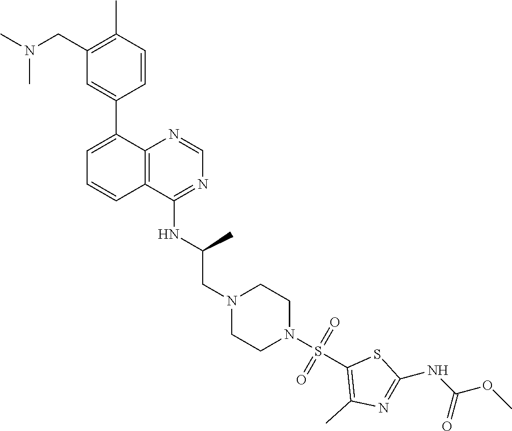 | methyl N-[5-({4-[(2S)-2-[(8-{3- [(dimethylamino)methyl]-4- methylphenyl}quinazolin-4- yl)amino]propyl]piperazin-1-yl}sulfonyl)-4- methyl-1,3-thiazol-2-yl]carbamate |
| 92 | 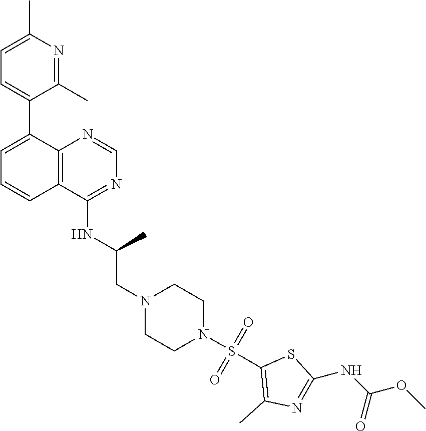 [SMILES: COC(=O)Nc1nc(C)c(S(=O)(=O)N2CCN(C[C@H](C)Nc3ncnc4c(-c5ccc(C)nc5C)cccc34)CC2)s1] | methyl N-[5-({4-[(2S)-2-{[8-(2,6- dimethylpyridin-3-yl)quinazolin-4- yl]amino}propyl]piperazin-1-yl}sulfonyl)- 4-methyl-1,3-thiazol-2-yl]carbamate |
| 93 | 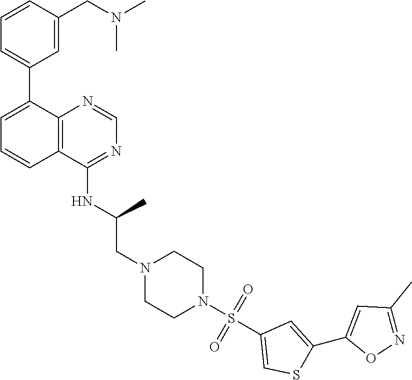 [SMILES: Cc1cc(-c2cc(S(=O)(=O)N3CCN(C[C@H](C)Nc4ncnc5c(-c6cccc(CN(C)C)c6)cccc45)CC3)cs2)on1] | 8-{3-[(dimethylamino)methyl]phenyl}-N- [(2S)-1-(4-{[5-(3-methyl-1,2-oxazol-5- yl)thiophen-3-yl]sulfonyl}piperazin-1- yl)propan-2-yl]quinazolin-4-amine |
| 94 | 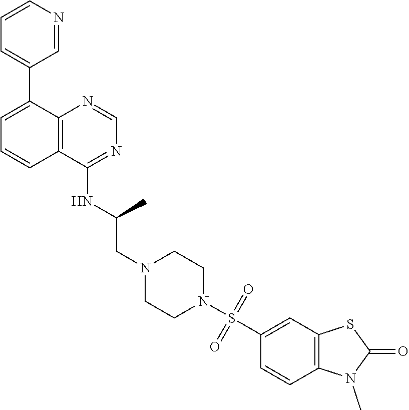 [SMILES: C[C@@H](CN1CCN(S(=O)(=O)c2ccc3c(c2)sc(=O)n3C)CC1)Nc1ncnc2c(-c3cccnc3)cccc12] | 3-methyl-6-({4-[(2S)-2-{[8-(pyridin-3- yl)quinazolin-4-yl]amino}propyl]piperazin- 1-yl}sulfonyl)-2,3-dihydro-1,3- benzothiazol-2-one |
| 95 | 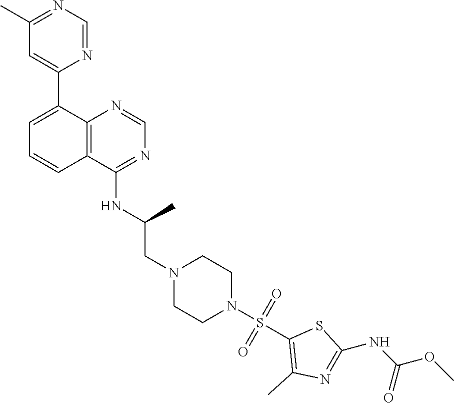 | methyl N-[4-methyl-5-({4-[(2S)-2-{[8-(6- methylpyrimidin-4-yl)quinazolin-4- yl]amino}propyl]piperazin-1-yl}sulfonyl)- 1,3-thiazol-2-yl]carbamate |
| 96 | 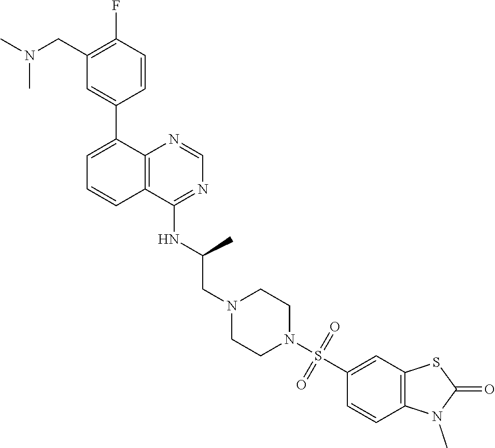 [SMILES: C[C@@H](CN1CCN(S(=O)(=O)c2ccc3c(c2)sc(=O)n3C)CC1)Nc1ncnc2c(-c3ccc(F)c(CN(C)C)c3)cccc12] | 6-({4-[(2S)-2-[(8-{3- [(dimethylamino)methyl]-4- fluorophenyl}quinazolin-4- yl)amino]propyl]piperazin-1-yl}sulfonyl)-3- methyl-2,3-dihydro-1,3-benzothiazol-2-one |
| 97 | 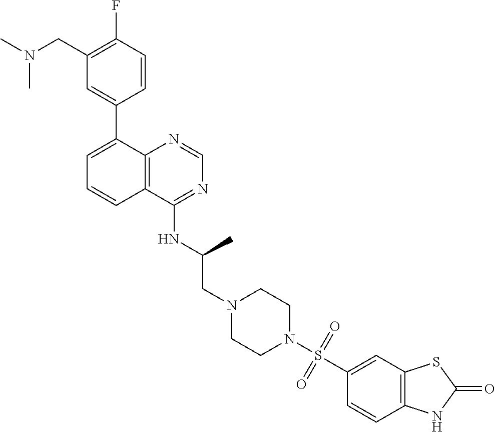 [SMILES: C[C@@H](CN1CCN(S(=O)(=O)c2ccc3[nH]c(=O)sc3c2)CC1)Nc1ncnc2c(-c3ccc(F)c(CN(C)C)c3)cccc12] | 6-({4-[(2S)-2-[(8-{3- [(dimethylamino)methyl]-4- fluorophenyl}quinazolin-4- yl)amino]propyl]piperazin-1-yl}sulfonyl)- 2,3-dihydro-1,3-benzothiazol-2-one |
| 98 | 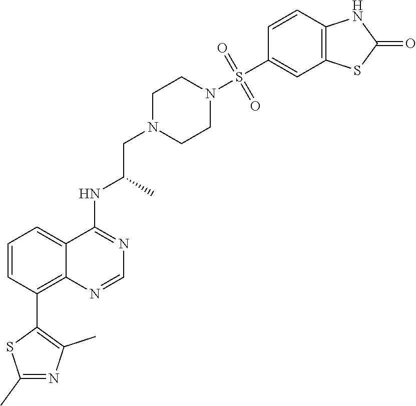 [SMILES: Cc1nc(C)c(-c2cccc3c(N[C@@H](C)CN4CCN(S(=O)(=O)c5ccc6[nH]c(=O)sc6c5)CC4)ncnc23)s1] | 6-({4-[(2S)-2-{[8-(2,4-dimethyl-1,3-thiazol- 5-yl)quinazolin-4- yl]amino}propyl]piperazin-1-yl}sulfonyl)- 2,3-dihydro-1,3-benzothiazol-2-one |
| 99 | 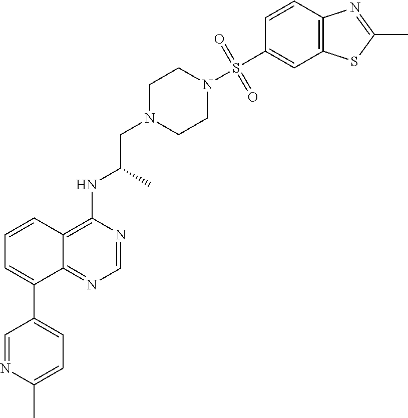 [SMILES: Cc1ccc(-c2cccc3c(N[C@@H](C)CN4CCN(S(=O)(=O)c5ccc6nc(C)sc6c5)CC4)ncnc23)cn1] | N-[(2S)-1-{4-[(2-methyl-1,3-benzothiazol- 6-yl)sulfonyl]piperazin-1-yl}propan-2-yl]- 8-(6-methylpyridin-3-yl)quinazolin-4-amine |
| 100 | 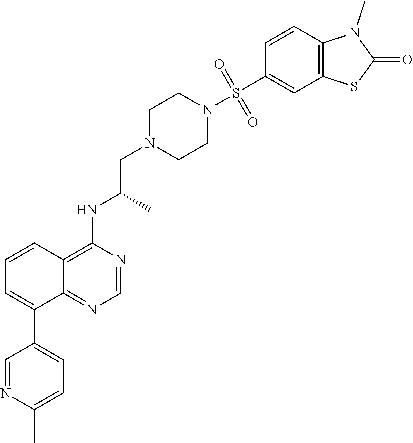 [SMILES: Cc1ccc(-c2cccc3c(N[C@@H](C)CN4CCN(S(=O)(=O)c5ccc6c(c5)sc(=O)n6C)CC4)ncnc23)cn1] | 3-methyl-6-({4-[(2S)-2-{[8-(6- methylpyridin-3-yl)quinazolin-4- yl]amino}propyl]piperazin-1-yl}sulfonyl)- 2,3-dihydro-1,3-benzothiazol-2-one |
| 101 | 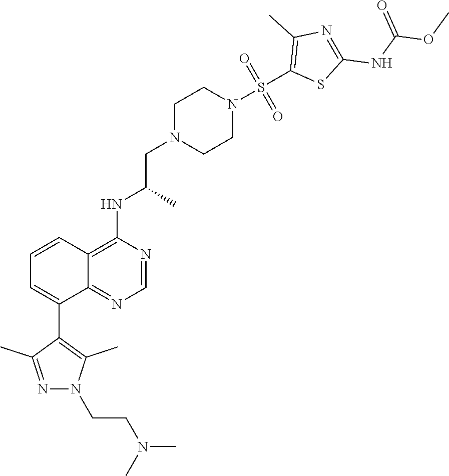 | methyl N-[5-({4-[(2S)-2-[(8-{1-[2- (dimethylamino)ethyl]-3,5-dimethyl-1H- pyrazol-4-yl}quinazolin-4- yl)amino]propyl]piperazin-1-yl}sulfonyl)- 4-methyl-1,3-thiazol-2-yl]carbamate |
| 102 | 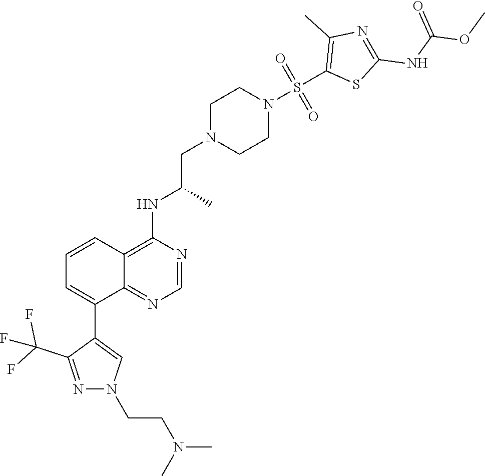 [SMILES: COC(=O)Nc1nc(C)c(S(=O)(=O)N2CCN(C[C@H](C)Nc3ncnc4c(-c5cn(CCN(C)C)nc5C(F)(F)F)cccc34)CC2)s1] | methyl N-[5-({4-[(2S)-2-[(8-{1-[2- (dimethylamino)ethyl]-3-(trifluoromethyl)- 1H-pyrazol-4-yl}quinazolin-4- yl)amino]propyl]piperazin-1-yl}sulfonyl)-4- methyl-1,3-thiazol-2-yl]carbamate |
| 103 | 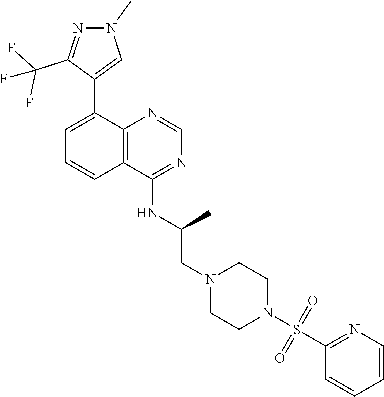 [SMILES: C[C@@H](CN1CCN(S(=O)(=O)c2ccccn2)CC1)Nc1ncnc2c(-c3cn(C)nc3C(F)(F)F)cccc12] | 8-[1-methyl-3-(trifluoromethyl)-1H-pyrazol- 4-yl]-N-[(2S)-1-[4-(pyridine-2- sulfonyl)piperazin-1-yl]propan-2- yl]quinazolin-4-amine |
| 104 | 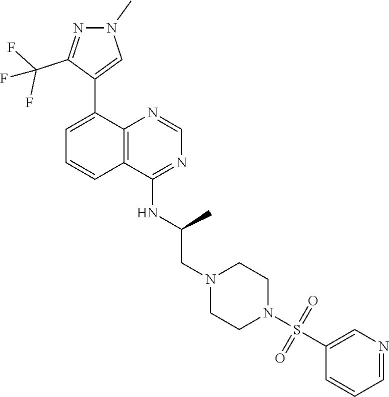 [SMILES: C[C@@H](CN1CCN(S(=O)(=O)c2cccnc2)CC1)Nc1ncnc2c(-c3cn(C)nc3C(F)(F)F)cccc12] | 8-[1-methyl-3-(trifluoromethyl)-1H-pyrazol- 4-yl]-N-[(2S)-1-[4-(pyridine-3- sulfonyl)piperazin-1-yl]propan-2- yl]quinazolin-4-amine |
| 105 | 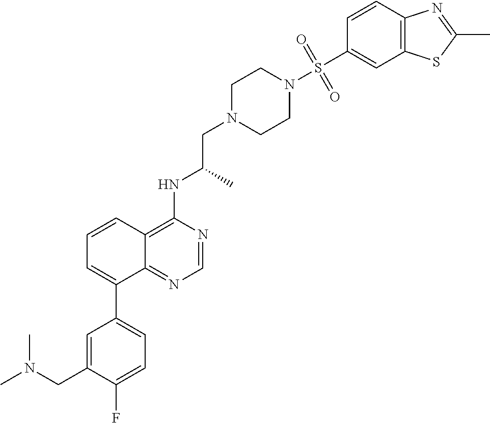 | 8-{3-[(dimethylamino)methyl]-4- fluorophenyl}-N-[(2S)-1-{4-[(2-methyl- 1,3-benzothiazol-6-yl)sulfonyl]piperazin-1- yl}propan-2-yl]quinazolin-4-amine |
| 106 | 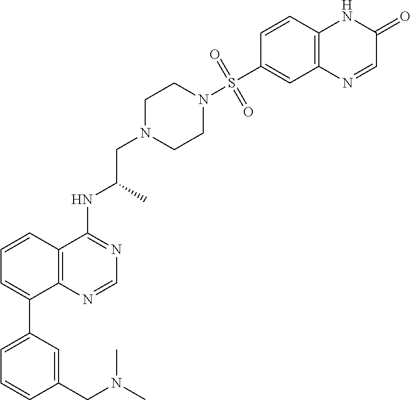 [SMILES: C[C@@H](CN1CCN(S(=O)(=O)c2ccc3[nH]c(=O)cnc3c2)CC1)Nc1ncnc2c(-c3cccc(CN(C)C)c3)cccc12] | 6-({4-[(2S)-2-[(8-{3- [(dimethylamino)methyl]phenyl}quinazolin- 4-yl)amino]propyl]piperazin-1-yl}sulfonyl)- 1,2-dihydroquinoxalin-2-one |
| 107 | 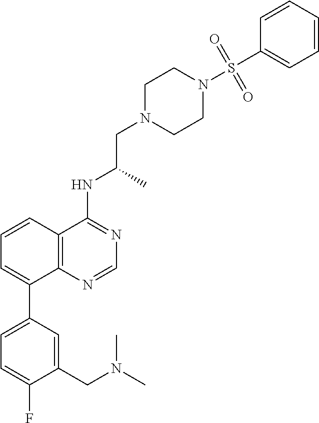 [SMILES: C[C@@H](CN1CCN(S(=O)(=O)c2ccccc2)CC1)Nc1ncnc2c(-c3ccc(F)c(CN(C)C)c3)cccc12] | N-[(2S)-1-[4-(benzenesulfonyl)piperazin-1- yl]propan-2-yl]-8-{3- [(dimethylamino)methyl]-4- fluorophenyl}quinazolin-4-amine |
| 108 | 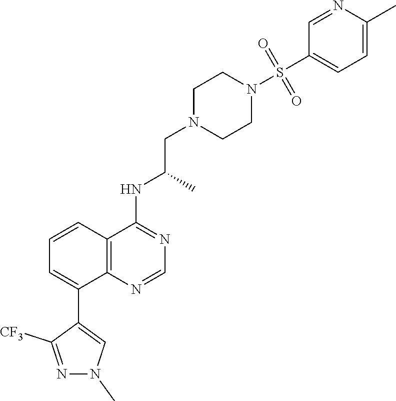 [SMILES: Cc1ccc(S(=O)(=O)N2CCN(C[C@H](C)Nc3ncnc4c(-c5cn(C)nc5C(F)(F)F)cccc34)CC2)cn1] | 8-[1-methyl-3-(trifluoromethyl)-1H-pyrazol- 4-yl]-N-[(2S)-1-{4-[(6-methylpyridin-3- yl)sulfonyl]piperazin-1-yl}propan-2- yl]quinazolin-4-amine |
| 109 | 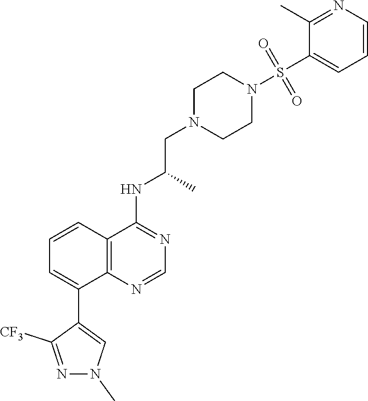 [SMILES: Cc1ncccc1S(=O)(=O)N1CCN(C[C@H](C)Nc2ncnc3c(-c4cn(C)nc4C(F)(F)F)cccc23)CC1] | 8-[1-methyl-3-(trifluoromethyl)-1H-pyrazol- 4-yl]-N-[(2S)-1-{4-[(2-methylpyridin-3- yl)sulfonyl]piperazin-1-yl}propan-2- yl]quinazolin-4-amine |

The compounds of the present invention can be prepared in a number of ways known to one skilled in the art of organic synthesis. It will be understood by those skilled in the art of organic synthesis that the functionality present on the molecule should be consistent with the transformation proposed. This will sometimes require a modification of the order of synthetic steps in order to obtain a desired compound of the invention. The compounds of formula (I), including all the compounds here above listed, can be generally prepared according to the procedure outlined in Schemes shown below using generally known methods.

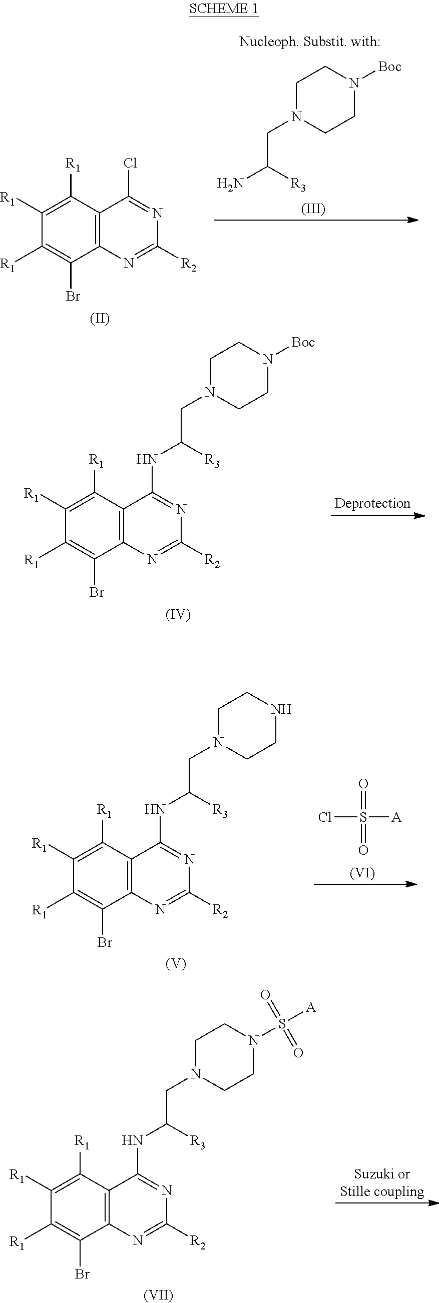

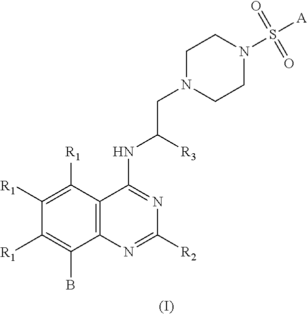

Compound of formula (II) can be reacted with a nitrogen based nucleophile of formula (III), in the presence of a suitable base e.g. N,N-diisopropylethylamine in a suitable solvent such as Acetonitrile, to provide compound (IV), containing a Boc-protected amino group. Deprotection under well-known procedures and reaction with a suitable sulphonyl chloride (VI) led to compound (VII). Final compound (I) can be obtained through a Suzuki coupling with commercially available boronic acid or ester in the presence of a suitable catalyst such as [1,1′-Bis(diphenylphosphino)ferrocene]dichloropalladium(II) or, alternatively, through a Stille coupling with a commercially available stannane in the presence of a suitable catalyst such as Tetrakis(triphenylphosphine)-palladium(O).

Alternatively, according to Scheme 2, compounds of formula (IV) undergoes Miyaura borylation followed by Suzuki coupling with a commercially available aryl bromide to provide intermediate (VIII). Deprotection under well-known procedures give compound (IX) and final reaction with a suitable sulphonyl chloride (VI) led to compound of formula (I).

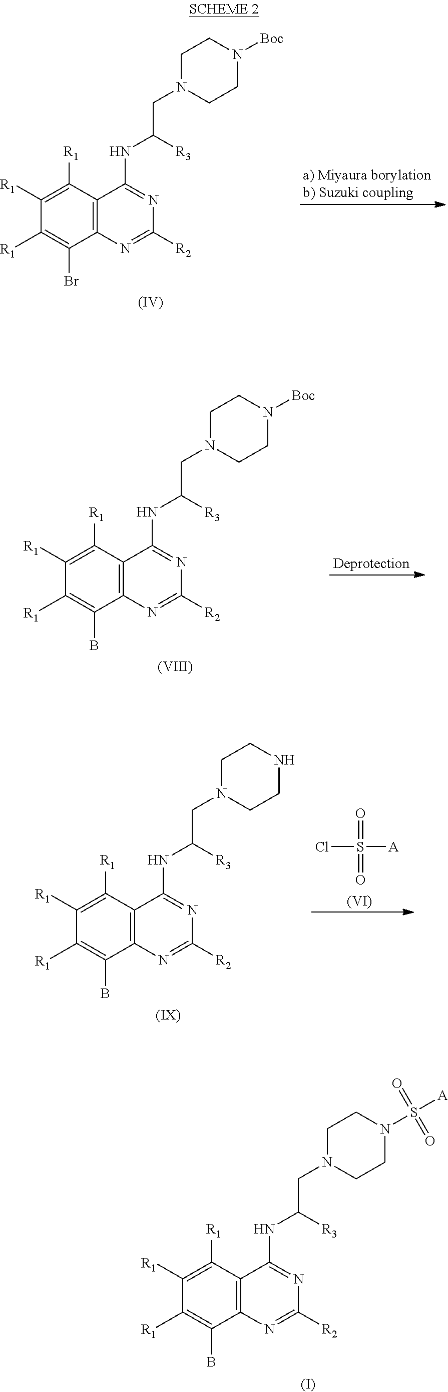

In another embodiment of the present invention, wherein A is an N-acylated aminothiazole wherein R₄ and R₅ are H and —CH₃, compound (XIV) may be obtained according to Scheme 3.

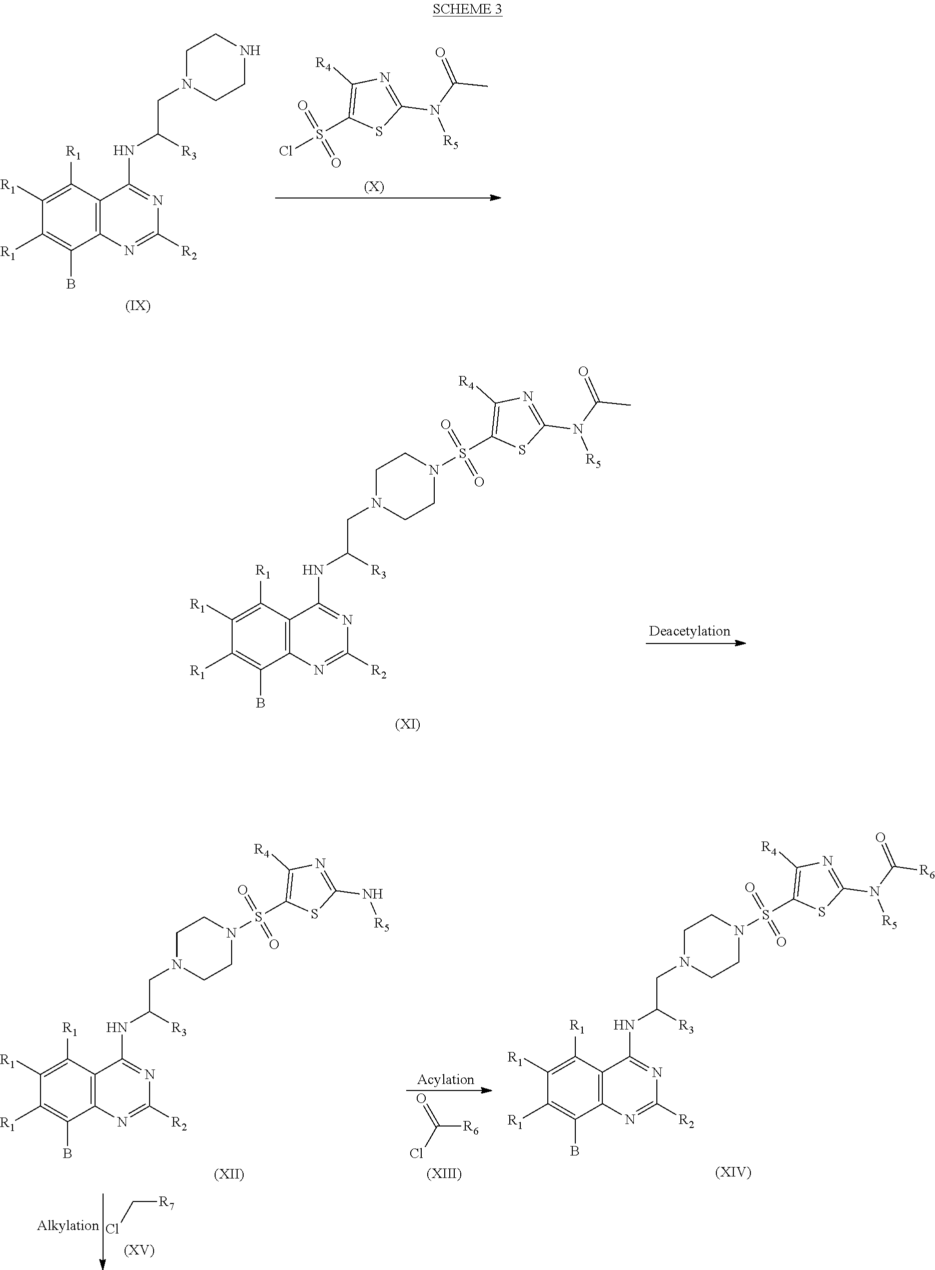

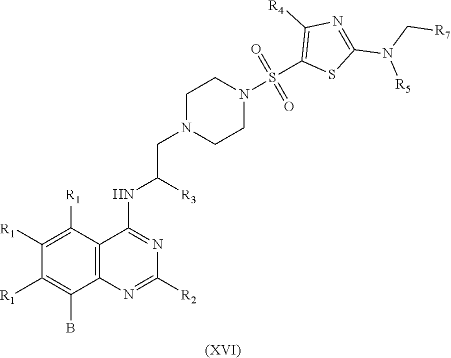

Reaction of intermediate (IX) with a N-acetyl thiazole sulfonyl chloride (X) followed by deacetylation under acid condition provides intermediate (XII). Final acetylation with a suitable acyl chloride in presence of a base, such as N,N-dimethyl-4-pyridinamine, led to final compound (XIV). Alternatively, alkylation with a commercially available alkyl chloride provided compound (XVI).

In another embodiment of the present invention, compound (XVII), wherein X is an aryl or a heteroaryl group substituted with an aldehyde moiety, can be prepared from compound (VII) similarly to Scheme 1, by means of a Suzuki coupling with a suitable boronic acid or ester. Compound (XVII) can be converted into compound (XVIII) by reductive amination with a suitable amine.

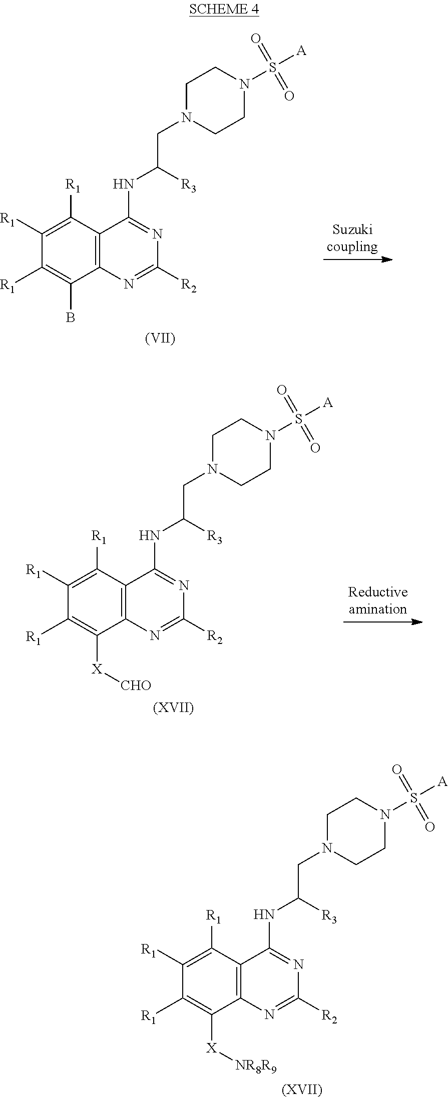

The compounds of formula (I) of the present invention have surprisingly been found to effectively inhibit receptor LPA2. Advantageously, the inhibition of LPA2 may result in efficacious treatment of the diseases or condition wherein the LPA receptors are involved.

In this respect, it has now been found that the compounds of formula (I) of the present invention have an antagonist drug potency expressed as half maximal inhibitory concentration (IC₅₀) on LPA2 lesser or equal than 1000 nM as shown in the present experimental part.

Preferably, the compounds of the present invention have an IC₅₀ on LPA2 lesser or equal than 100 nM.

More preferably, the compounds of the present invention have an IC₅₀ on LPA2 lesser or equal than 10 nM.

In one aspect, the present invention refers to a compound of formula (I) for use as a medicament.

In a preferred embodiment, the invention refers to a compound of formula (I) for use in the treatment of disorders associated with LPA receptors mechanism.

In a further embodiment, the present invention refers to a compound of formula (I) for use in the treatment of a disease, disorder or condition associated with dysregulation of lysophosphatidic acid receptor 2 (LPA2).

In one embodiment, the present invention refers to a compound of formula (I) useful for the prevention and/or treatment of fibrosis and/or diseases, disorders, or conditions that involve fibrosis.

The terms “fibrosis” or “fibrosing disorder,” as used herein, refers to conditions that are associated with the abnormal accumulation of cells and/or fibronectin and/or collagen and/or increased fibroblast recruitment and include but are not limited to fibrosis of individual organs or tissues such as the heart, kidney, liver, joints, lung, pleural tissue, peritoneal tissue, skin, cornea, retina, musculoskeletal and digestive tract.

Preferably, the compounds of formula (I) of the present invention are useful for the treatment and/or prevention of fibrosis such as pulmonary fibrosis, idiopathic pulmonary fibrosis (IPF), hepatic fibrosis, renal fibrosis, ocular fibrosis, cardiac fibrosis, arterial fibrosis and systemic sclerosis.

More preferably, the compounds of formula (I) of the present invention are useful for the treatment of idiopathic pulmonary fibrosis (IPF).

In one aspect, the invention also refers to a method for the prevention and/or treatment of disorders associated with LPA receptors mechanisms, said method comprises administering to a patient in need of such treatment a therapeutically effective amount of a compound of formula (I).

In one aspect, the invention refers to the use of a compound of formula (I) in the preparation of a medicament for the treatment of disorders associated with LPA receptors mechanism.

In a further aspect, the invention refers to a method for the prevention and/or treatment of disorder or condition associated with dysregulation of lysophosphatidic acid receptor 2 (LPA2) administering a patient in need of such treatment a therapeutically effective amount of a compound of formula (I).

In a further aspect, the invention refers to the use of a compound of formula (I) according to the invention, for the treatment of disorders associated with LPA receptors mechanism.

In a further aspect, the present invention refers to the use of a compound of formula (I) for the treatment of a disease, disorder or condition associated with dysregulation of receptor 2 (LPA2).

As used herein, “safe and effective amount” in reference to a compound of formula (I) or a pharmaceutically acceptable salt thereof or other pharmaceutically-active agent means an amount of the compound sufficient to treat the patient's condition but low enough to avoid serious side effects and it can nevertheless be routinely determined by the skilled artisan.

The compounds of formula (I) may be administered once or according to a dosing regimen wherein a number of doses are administered at varying intervals of time for a given period of time. Typical daily dosages may vary depending upon the route of administration chosen.

The present invention also refers to a pharmaceutical composition comprising a compound of formula (I) in admixture with at least one or more pharmaceutically acceptable carrier or excipient.

In one embodiment, the invention refers to a pharmaceutical composition of compounds of formula (I) in admixture with one or more pharmaceutically acceptable carrier or excipient, for example those described in Remington's Pharmaceutical Sciences Handbook, XVII Ed., Mack Pub., N.Y., U.S.A.

Administration of the compounds of the invention and their pharmaceutical compositions may be accomplished according to patient needs, for example, orally, nasally, parenterally (subcutaneously, intravenously, intramuscularly, intrasternally and by infusion) and by inhalation.

Preferably, the compounds of the present invention are administered orally or by inhalation.

More preferably, the compounds of the present invention are administered orally.

In one preferred embodiment, the pharmaceutical composition comprising the compound of formula (I) is a solid oral dosage form such as tablets, gelcaps, capsules, caplets, granules, lozenges and bulk powders.

In one embodiment, the pharmaceutical composition comprising the compound of formula (I) is a tablet.

The compounds of the invention can be administered alone or combined with various pharmaceutically acceptable carriers, diluents (such as sucrose, mannitol, lactose, starches) and known excipients, including suspending agents, solubilizers, buffering agents, binders, disintegrants, preservatives, colorants, flavorants, lubricants and the like.

In a further embodiment, the pharmaceutical composition comprising a compound of formula (I) is a liquid oral dosage forms such as aqueous and non-aqueous solutions, emulsions, suspensions, syrups, and elixirs. Such liquid dosage forms can also contain suitable known inert diluents such as water and suitable known excipients such as preservatives, wetting agents, sweeteners, flavorants, as well as agents for emulsifying and/or suspending the compounds of the invention.

In a further embodiment, the pharmaceutical composition comprising the compound of formula (I) is an inhalable preparation such as inhalable powders, propellant-containing metering aerosols or propellant-free inhalable formulations.

For administration as a dry powder, single- or multi-dose inhalers known from the prior art may be utilized. In that case the powder may be filled in gelatine, plastic or other capsules, cartridges or blister packs or in a reservoir.

A diluent or carrier chemically inert to the compounds of the invention, e.g. lactose or any other additive suitable for improving the respirable fraction may be added to the powdered compounds of the invention.

Inhalation aerosols containing propellant gas such as hydrofluoroalkanes may contain the compounds of the invention either in solution or in dispersed form. The propellant-driven formulations may also contain other ingredients such as co-solvents, stabilizers and optionally other excipients.

The propellant-free inhalable formulations comprising the compounds of the invention may be in form of solutions or suspensions in an aqueous, alcoholic or hydroalcoholic medium and they may be delivered by jet or ultrasonic nebulizers known from the prior art or by soft-mist nebulizers.

The compounds of the invention can be administered as the sole active agent or in combination with other pharmaceutical active ingredients.

The dosages of the compounds of the invention depend upon a variety of factors including among others the particular disease to be treated, the severity of the symptoms, the route of administration and the like.

The invention is also directed to a device comprising a pharmaceutical composition comprising a compound of Formula (I) according to the invention, in form of a single- or multi-dose dry powder inhaler or a metered dose inhaler.

All preferred groups or embodiments described above for compounds of formula I may be combined among each other and apply as well mutatis mutandis.

The various aspects of the invention described in this application are illustrated by the following examples which are not meant to limit the invention in any way.

### PREPARATIONS OF INTERMEDIATES AND EXAMPLES

Chemical Names of the compounds were generated with Structure To Name Enterprise 10.0 Cambridge Software.

All reagents, for which the synthesis is not described in the experimental part, are either commercially available, or are known compounds or may be formed from known compounds by known methods by a person skilled in the art.

### ABBREVIATION—MEANING

AcOEt=Ethyl acetate
AcOK=Potassium acetate
Cs₂CO₃=Cesium carbonate
DCM=Dichloromethane
DIPEA=N,N-diisopropylethylamine
DMAP=4-Dimethylaminopyridine
DMSO=Dimethylsulfoxide
Et₃N=Triethylamine
Et₂O=Diethyl ether
Fe⁰=Metallic iron
h=hour
HCl=Hydrochloric acid
HCOOH=Formic acid
H₂O=Water
K₂CO₃=Potassium carbonate
K₃PO₄=Potassium phosphate tribasic
LC-MS=Liquid chromatography/mass spectrometry
MeCN=Acetonitrile
MeOH=Methanol
MW=Microwave
NaOH=Sodium hydroxide
Na₂SO₄=Sodium sulfate
NH₃=Ammonia
NH₄Cl=Ammonium chloride
NH₄HCO₃=Ammonium bicarbonate
NaHCO₃=Sodium bicarbonate
Na₂SO₄=Sodium sulfate
Pd(dppf)Cl₂=[1,1′-Bis(diphenylphosphino)ferrocene]dichloropalladium(II)
rac-BINAP=(±)-2,2′-Bis(diphenylphosphino)-1,1′-binaphthalene
r.t.=room temperature
SCX=strong cation exchange
SPhos Pd G2=chloro(2-dicyclohexylphosphino-2′,6′-dimethoxy-1,1′-biphenyl)[2-(2′-amino-1,1′-biphenyl)]palladium(II)
STAB=Sodium triacetoxyborohydride
THF=Tetrahydrofuran

Analytical Method

Instruments, Materials and Methods Employed for Analyses

¹H-NMR

¹H-NMR spectra were performed on a Varian MR-400 spectrometer operating at 400 MHZ (proton frequency), equipped with: a self-shielded Z-gradient coil 5 mm 1H/nX broadband probe head for reverse detection, deuterium digital lock channel unit, quadrature digital detection unit with trans mitter offset frequency shift, or on AgilentVNMRS-500 or on a Bruker Avance 400 spectrometers. Chemical shift are reported as 6 values in ppm relative to trimethylsilane (TMS) as an internal standard. Coupling constants (J values) are given in hertz (Hz) and multiplicities are reported using the following abbreviation (s=singlet, d=doublet, t=triplet, q=quartet, m=multiplet, br. s=broad singlet, nd=not determined).

LC/UV/MS

LC/MS retention times are estimated to be affected by an experimental error of +0.5 min. LCMS may be recorded under the following conditions: diode array DAD chromatographic traces, mass chromatograms and mass spectra may be taken on UPLC/PDA/MS Acquity™ system coupled with Micromass ZQ™ or Waters SQD single quadrupole mass spectrometer operated in positive and/or negative electron spray ES ionization mode and/or Fractionlynx system used in analytical mode coupled with ZQ™ single quadrupole operated in positive and/or negative ES ionisation mode. Quality Control methods used operated under low pH conditions or under high pH conditions:

Method 1, low pH conditions: column: Acquity CSH C18 2.1×50 mm 1.7 um, the column temperature was 40° C.; mobile phase solvent A was milliQ water+0.1% HCOOH, mobile phase solvent B MeCN+0.1% HCOOH. The flow rate was 1 mL/min. The gradient table was t=0 min 97% A 3% B, t=1.5 min 0.1% A 99.9% B, t=1.9 min 0.1% A 99.9% B and t=2 min 97% A 3% B. The UV detection range was 210-350 nm and ES+/ES− range was 100 to 1500 AMU.

Method 2, high pH conditions: column: Acquity Kinetex 1.7 um EVO C18 100A, 2.1×50 mm, the column temperature was 40° C.; mobile phase solvent A was 10 mM aqueous solution of NH₄HCO₃ adjusted to pH=10 with ammonia, mobile phase solvent B MeCN. The flow rate was 1 mL/min. The gradient table was t=0 min 97% A 3% B, t=1.5 min 0.1% A 99.9% B, t=1.9 min 0.1% A 99.9% B and t=2 min 97% A 3% B. The UV detection range was 210-350 nm and ES+/ES− range was 100 to 1500 AMU.

Method 3, low pH conditions: column: Acquity CSH C18 2.1×50 mm 1.7 μm, the column temperature was 40° C.; mobile phase solvent A was milliQ water/MeCN 95:5+0.05% HCOOH, mobile phase solvent B MeCN/milliQ water 95:5+0.05% HCOOH. The flow rate was 1 mL/min. The gradient table was t=0 min 99% A 1% B, t=1.5 min 0.1% A 99.9% B, t=1.9 min 0.1% A 99.9% B and t=2 min 99% A 1% B. The UV detection range was 210-400 nm and ES+/ES− range was 100 to 1200 AMU.

### Example 1

### methyl N-[5-({4-[(2S)-2-{[8-(2,4-dimethyl-1,3-thiazol-5-yl)quinazolin-4-yl]amino}propyl]piperazin-1-yl}sulfonyl)-4-methyl-1,3-thiazol-2-yl]carbamate

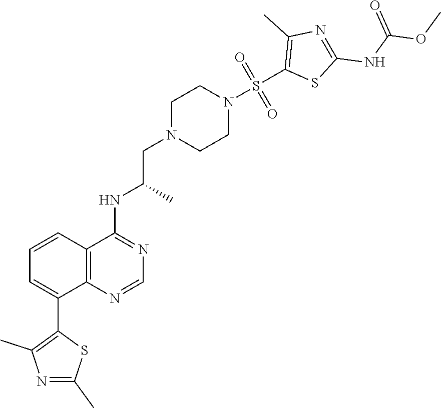

### Step 1: Preparation of tert-butyl 4-[(2S)-2-[(8-bromoquinazolin-4-yl)amino]propyl]piperazine-1-carboxylate (Intermediate 1)

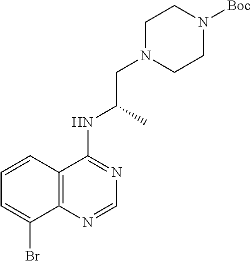 [SMILES: C[C@@H](CN1CCN(C(=O)OC(C)(C)C)CC1)Nc1ncnc2c(Br)cccc12]

To a solution of 8-bromo-4-chloroquinazoline (10 g, 41 mmol) in MeCN (100 mL) tert-butyl 4-[(2S)-2-aminopropyl]piperazine-1-carboxylate hydrochloride (11.49 g, 41.07 mmol) and then DIPEA (13.27 g, 102.67 mmol) were added. The solution was heated at 40° C. for 2 h. After cooling the mixture was concentrated to about 25 mL, water was added (90 mL) and the precipitate was filtered, washed with water (15 mL) and dried under vacuum to afford tert-butyl 4-[(2S)-2-[(8-bromoquinazolin-4-yl)amino]propyl]piperazine-1-carboxylate (17 g, 37.7 mmol, 92% yield) as a white solid.

LC-MS (ESI): m/z (M+1): 452.1 (Method 2)

¹H NMR (500 MHz, DMSO-d₆) δ ppm 8.52 (s, 1H), 8.32 (dd, J=8.4, 1.3 Hz, 1H), 8.12 (dd, J=7.6, 1.1 Hz, 1H), 8.06 (d, J=8.0 Hz, 1H), 7.42 (t, J=7.9 Hz, 1H), 4.67 (spt, J=7.0 Hz, 1H), 3.25-3.23 (m, 5H), 2.62-2.57 (m, 2H), 2.43-2.38 (m, 5H), 1.38 (s, 9H), 1.24 (d, J=6.5 Hz, 3H)

### Step 2: Preparation of 8-bromo-N-[(2S)-1-piperazin-1-ylpropan-2-yl]quinazolin-4-amine hydrochloride (Intermediate 2)

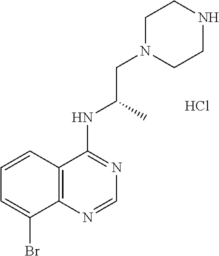 [SMILES: C[C@@H](CN1CCNCC1)Nc1ncnc2c(Br)cccc12 ; Cl]

To a suspension of Intermediate 1 (17 g, 37.7 mmol) in 1,4-Dioxane (50 mL), HCl 4M in 1,4-dioxane (51 mL, 204 mmol) was added. The suspension was stirred at 20° C. for 4 hours. The mixture was concentrated under reduced pressure, then Et₂O was added and the mixture was stirred for 1 h at room temperature. The slurry was filtered and the solid obtained was dried under vacuum at 35° C. overnight to afford the title compound 8-bromo-N-[(2S)-1-piperazin-1-ylpropan-2-yl]quinazolin-4-amine hydrochloride (Intermediate 2, 18 g, 39 mmol, crude) as a whitish solid.

LC-MS (ESI): m/z (M+1): 352.1 (Method 1)

### Step 3: Preparation of methyl N-[5-[4-[(2S)-2-[(8-bromoquinazolin-4-yl)amino]propyl]piperazin-1-yl]sulfonyl-4-methyl-1,3-thiazol-2-yl]carbamate (Intermediate 3)

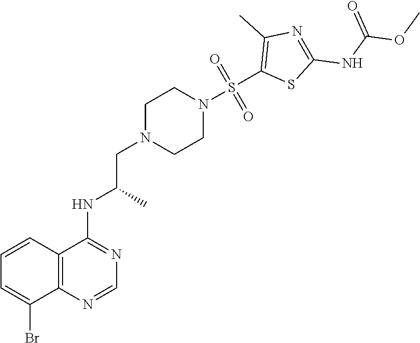 [SMILES: COC(=O)Nc1nc(C)c(S(=O)(=O)N2CCN(C[C@H](C)Nc3ncnc4c(Br)cccc34)CC2)s1]

To a suspension of Intermediate 2 (18.0 g, 39 mmol) in MeCN (200 mL), triethylamine (24.5 mL, 176 mmol) was added under stirring; after 15 minutes the mixture was cooled to 10° C. and methyl N-[5-(chlorosulfonyl)-4-methyl-1,3-thiazol-2-yl]carbamate (10.6 g, 39 mmol) was added. After 15 min the mixture was left to reach r.t. and the resulting slurry was stirred for 1 h. H₂O (900 mL) was added and the mixture was stirred at r.t. for 1 h. The precipitate was filtered, washed with water (30 mL) and dried under vacuum to afford methyl N-[5-[4-[(2S)-2-[(8-bromoquinazolin-4-yl)amino]propyl]piperazin-1-yl]sulfonyl-4-methyl-1,3-thiazol-2-yl]carbamate (20 g, 34 mmol, 91% yield) as a white solid.

LC-MS (ESI): m/z (M+1): 585.9 (Method 1)

### Step 4: Preparation of methyl N-[5-({4-[(2S)-2-{[8-(2,4-dimethyl-1,3-thiazol-5-yl)quinazolin-4-yl]amino}propyl]piperazin-1-yl}sulfonyl)-4-methyl-1,3-thiazol-2-yl]carbamate (Example 1)

To a suspension of Pd(dppf)Cl₂ (38 mg, 0.05 mmol) in water (0.3 mL) and THE (1 mL), Intermediate 3 (150 mg, 0.26 mmol) and 2,4-dimethylthiazole-5-boronic acid pinacol ester (92 mg, 0.38 mmol) were added. After degassing with nitrogen, K₃PO₄ (109 mg, 0.51 mmol) was added and the tube was sealed. The reaction was heated at 80° C. for 24 h, then the mixture was diluted with AcOEt and the solid precipitate was removed by filtration. The organic layer was concentrated under reduced pressure and the crude was purified by flash chromatography eluting with MeOH in DCM from 000 to 700 providing title compound (74.5 mg, 0.12 mmol, 47 n yield) as a yellowish solid.

LC-MS (ESI): m/z (M+1): 617.2 (Method 2)

¹H NMR (400 MHz, DMSO-d₆) δ ppm 1.21 (d, J=6.4 Hz, 3H), 2.26 (s, 3H), 2.38-2.46 (i, 4H), 2.57 (br s, 4H), 2.59-2.66 (i, 4H), 2.99 (br s, 4H), 3.75 (s, 3H), 4.53-4.68 (m, 1H), 7.53 (t, J=7.8 Hz, 1H), 7.80 (dd, J=7.2, 1.1 Hz, 1H), 7.95 (d, J=7.9 Hz, 1H), 8.28 (d, J=7.7 Hz, 1H), 8.43 (s, 1H), 12.33 (br s, 1H)

The Examples in the following table were prepared from commercially available reagents by using methods analogous to Example 1.

| Example | | |
|---|---|---|
| No. | Structure & Name | Analytical data |
| 3 | 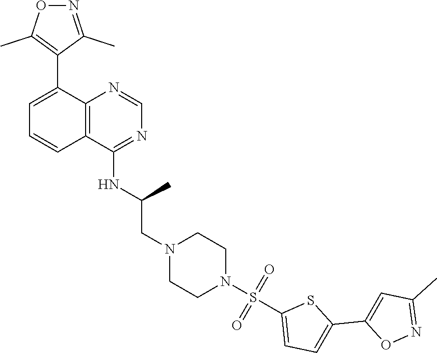 | LC-MS (ESI): m/z (M + 1): 594.2 (Method 2) ¹H NMR (400 MHz, DMSO-d₆) δ ppm 1.20 (d, J = 6.6 Hz, 3 H), 2.04 (s, 3 H), 2.23 (s, 3 H), 2.29 (s, 3 H), 2.42 (br dd, J = 12.4, 6.9 Hz, 1 H), 2.55-2.62 (m, 4 H), 2.60-2.66 (m, 1 H), 2.90-3.06 (m, 4 H), 4.60 (spt, J = 6.9 Hz, 1 H), 6.97 (s, 1 H), 7.53 (t, J = 7.7 Hz, 1 H), 7.65-7.71 (m, 2 H), 7.76 (d, J = 3.9 Hz, 1 H), 7.93 (d, J = 8.1 Hz, 1 H), 8.29 (d, J = 8.3 Hz, 1 H), 8.40 (s, 1 H) |
| | 8-(3,5-dimethyl-1,2-oxazol-4-yl)-N-[(2S)-1-(4-{[5-(3-methyl-1,2- | |
| | oxazol-5-yl)thiophen-2-yl]sulfonyl}piperazin-1-yl)propan-2-yl]- | |
| | quinazolin-4-amine | |
| 4 | 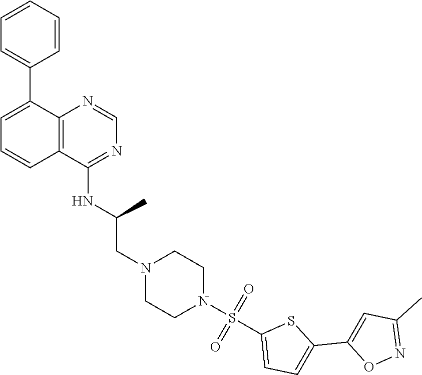 [SMILES: Cc1cc(-c2ccc(S(=O)(=O)N3CCN(C[C@H](C)Nc4ncnc5c(-c6ccccc6)cccc45)CC3)s2)on1] | LC-MS (ESI): m/z (M + 1): 575.2 (Method 2) ¹H NMR (400 MHz, DMSO-d₆) δ ppm 1.21 (d, J = 6.6 Hz, 3 H), 2.29 (s, 3 H), 2.44 (br dd, J = 12.5, 6.8 Hz, 1 H), 2.56-2.66 (m, 5 H), 2.84-3.12 (m, 4 H), 4.52- 4.72 (m, 1 H), 6.96 (s, 1 H), 7.31-7.40 (m, 1 H), 7.40-7.49 (m, 2 H), 7.50-7.57 (m, 1 H), 7.57-7.65 (m, 2 H), 7.67-7.80 (m, 3 H), 7.80-7.95 (m, 1 H), 8.18-8.32 (m, 1 H), 8.42 (s, 1 H) |
| | N-[(2S)-1-(4-{[5-(3-methyl-1,2-oxazol-5-yl)thiophen-2-yl]sulfonyl}- | |
| | piperazin-1-yl)propan-2-yl]-8-phenylquinazolin-4-amine | |
| 5 | 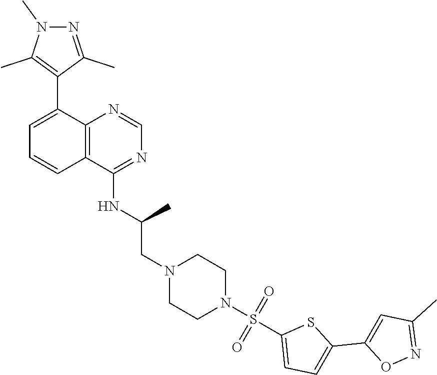 | LC-MS (ESI): m/z (M + 1): 607.2 (Method 2) ¹H NMR (500 MHz, DMSO-d₆) δ ppm 1.19 (d, J = 6.6 Hz, 3 H), 1.93 (s, 3 H), 2.00 (s, 3 H), 2.29 (s, 3 H), 2.41 (br dd, J = 12.4, 6.9 Hz, 1 H), 2.55-2.63 (m, 5 H), 2.98 (br s, 4 H), 3.71 (s, 3 H), 4.52-4.69 (m, 1 H), 6.97 (s, 1 H), 7.41-7.50 (m, 1 H), 7.51- 7.58 (m, 1 H), 7.70 (d, J = 4.1 Hz, 1 H), 7.76 (d, J = 4.1 Hz, 1 H), 7.81 (d, J = 8.0 Hz, 1 H), 8.18 (dd, J = 8.4, 1.2 Hz, 1 H), 8.32-8.38 (m, 1 H) |
| | N-[(2S)-1-(4-{[5-(3-methyl-1,2-oxazol-5-yl)thiophen-2-yl]sulfonyl}- | |
| | piperazin-1-yl)propan-2-yl]-8-(1,3,5-trimethyl-1H-pyrazol-4-yl)- | |
| | quinazolin-4-amine | |
| 6 | 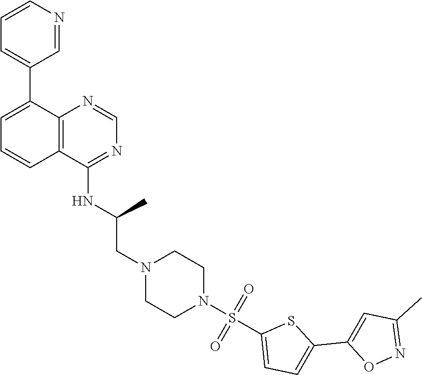 [SMILES: Cc1cc(-c2ccc(S(=O)(=O)N3CCN(C[C@H](C)Nc4ncnc5c(-c6cccnc6)cccc45)CC3)s2)on1] | LC-MS (ESI): m/z (M + 1): 576.2 (Method 2) ¹H NMR (400 MHz, DMSO-d₆) δ ppm 1.17 (d, J = 6.6 Hz, 3 H), 2.25 (s, 3 H), 2.34-2.44 (m, 1 H), 2.52-2.62 (m, 5 H), 2.94 (br s, 4 H), 4.51-4.72 (m, 1 H), 6.92 (s, 1 H), 7.36-7.50 (m, 1 H), 7.50-7.58 (m, 1 H), 7.66 (d, J = 3.9 Hz, 1 H), 7.72 (d, J = 3.9 Hz, 1 H), 7.78 (dd, J = 7.2, 1.1 Hz, 1 H), 7.90 (d, J = 7.9 Hz, 1 H), 7.98 (dt, J = 8.0, 1.9 Hz, 1 H), 8.26 (d, J = 7.5 Hz, 1 H), 8.39 (s, 1 H), 8.53 (dd, J = 4.8, 1.8 Hz, 1 H), 8.74 (d, J = 1.5 Hz, 1 H) |
| | N-[(2S)-1-(4-{[5-(3-methyl-1,2-oxazol-5-yl)thiophen-2-yl]sulfonyl}- | |
| | piperazin-1-yl)propan-2-yl]-8-(pyridin-3-yl)quinazolin-4-amine | |
| 7 | 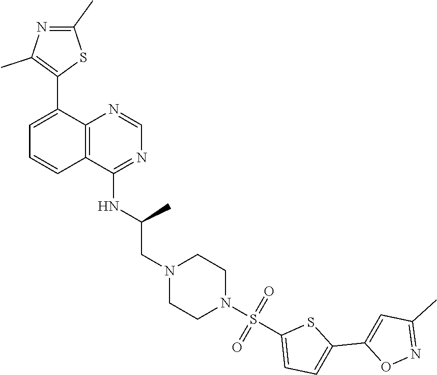 | LC-MS (ESI): m/z (M + 1): 610.08 (Method 2) ¹H NMR (400 MHz, DMSO-d₆) δ ppm 1.19 (d, J = 6.6 Hz, 3 H), 2.24 (s, 3 H), 2.28 (s, 3 H), 2.42 (dd, J = 12.5, 6.6 Hz, 1 H), 2.55-2.65 (m, 5 H), 2.63 (s, 3 H), 2.90-3.05 (m, 4 H), 4.61 (dquin, J = 14.0, 7.1, 7.1, 7.1, 7.1 Hz, 1 H), 6.95 (s, 1 H), 7.49-7.54 (m, 1 H), 7.69 (d, J = 4.0 Hz, 1 H), 7.75 (d, J = 4.0 Hz, 1 H), 7.77 (dd, J = 7.3, 1.1 Hz, 1 H), 7.93 (d, J = 7.7 Hz, 1 H), 8.26 (dd, J = 8.4, 1.1 Hz, 1 H), 8.42 (s, 1 H) |
| | 8-(2,4-dimethyl-1,3-thiazol-5-yl)-N-[(2S)-1-(4-{[5-(3-methyl- | |
| | 1,2-oxazol-5-yl)thiophen-2-yl]sulfonyl}piperazin-1-yl)propan- | |
| | 2-yl]quinazolin-4-amine | |
| 8 | 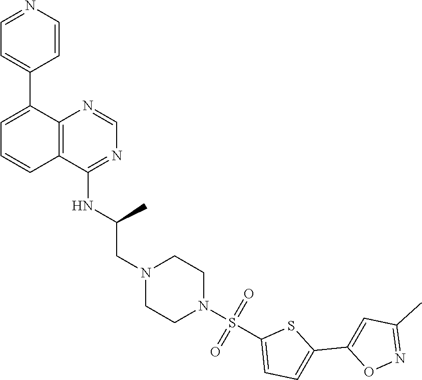 [SMILES: Cc1cc(-c2ccc(S(=O)(=O)N3CCN(C[C@H](C)Nc4ncnc5c(-c6ccncc6)cccc45)CC3)s2)on1] | LC-MS (ESI): m/z (M + 1): 576.1 (Method 2) ¹H NMR (400 MHz, DMSO-d₆) δ ppm 1.20 (d, J = 6.1 Hz, 3 H), 2.28 (s, 3 H), 2.40-2.46 (m, 1 H), 2.56-2.65 (m, 5 H), 2.90-3.04 (m, 4 H), 4.56-4.66 (m, 1 H), 6.96 (s, 1 H), 7.57 (t, J = 7.7 Hz, 1 H), 7.61-7.64 (m, 2 H), 7.69 (d, J = 3.9 Hz, 1 H), 7.75 (d, J = 3.9 Hz, 1 H), 7.82 (dd, J = 7.5, 0.9 Hz, 1 H), 7.95 (d, J = 7.9 Hz, 1 H), 8.30-8.36 (m, 1 H), 8.43 (s, 1 H), 8.59- 8.65 (m, 2 H) |
| | N-[(2S)-1-(4-{[5-(3-methyl-1,2-oxazol-5-yl)thiophen-2-yl]sulfonyl}- | |
| | piperazin-1-yl)propan-2-yl]-8-(pyridin-4-yl)quinazolin-4-amine | |
| 9 | 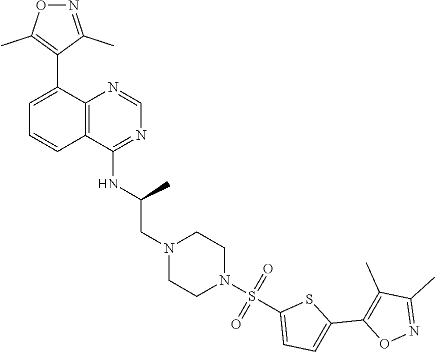 | LC-MS (ESI): m/z (M + 1): 608.1 (Method 2) ¹H NMR (400 MHz, DMSO-d₆) δ ppm 1.20 (d, J = 6.4 Hz, 3 H), 2.00-2.25 (m, 12 H), 2.42 (br dd, J = 12.2, 6.7 Hz, 1 H), 2.60 (br d, J = 5.3 Hz, 5 H), 2.98 (br s, 4 H), 4.60 (dt, J = 14.3, 6.8 Hz, 1 H), 7.53 (dd, J = 8.3, 7.4 Hz, 1 H), 7.64- 7.73 (m, 2 H), 7.68 (dd, J = 7.2, 1.2 Hz, 1 H), 7.92 (d, J = 7.9 Hz, 1 H), 8.29 (dd, J = 8.5, 1.2 Hz, 1 H), 8.40 (s, 1 H) |
| | 8-(3,5-dimethyl-1,2-oxazol-4-yl)-N-[(2S)-1-(4-{[5-(3,4-dimethyl- | |
| | 1,2-oxazol-5-yl)thiophen-2-yl]sulfonyl}piperazin-1-yl)propan- | |
| | 2-yl]quinazolin-4-amine | |
| 10 | 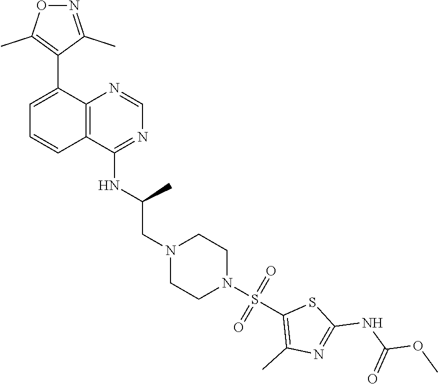 | LC-MS (ESI): m/z (M + 1): 601.15 (Method 2) ¹H NMR (400 MHz, DMSO-d₆) δ ppm 1.21 (d, J = 6.6 Hz, 3 H), 2.02-2.42 (m, 9 H), 2.37-2.47 (m, 1 H), 2.55-2.65 (m, 5 H), 2.93-3.05 (m, 4 H), 3.73 (br s, 3 H), 4.55-4.68 (m, 1 H), 7.51- 7.58 (m, 1 H), 7.70 (dd, J = 7.3, 1.1 Hz, 1 H), 7.94 (d, J = 7.9 Hz, 1 H), 8.29-8.34 (m, 1 H), 8.41 (s, 1 H), 12.33 (br s, 1 H) |
| | methyl N-[5-({4-[(2S)-2-{[8-(3,5-dimethyl-1,2-oxazol-4-yl)- | |
| | quinazolin-4-yl]amino}propyl]piperazin-1-yl}sulfonyl)-4- | |
| | methyl-1,3-thiazol-2-yl]carbamate | |
| 11 | 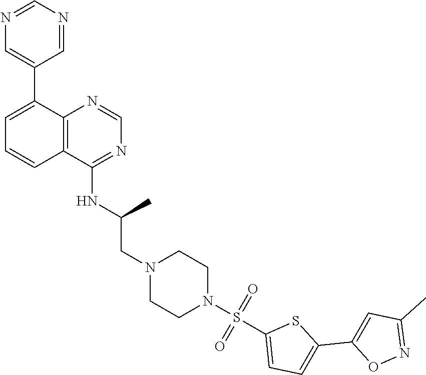 [SMILES: Cc1cc(-c2ccc(S(=O)(=O)N3CCN(C[C@H](C)Nc4ncnc5c(-c6cncnc6)cccc45)CC3)s2)on1] | LC-MS (ESI): m/z (M + 1): 577.13 (Method 2) ¹H NMR (400 MHz, DMSO-d₆) δ ppm 1.20 (d, J = 6.6 Hz, 3 H), 2.28 (s, 3 H), 2.40-2.47 (m, 1 H), 2.55-2.65 (m, 5 H), 2.89-3.07 (m, 4 H), 4.54-4.70 (m, 1 H), 6.95 (s, 1 H), 7.56-7.63 (m, 1 H), 7.69 (d, J = 4.0 Hz, 1 H), 7.75 (d, J = 4.0 Hz, 1 H), 7.92 (dd, J = 7.3, 1.1 Hz, 1 H), 7.99 (d, J = 7.9 Hz, 1 H), 8.35 (dd, J = 8.6, 1.1 Hz, 1 H), 8.45 (s, 1 H), 9.05 (s, 2 H), 9.18 (s, 1 H) |
| | N-[(2S)-1-(4-{[5-(3-methyl-1,2-oxazol-5-yl)thiophen-2-yl]sulfonyl}- | |
| | piperazin-1-yl)propan-2-yl]-8-(pyrimidin-5- | |
| | yl)quinazolin-4-amine | |
| 13 | 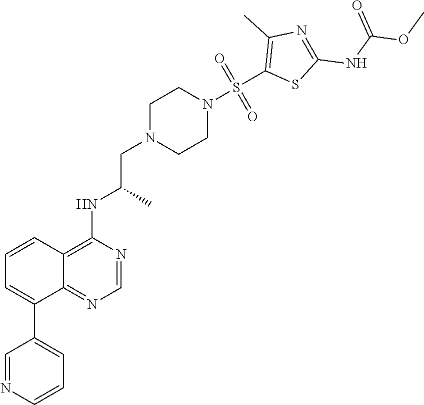 [SMILES: COC(=O)Nc1nc(C)c(S(=O)(=O)N2CCN(C[C@H](C)Nc3ncnc4c(-c5cccnc5)cccc34)CC2)s1] | LC-MS (ESI): m/z (M + 1): 583.4 (Method 1) ¹H NMR (400 MHz, DMSO-d₆) δ ppm 1.22 (d, J = 6.6 Hz, 3 H), 2.37-2.47 (m, 4 H), 2.53-2.66 (m, 5 H), 2.99 (br s, 4 H), 3.74 (s, 3 H), 4.61 (dt, J = 14.1, 6.9 Hz, 1 H), 7.48 (ddd, J = 7.9, 4.8, 0.7 Hz, 1 H), 7.58 (dd, J = 8.3, 7.4 Hz, 1 H), 7.83 (dd, J = 7.3, 1.3 Hz, 1 H), 7.93 (d, J = 7.9 Hz, 1 H), 8.03 (dt, J = 8.0, 1.9 Hz, 1 H), 8.32 (dd, J = 8.5, 1.2 Hz, 1 H), 8.44 (s, 1 H), 8.56 (dd, J = 4.7, 1.7 Hz, 1 H), 8.79 (dd, J = 2.2, 0.7 Hz, 1 H), 12.31 (br s, 1 H) |
| | methyl N-[4-methyl-5-({4-[(2S)-2-{[8-(pyridin-3-yl)quinazolin- | |
| | 4-yl]amino}propyl]piperazin-1-yl}sulfonyl)-1,3-thiazol-2- | |
| | yl]carbamate | |
| 14 | 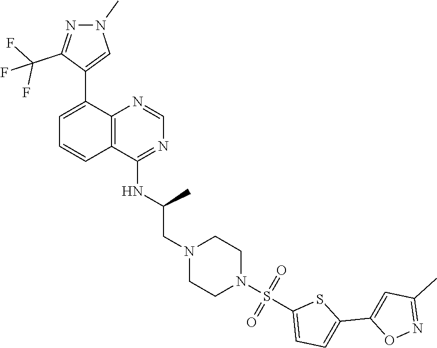 [SMILES: Cc1cc(-c2ccc(S(=O)(=O)N3CCN(C[C@H](C)Nc4ncnc5c(-c6cn(C)nc6C(F)(F)F)cccc45)CC3)s2)on1] | LC-MS (ESI): m/z (M + 1): 647.1 (Method 2) ¹H NMR (500 MHz, DMSO-d₆) δ ppm 1.19 (d, J = 6.6 Hz, 3 H), 2.28 (s, 3 H), 2.42 (br dd, J = 12.3, 6.9 Hz, 1 H), 2.54-2.65 (m, 5 H), 2.89-3.07 (m, 4 H), 3.99 (s, 3 H), 4.60 (spt, J = 6.9 Hz, 1 H), 6.96 (s, 1 H), 7.49 (t, J = 7.8 Hz, 1 H), 7.65 (d, J = 7.1 Hz, 1 H), 7.69 (d, J = 3.8 Hz, 1 H), 7.76 (d, J = 3.8 Hz, 1 H), 7.89 (br d, J = 8.0 Hz, 1 H), 8.05 (s, 1 H), 8.25 (d, J = 8.2 Hz, 1 H), 8.39 (s, 1 H) |
| | N-[(2S)-1-(4-{[5-(3-methyl-1,2-oxazol-5-yl)thiophen-2-yl]- | |
| | sulfonyl}piperazin-1-yl)propan-2-yl]-8-[1-methyl-3-(trifluoro- | |
| | methyl)-1H-pyrazol-4-yl]quinazolin-4-amine | |
| 15 | 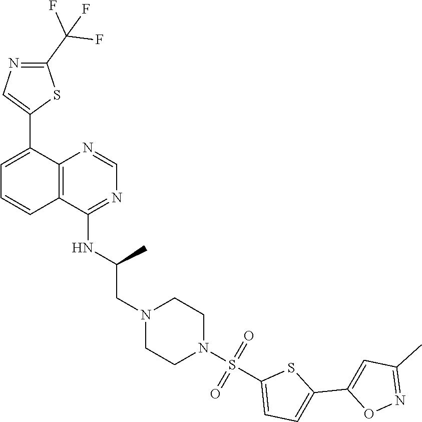 [SMILES: Cc1cc(-c2ccc(S(=O)(=O)N3CCN(C[C@H](C)Nc4ncnc5c(-c6cnc(C(F)(F)F)s6)cccc45)CC3)s2)on1] | LC-MS (ESI): m/z (M + 1): 649.98 (Method 2) ¹H NMR (400 MHz, DMSO-d₆) δ ppm 1.20 (d, J = 6.6 Hz, 3 H), 2.27 (s, 3 H), 2.40-2.47 (m, 1 H), 2.55-2.66 (m, 5 H), 2.96 (br s, 4 H), 4.59-4.71 (m, 1 H), 6.92 (s, 1 H), 7.61 (t, J = 7.9 Hz, 1 H), 7.68 (d, J = 3.9 Hz, 1 H), 7.73 (d, J = 4.0 Hz, 1 H), 8.15 (d, J = 8.0 Hz, 1 H), 8.34 (d, J = 7.8 Hz, 1 H), 8.58 (d, J = 7.7 Hz, 1 H), 8.61 (s, 1 H), 8.89 (d, J = 0.9 Hz, 1 H) |
| | N-[(2S)-1-(4-{[5-(3-methyl-1,2-oxazol-5-yl)thiophen-2-yl]- | |
| | sulfonyl}piperazin-1-yl)propan-2-yl]-8-[2-(trifluoromethyl)- | |
| | 1,3-thiazol-5-yl]quinazolin-4-amine | |
| 18 | 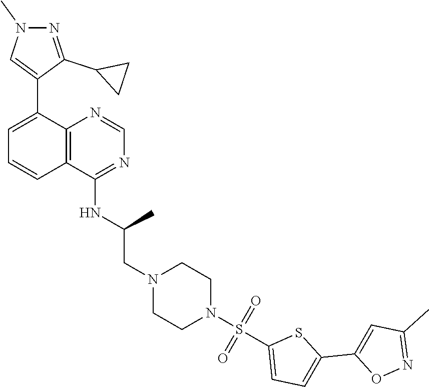 | LC-MS (ESI): m/z (M + 1): 619.2 (Method 2) ¹H NMR (400 MHz, DMSO-d₆) δ ppm 0.68-0.85 (m, 4 H), 1.19 (d, J = 6.6 Hz, 3 H), 1.74-1.86 (m, 1 H), 2.28 (s, 3 H), 2.42 (br dd, J = 12.5, 6.8 Hz, 1 H), 2.55-2.66 (m, 5 H), 2.98 (br s, 4 H), 3.79 (s, 3 H), 4.52-4.66 (m, 1 H), 6.96 (s, 1 H), 7.48 (t, J = 7.9 Hz, 1 H), 7.69 (d, J = 3.9 Hz, 1 H), 7.75 (d, J = 3.9 Hz, 1 H), 7.79 (d, J = 7.9 Hz, 1 H), 7.87 (d, J = 6.6 Hz, 1 H), 7.94 (s, 1 H), 8.12 (d, J = 7.9 Hz, 1 H), 8.44 (s, 1 H) |
| | 8-(3-cyclopropyl-1-methyl-1H-pyrazol-4-yl)-N-[(2S)-1-(4-{[5-(3- | |
| | methyl-1,2-oxazol-5-yl)thiophen-2-yl]sulfonyl}piperazin-1-yl)- | |
| | propan-2-yl]quinazolin-4-amine | |
| 19 | 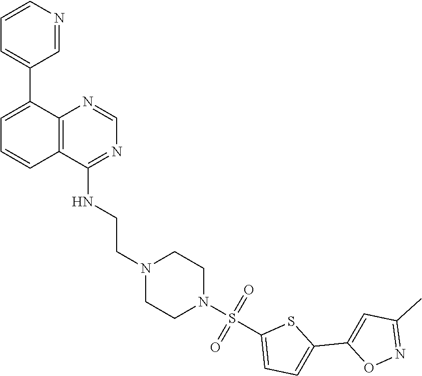 [SMILES: Cc1cc(-c2ccc(S(=O)(=O)N3CCN(CCNc4ncnc5c(-c6cccnc6)cccc45)CC3)s2)on1] | LC-MS (ESI): m/z (M + 1): 562.04 (Method 2) ¹H NMR (400 MHz, DMSO-d₆) δ ppm 2.30 (s, 3 H), 2.56-2.76 (m, 6 H), 3.03 (br s, 4 H), 3.65 (q, J = 6.4 Hz, 2 H), 6.99 (s, 1 H), 7.47 (dd, J = 7.7, 4.8 Hz, 1 H), 7.58 (t, J = 7.8 Hz, 1 H), 7.73 (d, J = 3.9 Hz, 1 H), 7.80 (d, J = 3.9 Hz, 1 H), 7.81-7.87 (m, 1 H), 8.03 (dt, J = 7.8, 1.9 Hz, 1 H), 8.22 (d, J = 8.3 Hz, 1 H), 8.28 (t, J = 5.4 Hz, 1 H), 8.44 (s, 1 H), 8.56 (dd, J = 4.8, 1.3 Hz, 1 H), 8.78 (d, J = 1.8 Hz, 1 H) |
| | N-[2-(4-{[5-(3-methyl-1,2-oxazol-5-yl)thiophen-2-yl]sulfonyl}- | |
| | piperazin-1-yl)ethyl]-8-(pyridin-3-yl)quinazolin-4-amine | |
| 26 | 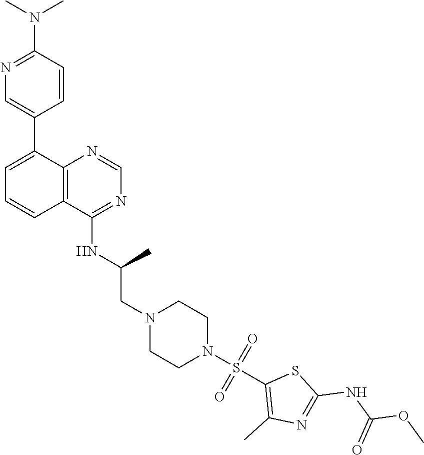 [SMILES: COC(=O)Nc1nc(C)c(S(=O)(=O)N2CCN(C[C@H](C)Nc3ncnc4c(-c5ccc(N(C)C)nc5)cccc34)CC2)s1] | LC-MS (ESI): m/z (M + 1): 626.2 (Method 2) ¹H NMR (500 MHz, DMSO-d₆) δ ppm 1.21 (d, J = 6.6 Hz, 3 H), 2.39-2.67 (m, 6 H), 2.42-2.44 (m, 3 H), 2.99 (br s, 4 H), 3.08 (s, 6 H), 3.67-3.72 (m, 1 H), 3.75 (s, 2 H), 4.60 (dt, J = 14.0, 7.0 Hz, 1 H), 6.71 (d, J = 9.1 Hz, 1 H), 7.51 (t, J = 7.7 Hz, 1 H), 7.74 (dd, J = 7.3, 1.0 Hz, 1 H), 7.81-7.90 (m, 2 H), 8.18 (d, J = 7.7 Hz, 1 H), 8.35 (d, J = 2.5 Hz, 1 H), 8.42 (s, 1 H), 12.10-12.54 (m, 1 H) |
| | methyl N-[5-({4-[(2S)-2-({8-[6-(dimethylamino)pyridin-3-yl]- | |
| | quinazolin-4-yl}amino)propyl]piperazin-1-yl}sulfonyl)-4- | |
| | methyl-1,3-thiazol-2-yl]carbamate | |
| 27 | 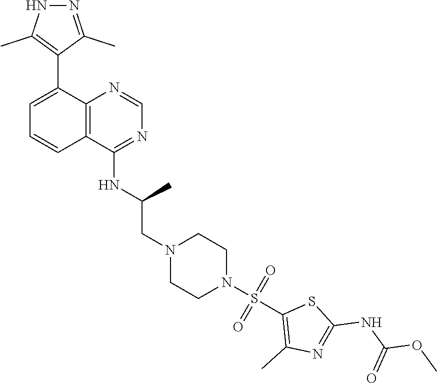 | LC-MS (ESI): m/z (M + 1): 600.1 (Method 2) ¹H NMR (400 MHz, DMSO-d₆) δ ppm 1.21 (d, J = 6.6 Hz, 3 H), 2.01 (br s, 6 H), 2.36-2.46 (m, 1 H), 2.42 (s, 3 H), 2.58 (br s, 4 H), 2.60-2.66 (m, 1 H), 2.99 (br s, 4 H), 3.73 (s, 3 H), 4.50-4.73 (m, 1 H), 7.42-7.52 (m, 1 H), 7.52-7.61 (m, 1 H), 7.80 (d, J = 7.9 Hz, 1 H), 8.19 (dd, J = 8.3, 1.1 Hz, 1 H), 8.38 (s, 1 H), 11.94-12.49 (m, 2 H) |
| | methyl N-[5-({4-[(2S)-2-{[8-(3,5-dimethyl-1H-pyrazol-4-yl)- | |
| | quinazolin-4-yl]amino}propyl]piperazin-1-yl}sulfonyl)-4- | |
| | methyl-1,3-thiazol-2-yl]carbamate | |
| 28 | 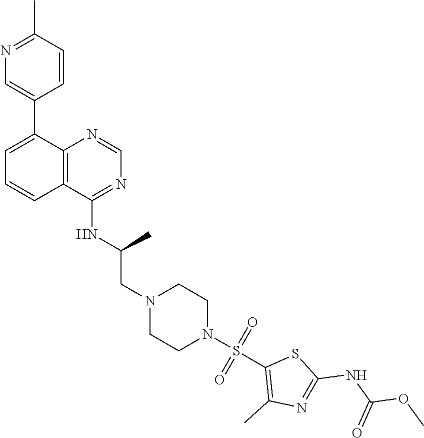 [SMILES: COC(=O)Nc1nc(C)c(S(=O)(=O)N2CCN(C[C@H](C)Nc3ncnc4c(-c5ccc(C)nc5)cccc34)CC2)s1] | LC-MS (ESI): m/z (M + 1): 597.1 (Method 2) ¹H NMR (500 MHz, DMSO-d₆) δ ppm 1.23 (br s, 3 H), 2.43 (s, 4 H), 2.52-2.63 (m, 8 H), 2.88-3.21 (m, 4 H), 3.76 (s, 3 H), 4.34- 5.10 (m, 1 H), 7.36 (br d, J = 7.7 Hz, 1 H), 7.59 (br s, 1 H), 7.76-8.17 (m, 3 H), 8.30 (br d, J = 8.2 Hz, 1 H), 8.44 (br s, 1 H), 8.66 (s, 1 H), 12.34 (br s, 1 H) |
| | methyl N-[4-methyl-5-({4-[(2S)-2-{[8-(6-methylpyridin-3-yl)- | |
| | quinazolin-4-yl]amino}propyl]piperazin-1-yl}sulfonyl)- | |
| | 1,3-thiazol-2-yl]carbamate | |
| 30 | 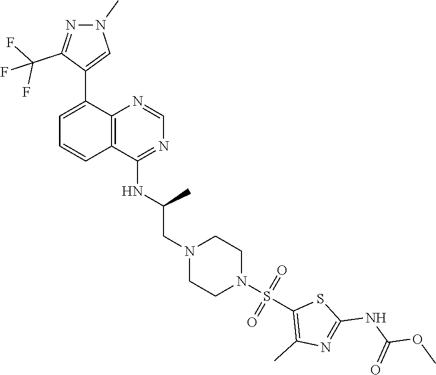 [SMILES: COC(=O)Nc1nc(C)c(S(=O)(=O)N2CCN(C[C@H](C)Nc3ncnc4c(-c5cn(C)nc5C(F)(F)F)cccc34)CC2)s1] | LC-MS (ESI): m/z (M + 1): 654.1 (Method 2) ¹H NMR (400 MHz, DMSO-d₆) δ ppm 1.21 (d, J = 6.6 Hz, 3 H), 2.37-2.46 (m, 1 H), 2.42 (s, 3 H), 2.53-2.67 (m, 1 H), 2.57 (br s, 4 H), 2.99 (br s, 4 H), 3.75 (s, 3 H), 3.99 (s, 3 H), 4.60 (spt, J = 6.8 Hz, 1 H), 7.51 (t, J = 7.8 Hz, 1 H), 7.67 (d, J = 7.1 Hz, 1 H), 7.90 (d, J = 7.9 Hz, 1 H), 8.06 (s, 1 H), 8.26 (d, J = 8.0 Hz, 1 H), 8.40 (s, 1 H), 12.32 (br s, 1 H) |
| | methyl N-[4-methyl-5-({4-[(2S)-2-({8-[1-methyl-3-(trifluoromethyl)- | |
| | 1H-pyrazol-4-yl]quinazolin-4-yl}amino)propyl]piperazin-1-yl}- | |
| | sulfonyl)-1,3-thiazol-2-yl]carbamate | |
| 31 | 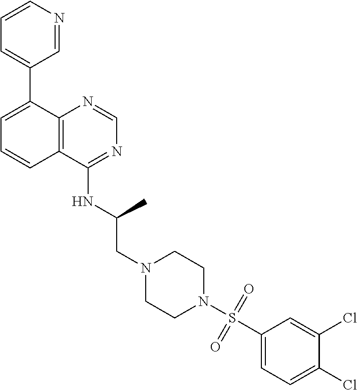 [SMILES: C[C@@H](CN1CCN(S(=O)(=O)c2ccc(Cl)c(Cl)c2)CC1)Nc1ncnc2c(-c3cccnc3)cccc12] | LC-MS (ESI): m/z (M + 1): 557.1 (Method 2) ¹H NMR (400 MHz, DMSO-d₆) δ ppm 1.19 (d, J = 6.6 Hz, 3 H), 2.41 (dd, J = 12.4, 6.7 Hz, 1 H), 2.55 (br t, J = 4.6 Hz, 4 H), 2.62 (dd, J = 12.3, 7.5 Hz, 1 H), 2.93 (br s, 4 H), 4.53-4.69 (m, 1 H), 7.48 (ddd, J = 7.9, 4.8, 0.9 Hz, 1 H), 7.57 (dd, J = 8.3, 7.4 Hz, 1 H), 7.68 (dd, J = 8.5, 2.1 Hz, 1 H), 7.83 (dd, J = 7.3, 1.3 Hz, 1 H), 7.86-7.94 (m, 3 H), 8.03 (dt, J = 7.9, 1.9 Hz, 1 H), 8.30 (dd, J = 8.5, 1.2 Hz, 1 H), 8.42 (s, 1 H), 8.56 (dd, J = 4.7, 1.7 Hz, 1 H), 8.79 (dd, J = 2.3, 0.8 Hz, 1 H) |
| | N-[(2S)-1-[4-(3,4-dichlorobenzenesulfonyl)- | |
| | piperazin-1-yl]propan-2-yl]-8-(pyridin-3-yl)- | |
| | quinazolin-4-amine | |
| 32 | 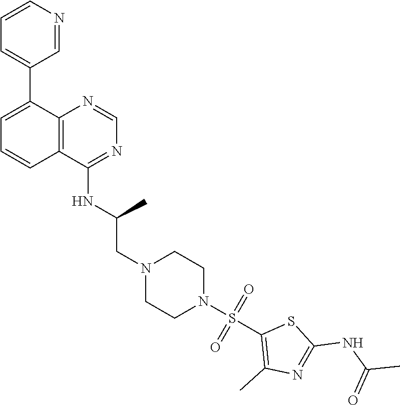 [SMILES: CC(=O)Nc1nc(C)c(S(=O)(=O)N2CCN(C[C@H](C)Nc3ncnc4c(-c5cccnc5)cccc34)CC2)s1] | LC-MS (ESI): m/z (M + 1): 567.1 (Method 2) ¹H NMR (400 MHz, DMSO-d₆) δ ppm 1.21 (d, J = 6.6 Hz, 3 H), 2.15 (s, 3 H), 2.39-2.46 (m, 1 H), 2.45 (s, 3 H), 2.58 (br s, 4 H), 2.64 (dd, J = 12.5, 7.0 Hz, 1 H), 2.98 (br s, 4 H), 4.55-4.67 (m, 1 H), 7.48 (ddd, J = 7.9, 4.8, 0.9 Hz, 1 H), 7.58 (dd, J = 8.3, 7.4 Hz, 1 H), 7.84 (dd, J = 7.3, 1.3 Hz, 1 H), 7.93 (d, J = 8.1 Hz, 1 H), 8.03 (dt, J = 7.9, 2.0 Hz, 1 H), 8.31 (dd, J = 8.5, 1.2 Hz, 1 H), 8.43 (s, 1 H), 8.56 (dd, J = 4.8, 1.8 Hz, 1 H), 8.79 (dd, J = 2.2, 0.9 Hz, 1 H), 12.59 (br s, 1 H) |
| | N-[4-methyl-5-({4-[(2S)-2-{[8-(pyridin-3-yl)quinazolin-4- | |
| | yl]aminopropyl]piperazin-1-yl}sulfonyl)-1,3-thiazol-2- | |
| | yl]acetamide | |
| 34 | 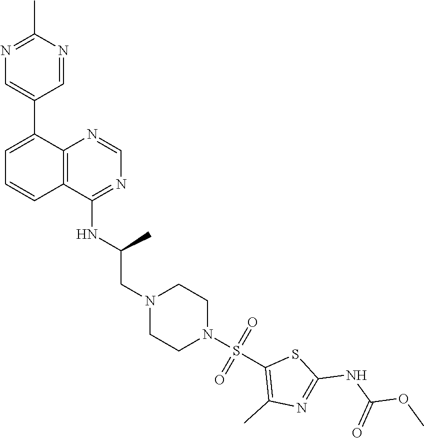 [SMILES: COC(=O)Nc1nc(C)c(S(=O)(=O)N2CCN(C[C@H](C)Nc3ncnc4c(-c5cnc(C)nc5)cccc34)CC2)s1] | LC-MS (ESI): m/z (M + 1): 598.1 (Method 2) ¹H NMR (400 MHz, DMSO-d₆) δ ppm 1.22 (d, J = 6.6 Hz, 3 H), 2.40 (s, 4 H), 2.41-2.47 (m, 1 H), 2.57 (br s, 4 H), 2.63 (br dd, J = 12.4, 7.1 Hz, 1 H), 2.68 (s, 3 H), 2.97 (br s, 4 H), 3.72 (s, 3 H), 4.62 (dt, J = 14.0, 6.9 Hz, 1 H), 7.60 (t, J = 7.8 Hz, 1 H), 7.91 (dd, J = 7.2, 1.0 Hz, 1 H), 7.98 (d, J = 7.9 Hz, 1 H), 8.34 (d, J = 7.6 Hz, 1 H), 8.44 (s, 1 H), 8.94 (s, 2 H), 12.31 (br s, 1 H) |
| | methyl N-[4-methyl-5-({4-[(2S)-2-{[8-(2-methylpyrimidin-5-yl)- | |
| | quinazolin-4-yl]amino}propyl]piperazin-1-yl}sulfonyl)-1,3- | |
| | thiazol-2-yl]carbamate | |
| 35 | 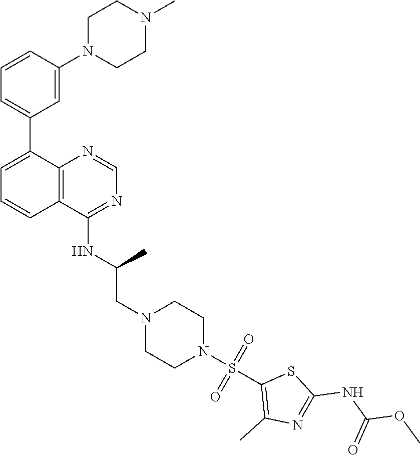 [SMILES: COC(=O)Nc1nc(C)c(S(=O)(=O)N2CCN(C[C@H](C)Nc3ncnc4c(-c5cccc(N6CCN(C)CC6)c5)cccc34)CC2)s1] | LC-MS (ESI): m/z (M + 1): 680.2 (Method 2) ¹H NMR (400 MHz, DMSO-d₆) δ ppm 1.21 (d, J = 6.6 Hz, 3 H), 2.23 (s, 3 H), 2.39-2.45 (m, 1 H), 2.42 (s, 3 H), 2.45-2.53 (m, 4 H), 2.54-2.60 (m, 4 H), 2.60- 2.66 (m, 1 H), 2.99 (br s, 4 H), 3.13-3.19 (m, 4 H), 3.74 (s, 3 H), 4.53-4.66 (m, 1 H), 6.95 (dd, J = 8.3, 1.8 Hz, 1 H), 7.00 (d, J = 7.7 Hz, 1 H), 7.07-7.12 (m, 1 H), 7.26 (t, J = 7.9 Hz, 1 H), 7.51 (t, J = 7.4 Hz, 1 H), 7.73 (dd, J = 7.2, 1.1 Hz, 1 H), 7.85 (d, J = 7.9 Hz, 1 H), 8.23 (dd, J = 8.3, 1.1 Hz, 1 H), 8.40 (s, 1 H), 12.29 (br s, 1 H) |
| | methyl N-[4-methyl-5-({4-[(2S)-2-({8-[3-(4-methylpiperazin-1-yl)- | |
| | phenyl]quinazolin-4-yl}amino)propyl]piperazin-1-yl}sulfonyl)- | |
| | 1,3-thiazol-2-yl]carbamate | |
| 36 | 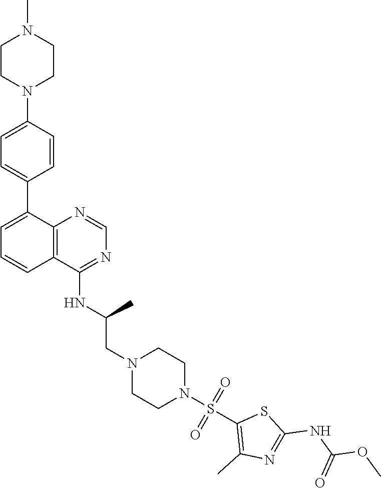 [SMILES: COC(=O)Nc1nc(C)c(S(=O)(=O)N2CCN(C[C@H](C)Nc3ncnc4c(-c5ccc(N6CCN(C)CC6)cc5)cccc34)CC2)s1] | LC-MS (ESI): m/z (M + 1): 680.2 (Method 2) ¹H NMR (500 MHz, DMSO-d₆) δ ppm 1.21 (d, J = 6.3 Hz, 3 H), 2.24 (s, 3 H), 2.40-2.46 (m, 1 H), 2.42 (s, 3 H), 2.46-2.53 (m, 4 H), 2.53-2.67 (m, 5 H), 2.94- 3.07 (m, 4 H), 3.16-3.24 (m, 4 H), 3.74 (s, 3 H), 4.54-4.67 (m, 1 H), 7.00 (d, J = 9.1 Hz, 2 H), 7.47-7.54 (m, 3 H), 7.70 (dd, J = 7.1, 1.1 Hz, 1 H), 7.81 (d, J = 8.0 Hz, 1 H), 8.17 (dd, J = 8.4, 1.0 Hz, 1 H), 8.41 (s, 1 H), 12.31 (br s, 1 H) |
| | methyl N-[4-methyl-5-({4-[(2S)-2-({8-[4-(4-methylpiperazin- | |
| | 1-yl)phenyl]quinazolin-4-yl}amino)propyl]piperazin-1-yl}- | |
| | sulfonyl)-1,3-thiazol-2-yl]carbamate | |
| 37 | 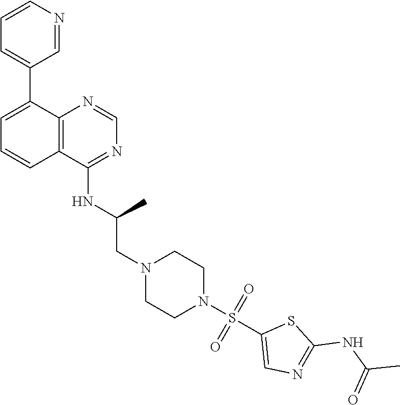 [SMILES: CC(=O)Nc1ncc(S(=O)(=O)N2CCN(C[C@H](C)Nc3ncnc4c(-c5cccnc5)cccc34)CC2)s1] | LC-MS (ESI): m/z (M + 1): 553.1 (Method 2) ¹H NMR (400 MHz, DMSO-d₆) δ ppm 1.21 (d, J = 6.6 Hz, 3 H), 2.17 (s, 3 H), 2.43 (dd, J = 12.4, 6.8 Hz, 1 H), 2.58 (br s, 4 H), 2.61-2.68 (m, 1 H), 2.93 (br s, 4 H), 4.61 (dt, J = 14.0, 6.9 Hz, 1 H), 7.48 (ddd, J = 7.8, 4.8, 0.8 Hz, 1 H), 7.58 (dd, J = 8.3, 7.4 Hz, 1 H), 7.83 (dd, J = 7.3, 1.2 Hz, 1 H), 7.90-7.96 (m, 1 H), 7.93 (s, 1 H), 8.03 (dt, J = 7.9, 2.0 Hz, 1 H), 8.31 (dd, J = 8.5, 1.1 Hz, 1 H), 8.43 (s, 1 H), 8.56 (dd, J = 4.7, 1.7 Hz, 1 H), 8.78 (dd, J = 2.3, 0.7 Hz, 1 H), 12.70 (br s, 1 H) |
| | N-[5-({4-[(2S)-2-{[8-(pyridin-3-yl)quinazolin-4-yl]amino}- | |
| | propyl]piperazin-1-yl}sulfonyl)-1,3-thiazol-2-yl]acetamide | |
| 38 | 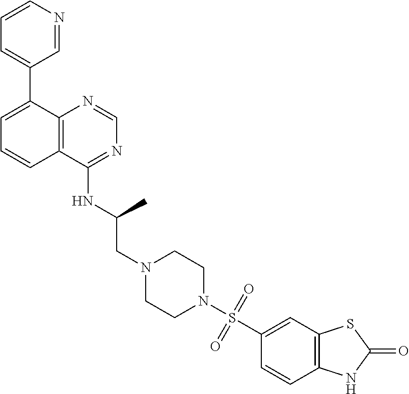 [SMILES: C[C@@H](CN1CCN(S(=O)(=O)c2ccc3[nH]c(=O)sc3c2)CC1)Nc1ncnc2c(-c3cccnc3)cccc12] | LC-MS (ESI): m/z (M + 1): 562.1 (Method 2) ¹H NMR (400 MHz, DMSO-d₆) δ ppm 1.18 (d, J = 6.6 Hz, 3 H), 2.41 (br dd, J = 12.3, 6.4 Hz, 1 H), 2.55 (br s, 5 H), 2.86 (br s, 4 H), 4.60 (dt, J = 13.9, 7.0 Hz, 1 H), 7.27 (d, J = 8.3 Hz, 1 H), 7.48 (dd, J = 7.9, 4.8 Hz, 1 H), 7.52-7.66 (m, 2 H), 7.83 (dd, J = 7.1, 1.0 Hz, 1 H), 7.92 (d, J = 7.9 Hz, 1 H), 7.99-8.10 (m, 2 H), 8.28 (d, J = 7.5 Hz, 1 H), J = 7.9 Hz, 1 H), 7.99- 8.10 (m, 8.42 (s, 1 H), 8.56 (dd, J = 4.8, 1.8 Hz, 1 H), 8.78 (d, J = 1.5 Hz, 1 H), 12.17-12.66 (m, 1 H) |
| | 6-({4-[(2S)-2-{[8-(pyridin-3-yl)quinazolin-4-yl]amino}propyl]- | |
| | piperazin-1-yl}sulfonyl)-2,3-dihydro-1,3-benzothiazol-2-one | |
| 40 | 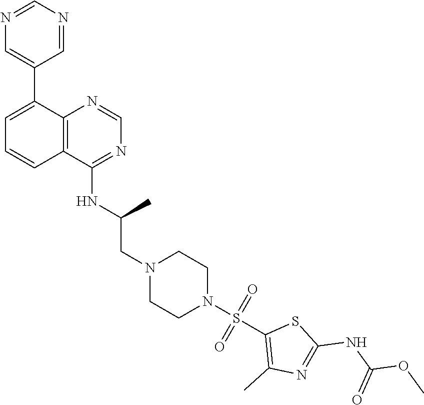 [SMILES: COC(=O)Nc1nc(C)c(S(=O)(=O)N2CCN(C[C@H](C)Nc3ncnc4c(-c5cncnc5)cccc34)CC2)s1] | LC-MS (ESI): m/z (M + 1): 584.1 (Method 2) ¹H NMR (400 MHz, DMSO-d₆) δ ppm 1.22 (d, J = 6.6 Hz, 3 H), 2.35- 2.48 (m, 4 H), 2.54-2.66 (m, 5 H), 2.97 (br s, 4 H), 3.70 (br s, 3 H), 4.62 (dt, J = 13.6, 7.0 Hz, 1 H), 7.62 (dd, J = 8.3, 7.4 Hz, 1 H), 7.95 (dd, J = 7.4, 1.2 Hz, 1 H), 8.00 (d, J = 7.9 Hz, 1 H), 8.37 (dd, J = 8.5, 1.2 Hz, 1 H), 8.46 (s, 1 H), 9.07 (s, 2 H), 9.18 (s, 1 H), 12.15-12.51 (m, 1 H) |
| | methyl N-[4-methyl-5-({4-[(2S)-2-{[8-(pyrimidin-5-yl)quinazolin-4- | |
| | yl]amino}propyl]piperazin-1-yl}sulfonyl)-1,3-thiazol-2-yl]carbamate | |
| 41 | 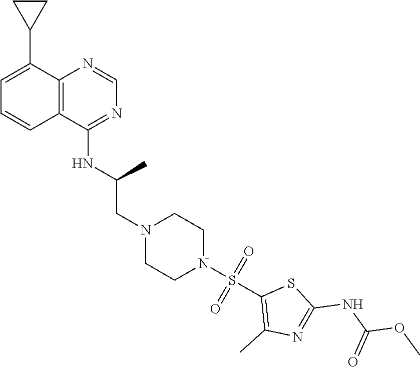 [SMILES: COC(=O)Nc1nc(C)c(S(=O)(=O)N2CCN(C[C@H](C)Nc3ncnc4c(C5CC5)cccc34)CC2)s1] | LC-MS (ESI): m/z (M + 1): 546.2 (Method 2) ¹H NMR (400 MHz, DMSO-d₆) δ ppm 0.68-0.84 (m, 2 H), 0.98-1.09 (m, 2 H), 1.19 (d, J = 6.6 Hz, 3 H), 2.37-2.45 (m, 4 H), 2.53- 2.65 (m, 5 H), 2.88-3.11 (m, 5 H), 3.76 (s, 3 H), 4.59 (dt, J = 13.5, 6.7 Hz, 1 H), 7.19 (d, J = 7.2 Hz, 1 H), 7.34 (t, J = 7.8 Hz, 1 H), 7.72 (d, J = 7.9 Hz, 1 H), 8.00 (d, J = 7.9 Hz, 1 H), 8.48 (s, 1 H), 12.33 (br s, 1 H) |
| | methyl N-[5-({4-[(2S)-2-[(8-cyclopropylquinazolin-4-yl)- | |
| | amino]propyl]piperazin-1-yl}sulfonyl)-4-methyl-1,3- | |
| | thiazol-2-yl]carbamate | |
| 42 | 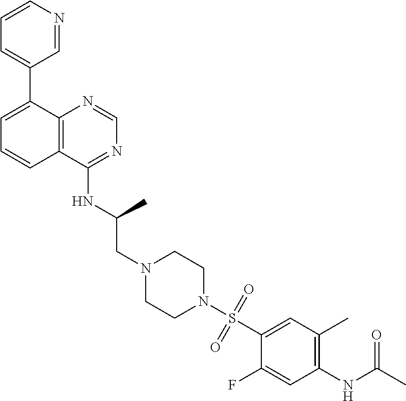 [SMILES: CC(=O)Nc1cc(F)c(S(=O)(=O)N2CCN(C[C@H](C)Nc3ncnc4c(-c5cccnc5)cccc34)CC2)cc1C] | LC-MS (ESI): m/z (M + 1): 578.2 (Method 2) ¹H NMR (400 MHz, DMSO-d₆) δ ppm 1.20 (d, J = 6.6 Hz, 3 H), 2.14 (s, 3 H), 2.27 (s, 3 H), 2.42 (dd, J = 12.3, 6.8 Hz, 1 H), 2.54 (br s, 4 H), 2.61 (dd, J = 12.4, 7.2 Hz, 1 H), 2.97 (br s, 4 H), 4.61 (dt, J = 14.2, 7.0 Hz, 1 H), 7.48 (ddd, J = 7.8, 4.8, 0.8 Hz, 1 H), 7.53 (d, J = 7.9 Hz, 1 H), 7.58 (dd, J = 8.1, 7.5 Hz, 1 H), 7.77-7.96 (m, 3 H), 7.99-8.07 (m, 1 H), 8.31 (dd, J = 8.6, 1.1 Hz, 1 H), 8.43 (s, 1 H), 8.56 (dd, J = 4.8, 1.8 Hz, 1 H), 8.78 (dd, J = 2.2, 0.7 Hz, 1 H), 9.44 (s, 1 H) |
| | N-[5-fluoro-2-methyl-4-({4-[(2S)-2-{[8-(pyridin-3-yl)quinazolin- | |
| | 4-yl]amino}propyl]piperazin-1-yl}sulfonyl)phenyl]acetamide | |
| 43 | 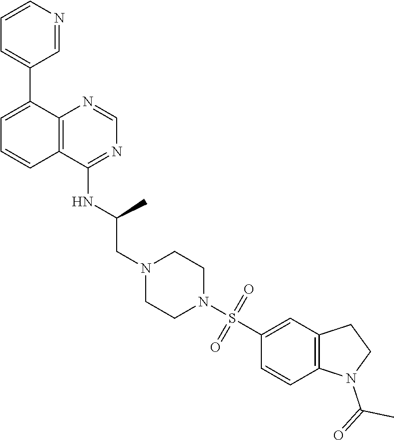 [SMILES: CC(=O)N1CCc2cc(S(=O)(=O)N3CCN(C[C@H](C)Nc4ncnc5c(-c6cccnc6)cccc45)CC3)ccc21] | LC-MS (ESI): m/z (M + 1): 572.2 (Method 2) ¹H NMR (400 MHz, DMSO-d₆) δ ppm 1.18 (d, J = 6.6 Hz, 3 H), 2.17 (s, 3 H), 2.39 (dd, J = 12.5, 6.6 Hz, 1 H), 2.52-2.58 (m, 4 H), 2.61 (dd, J = 12.5, 7.5 Hz, 1 H), 2.82 (br s, 4 H), 3.19 (br t, J = 8.4 Hz, 2 H), 4.13 (br t, J = 8.6 Hz, 2 H), 4.59 (spt, J = 6.7 Hz, 1 H), 7.45-7.53 (m, 3 H), 7.57 (dd, J = 8.3, 7.4 Hz, 1 H), 7.83 (dd, J = 7.3, 1.3 Hz, 1 H), 7.91 (d, J = 7.9 Hz, 1 H), 8.02 (dt, J = 7.9, 2.0 Hz, 1 H), 8.16 (br d, J = 8.4 Hz, 1 H), 8.29 (dd, J = 8.5, 1.2 Hz, 1 H), 8.42 (s, 1 H), 8.56 (dd, J = 4.8, 1.8 Hz, 1 H), 8.78 (dd, J = 2.2, 0.9 Hz, 1 H) |
| | 1-[5-({4-[(2S)-2-{[8-(pyridin-3-yl)quinazolin-4-yl]amino}- | |
| | propyl]piperazin-1-yl}sulfonyl)-2,3-dihydro-1H-indol-1- | |
| | yl]ethan-1-one | |
| 44 | 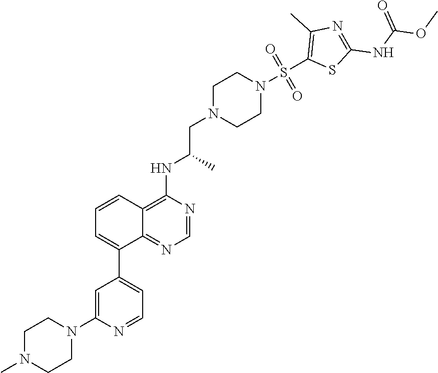 | LC-MS (ESI): m/z (M + 1): 680.8 (Method 3) ¹H NMR (400 MHz, DMSO-d₆) δ ppm 8.39 (s, 1 H), 8.26 (d, J = 7.89 Hz, 1 H), 8.08-8.15 (m, 2 H), 7.87 (br d, J = 7.89 Hz, 1 H), 7.75 (d, J = 7.02 Hz, 1 H), 7.51 (t, J = 7.78 Hz, 1 H), 6.93 (s, 1 H), 6.84 (d, J = 5.04 Hz, 1 H), 4.52-4.63 (m, 1 H), 3.7 (s, 3 H), 3.42-3.53 (m, 4 H), 2.96 (br s, 4 H), 2.39-2.63 (m, 11 H), 2.20 (s, 3 H), 2.07 (s, 1H), 1.18 (d, J = 6.58 Hz, 3 H) |
| | methyl N-[4-methyl-5-({4-[(2S)-2-({8-[2-(4-methylpiperazin-1- | |
| | yl)pyridin-4-yl]quinazolin-4-yl}amino)propyl]piperazin-1-yl}- | |
| | sulfonyl)-1,3-thiazol-2-yl]carbamate | |
| 48 | 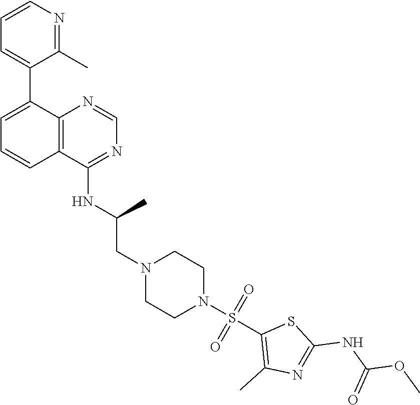 [SMILES: COC(=O)Nc1nc(C)c(S(=O)(=O)N2CCN(C[C@H](C)Nc3ncnc4c(-c5cccnc5C)cccc34)CC2)s1] | LC-MS (ESI): m/z (M + 1): 597.2 (Method 2) ¹H NMR (400 MHz, DMSO-d₆) δ ppm 1.22 (d, J = 6.6 Hz, 3 H), 2.15 (s, 3 H), 2.38-2.47 (m, 1 H), 2.42 (s, 3 H), 2.54-2.67 (m, 5 H), 2.99 (br s, 4 H), 3.75 (s, 3 H), 4.61 (dt, J = 14.0, 7.0 Hz, 1 H), 7.28 (dd, J = 7.6, 5.0 Hz, 1 H), 7.49-7.60 (m, 2 H), 7.61- 7.71 (m, 1 H), 7.92 (d, J = 7.7 Hz, 1 H), 8.32 (dd, J = 8.4, 1.3 Hz, 1 H), 8.35 (s, 1 H), 8.46 (dd, J = 4.8, 1.8 Hz, 1 H), 12.32 (br s, 1 H) |
| | methyl N-[4-methyl-5-({4-[(2S)-2-{[8-(2-methylpyridin-3-yl) | |
| | quinazolin-4-yl]amino}propyl]piperazin-1-yl}sulfonyl)-1,3- | |
| | thiazol-2-yl]carbamate | |
| 51 | 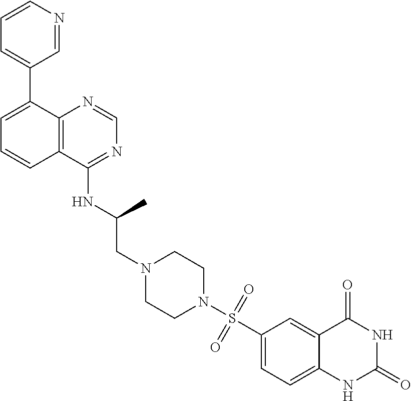 [SMILES: C[C@@H](CN1CCN(S(=O)(=O)c2ccc3[nH]c(=O)[nH]c(=O)c3c2)CC1)Nc1ncnc2c(-c3cccnc3)cccc12] | LC-MS (ESI): m/z (M + 1): 573.1 (Method 2) ¹H NMR (400 MHz, DMSO-d₆) δ ppm 1.18 (d, J = 6.5 Hz, 3 H), 2.40 (dd, J = 12.5, 6.6 Hz, 1 H), 2.55 (br s, 4 H), 2.62 (dd, J = 12.3, 7.4 Hz, 1 H), 2.87 (br s, 4 H), 4.59 (dt, J = 13.9, 6.7 Hz, 1 H), 7.31 (d, J = 8.7 Hz, 1 H), 7.47 (ddd, J = 7.9, 4.8, 0.7 Hz, 1 H), 7.56 (dd, J = 8.2, 7.4 Hz, 1 H), 7.82 (dd, J = 7.2, 1.2 Hz, 1 H), 7.86-7.95 (m, 2 H), 8.02 (dt, J = 7.9, 1.9 Hz, 1 H), 8.09 (d, J = 2.2 Hz, 1 H), 8.28 (dd, J = 8.5, 1.1 Hz, 1 H), 8.42 (s, 1 H), 8.56 (dd, J = 4.8, 1.6 Hz, 1 H), 8.78 (dd, J = 2.3, 0.7 Hz, 1 H), 11.54 (br s, 2 H) |
| | 6-({4-[(2S)-2-{[8-(pyridin-3-yl)quinazolin-4-yl]amino}propyl]- | |
| | piperazin-1-yl}sulfonyl)-1,2,3,4-tetrahydroquinazoline-2,4-dione | |
| 52 | 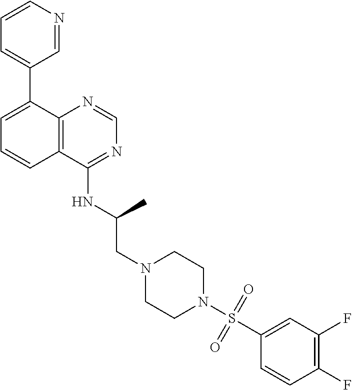 [SMILES: C[C@@H](CN1CCN(S(=O)(=O)c2ccc(F)c(F)c2)CC1)Nc1ncnc2c(-c3cccnc3)cccc12] | LC-MS (ESI): m/z (M + 1): 525.2 (Method 2) ¹H NMR (400 MHz, DMSO-d₆) δ ppm 1.19 (d, J = 6.6 Hz, 3 H), 2.41 (dd, J = 12.4, 6.6 Hz, 1 H), 2.54 (br t, J = 4.5 Hz, 4 H), 2.62 (dd, J = 12.5, 7.5 Hz, 1 H), 2.90 (br s, 4 H), 4.61 (dt, J = 14.0, 7.0 Hz, 1 H), 7.48 (ddd, J = 7.9, 4.8, 0.8 Hz, 1 H), 7.54-7.63 (m, 2 H), 7.69 (dt, J = 10.1, 8.2 Hz, 1 H), 7.76-7.86 (m, 2 H), 7.92 (d, J = 8.0 Hz, 1 H), 8.02 (dt, J = 8.0, 1.9 Hz, 1 H), 8.30 (dd, J = 8.4, 1.1 Hz, 1 H), 8.42 (s, 1 H), 8.56 (dd, J = 4.8, 1.6 Hz, 1 H), 8.78 (dd, J = 2.3, 0.7 Hz, 1 H) |
| | N-[(2S)-1-[4-(3,4-difluorobenzenesulfonyl)piperazin- | |
| | 1-yl]propan-2-yl]-8-(pyridin-3-yl)quinazolin-4-amine | |
| 56 | 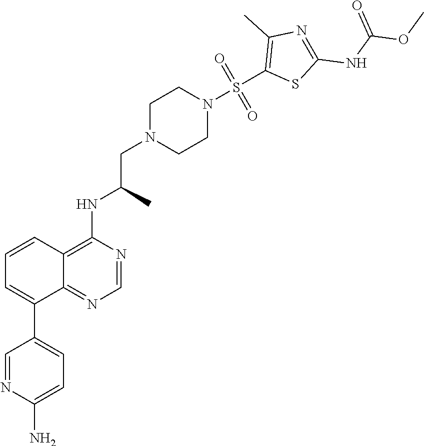 [SMILES: COC(=O)Nc1nc(C)c(S(=O)(=O)N2CCN(C[C@@H](C)Nc3ncnc4c(-c5ccc(N)nc5)cccc34)CC2)s1] | LC-MS (ESI): m/z (M + 1): 598.7 (Method 3) ¹H NMR (400 MHz, d6-Acetone) δ ppm 8.59 (s, 2H), 8.41-8.49 (m, 1H), 8.02 (d, J = 7.45 Hz, 1H), 7.70-7.83 (m, 1H), 7.38- 7.56 (m, 1H), 7.12-7.25 (m, 1H), 5.89-6.10 (m, 2H), 4.54- 4.77 (m, 1H), 3.75-3.84 (m, 3H), 2.98-3.13 (m, 5H), 2.65 (m, 6H), 2.40 (s, 3H), 1.26- 1.31 (m, 3H) |
| | methyl N-[5-({4-[(2S)-2-{[8-(6-aminopyridin-3-yl)quinazolin-4- | |
| | yl]amino}propyl]piperazin-1-yl}sulfonyl)-4-methyl-1,3-thiazol- | |
| | 2-yl]carbamate | |
| 57 | 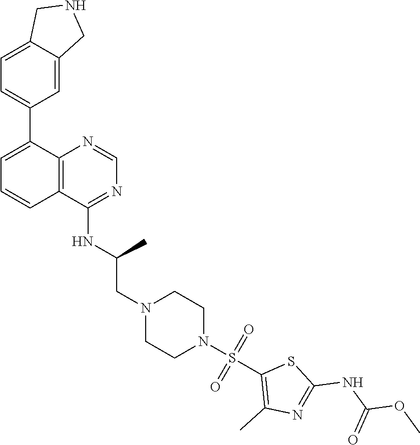 [SMILES: COC(=O)Nc1nc(C)c(S(=O)(=O)N2CCN(C[C@H](C)Nc3ncnc4c(-c5ccc6c(c5)CNC6)cccc34)CC2)s1] | LC-MS (ESI): m/z (M + 1): 623.2 (Method 2) ¹H NMR (400 MHz, DMSO-d₆) δ ppm 1.21 (d, J = 6.6 Hz, 3 H), 2.38 (s, 3 H), 2.43 (br dd, J = 12.3, 7.0 Hz, 1 H), 2.53-2.65 (m, 5 H), 2.97 (br s, 4 H), 3.67 (s, 3 H), 4.27 (s, 4 H), 4.56-4.65 (m, 1 H), 7.36 (d, J = 7.9 Hz, 1 H), 7.41-7.47 (m, 1 H), 7.49-7.58 (m, 1 H), 7.51 (s, 1 H), 7.72 (dd, J = 7.1, 0.8 Hz, 1 H), 7.87 (br d, J = 7.9 Hz, 1 H), 9.17 (br s, 1 H), 8.25 (d, J = 7.9 Hz, 1 H), 8.41 (s, 1 H) |
| | methyl N-[5-({4-[(2S)-2-{[8-(2,3-dihydro-1H-isoindol-5-yl)- | |
| | quinazolin-4-yl]amino}propyl]piperazin-1-yl}sulfonyl)-4- | |
| | methyl-1,3-thiazol-2-yl]carbamate | |
| 58 | 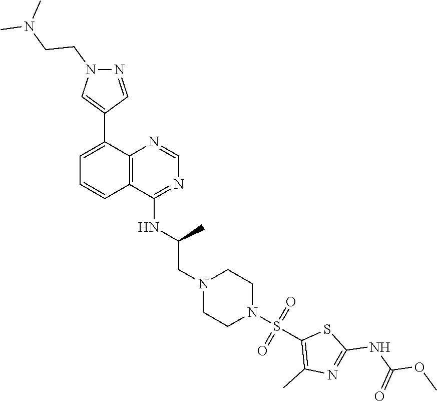 | LC-MS (ESI): m/z (M + 1): 643.2 (Method 2) ¹H NMR (400 MHz, DMSO-d₆) δ ppm 1.21 (d, J = 6.3 Hz, 3 H), 2.19 (s, 6 H), 2.38-2.46 (m, 4 H), 2.53-2.65 (m, 5 H), 2.71 (t, J = 6.7 Hz, 2 H), 2.98 (br s, 4 H), 3.73 (s, 3 H), 4.26 (t, J = 6.5 Hz, 2 H), 4.55-4.67 (m, 1 H), 7.45 (t, J = 7.8 Hz, 1 H), 7.79 (d, J = 7.8 Hz, 1 H), 8.01 (d, J = 7.0 Hz, 1 H), 8.07 (d, J = 8.2 Hz, 1 H), 8.15 (s, 1 H), 8.51 (s, 1 H), 8.61 (s, 1 H), 11.60-12.89 (m, 1 H) |
| | methyl N-[5-({4-[(2S)-2-[(8-{1-[2-(dimethylamino)ethyl]-1H-pyrazol- | |
| | 4-yl}quinazolin-4-yl)amino]propyl]piperazin-1-yl}sulfonyl)-4- | |
| | methyl-1,3-thiazol-2-yl]carbamate | |
| 59 | 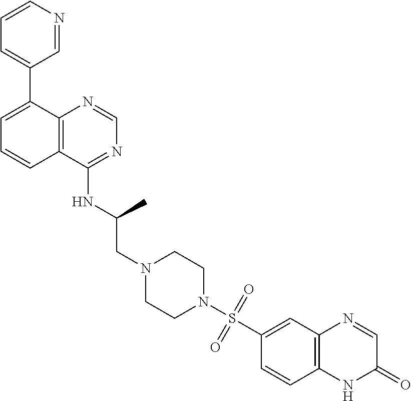 [SMILES: C[C@@H](CN1CCN(S(=O)(=O)c2ccc3[nH]c(=O)cnc3c2)CC1)Nc1ncnc2c(-c3cccnc3)cccc12] | LC-MS (ESI): m/z (M + 1): 557.2 (Method 2) ¹H NMR (400 MHz, DMSO-d₆) δ ppm 1.17 (d, J = 6.5 Hz, 3 H), 2.39 (dd, J = 12.4, 6.7 Hz, 1 H), 2.54 (br s, 4 H), 2.61 (dd, J = 12.4, 7.4 Hz, 1 H), 2.89 (br s, 4 H), 4.59 (dt, J = 13.9, 7.1 Hz, 1 H), 7.43 (d, J = 8.7 Hz, 1 H), 7.47 (ddd, J = 7.9, 4.8, 0.8 Hz, 1 H), 7.55 (dd, J = 8.2, 7.4 Hz, 1 H), 7.74-7.85 (m, 2 H), 7.90 (d, J = 8.0 Hz, 1 H), 7.98 (d, J = 2.0 Hz, 1 H), 8.01 (dt, J = 8.0, 1.9 Hz, 1 H), 8.21 (s, 1 H), 8.27 (dd, J = 8.4, 1.2 Hz, 1 H), 8.41 (s, 1 H), 8.56 (dd, J = 4.8, 1.7 Hz, 1 H), 8.77 (dd, J = 2.3, 0.7 Hz, 1 H), 12.68 (br s, 1 H) |
| | 6-({4-[(2S)-2-{[8-(pyridin-3-yl)quinazolin-4-yl]amino}propyl]- | |
| | piperazin-1-yl}sulfonyl)-1,2-dihydroquinoxalin-2-one | |
| 61 | 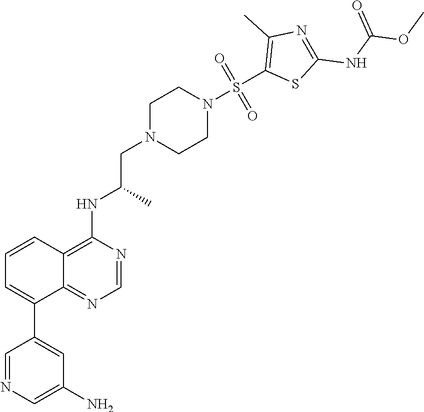 [SMILES: COC(=O)Nc1nc(C)c(S(=O)(=O)N2CCN(C[C@H](C)Nc3ncnc4c(-c5cncc(N)c5)cccc34)CC2)s1] | LC-MS (ESI): m/z (M + 1): 597.8 (Method 3) ¹H NMR (400 MHz, DMSO-d₆) δ ppm 8.43 (s, 1 H) 8.27 (d, J = 7.67 Hz, 1 H) 8.15 (s, 2 H) 7.86- 7.95 (m, 3 H) 7.73 (d, J = 6.98 Hz, 1 H) 7.54 (t, J = 7.78 Hz, 1 H) 7.16 (t, J = 2.19 Hz, 1 H) 4.61 (spt, J = 6.76 Hz, 1 H) 3.75 (s, 3 H) 2.95-3.05 (m, 4 H) 2.54-2.68 (m, 6 H) 2.43 (s, 3 H) 1.21 (d, J = 6.58 Hz, 3 H) |
| | methyl N-[5-({4-[(2S)-2-{[8-(5-aminopyridin-3-yl)quinazolin- | |
| | 4-yl]amino}propyl]piperazin-1-yl}sulfonyl)-4-methyl-1,3- | |
| | thiazol-2-yl]carbamate | |
| 63 | 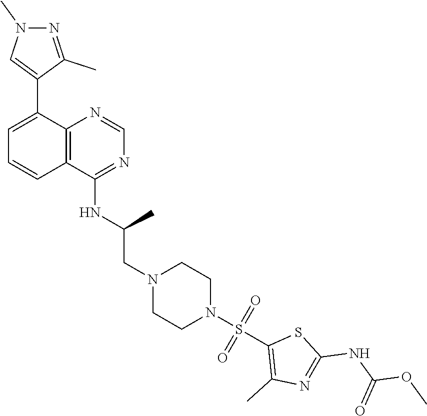 | LC-MS (ESI): m/z (M + 1): 600.1 (Method 2) ¹H NMR (500 MHz, DMSO-d₆) δ ppm 1.20 (d, J = 6.6 Hz, 3 H), 2.17 (s, 3 H), 2.39-2.46 (m, 4 H), 2.53-2.59 (m, 4 H), 2.62 (br dd, J = 12.4, 6.9 Hz, 1 H), 2.99 (br s, 4 H), 3.75 (s, 3 H), 3.82 (s, 3 H), 4.54-4.66 (m, 1 H), 7.47 (t, J = 7.8 Hz, 1 H), 7.68 (dd, J = 7.1, 1.1 Hz, 1 H), 7.81 (d, J = 8.0 Hz, 1 H), 7.93 (s, 1 H), 8.10-8.19 (m, 1 H), 8.43 (s, 1 H), 12.33 (br s, 1 H) |
| | methyl N-[5-({4-[(2S)-2-{[8-(1,3-dimethyl-1H-pyrazol-4-yl)- | |
| | quinazolin-4-yl]amino}propyl]piperazin-1-yl}sulfonyl)-4- | |
| | methyl-1,3-thiazol-2-yl]carbamate | |
| 64 | 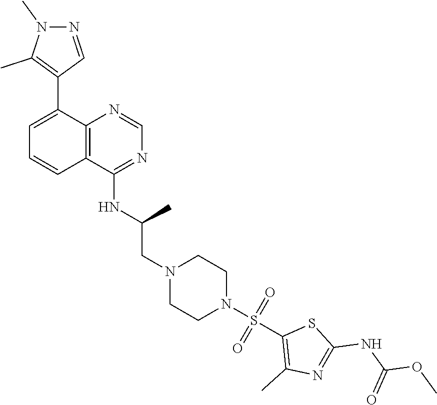 | LC-MS (ESI): m/z (M + 1): 600.2 (Method 2) ¹H NMR (500 MHz, DMSO-d₆) δ ppm 1.21 (d, J = 6.6 Hz, 3 H), 2.20 (s, 3 H), 2.39-2.45 (m, 1 H), 2.42 (s, 3 H), 2.53-2.65 (m, 1 H), 2.57 (br s, 4 H), 2.99 (br s, 4 H), 3.75 (s, 3 H), 3.80 (s, 3 H), 4.60 (spt, J = 6.9 Hz, 1 H), 7.48 (t, J = 7.8 Hz, 1 H), 7.54 (s, 1 H), 7.61 (dd, J = 7.1, 1.1 Hz, 1 H), 7.81 (d, J = 8.0 Hz, 1 H), 8.16 (dd, J = 8.3, 0.9 Hz, 1 H), 8.41 (s, 1 H), 12.33 (br s, 1 H) |
| | methyl N-[5-({4-[(2S)-2-{[8-(1,5-dimethyl-1H-pyrazol-4-yl)- | |
| | quinazolin-4-yl]amino}propyl]piperazin-1-yl}sulfonyl)-4- | |
| | methyl-1,3-thiazol-2-yl]carbamate | |
| 65 | 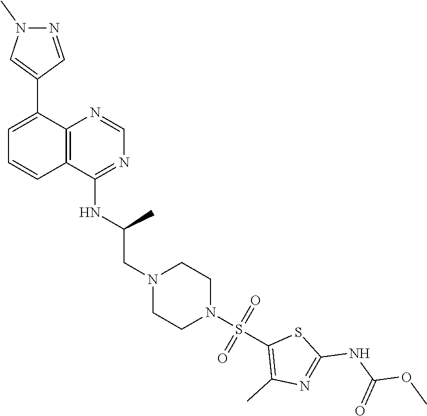 | LC-MS (ESI): m/z (M + 1): 586.2 (Method 2) ¹H NMR (400 MHz, DMSO-d₆) δ ppm 1.21 (d, J = 6.6 Hz, 3 H), 2.38-2.46 (m, 4 H), 2.53-2.66 (m, 5 H), 2.99 (br s, 4 H), 3.75 (s, 3 H), 3.91 (s, 3 H), 4.61 (dt, J = 13.8, 6.8 Hz, 1 H), 7.45 (t, J = 7.9 Hz, 1 H), 7.80 (d, J = 7.9 Hz, 1 H), 8.01 (dd, J = 7.5, 0.9 Hz, 1 H), 8.08 (d, J = 8.1 Hz, 1 H), 8.13 (s, 1 H), 8.51 (s, 1 H), 8.58 (s, 1 H), 12.32 (br s, 1 H) |
| | methyl N-[4-methyl-5-({4-[(2S)-2-{[8-(1-methyl-1H-pyrazol-4- | |
| | yl)quinazolin-4-yl]amino}propyl]piperazin-1-yl}sulfonyl)- | |
| | 1,3-thiazol-2-yl]carbamate | |
| 66 | 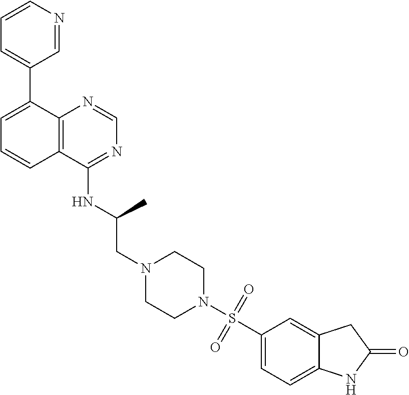 [SMILES: C[C@@H](CN1CCN(S(=O)(=O)c2ccc3c(c2)CC(=O)N3)CC1)Nc1ncnc2c(-c3cccnc3)cccc12] | LC-MS (ESI): m/z (M + 1): 544.2 (Method 2) ¹H NMR (400 MHz, DMSO-d₆) δ ppm 1.18 (d, J = 6.5 Hz, 3 H), 2.39 (dd, J = 12.3, 6.5 Hz, 1 H), 2.54 (br s, 4 H), 2.62 (dd, J = 12.3, 7.4 Hz, 1 H), 2.83 (br s, 4 H), 3.57 (s, 2 H), 4.54-4.65 (m, 1 H), 6.97 (d, J = 8.1 Hz, 1 H), 7.42-7.63 (m, 4 H), 7.83 (d, J = 7.2 Hz, 1 H), 7.92 (d, J = 7.9 Hz, 1 H), 8.02 (dt, J = 7.8, 1.9 Hz, 1 H), 8.29 (d, J = 7.6 Hz, 1 H), 8.42 (s, 1 H), 8.56 (dd, J = 4.8, 1.6 Hz, 1 H), 8.78 (d, J = 2.1 Hz, 1 H), 10.79 (s, 1 H) |
| | 5-({4-[(2S)-2-{[8-(pyridin-3-yl)quinazolin-4-yl]amino}propyl]- | |
| | piperazin-1-yl}sulfonyl)-2,3-dihydro-1H-indol-2-one | |
| 67 | 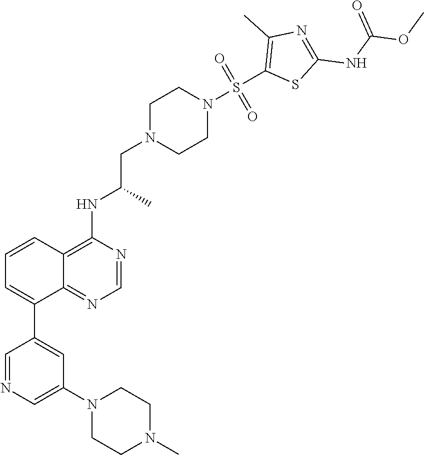 [SMILES: COC(=O)Nc1nc(C)c(S(=O)(=O)N2CCN(C[C@H](C)Nc3ncnc4c(-c5cncc(N6CCN(C)CC6)c5)cccc34)CC2)s1] | LC-MS (ESI): m/z (M + 1): 680.8 (Method 3) ¹H NMR (400 MHz, DMSO-d₆) δ ppm 8.39 (s, 1 H), 8.33-8.35 (m, 1 H), 8.17 (br d, J = 7.67 Hz, 1 H), 8.12 (s, 2 H), 7.79-7.87 (m, 2 H), 7.69-7.75 (m, 1 H), 7.48 (t, J = 7.78 Hz, 1 H), 6.87 (d, J = 8.77 Hz, 1 H), 4.52- 4.62 (m, 1 H), 3.72 (s, 3 H), 3.47-3.63 (m, 4 H), 2.92-3.06 (m, 6 H), 2.48-2.64 (m, 4 H), 2.37-2.43 (m, 7 H), 2.21 (s, 3 H), 1.18 (d, J = 6.36 Hz, 3 H) |
| | methyl N-[4-methyl-5-({4-[(2S)-2-({8-[5-(4-methylpiperazin- | |
| | 1-yl)pyridin-3-yl]quinazolin-4-yl}amino)propyl]piperazin-1- | |
| | yl}sulfonyl)-1,3-thiazol-2-yl]carbamate | |
| 68 | 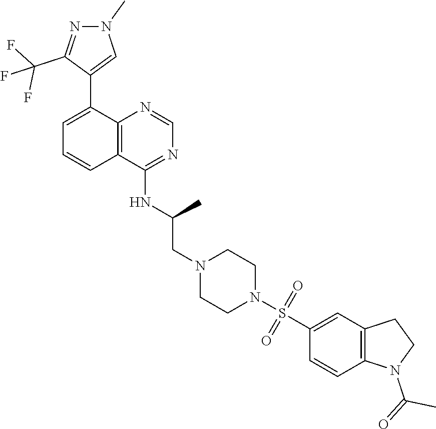 [SMILES: CC(=O)N1CCc2cc(S(=O)(=O)N3CCN(C[C@H](C)Nc4ncnc5c(-c6cn(C)nc6C(F)(F)F)cccc45)CC3)ccc21] | LC-MS (ESI): m/z (M + 1): 643.3 (Method 1) ¹H NMR (500 MHz, DMSO-d₆) δ ppm 1.17 (d, J = 6.6 Hz, 3 H), 2.17 (s, 3 H), 2.38 (dd, J = 12.5, 6.9 Hz, 1 H), 2.49-2.54 (m, 4 H), 2.60 (dd, J = 12.5, 7.3 Hz, 1 H), 2.82 (br s, 4 H), 3.15- 3.23 (m, 2 H), 3.99 (s, 3 H), 4.14 (br t, J = 8.6 Hz, 2 H), 4.57 (spt, J = 6.8 Hz, 1 H), 7.46-7.54 (m, 3 H), 7.66 (d, J = 7.0 Hz, 1 H), 7.87 (d, J = 7.8 Hz, 1 H), 8.05 (s, 1 H), 8.16 (br d, J = 8.4 Hz, 1 H), 8.24 (dd, J = 8.4, 1.2 Hz, 1 H), 8.38 (s, 1 H) |
| | 1-[5-({4-[(2S)-2-({8-[1-methyl-3-(trifluoromethyl)-1H-pyrazol-4-yl]- | |
| | quinazolin-4-yl}amino)propyl]piperazin-1-yl}sulfonyl)-2,3-dihydro- | |
| | 1H-indol-1-yl]ethan-1-one | |
| 69 | 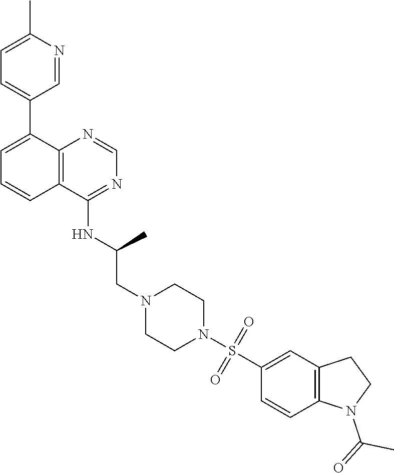 [SMILES: CC(=O)N1CCc2cc(S(=O)(=O)N3CCN(C[C@H](C)Nc4ncnc5c(-c6ccc(C)nc6)cccc45)CC3)ccc21] | LC-MS (ESI): m/z (M + 1): 586.3 (Method 2) ¹H NMR (500 MHz, DMSO-d₆) δ ppm 1.18 (d, J = 6.6 Hz, 3 H), 2.17 (s, 3 H), 2.39 (dd, J = 12.5, 6.7 Hz, 1 H), 2.51-2.55 (m, 4 H), 2.53 (s, 3 H), 2.61 (dd, J = 12.4, 7.5 Hz, 1 H), 2.81 (br s, 4 H), 3.15-3.22 (m, 2 H), 4.13 (br t, J = 8.6 Hz, 2 H), 4.59 (spt, J = 6.9 Hz, 1 H), 7.33 (d, J = 8.1 Hz, 1 H), 7.48-7.53 (m, 2 H), 7.55 (dd, J = 8.2, 7.5 Hz, 1 H), 7.79 (dd, J = 7.3, 1.2 Hz, 1 H), 7.89 (d, J = 8.1 Hz, 1 H), 7.92 (dd, J = 8.0, 2.3 Hz, 1 H), 8.16 (br d, J = 8.2 Hz, 1 H), 8.26 (dd, J = 8.5, 1.1 Hz, 1 H), 8.41 (s, 1 H), 8.63 (d, J = 2.1 Hz, 1 H) |
| | 1-[5-({4-[(2S)-2-{[8-(6-methylpyridin-3-yl)quinazolin-4- | |
| | yl]amino}propyl]piperazin-1-yl}sulfonyl)-2,3-dihydro- | |
| | 1H-indol-1-yl]ethan-1-one | |
| 72 | 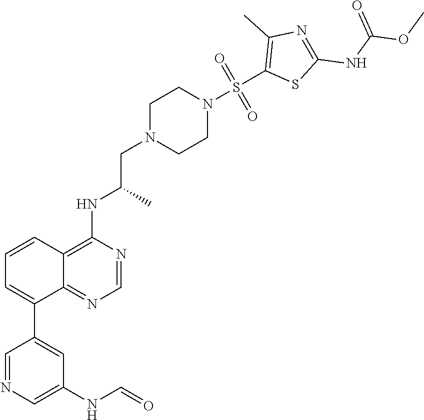 [SMILES: COC(=O)Nc1nc(C)c(S(=O)(=O)N2CCN(C[C@H](C)Nc3ncnc4c(-c5cncc(NC=O)c5)cccc34)CC2)s1] | LC-MS (ESI): m/z (M + 1): 625.7 (Method 3) ¹H NMR (400 MHz, d6-Acetone) δ ppm 9.52 (br s, 1 H) 8.88 (d, J = 1.97 Hz, 1 H) 8.58 (s, 1 H) 8.47 (d, J = 6.58 Hz, 1 H) 8.39 (s, 1 H) 8.13 (s, 2 H) 7.81 (br d, J = 7.45 Hz, 1 H) 7.51 (td, J = 7.56, 2.41 Hz, 1 H) 7.27 (br d, J = 6.80 Hz, 1 H) 4.66- 4.80 (m, 1 H) 3.83 (s, 3 H) 2.60-2.79 (m, 8 H) 2.52 (br dd, J = 12.50, 5.92 Hz, 1 H) 2.41 (d, J = 3.29 Hz, 3 H) 1.32 (br d, J = 6.36 Hz, 3 H). |
| | methyl N-[5-({4-[(2S)-2-{[8-(5-formamidopyridin-3-yl)quinazolin- | |
| | 4-yl]amino}propyl]piperazin-1-yl}sulfonyl)-4-methyl-1,3-thiazol- | |
| | 2-yl]carbamate | |
| 73 | 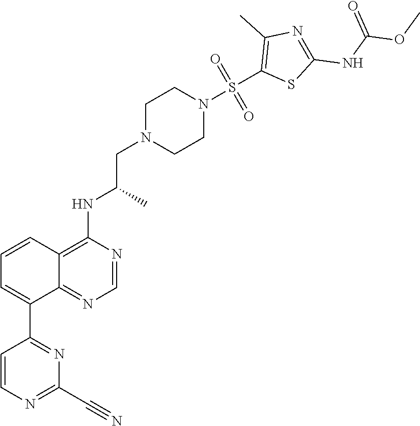 [SMILES: COC(=O)Nc1nc(C)c(S(=O)(=O)N2CCN(C[C@H](C)Nc3ncnc4c(-c5ccnc(C#N)n5)cccc34)CC2)s1] | LC-MS (ESI): m/z (M + 1): 608.7 (Method 3) ¹H NMR (400 MHz, DMSO-d₆) δ ppm 9.30 (s, 2 H), 8.36-8.47 (m, 2 H), 7.97-8.08 (m, 2 H), 7.62 (t, J = 7.89 Hz, 1 H), 4.54-4.67 (m, 1 H), 3.72 (s, 3 H), 2.94 (br s, 4 H), 2.51-2.64 (m, 6 H), 2.51-2.65 (m, 1 H), 2.38 (s, 3 H), 1.19 (d, J = 6.36 Hz, 3 H) |
| | methyl N-[5-({4-[(2S)-2-{[8-(2-cyanopyrimidin-4-yl)quinazolin- | |
| | 4-yl]amino}propyl]piperazin-1-yl}sulfonyl)-4-methyl-1,3- | |
| | thiazol-2-yl]carbamate | |
| 74 | 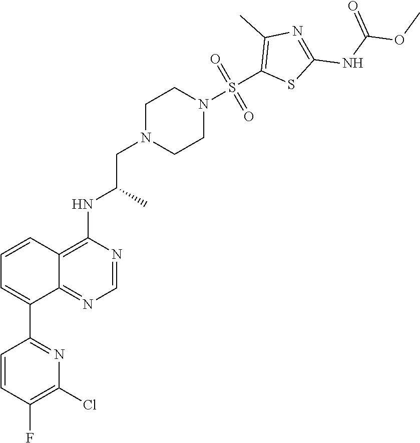 [SMILES: COC(=O)Nc1nc(C)c(S(=O)(=O)N2CCN(C[C@H](C)Nc3ncnc4c(-c5ccc(F)c(Cl)n5)cccc34)CC2)s1] | LC-MS (ESI): m/z (M + 1): 634.7 (Method 3) ¹H NMR (400 MHz, DMSO-d₆) δ ppm 12.21-12.49 (bs, 1 H), 8.50- 8.54 (m, 1 H), 8.43 (s, 1 H), 8.33 (d, J = 8.33 Hz, 1 H), 8.24 (dd, J = 9.87, 1.75 Hz, 1 H), 7.97 (br d, J = 7.89 Hz, 1 H), 7.90 (d, J = 7.23 Hz, 1 H), 7.57 (t, J = 7.78 Hz, 1 H), 4.59 (dt, J = 13.92, 6.85 Hz, 1 H), 3.72 (s, 3 H), 2.95 (br s, 4 H), 2.48-2.64 (m, 6 H), 2.38 (s, 3 H), 1.18 (d, J = 6.58 Hz, 3 H) |
| | methyl N-[5-({4-[(2S)-2-{[8-(6-chloro-5-fluoropyridin-2-yl)- | |
| | quinazolin-4-yl]amino}propyl]piperazin-1-yl}sulfonyl)-4- | |
| | methyl-1,3-thiazol-2-yl]carbamate | |
| 78 | 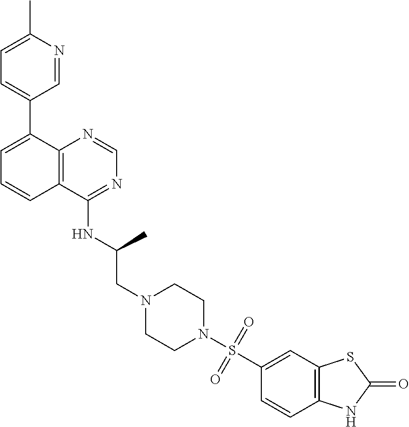 [SMILES: Cc1ccc(-c2cccc3c(N[C@@H](C)CN4CCN(S(=O)(=O)c5ccc6[nH]c(=O)sc6c5)CC4)ncnc23)cn1] | LC-MS (ESI): m/z (M + 1): 576.2 (Method 2) ¹H NMR (400 MHz, DMSO-d₆) δ ppm 1.18 (d, J = 6.6 Hz, 3 H), 2.40 (dd, J = 12.4, 6.7 Hz, 1 H), 2.51-2.56 (m, 4 H), 2.53 (s, 3 H), 2.61 (dd, J = 12.3, 7.3 Hz, 1 H), 2.85 (br s, 4 H), 4.51- 4.67 (m, 1 H), 7.25 (d, J = 8.6 Hz, 1 H), 7.32 (d, J = 7.9 Hz, 1 H), 7.52-7.56 (m, 1 H), 7.56-7.59 (m, 1 H), 7.79 (dd, J = 7.2, 1.2 Hz, 1 H), 7.88 (d, J = 7.9 Hz, 1 H), 7.91 (dd, J = 7.9, 2.2 Hz, 1 H), 7.99 (d, J = 1.8 Hz, 1 H), 8.26 (dd, J = 8.6, 1.1 Hz, 1 H), 8.41 (s, 1 H), 8.64 (d, J = 2.2 Hz, 1 H), 12.34 (br s, 1 H) |
| | 6-({4-[(2S)-2-{[8-(6-methylpyridin-3-yl)quinazolin-4-yl]- | |
| | amino}propyl]piperazin-1-yl}sulfonyl)-2,3-dihydro-1,3- | |
| | benzothiazol-2-one | |
| 79 | 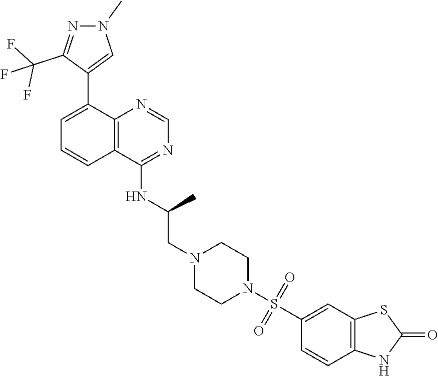 [SMILES: C[C@@H](CN1CCN(S(=O)(=O)c2ccc3[nH]c(=O)sc3c2)CC1)Nc1ncnc2c(-c3cn(C)nc3C(F)(F)F)cccc12] | LC-MS (ESI): m/z (M + 1): 633.2 (Method 2) ¹H NMR (400 MHz, DMSO-d₆) δ ppm 1.17 (d, J = 6.4 Hz, 3 H), 2.39 (dd, J = 12.4, 6.7 Hz, 1 H), 2.53 (br s, 4 H), 2.60 (dd, J = 12.5, 7.2 Hz, 1 H), 2.85 (br s, 4 H), 3.99 (s, 3 H), 4.58 (ddd, J = 13.9, 6.9, 6.8 Hz, 1 H), 7.26 (d, J = 8.6 Hz, 1 H), 7.49 (t, J = 7.8 Hz, 1 H), 7.59 (dd, J = 8.4, 1.9 Hz, 1 H), 7.66 (d, J = 7.0 Hz, 1 H), 7.87 (d, J = 7.9 Hz, 1 H), 8.01 (d, J = 1.8 Hz, 1 H), 8.05 (s, 1 H), 8.20- 8.28 (m, 1 H), 8.38 (s, 1 H), 12.34 (br s, 1 H) |
| | 6-({4-[(2S)-2-({8-[1-methyl-3-(trifluoromethyl)-1H-pyrazol-4- | |
| | yl]quinazolin-4-yl}amino)propyl]piperazin-1-yl}sulfonyl)-2,3- | |
| | dihydro-1,3-benzothiazol-2-one | |
| 80 | 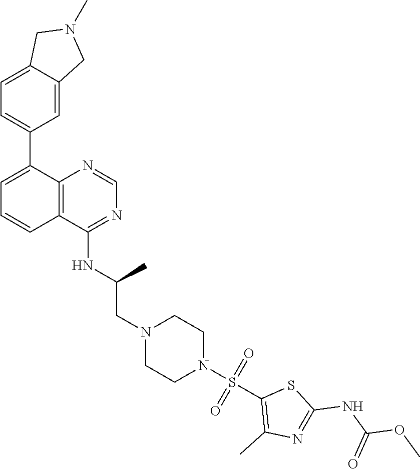 [SMILES: COC(=O)Nc1nc(C)c(S(=O)(=O)N2CCN(C[C@H](C)Nc3ncnc4c(-c5ccc6c(c5)CN(C)C6)cccc34)CC2)s1] | LC-MS (ESI): m/z (M + 1): 637.3 (Method 2) ¹H NMR (400 MHz, DMSO-d₆) δ ppm 1.21 (d, J = 6.6 Hz, 3 H), 2.39- 2.47 (m, 1 H), 2.42 (s, 3 H), 2.53 (s, 3 H), 2.54-2.60 (m, 4 H), 2.60-2.66 (m, 1 H), 2.99 (br s, 4 H), 3.74 (s, 3 H), 3.88 (s, 4 H), 4.60 (dt, J = 14.0, 7.0 Hz, 1 H), 7.28 (d, J = 7.7 Hz, 1 H), 7.36-7.41 (m, 1 H), 7.43 (s, 1 H), 7.48-7.58 (m, 1 H), 7.72 (dd, J = 7.3, 1.2 Hz, 1 H), 7.85 (d, J = 7.9 Hz, 1 H), 8.24 (dd, J = 8.4, 1.2 Hz, 1 H), 8.41 (s, 1 H) |
| | methyl N-[4-methyl-5-({4-[(2S)-2-{[8-(2-methyl-2,3-dihydro-1H- | |
| | isoindol-5-yl)quinazolin-4-yl]amino}propyl]piperazin-1-yl}- | |
| | sulfonyl)-1,3-thiazol-2-yl]carbamate | |
| 82 | 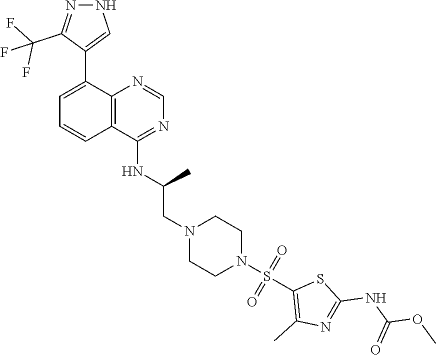 [SMILES: COC(=O)Nc1nc(C)c(S(=O)(=O)N2CCN(C[C@H](C)Nc3ncnc4c(-c5c[nH]nc5C(F)(F)F)cccc34)CC2)s1] | LC-MS (ESI): m/z (M + 1): 640.2 (Method 2) ¹H NMR (400 MHz, DMSO-d₆) δ ppm 1.21 (d, J = 6.5 Hz, 3 H), 2.38- 2.46 (m, 1 H), 2.42 (s, 3 H), 2.58 (br s, 4 H), 2.60-2.66 (m, 1 H), 2.99 (br s, 4 H), 3.75 (s, 3 H), 4.60 (dt, J = 13.9, 6.7 Hz, 1 H), 7.50 (t, J = 7.8 Hz, 1 H), 7.68 (d, J = 7.1 Hz, 1 H), 7.88 (br d, J = 7.9 Hz, 1 H), 8.07 (s, 1 H), 8.26 (d, J = 8.0 Hz, 1 H), 8.39 (s, 1 H), 12.32 (br s, 1 H), 13.70 (br s, 1 H) |
| | methyl N-[4-methyl-5-({4-[(2S)-2-({8-[3-(trifluoromethyl)-1H- | |
| | pyrazol-4-yl]quinazolin-4-yl}amino)propyl]piperazin-1-yl}- | |
| | sulfonyl)-1,3-thiazol-2-yl]carbamate | |
| 83 | 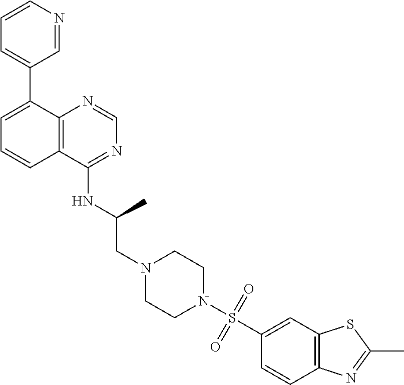 [SMILES: Cc1nc2ccc(S(=O)(=O)N3CCN(C[C@H](C)Nc4ncnc5c(-c6cccnc6)cccc45)CC3)cc2s1] | LC-MS (ESI): m/z (M + 1): 560.2 (Method 2) ¹H NMR (500 MHz, METHANOL-d4) δ ppm 1.26 (d, J = 6.6 Hz, 3 H), 2.47 (dd, J = 12.7, 5.6 Hz, 1 H), 2.55-2.63 (m, 2 H), 2.63- 2.74 (m, 3 H), 2.83-2.88 (m, 3 H), 3.00 (br d, J = 4.4 Hz, 4 H), 4.64-4.72 (m, 1 H), 7.47- 7.52 (m, 1 H), 7.55 (dd, J = 7.8, 4.9 Hz, 1 H), 7.72-7.77 (m, 1 H), 7.78-7.83 (m, 1 H), 8.00-8.06 (m, 2 H), 8.10-8.14 (m, 1 H), 8.36 (s, 1 H), 8.38- 8.40 (m, 1 H), 8.56 (dd, J = 5.0, 1.6 Hz, 1 H), 8.68-8.75 (m, 1 H) |
| | N-[(2S)-1-{4-[(2-methyl-1,3-benzothiazol-6-yl)sulfonyl]- | |
| | piperazin-1-yl}propan-2-yl]-8-(pyridin-3-yl)quinazolin- | |
| | 4-amine | |
| 85 | 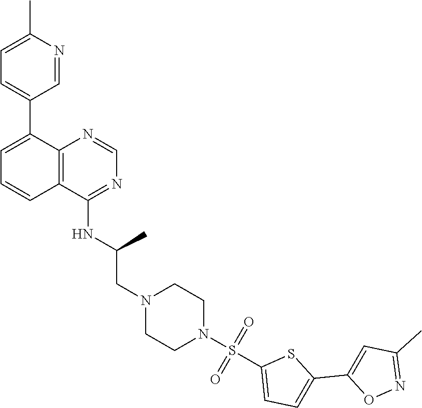 [SMILES: Cc1ccc(-c2cccc3c(N[C@@H](C)CN4CCN(S(=O)(=O)c5ccc(-c6cc(C)no6)s5)CC4)ncnc23)cn1] | LC-MS (ESI): m/z (M + 1): 590.2 (Method 2) ¹H NMR (500 MHz, DMSO-d₆) δ ppm 1.20 (d, J = 6.3 Hz, 3 H), 2.28 (s, 3 H), 2.43 (br dd, J = 12.5, 6.7 Hz, 1 H), 2.53 (s, 3 H), 2.55-2.70 (m, 5 H), 2.97 (br s, 4 H), 4.61 (dt, J = 13.9, 6.9 Hz, 1 H), 6.96 (s, 1 H), 7.32 (d, J = 8.0 Hz, 1 H), 7.54 (t, J = 7.8 Hz, 1 H), 7.69 (d, J = 3.8 Hz, 1 H), 7.73-7.81 (m, 2 H), 7.90 (dd, J = 8.0, 2.2 Hz, 2 H), 8.23-8.31 (m, 1 H), 8.41 (s, 1 H), 8.63 (d, J = 1.9 Hz, 1 H) |
| | N-[(2S)-1-(4-{[5-(3-methyl-1,2-oxazol-5-yl)thiophen-2-yl]- | |
| | sulfonyl}piperazin-1-yl)propan-2-yl]-8-(6-methylpyridin- | |
| | 3-yl)quinazolin-4-amine | |
| 87 | 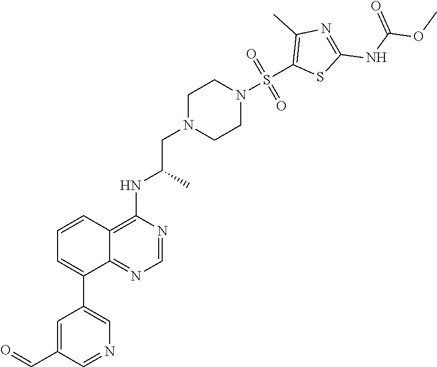 [SMILES: COC(=O)Nc1nc(C)c(S(=O)(=O)N2CCN(C[C@H](C)Nc3ncnc4c(-c5cncc(C=O)c5)cccc34)CC2)s1] | LC-MS (ESI): m/z (M + 1): 610.8 (Method 3) ¹H NMR (400 MHz, DMSO-d₆) δ ppm 10.20 (s, 1 H) 9.08 (dd, J = 4.71, 2.08 Hz, 2 H) 8.49 (t, J = 2.08 Hz, 1 H) 8.45 (s, 1 H) 8.36 (d, J = 7.89 Hz, 1 H) 7.99 (br d, J = 7.89 Hz, 1 H) 7.94 (d, J = 7.23 Hz, 1 H) 7.62 (t, J = 7.78 Hz, 1 H) 4.56-4.69 (m, 1 H) 3.75 (s, 3 H) 2.99 (br s, 4 H) 2.54-2.69 (m, 6 H) 2.42 (s, 3 H) 1.22 (d, J = 6.36 Hz, 3 H) |
| | methyl N-[5-({4-[(2S)-2-{[8-(5-formylpyridin-3-yl)quinazolin- | |
| | 4-yl]amino}propyl]piperazin-1-yl}sulfonyl)-4-methyl-1,3- | |
| | thiazol-2-yl]carbamate | |
| 88 | 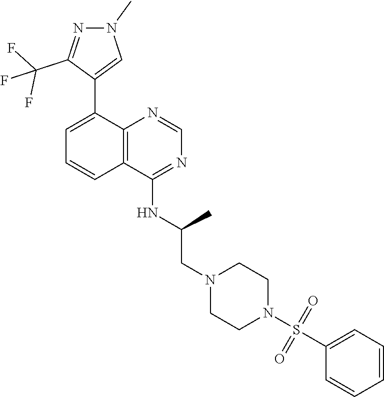 [SMILES: C[C@@H](CN1CCN(S(=O)(=O)c2ccccc2)CC1)Nc1ncnc2c(-c3cn(C)nc3C(F)(F)F)cccc12] | LC-MS (ESI): m/z (M + 1): 560.3 (Method 2) ¹H NMR (500 MHz, DMSO-d₆) δ ppm 1.17 (d, J = 6.6 Hz, 3 H), 2.37- 2.42 (m, 1 H), 2.51-2.55 (m, 4 H), 2.58 (dd, J = 12.5, 7.3 Hz, 1 H), 2.85 (br s, 4 H), 3.99 (s, 3 H), 4.57 (dt, J = 14.2, 7.0 Hz, 1 H), 7.49 (dd, J = 8.2, 7.1 Hz, 1 H), 7.58-7.74 (m, 6 H), 7.87 (d, J = 7.7 Hz, 1 H), 8.05 (s, 1 H), 8.23 (dd, J = 8.5, 1.4 Hz, 1 H), 8.38 (s, 1 H) |
| | N-[(2S)-1-[4-(benzenesulfonyl)piperazin-1-yl]propan-2- | |
| | yl]-8-[1-methyl-3-(trifluoromethyl)-1H-pyrazol-4-yl]- | |
| | quinazolin-4-amine | |
| 92 | 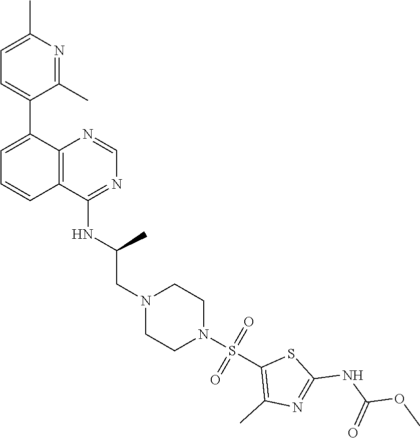 [SMILES: COC(=O)Nc1nc(C)c(S(=O)(=O)N2CCN(C[C@H](C)Nc3ncnc4c(-c5ccc(C)nc5C)cccc34)CC2)s1] | LC-MS (ESI): m/z (M + 1): 611.3 (Method 2) ¹H NMR (400 MHz, DMSO-d₆) δ ppm 1.21 (d, J = 6.6 Hz, 3 H), 2.11 (s, 3 H), 2.38-2.47 (m, 4 H), 2.49 (br s, 3 H), 2.54-2.72 (m, 5 H), 2.90-3.06 (m, 4 H), 3.75 (s, 3 H), 4.47-4.70 (m, 1 H), 7.12 (d, J = 7.7 Hz, 1 H), 7.40 (d, J = 7.7 Hz, 1 H), 7.50-7.57 (m, 1 H), 7.58-7.63 (m, 1 H), 7.91 (d, J = 7.7 Hz, 1 H), 8.29 (dd, J = 8.6, 1.3 Hz, 1 H), 8.34 (s, 1 H), 12.28-12.37 (m, 1 H) |
| | methyl N-[5-({4-[(2S)-2-{[8-(2,6-dimethylpyridin-3-yl)- | |
| | quinazolin-4-yl]amino}propyl]piperazin-1-yl}sulfonyl)- | |
| | 4-methyl-1,3-thiazol-2-yl]carbamate | |
| 94 | 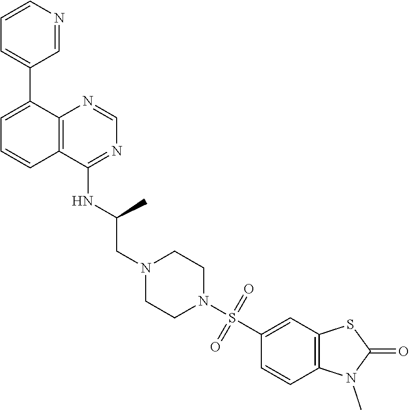 [SMILES: C[C@@H](CN1CCN(S(=O)(=O)c2ccc3c(c2)sc(=O)n3C)CC1)Nc1ncnc2c(-c3cccnc3)cccc12] | LC-MS (ESI): m/z (M + 1): 576.2 (Method 2) ¹H NMR (400 MHz, DMSO-d₆) δ ppm 1.17 (d, J = 6.6 Hz, 3 H), 2.39 (dd, J = 12.3, 6.6 Hz, 1 H), 2.52-2.58 (m, 4 H), 2.62 (dd, J = 12.5, 7.5 Hz, 1 H), 2.85 (br s, 4 H), 3.41 (s, 3 H), 4.60 (dt, J = 13.8, 6.9 Hz, 1 H), 7.43- 7.51 (m, 2 H), 7.56 (t, J = 7.8 Hz, 1 H), 7.69 (dd, J = 8.4, 1.9 Hz, 1 H), 7.82 (dd, J = 7.2, 0.9 Hz, 1 H), 7.91 (d, J = 8.1 Hz, 1 H), 8.02 (dt, J = 7.9, 2.0 Hz, 1 H), 8.12 (d, J = 2.0 Hz, 1 H), 8.28 (dd, J = 8.3, 0.9 Hz, 1 H), 8.41 (s, 1 H), 8.56 (dd, J = 4.8, 1.5 Hz, 1 H), 8.77 (d, J = 1.5 Hz, 1 H) |
| | 3-methyl-6-({4-[(2S)-2-{[8-(pyridin-3-yl)quinazolin-4-yl]amino}- | |
| | propyl]piperazin-1-yl}sulfonyl)-2,3-dihydro-1,3-benzothiazol- | |
| | 2-one | |
| 98 | 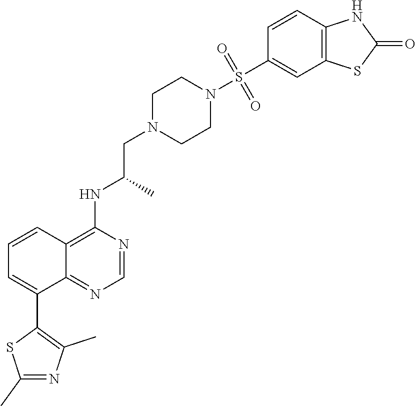 [SMILES: Cc1nc(C)c(-c2cccc3c(N[C@@H](C)CN4CCN(S(=O)(=O)c5ccc6[nH]c(=O)sc6c5)CC4)ncnc23)s1] | LC-MS (ESI): m/z (M + 1): 596.4 (Method 2) ¹H NMR (500 MHz, DMSO-d₆) δ ppm 1.17 (d, J = 6.6 Hz, 3 H), 2.25 (s, 3 H), 2.37-2.43 (m, 1 H), 2.51-2.54 (m, 4 H), 2.58-2.61 (m, 1 H), 2.64 (s, 3 H), 2.83 (br s, 4 H), 4.41-4.71 (m, 1 H), 7.20 (d, J = 8.2 Hz, 1 H), 7.46- 7.55 (m, 2 H), 7.79 (dd, J = 7.4, 1.1 Hz, 1 H), 7.83-7.98 (m, 2 H), 8.25 (dd, J = 8.5, 1.1 Hz, 1 H), 8.41 (s, 1 H), 12.18- 12.54 (m, 1 H) |
| | 6-({4-[(2S)-2-{[8-(2,4-dimethyl-1,3-thiazol-5-yl)quinazolin-4- | |
| | yl]amino}propyl]piperazin-1-yl}sulfonyl)-2,3-dihydro-1,3- | |
| | benzothiazol-2-one | |
| 99 | 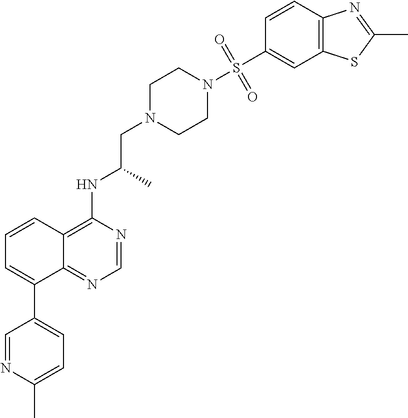 [SMILES: Cc1ccc(-c2cccc3c(N[C@@H](C)CN4CCN(S(=O)(=O)c5ccc6nc(C)sc6c5)CC4)ncnc23)cn1] | LC-MS (ESI): m/z (M + 1): 574.5 (Method 1) ¹H NMR (400 MHz, DMSO-d₆) δ ppm 1.16 (d, J = 6.6 Hz, 3 H), 2.39 (dd, J = 12.4, 6.7 Hz, 1 H), 2.52 (br s, 4 H), 2.53 (s, 3 H), 2.59 (dd, J = 12.4, 7.3 Hz, 1 H), 2.85 (s, 3 H), 2.89 (br s, 4 H), 4.57 (dt, J = 13.9, 6.9 Hz, 1 H), 7.32 (d, J = 8.0 Hz, 1 H), 7.52 (t, J = 7.8 Hz, 1 H), 7.71- 7.81 (m, 2 H), 7.87 (d, J = 7.9 Hz, 1 H), 7.90 (dd, J = 8.0, 2.3 Hz, 1 H), 8.08 (d, J = 8.6 Hz, 1 H), 8.18-8.30 (m, 1 H), 8.40 (s, 1 H), 8.52 (d, J = 1.6 Hz, 1 H), 8.63 (d, J = 2.0 Hz, 1 H) |
| | N-[(2S)-1-{4-[(2-methyl-1,3-benzothiazol-6-yl)sulfonyl]- | |
| | piperazin-1-yl}propan-2-yl]-8-(6-methylpyridin-3-yl)- | |
| | quinazolin-4-amine | |
| 100 | 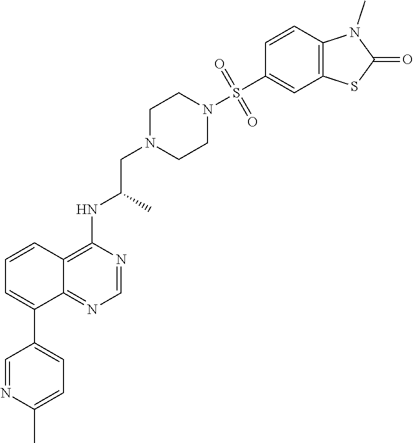 [SMILES: Cc1ccc(-c2cccc3c(N[C@@H](C)CN4CCN(S(=O)(=O)c5ccc6c(c5)sc(=O)n6C)CC4)ncnc23)cn1] | LC-MS (ESI): m/z (M + 1): 590.4 (Method 1) ¹H NMR (400 MHz, DMSO-d₆) δ ppm 1.17 (d, J = 6.6 Hz, 3 H), 2.39 (dd, J = 12.4, 6.5 Hz, 1 H), 2.52 (s, 3 H), 2.54 (br s, 4 H), 2.62 (dd, J = 12.3, 7.6 Hz, 1 H), 2.85 (br s, 4 H), 3.41 (s, 3 H), 4.59 (ddd, J = 13.8, 7.1, 7.0 Hz, 1 H), 7.32 (d, J = 8.0 Hz, 1 H), 7.48 (d, J = 8.6 Hz, 1 H), 7.54 (t, J = 7.8 Hz, 1 H), 7.69 (dd, J = 8.6, 1.9 Hz, 1 H), 7.76-7.82 (m, 1 H), 7.85-7.90 (m, 1 H), 7.89-7.94 (m, 1 H), 8.11 (d, J = 1.9 Hz, 1 H), 8.25 (d, J = 7.7 Hz, 1 H), 8.40 (s, 1 H), 8.63 (d, J = 2.1 Hz, 1 H) |
| | 3-methyl-6-({4-[(2S)-2-{[8-(6-methylpyridin-3-yl)quinazolin- | |
| | 4-yl]amino}propyl]piperazin-1-yl}sulfonyl)-2,3-dihydro- | |
| | 1,3-benzothiazol-2-one | |
| 103 | 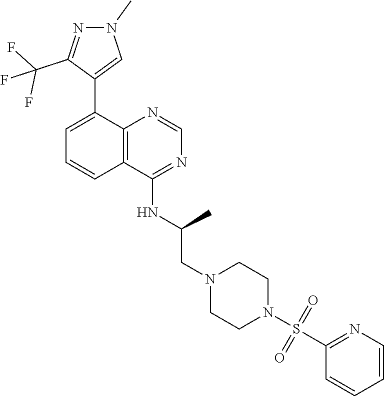 [SMILES: C[C@@H](CN1CCN(S(=O)(=O)c2ccccn2)CC1)Nc1ncnc2c(-c3cn(C)nc3C(F)(F)F)cccc12] | LC-MS (ESI): m/z (M + 1): 561.2 (Method 2) ¹H NMR (500 MHz, DMSO-d₆) δ ppm 1.19 (d, J = 6.3 Hz, 3 H), 2.40 (dd, J = 12.5, 6.7 Hz, 1 H), 2.50-2.51 (m, 4 H), 2.57-2.64 (m, 1 H), 3.05-3.20 (m, 4 H), 4.00 (s, 3 H), 4.61 (dt, J = 14.0, 7.0 Hz, 1 H), 7.49-7.54 (m, 1 H), 7.64-7.69 (m, 2 H), 7.88-7.92 (m, 2 H), 8.06 (s, 1 H), 8.09 (td, J = 7.7, 1.6 Hz, 1 H), 8.26 (dd, J = 8.4, 1.0 Hz, 1 H), 8.39 (s, 1 H), 8.71 (d, J = 4.4 Hz, 1 H) |
| | 8-[1-methyl-3-(trifluoromethyl)-1H-pyrazol-4-yl]-N-[(2S)- | |
| | 1-[4-(pyridine-2-sulfonyl)piperazin-1-yl]propan-2-yl]- | |
| | quinazolin-4-amine | |
| 104 | 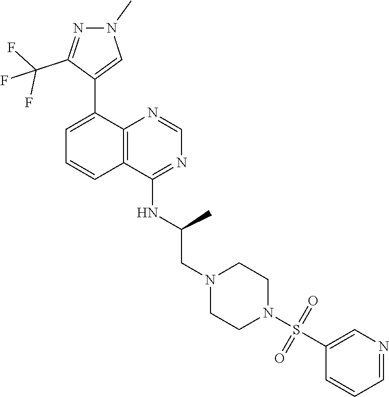 [SMILES: C[C@@H](CN1CCN(S(=O)(=O)c2cccnc2)CC1)Nc1ncnc2c(-c3cn(C)nc3C(F)(F)F)cccc12] | LC-MS (ESI): m/z (M + 1): 561.3 (Method 2) ¹H NMR (400 MHz, DMSO-d₆) δ ppm 1.18 (d, J = 6.6 Hz, 3 H), 2.40 (dd, J = 12.4, 6.7 Hz, 1 H), 2.52-2.56 (m, 4 H), 2.60 (dd, J = 12.4, 7.4 Hz, 1 H), 2.92 (br s, 4 H), 3.99 (s, 3 H), 4.58 (m, J = 14.0, 7.0, 7.0 Hz, 1 H), 7.49 (dd, J = 8.1, 7.3 Hz, 1 H), 7.60-7.72 (m, 2 H), 7.86 (d, J = 7.9 Hz, 1 H), 8.05 (s, 1 H), 8.09-8.17 (m, 1 H), 8.24 (dd, J = 8.5, 1.2 Hz, 1 H), 8.38 (s, 1 H), 8.78-8.93 (m, 2 H) |
| | 8-[1-methyl-3-(trifluoromethyl)-1H-pyrazol-4-yl]-N-[(2S)- | |
| | 1-[4-(pyridine-3-sulfonyl)piperazin-1-yl]propan-2-yl]- | |
| | quinazolin-4-amine | |
| 108 | 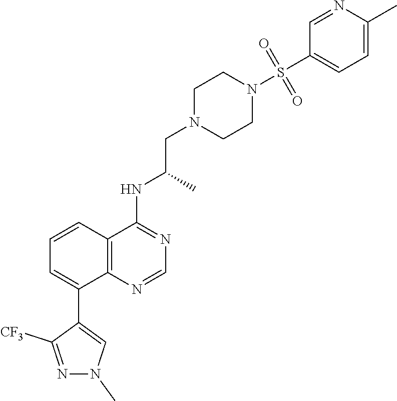 [SMILES: Cc1ccc(S(=O)(=O)N2CCN(C[C@H](C)Nc3ncnc4c(-c5cn(C)nc5C(F)(F)F)cccc34)CC2)cn1] | LC-MS (ESI): m/z (M + 1): 575.2 (Method 2) ¹H NMR (400 MHz, DMSO-d₆) δ ppm 1.18 (d, J = 6.5 Hz, 3 H), 2.39 (dd, J = 12.4, 6.7 Hz, 1 H), 2.52-2.55 (m, 4 H), 2.56 (s, 3 H), 2.60 (br dd, J = 12.4, 7.3 Hz, 1 H), 2.88 (br s, 4 H), 3.99 (s, 3 H), 4.58 (spt, J = 6.9 Hz, 1 H), 7.43-7.55 (m, 2 H), 7.66 (d, J = 7.0 Hz, 1 H), 7.87 (d, J = 8.0 Hz, 1 H), 7.98 (dd, J = 8.2, 2.4 Hz, 1 H), 8.05 (s, 1 H), 8.24 (d, J = 8.3 Hz, 1 H), 8.38 (s, 1 H), 8.72 (d, J = 2.1 Hz, 1 H) |
| | 8-[1-methyl-3-(trifluoromethyl)-1H-pyrazol-4-yl]-N-[(2S)- | |
| | 1-{4-[(6-methylpyridin-3-yl)sulfonyl]piperazin-1-yl}- | |
| | propan-2-yl]quinazolin-4-amine | |
| 109 | 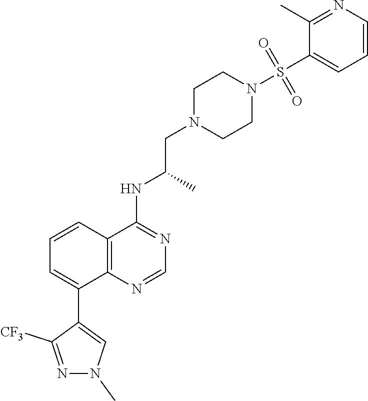 [SMILES: Cc1ncccc1S(=O)(=O)N1CCN(C[C@H](C)Nc2ncnc3c(-c4cn(C)nc4C(F)(F)F)cccc23)CC1] | LC-MS (ESI): m/z (M + 1): 575.2 (Method 2) ¹H NMR (500 MHz, DMSO-d₆) δ ppm 1.21 (d, J = 6.6 Hz, 3 H), 2.41 (dd, J = 12.5, 6.7 Hz, 1 H), 2.53 (br t, J = 4.5 Hz, 4 H), 2.62 (dd, J = 12.4, 7.3 Hz, 1 H), 2.73 (s, 3 H), 3.05 (br s, 4 H), 3.99 (s, 3 H), 4.61 (quin, J = 7.0 Hz, 1 H), 7.46 (dd, J = 8.0, 4.8 Hz, 1 H), 7.51 (dd, J = 8.2, 7.3 Hz, 1 H), 7.67 (d, J = 7.1 Hz, 1 H), 7.90 (d, J = 8.0 Hz, 1 H), 8.06 (s, 1 H), 8.12 (dd, J = 8.0, 1.6 Hz, 1 H), 8.26 (dd, J = 8.5, 1.2 Hz, 1 H), 8.39 (s, 1 H), 8.68 (dd, J = 4.7, 1.6 Hz, 1 H) |
| | 8-[1-methyl-3-(trifluoromethyl)-1H-pyrazol-4-yl]-N-[(2S)- | |
| | 1-{4-[(2-methylpyridin-3-yl)sulfonyl]piperazin-1-yl}- | |
| | propan-2-yl]quinazolin-4-amine | |

### Example 2

### methyl N-[4-methyl-5-({4-[(2S)-2-{[8-(5-methylpyrazin-2-yl)quinazolin-4-yl]amino}propyl]piperazin-1-yl}sulfonyl)-1,3-thiazol-2-yl]carbamate

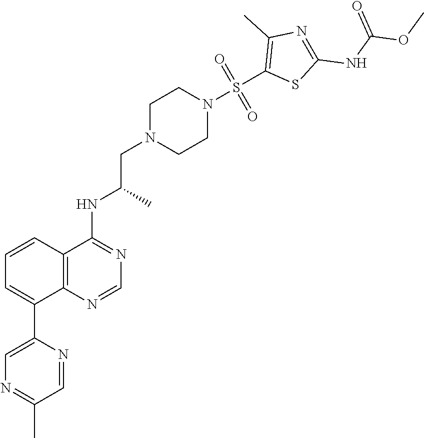 [SMILES: COC(=O)Nc1nc(C)c(S(=O)(=O)N2CCN(C[C@H](C)Nc3ncnc4c(-c5cnc(C)cn5)cccc34)CC2)s1]

### Step 1: Preparation of tert-butyl 4-[(2S)-2-[[8-(5-methylpyrazin-2-yl)quinazolin-4-yl]amino]propyl]piperazine-1-carboxylate (Intermediate 4)

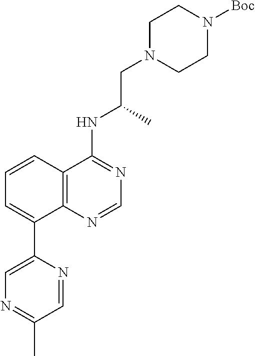 [SMILES: Cc1cnc(-c2cccc3c(N[C@@H](C)CN4CCN(C(=O)OC(C)(C)C)CC4)ncnc23)cn1]

A 5 mL microwave vial equipped with stir bar and septa cap was charged with Intermediate 1 (100 mg, 0.22 mmol), AcOK (65 mg, 0.67 mmol), diboronic acid (40 mg, 0.44 mmol), SPhos Pd G2 (16 mg, 0.02 mmol) and dicyclohexyl-[2-(2,6-dimethoxyphenyl)phenyl]phosphine (18 mg, 0.04 mmol). The tube was sealed and three cycles of vacuum/nitrogen were applied. Ethanol (1.5 mL) and ethane-1,2-diol (0.06 mL, 1.11 mmol) were added and three cycles of vacuum/nitrogen were repeated. The mixture was heated at 80° C. for 2 h and then cooled under a stream of nitrogen. K₃PO₄ (0.14 mL, 0.67 mmol) and H₂O (0.4 mL) were added, followed by 2-bromo-5-methylpyrazine (38 mg, 0.22 mmol). Three cycles of vacuum/nitrogen were repeated and the mixture was heated at 80° C. for 2 h. The mixture was cooled to r.t., diluted with AcOEt (5 mL), filtered through a plug of celite and washed with AcOEt (3×3 mL). The filtrate was washed with sat. aq. NaHCO₃ (15 mL) and the aqueous phase was extracted with AcOEt (2×15 mL). The organic phases were collected, washed with brine (30 mL), dried over Na₂SO₄, filtered, and concentrated under reduced pressure. The solid residue was purified by flash chromatography eluting with AcOEt in cyclohexane from 20% to 50% yielding title compound (Intermediate 4, 50 mg, 0.11 mmol, 49% yield) as a yellow solid.

LC-MS (ESI): m/z (M+1): 464.47 (Method 1)

### Step 2: Preparation of 8-(5-methylpyrazin-2-yl)-N-[(2S)-1-piperazin-1-ylpropan-2-yl]quinazolin-4-amine hydrochloride (Intermediate 5)

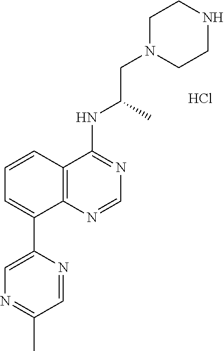 [SMILES: Cc1cnc(-c2cccc3c(N[C@@H](C)CN4CCNCC4)ncnc23)cn1 ; Cl]

Title compound was prepared following the procedure used for the synthesis of Intermediate 2, starting from tert-butyl 4-[(2S)-2-[[8-(5-methylpyrazin-2-yl)quinazolin-4-yl]amino]propyl]piperazine-1-carboxylate (Intermediate 4, 50 mg, 0.11 mmol) to afford Intermediate 5 (30 mg, crude) that was used in the next step without further purification.

LC-MS (ESI): m/z (M+1): 364.45 (Method 2)

### Step 3: Preparation of methyl N-[4-methyl-5-({4-[(2S)-2-{[8-(5-methylpyrazin-2-yl)quinazolin-4-yl]amino}propyl]piperazin-1-yl}sulfonyl)-1,3-thiazol-2-yl]carbamate

### Example 2

A solution of Intermediate 5 (30 mg, crude) in MeCN (2 mL) was cooled at 0° C., then methyl N-[5-(chlorosulfonyl)-4-methyl-1,3-thiazol-2-yl]carbamate (19.4 mg, 0.07 mmol) was added followed by Et₃N (0.1 mL, 0.7 mmol). The reaction was stirred at 0° C. for 1 hour, then NaHCO₃ (0.2 mL) was added and the mixture was concentrated under reduced pressure. The crude was purified by reverse phase flash chromatography eluting with MeCN/0.1% aqueous HCOOH from 3% to 30% to afford the title compound (20 mg, 0.033 mmol, 94% yield) as a white solid.

LC-MS (ESI): m/z (M+1): 598.3 (Method 2)

¹H NMR (500 MHz, DMSO-d₆) δ ppm 1.22 (d, J=6.6 Hz, 3H), 2.39-2.47 (m, 4H), 2.52-2.61 (m, 7H), 2.61-2.65 (m, 1H), 2.99 (br d, J=4.4 Hz, 4H), 3.74 (s, 3H), 4.55-4.70 (m, 1H), 7.57-7.68 (m, 1H), 8.00 (d, J=7.7 Hz, 1H), 8.16 (dd, J=7.3, 1.2 Hz, 1H), 8.37 (dd, J=8.4, 1.2 Hz, 1H), 8.49 (s, 1H), 8.65 (d, J=0.9 Hz, 1H), 9.26 (d, J=1.3 Hz, 1H), 12.11-12.45 (m, 1H)

The Example in the following table was prepared from commercially available reagents by using methods analogous to Example 2.

| Example | | |
|---|---|---|
| No. | Structure & Name | Analytical data |
| 95 | 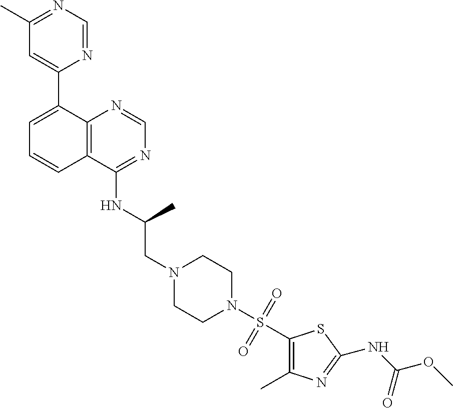 | LC-MS (ESI): m/z (M + 1): 598.3 (Method 2) ¹H NMR (400 MHz, DMSO-d₆) δ ppm 1.22 (d, J = 6.6 Hz, 3 H), 2.39-2.47 (m, 4 H), 2.54 (s, 3 H), 2.55-2.65 (m, 5 H), 2.99 (br s, 4 H), 3.73 (s, 3 H), 4.57- 4.69 (m, 1 H), 7.62 (t, J = 7.9 Hz, 1 H), 8.02 (d, J = 7.9 Hz, 1 H), 8.23 (s, 1 H), 8.26 (dd, J = 7.3, 1.0 Hz, 1 H), 8.38-8.45 (m, 1 H), 8.52 (s, 1 H), 9.13 (d, J = 1.1 Hz, 1 H), 12.32 (br s, 1 H) |
| | methyl N-[4-methyl-5-({4-[(2S)-2-{[8- | |
| | (6-methylpyrimidin-4-yl)quinazolin-4- | |
| | yl]amino}propyl]piperazin-1- | |
| | yl}sulfonyl)-1,3-thiazol-2-yl]carbamate | |

### Example 12

### N-[(2S)-1-(4-{[5-(3-methyl-1,2-oxazol-5-yl)thiophen-2-yl]sulfonyl}piperazin-1-yl)propan-2-yl]-8-(pyridin-2-yl)quinazolin-4-amine

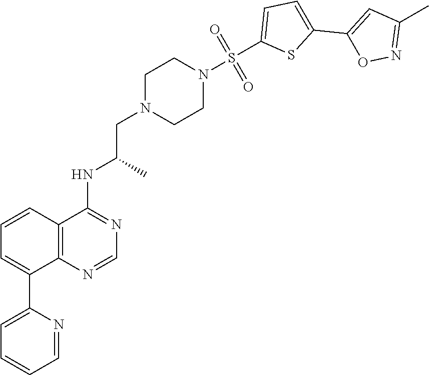 [SMILES: Cc1cc(-c2ccc(S(=O)(=O)N3CCN(C[C@H](C)Nc4ncnc5c(-c6ccccn6)cccc45)CC3)s2)on1]

### Step 1: Preparation of tert-butyl 4-[(2S)-2-[(8-pyridin-2-ylquinazolin-4-yl)amino]propyl]piperazine-1-carboxylate (Intermediate 6)

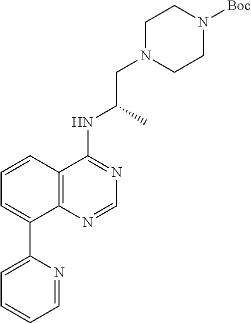 [SMILES: C[C@@H](CN1CCN(C(=O)OC(C)(C)C)CC1)Nc1ncnc2c(-c3ccccn3)cccc12]

To a suspension of tert-butyl 4-[(2S)-2-[(8-bromoquinazolin-4-yl)amino]propyl]piperazine-1-carboxylate (Intermediate 1, 110 mg, 0.24 mmol), in DMF (1.5 mL), 2-(Tributylstannyl)pyridine (0.09 mL, 0.29 mmol) and Tetrakis(triphenylphosphine)-palladium(0) (14 mg, 0.01 mmol) were added. After degassing with nitrogen the tube was sealed and the reaction was heated at 120° C. for 3 h. After cooling down the temperature, 10% potassium fluoride aqueous solution was added and the reaction mixture was stirred for 10 minutes. Water and ethyl acetate were then added, the organic layer was separated and solvent removed under reduced pressure. The crude was purified by flash chromatography using a gradient of EtOAc in Cyclohexane from 0% to 70% affording title compound (Intermediate 6, 35 mg, 0.078 mmol, 32% yield).

LC-MS (ESI): m/z (M+1): 449.2 (Method 1)

### Step 2: Preparation of N-[(2S)-1-piperazin-1-ylpropan-2-yl]-8-pyridin-2-ylquinazolin-4-amine hydrochloride (Intermediate 7)

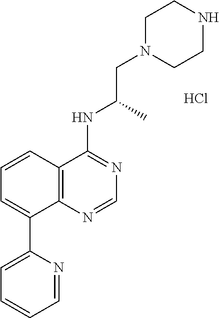 [SMILES: C[C@@H](CN1CCNCC1)Nc1ncnc2c(-c3ccccn3)cccc12 ; Cl]

Title compound was prepared following the procedure used for the synthesis of Intermediate 2, starting from tert-butyl 4-[(2S)-2-[(8-pyridin-2-ylquinazolin-4-yl)amino]propyl]piperazine-1-carboxylate (Intermediate 6, 35 mg, 0.078 mmol) to afford Intermediate 7 (40 mg, crude) that was used in the next step without further purification.

LC-MS (ESI): m/z (M+1): 349.2 (Method 2)

### Step 3: Preparation of N-[(2S)-1-(4-{[5-(3-methyl-1,2-oxazol-5-yl)thiophen-2-yl]sulfonyl}piperazin-1-yl)propan-2-yl]-8-(pyridin-2-yl)quinazolin-4-amine (Example 12)

Title compound was prepared following the procedure used for the synthesis of Intermediate 3, starting from N-[(2S)-1-piperazin-1-ylpropan-2-yl]-8-pyridin-2-ylquinazolin-4-amine hydrochloride (Intermediate 7, 35 mg, crude) and 5-(3-methyl-1,2-oxazol-5-yl)thiophene-2-sulfonyl chloride (23 mg, 0.086 mmol) to afford title compound (4.5 mg, 0.08 mmol, 10% yield).

LC-MS (ESI): m/z (M+1): 576.1 (Method 2)

¹H NMR (400 MHz, DMSO-d₆) δ ppm 1.21 (d, J=6.6 Hz, 3H), 2.28 (s, 3H), 2.40-2.47 (m, 1H), 2.56-2.66 (m, 5H), 2.91-3.04 (m, 4H), 4.56-4.67 (m, 1H), 6.95 (s, 1H), 7.37 (ddd, J=7.5, 4.8, 1.3 Hz, 1H), 7.57 (t, J=7.7 Hz, 1H), 7.69 (d, J=4.0 Hz, 1H), 7.75 (d, J=4.0 Hz, 1H), 7.84 (td, J=7.7, 1.8 Hz, 1H), 7.92 (d, J=7.9 Hz, 1H), 8.10-8.17 (m, 2H), 8.31 (dd, J=8.4, 1.3 Hz, 1H), 8.46 (s, 1H), 8.65-8.71 (m, 1H)

| Example | | |
|---|---|---|
| No. | Structure & Name | Analytical data |
| 29 | 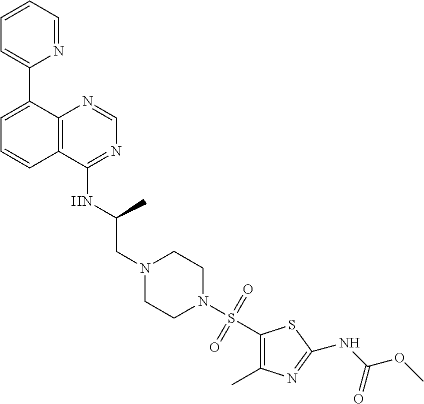 [SMILES: COC(=O)Nc1nc(C)c(S(=O)(=O)N2CCN(C[C@H](C)Nc3ncnc4c(-c5ccccn5)cccc34)CC2)s1] | LC-MS (ESI): m/z (M + 1): 583.1 (Method 2) ¹H NMR (500 MHz, DMSO-d₆) δ ppm 1.22 (d, J = 6.6 Hz, 3 H), 2.40-2.47 (m, 1 H), 2.43 (s, 3 H), 2.54-2.61 (m, 4 H), 2.64 (dd, J = 12.5, 7.1 Hz, 1 H), 3.00 (br s, 4 H), 3.75 (s, 3 H), 4.52- 4.71 (m, 1 H), 7.37 (ddd, J = 7.5, 4.9, 1.0 Hz, 1 H), 7.59 (t, J = 7.7 Hz, 1 H), 7.85 (td, J = 7.7, 1.8 Hz, 1 H), 7.93 (br d, J = 7.9 Hz, 1 H), 8.15 (t, J = 7.5 Hz, 2 H), 8.27-8.37 (m, 1 H), 8.47 (s, 1 H), 8.68 (dt, J = 4.9, 0.8 Hz, 1 H), 12.33 (br s, 1 H) |
| | methyl N-[4-methyl-5-({4-[(2S)-2-{[8- | |
| | (pyridin-2-yl)quinazolin-4- | |
| | yl]amino}propyl]piperazin-1- | |
| | yl}sulfonyl)-1,3-thiazol-2-yl]carbamate | |
| 71 | 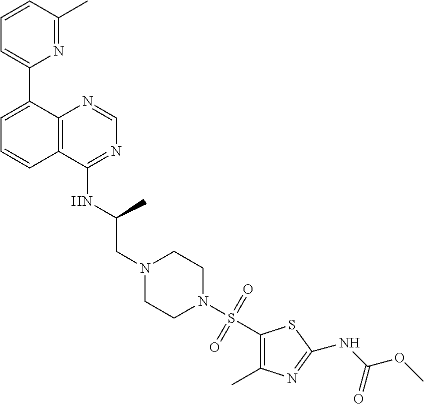 [SMILES: COC(=O)Nc1nc(C)c(S(=O)(=O)N2CCN(C[C@H](C)Nc3ncnc4c(-c5cccc(C)n5)cccc34)CC2)s1] | LC-MS (ESI): m/z (M + 1): 597.2 (Method 2) ¹H NMR (400 MHz, DMSO-d₆) δ ppm 1.22 (d, J = 6.6 Hz, 3 H), 2.40-2.47 (m, 1 H), 2.42 (s, 3 H), 2.54 (s, 3 H), 2.58 (br s, 4 H), 2.60-2.65 (m, 1 H), 2.99 (br s, 4 H), 3.74 (s, 3 H), 4.62 (spt, J = 7.0 Hz, 1 H), 7.23 (d, J = 7.6 Hz, 1 H), 7.58 (t, J = 7.8 Hz, 1 H), 7.73 (t, J = 7.7 Hz, 1 H), 7.88-7.98 (m, 2 H), 8.12 (dd, J = 7.3, 1.2 Hz, 1 H), 8.31 (dd, J = 8.4, 1.2 Hz, 1 H), 8.46 (s, 1 H), 12.34 (br s, 1 H) |
| | methyl N-[4-methyl-5-({4-[(2S)-2-{[8- | |
| | (6-methylpyridin-2-yl)quinazolin-4- | |
| | yl]amino}propyl]piperazin-1- | |
| | yl}sulfonyl)-1,3-thiazol-2-yl]carbamate | |

### Example 20

### N-[(2S)-1-(4-{[5-(3-methyl-1,2-oxazol-5-yl)thiophen-2-yl]sulfonyl}piperazin-1-yl)propan-2-yl]-8-(pyrrolidin-1-yl)quinazolin-4-amine

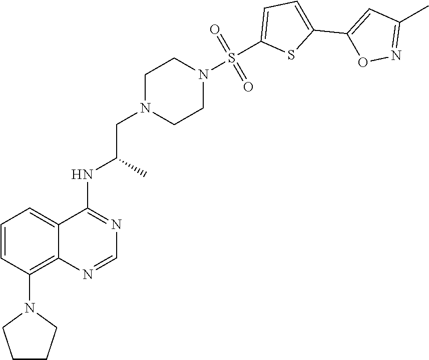 [SMILES: Cc1cc(-c2ccc(S(=O)(=O)N3CCN(C[C@H](C)Nc4ncnc5c(N6CCCC6)cccc45)CC3)s2)on1]

### Step 1: Preparation of tert-butyl 4-[(2S)-2-[(8-pyrrolidin-1-ylquinazolin-4-yl)amino]propyl]piperazine-1-carboxylate (Intermediate 8)

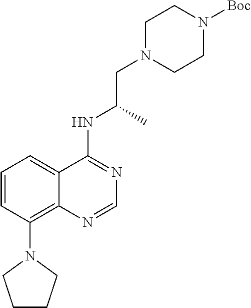 [SMILES: C[C@@H](CN1CCN(C(=O)OC(C)(C)C)CC1)Nc1ncnc2c(N3CCCC3)cccc12]

To a solution of tert-butyl 4-[(2S)-2-[(8-bromoquinazolin-4-yl)amino]propyl]piperazine-1-carboxylate (Intermediate 1, 100 mg, 0.22 mmol) in Toluene (1.5 mL), pyrrolidine (0.03 mL, 0.330 mmol) and Cs₂CO₃ (145.59 mg, 0.440 mmol) were added. After degassing with nitrogen for 10 min, Palladium(II) acetate (2.49 mg, 0.010 mmol) and rac-BINAP (6.91 mg, 0.010 mmol) were added. The tube was sealed and heated at 120° C. under MW irradiation for 1 h. The mixture was partitioned between water and AcOEt and the organic layer was concentrated under reduced pressure. The crude was purified by flash chromatography using a gradient of AcOEt in Cyclohexane from 0% to 30% affording title compound (60 mg, 0.136 mmol, 61% yield) as a colorless oil.

LC-MS (ESI): m/z (M+1): 441.2 (Method 2)

### Step 2: N-[(2S)-1-piperazin-1-ylpropan-2-yl]-8-pyrrolidin-1-ylquinazolin-4-amine hydrochloride (Intermediate 9)

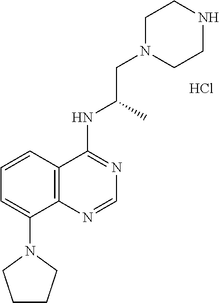 [SMILES: C[C@@H](CN1CCNCC1)Nc1ncnc2c(N3CCCC3)cccc12 ; Cl]

Title compound was prepared following the procedure used for the synthesis of Intermediate 2, starting from tert-butyl 4-[(2S)-2-[(8-pyrrolidin-1-ylquinazolin-4-yl)amino]propyl]piperazine-1-carboxylate (Intermediate 8, 60 mg, 0.136 mmol) to afford Intermediate 9 (56 mg, crude) that was used in the next step without further purification.

LC-MS (ESI): m/z (M+1): 341.1 (Method 2)

### Step 3: Preparation of N-[(2S)-1-(4-{[5-(3-methyl-1,2-oxazol-5-yl)thiophen-2-yl]sulfonyl}piperazin-1-yl)propan-2-yl]-8-(pyrrolidin-1-yl)quinazolin-4-amine

### Example 20

Title compound was prepared following the procedure used for the synthesis of Intermediate 3, starting from N-[(2S)-1-piperazin-1-ylpropan-2-yl]-8-pyrrolidin-1-ylquinazolin-4-amine hydrochloride (Intermediate 7, 56 mg, crude) and 5-(3-methyl-1,2-oxazol-5-yl)thiophene-2-sulfonyl chloride (36 mg, 0.14 mmol) to afford title compound (28 mg, 0.05 mmol, 36% yield).

LC-MS (ESI): m/z (M+1): 568.2 (Method 2)

¹H NMR (400 MHz, DMSO-d₆) δ ppm 1.16 (d, J=6.6 Hz, 3H), 1.80-1.99 (m, 4H), 2.29 (s, 3H), 2.40 (dd, J=12.4, 6.9 Hz, 1H), 2.53-2.64 (m, 5H), 2.97 (br s, 4H), 3.59 (br t, J=6.5 Hz, 4H), 4.42-4.67 (m, 1H), 6.77 (d, J=7.9 Hz, 1H), 6.97 (s, 1H), 7.22 (t, J=8.0 Hz, 1H), 7.31-7.53 (m, 2H), 7.69 (d, J=3.9 Hz, 1H), 7.76 (d, J=4.2 Hz, 1H), 8.31 (s, 1H)

### Example 21

### methyl N-[5-({4-[(2S)-2-[(8-{3-[(dimethylamino)methyl]phenyl}quinazolin-4-yl)amino]propyl]piperazin-1-yl}sulfonyl)-4-methyl-1,3-thiazol-2-yl]carbamate

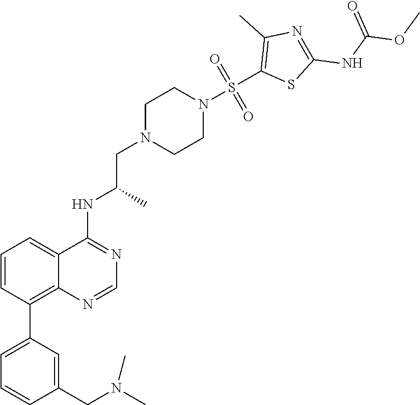 [SMILES: COC(=O)Nc1nc(C)c(S(=O)(=O)N2CCN(C[C@H](C)Nc3ncnc4c(-c5cccc(CN(C)C)c5)cccc34)CC2)s1]

### Step 1: Preparation of methyl N-[5-[4-[(2S)-2-[[8-(3-formylphenyl)quinazolin-4-yl]amino]propyl]piperazin-1-yl]sulfonyl-4-methyl-1,3-thiazol-2-yl]carbamate (Intermediate 10)

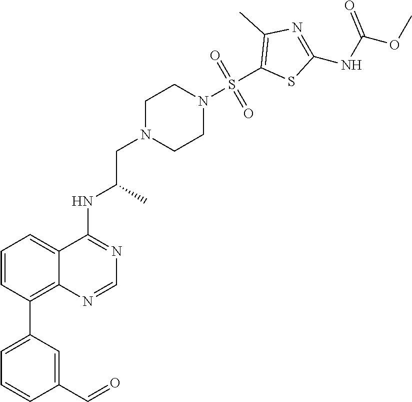 [SMILES: COC(=O)Nc1nc(C)c(S(=O)(=O)N2CCN(C[C@H](C)Nc3ncnc4c(-c5cccc(C=O)c5)cccc34)CC2)s1]

To a suspension of Pd(dppf)Cl₂ (1.13 g, 1.54 mmol) and methyl N-[5-[4-[(2S)-2-[(8-bromoquinazolin-4-yl)amino]propyl]piperazin-1-yl]sulfonyl-4-methyl-1,3-thiazol-2-yl]carbamate (Intermediate 3, 4.5 g, 7.7 mmol) in Water (9 mL) and THE (30 mL), (3-formylphenyl)boronic acid (1.73 g, 11 mmol) was added. After degassing with nitrogen K₃PO₄ (3.3 g, 15.4 mmol) was added and the tube was sealed. The reaction was heated at 80° C. for 12 h. After cooling down the temperature, the mixture was concentrated under reduced pressure affording methyl N-[5-[4-[(2S)-2-[[8-(3-formylphenyl)quinazolin-4-yl]amino]propyl]piperazin-1-yl]sulfonyl-4-methyl-1,3-thiazol-2-yl]carbamate (11 g, crude) that was used in the following step without further purifications.

LC-MS (ESI): m/z (M+1): 610.1 (Method 2)

### Step 2: Preparation of methyl N-[5-({4-[(2S)-2-[(8-{3-[(dimethylamino)methyl]phenyl}quinazolin-4-yl)amino]propyl]piperazin-1-yl}sulfonyl)-4-methyl-1,3-thiazol-2-yl]carbamate (Example 21)

To a solution of methyl N-[5-[4-[(2S)-2-[[8-(3-formylphenyl)quinazolin-4-yl]amino]propyl]piperazin-1-yl]sulfonyl-4-methyl-1,3-thiazol-2-yl]carbamate (Intermediate 8, 4.2 g, 6 mmol) in DCM (40 mL), Acetic acid (2 mL), Dimethylamine (7.7 mL, 15.4 mmol) and STAB (2.61 g, 12.3 mmol) were added and the reaction was stirred at rt overnight. To the resulting mixture sat. aq. NaHCO₃ was added till pH 7 and then extracted with DCM. The organic layer was concentrated under reduced pressure and the crude was purified by flash chromatography using a gradient of MeOH in DCM from 0% to 6% providing title compound (1.6 g, 2.5 mmol, 41% yield).

LC-MS (ESI): m/z (M+1): 639.2 (Method 2)

¹H NMR (400 MHz, DMSO-d₆) δ ppm 1.22 (d, J=6.6 Hz, 3H), 2.24 (s, 6H), 2.41 (s, 3H), 2.43-2.47 (m, 1H), 2.54-2.64 (m 5H), 2.94 (br s, 4H), 3.47-3.65 (m, 2H), 3.68 (s, 3H), 4.57-4.71 (m, 1H), 7.30 (d, J=7.5 Hz, 1H), 7.39 (t, J=7.6 Hz, 1H), 7.47-7.56 (m, 3H), 7.73 (dd, J=7.2, 1.1 Hz, 1H), 7.84 (d, J=7.9 Hz, 1H), 8.25 (d, J=7.5 Hz, 1H), 8.39 (s, 1H)

The Examples in the following table were prepared from commercially available reagents by using methods analogous to Example 21.

| Example | | |
|---|---|---|
| No. | Structure & Name | Analytical data |
| 16 | 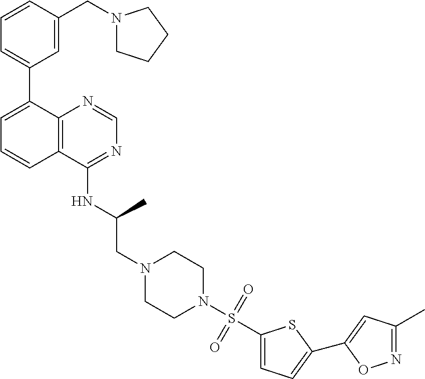 [SMILES: Cc1cc(-c2ccc(S(=O)(=O)N3CCN(C[C@H](C)Nc4ncnc5c(-c6cccc(CN7CCCC7)c6)cccc45)CC3)s2)on1] | LC-MS (ESI): m/z (M + 1): 658.2 (Method 2) ¹H NMR (500 MHz, DMSO-d₆) δ ppm 1.20 (d, J = 6.6 Hz, 3 H), 1.69 (br s, 4 H), 2.28 (s, 3 H), 2.40-2.45 (m, 1 H), 2.44-2.48 (m, 4 H), 2.59 (br s, 4 H), 2.60- 2.66 (m, 1 H), 2.98 (br s, 4 H), 3.63 (s, 2 H), 4.61 (spt, J = 6.9 Hz, 1 H), 6.96 (s, 1 H), 7.29 (br d, J = 7.5 Hz, 1 H), 7.36 (t, J = 7.5 Hz, 1 H), 7.44-7.49 (m, 2 H), 7.52 (t, J = 7.8 Hz, 1 H), 7.68- 7.73 (m, 2 H), 7.76 (d, J = 4.0 Hz, 1 H), 7.86 (br d, J = 8.8 Hz, 1 H), 8.23 (d, J = 8.2 Hz, 1 H), 8.40 (s, 1 H) |
| | N-[(2S)-1-(4-{[5-(3-methyl-1,2-oxazol-5- | |
| | yl)thiophen-2-yl]sulfonyl}piperidin-1- | |
| | yl)propan-2-yl]-8-{3-[(pyrrolidin-1- | |
| | yl)methyl]phenyl}quinazolin-4-amine | |
| 17 | 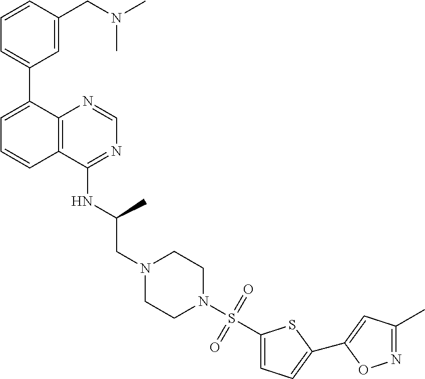 [SMILES: Cc1cc(-c2ccc(S(=O)(=O)N3CCN(C[C@H](C)Nc4ncnc5c(-c6cccc(CN(C)C)c6)cccc45)CC3)s2)on1] | LC-MS (ESI): m/z (M + 1): 632.1 (Method 2) ¹H NMR (500 MHz, DMSO-d₆) δ ppm 1.20 (d, J = 6.6 Hz, 3 H), 2.17 (s, 6 H), 2.28 (s, 3 H), 2.43 (br dd, J = 12.4, 6.9 Hz, 1 H), 2.55-2.66 (m, 4 H), 2.61-2.66 (m, 1 H), 2.98 (br s, 4 H), 3.44 (s, 2 H), 4.61 (spt, J = 6.8 Hz, 1 H), 6.96 (s, 1 H), 7.28 (d, J = 7.5 Hz, 1 H), 7.37 (t, J = 7.5 Hz, 1 H), 7.45-7.50 (m, 2 H), 7.52 (t, J = 7.8 Hz, 1 H), 7.68-7.73 (m, 2 H), 7.76 (d, J = 4.0 Hz, 1 H), 7.86 (br d, J = 7.8 Hz, 1 H), 8.23 (d, J = 8.2 Hz, 1 H), 8.40 (s, 1 H) |
| | 8-{3-[(dimethylamino)methyl]phenyl}- | |
| | N-[(2S)-1-(4-{[5-(3-methyl-1,2-oxazol-5- | |
| | yl)thiophen-2-yl]sulfonyl}piperazin-1- | |
| | yl)propan-2-yl]quinazolin-4-amine | |
| 22 | 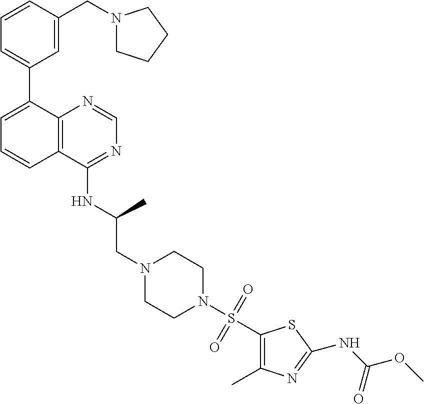 [SMILES: COC(=O)Nc1nc(C)c(S(=O)(=O)N2CCN(C[C@H](C)Nc3ncnc4c(-c5cccc(CN6CCCC6)c5)cccc34)CC2)s1] | LC-MS (ESI): m/z (M + 1): 665.2 (Method 2) ¹H NMR (400 MHz, DMSO-d₆) δ ppm 1.21 (d, J = 6.6 Hz, 3 H), 1.75 (br s, 4 H), 2.41 (s, 3 H), 2.44 (br d, J = 6.1 Hz, 1 H), 2.64 (br dd, J = 13.6, 7.2 Hz, 9 H), 2.89 (br s, 4 H), 3.65 (s, 3 H), 3.77 (d, J = 12.9 Hz, 1 H), 3.94 (br d, J = 12.9 Hz, 1 H), 4.65 (dt, J = 13.8, 6.8 Hz, 1 H), 7.32- 7.36 (m, 1 H), 7.40 (t, J = 7.5 Hz, 1 H), 7.45-7.50 (m, 1 H), 7.52 (t, J = 7.8 Hz, 1 H), 7.60 (s, 1 H), 7.72 (dd, J = 7.2, 0.9 Hz, 1 H), 7.81 (d, J = 8.3 Hz, 1 H), 8.24 (d, J = 7.7 Hz, 1 H), 8.37 (s, 1 H) |
| | methyl N-[4-methyl-5-({4-[(2S)-2-[(8- | |
| | {3-[(pyrrolidin-1- | |
| | yl)methyl]phenyl}quinazolin-4- | |
| | yl)amino]propyl]piperazin-1- | |
| | yl}sulfonyl)-1,3-thiazol-2-yl]carbamate | |
| 23 | 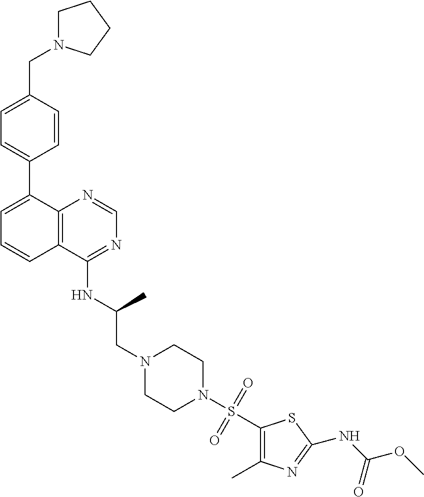 [SMILES: COC(=O)Nc1nc(C)c(S(=O)(=O)N2CCN(C[C@H](C)Nc3ncnc4c(-c5ccc(CN6CCCC6)cc5)cccc34)CC2)s1] | LC-MS (ESI): m/z (M + 1): 665.2 (Method 2) ¹H NMR (400 MHz, DMSO-d₆) δ ppm 1.21 (d, J = 6.6 Hz, 3 H), 1.73 (br t, J = 3.2 Hz, 4 H), 2.35- 2.46 (m, 4 H), 2.47-2.53 (m, 4 H), 2.53-2.66 (m, 5 H), 2.99 (br s, 4 H), 3.65 (s, 2 H), 3.72 (s, 3 H), 4.61 (quin, J = 6.8 Hz, 1 H), 7.37 (d, J = 8.1 Hz, 2 H), 7.48-7.61 (m, 3 H), 7.74 (dd, J = 7.1, 1.0 Hz, 1 H), 7.86 (d, J = 7.9 Hz, 1 H), 8.24 (d, J = 7.5 Hz, 1 H), 8.36-8.47 (m, 1 H), 11.10-13.11 (m, 1 H) |
| | methyl N-[4-methyl-5-({4-[(2S)-2-[(8- | |
| | {4-[(pyrrolidin-1- | |
| | yl)methyl]phenyl}quinazolin-4- | |
| | yl)amino]propyl]piperazin-1- | |
| | yl}sulfonyl)-1,3-thiazol-2-yl]carbamate | |
| 24 | 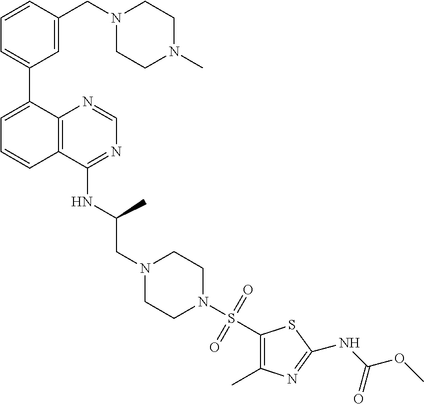 [SMILES: COC(=O)Nc1nc(C)c(S(=O)(=O)N2CCN(C[C@H](C)Nc3ncnc4c(-c5cccc(CN6CCN(C)CC6)c5)cccc34)CC2)s1] | LC-MS (ESI): m/z (M + 1): 694.2 (Method 2) ¹H NMR (500 MHz, DMSO-d₆) δ ppm 1.22 (d, J = 6.3 Hz, 3 H), 2.15 (s, 3 H), 2.24-2.49 (m, 9 H), 2.41 (s, 3 H), 2.53-2.62 (m, 5 H), 2.91-3.06 (m, 4 H), 3.53 (s, 2 H), 3.72 (s, 3 H), 4.61 (spt, J = 6.9 Hz, 1 H), 7.28 (d, J = 7.7 Hz, 1 H), 7.38 (t, J = 7.8 Hz, 1 H), 7.46-7.50 (m, 2 H), 7.54 (t, J = 7.7 Hz, 1 H), 7.73 (dd, J = 7.1, 1.1 Hz, 1 H), 7.87 (d, J = 8.0 Hz, 1 H), 8.25 (dd, J = 8.4, 1.0 Hz, 1 H), 8.40 (s, 1 H) |
| | methyl N-[4-methyl-5-({4-[(2S)-2-[(8- | |
| | {3-[(4-methylpiperazin-1- | |
| | yl)methyl]phenyl}quinazolin-4- | |
| | yl)amino]propyl]piperazin-1- | |
| | yl}sulfonyl)-1,3-thiazol-2-yl]carbamate | |
| 25 | 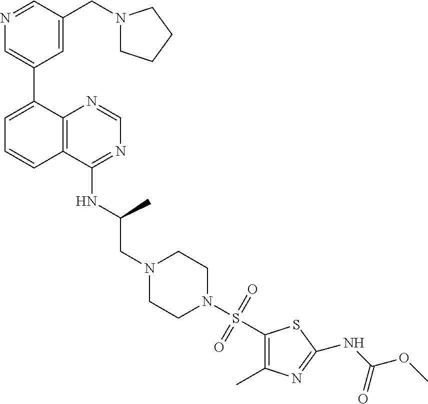 [SMILES: COC(=O)Nc1nc(C)c(S(=O)(=O)N2CCN(C[C@H](C)Nc3ncnc4c(-c5cncc(CN6CCCC6)c5)cccc34)CC2)s1] | LC-MS (ESI): m/z (M + 1): 666.2 (Method 2) ¹H NMR (500 MHz, DMSO-d₆) δ ppm 1.22 (d, J = 6.6 Hz, 3 H), 1.68-1.84 (m, 4 H), 2.42 (s, 3 H), 2.43-2.46 (m, 1 H), 2.47- 2.74 (m, 9 H), 2.92 (br s, 4 H), 3.70 (s, 3 H), 3.75-4.05 (m, 2 H), 4.59-4.73 (m, 1 H), 7.57 (t, J = 7.8 Hz, 1 H), 7.83 (d, J = 6.9 Hz, 1 H), 7.91 (br d, J = 8.0 Hz, 1 H), 8.00 (br s, 1 H), 8.31 (d, J = 8.0 Hz, 1 H), 8.40 (s, 1 H), 8.53 (d, J = 1.1 Hz, 1 H), 8.69 (d, J = 1.4 Hz, 1 H), 12.29 (br s, 1 H) |
| | methyl N-[4-methyl-5-({4-[(2S)-2-[(8- | |
| | {5-[(pyrrolidin-1-yl)methyl]pyridin-3- | |
| | yl}quinazolin-4- | |
| | yl)amino]propyl]piperazin-1- | |
| | yl}sulfonyl)-1,3-thiazol-2-yl]carbamate | |
| 45 | 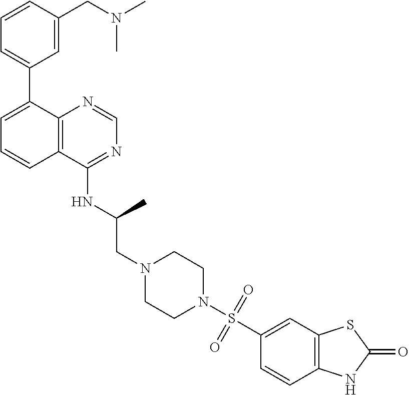 [SMILES: C[C@@H](CN1CCN(S(=O)(=O)c2ccc3[nH]c(=O)sc3c2)CC1)Nc1ncnc2c(-c3cccc(CN(C)C)c3)cccc12] | LC-MS (ESI): m/z (M + 1): 618.1 (Method 2) ¹H NMR (500 MHz, DMSO-d₆) δ ppm 1.18 (d, J = 6.6 Hz, 3 H), 2.23 (s, 6 H), 2.35-2.44 (m, 1 H), 2.52-2.63 (m, 5 H), 2.83 (br s, 4 H), 3.53 (s, 2 H), 4.59 (tt, J = 13.8, 6.8 Hz, 1 H), 7.20 (d, J = 8.2 Hz, 1 H), 7.29 (d, J = 7.7 Hz, 1 H), 7.39 (t, J = 7.5 Hz, 1 H), 7.45-7.59 (m, 4 H), 7.67-7.76 (m, 1 H), 7.48 (br d, J = 8.0 Hz, 1 H), 7.91 (d, J = 1.4 Hz, 1 H), 8.22 (d, J = 8.0 Hz, 1 H), 8.39 (s, 1 H), 10.15-13.70 (m, 1 H) |
| | 6-({4-[(2S)-2-[(8-{3- | |
| | [(dimethylamino)methyl]phenyl}quinazolin- | |
| | 4-yl)amino]propyl]piperazin-1- | |
| | yl}sulfonyl)-2,3-dihydro-1,3- | |
| | benzothiazol-2-one | |
| 46 | 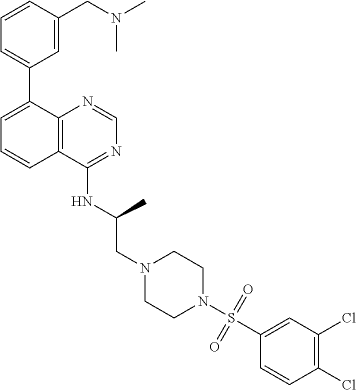 [SMILES: C[C@@H](CN1CCN(S(=O)(=O)c2ccc(Cl)c(Cl)c2)CC1)Nc1ncnc2c(-c3cccc(CN(C)C)c3)cccc12] | LC-MS (ESI): m/z (M + 1): 613.1 (Method 2) ¹H NMR (400 MHz, DMSO-d₆) δ ppm 1.19 (d, J = 6.4 Hz, 3 H), 2.24-2.46 (m, 7 H), 2.52-2.58 (m, 4 H), 2.62 (dd, J = 12.5, 7.5 Hz, 1 H), 2.93 (br s, 4 H), 3.46- 3.98 (m, 2 H), 4.60 (dt, J = 13.9, 6.8 Hz, 1 H), 7.35 (br d, J = 6.8 Hz, 1 H), 7.43 (br t, J = 7.3 Hz, 1 H), 7.49-7.61 (m, 3 H), 7.68 (dd, J = 8.5, 2.1 Hz, 1 H), 7.75 (d, J = 7.0 Hz, 1 H), 7.82- 7.94 (m, 3 H), 8.25 (d, J = 8.6 Hz, 1 H), 8.40 (s, 1 H) |
| | N-[(2S)-1-[4-(3,4- | |
| | dichlorobenzenesulfonyl)piperazin-1- | |
| | yl]propan-2-yl]-8-{3- | |
| | [(dimethylamino)methyl]phenyl}quinazolin- | |
| | 4-amine | |
| 50 | 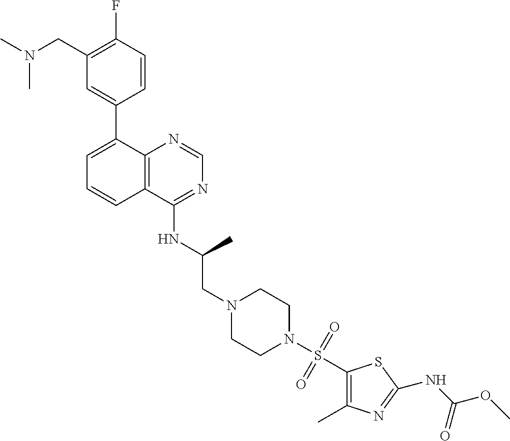 | LC-MS (ESI): m/z (M + 1): 657.2 (Method 2) ¹H NMR (400 MHz, DMSO-d₆) δ ppm 1.21 (d, J = 6.6 Hz, 3 H), 2.23 (s, 6 H), 2.37-2.47 (m, 1 H), 2.42 (s, 3 H), 2.53-2.65 (m, 5 H), 2.96 (br s, 4 H), 3.51- 3.65 (m, 2 H), 3.71 (s, 3 H), 4.62 (dt, J = 13.7, 6.7 Hz, 1 H), 7.24 (dd, J = 9.9, 8.6 Hz, 1 H), 7.48-7.58 (m, 2 H), 7.61 (dd, J = 7.2, 2.2 Hz, 1 H), 7.74 (dd, J = 7.1, 1.0 Hz, 1 H), 7.87 (d, J = 7.9 Hz, 1 H), 8.25 (d, J = 7.7 Hz, 1 H), 8.40 (s, 1 H), 10.83- 13.45 (m, 1 H) |
| | methyl N-[5-({4-[(2S)-2-[(8-{3- | |
| | [(dimethylamino)methyl]-4- | |
| | fluorophenyl}quinazolin-4- | |
| | yl)amino]propyl]piperazin-1- | |
| | yl}sulfonyl)-4-methyl-1,3-thiazol-2- | |
| | yl]carbamate | |
| 53 | 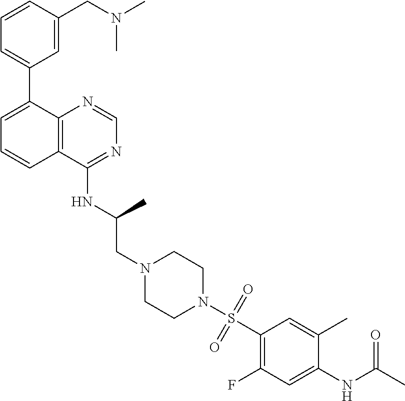 [SMILES: CC(=O)Nc1cc(F)c(S(=O)(=O)N2CCN(C[C@H](C)Nc3ncnc4c(-c5cccc(CN(C)C)c5)cccc34)CC2)cc1C] | LC-MS (ESI): m/z (M + 1): 634.2 (Method 2) ¹H NMR (400 MHz, DMSO-d₆) δ ppm 1.20 (d, J = 6.6 Hz, 3 H), 2.14 (s, 3 H), 2.17 (s, 6 H), 2.27 (s, 3 H), 2.42 (dd, J = 12.4, 6.9 Hz, 1 H), 2.52-2.57 (m, 4 H), 2.61 (dd, J = 12.4, 7.1 Hz, 1 H), 2.91-3.05 (m, 4 H), 3.44 (s, 2 H), 4.53-4.66 (m, 1 H), 7.28 (d, J = 7.7 Hz, 1 H), 7.38 (t, J = 7.6 Hz, 1 H), 7.47 (s, 1 H), 7.48-7.57 (m, 3 H), 7.73 (dd, J = 7.1, 1.0 Hz, 1 H), 7.82-7.87 (m, 1 H), 7.88 (d, J = 13.1 Hz, 1 H), 8.24 (d, J = 7.5 Hz, 1 H), 8.40 (s, 1 H), 9.44 (s, 1 H) |
| | N-[4-({4-[(2S)-2-[(8-{3- | |
| | [(dimethylamino)methyl]phenyl}quinazolin- | |
| | 4-yl)amino]propyl]piperazin-1- | |
| | yl}sulfonyl)-5-fluoro-2- | |
| | methylphenyl]acetamide | |
| 54 | 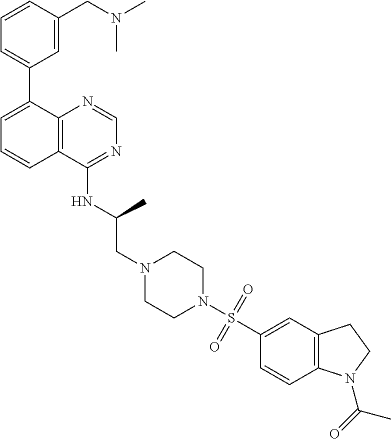 [SMILES: CC(=O)N1CCc2cc(S(=O)(=O)N3CCN(C[C@H](C)Nc4ncnc5c(-c6cccc(CN(C)C)c6)cccc45)CC3)ccc21] | LC-MS (ESI): m/z (M + 1): 628.2 (Method 2) ¹H NMR (400 MHz, DMSO-d₆) δ ppm 1.18 (d, J = 6.6 Hz, 3 H), 2.17 (s, 9 H), 2.39 (dd, J = 12.5, 6.6 Hz, 1 H), 2.52-2.57 (m, 4 H), 2.61 (dd, J = 12.3, 7.5 Hz, 1 H), 2.75-2.91 (m, 4 H), 3.19 (br t, J = 8.7 Hz, 2 H), 3.44 (s, 2 H), 4.13 (br t, J = 8.1 Hz, 2 H), 4.52-4.66 (m, 1 H), 7.28 (d, J = 7.7 Hz, 1 H), 7.38 (t, J = 7.6 Hz, 1 H), 7.47 (br s, 1 H), 7.48- 7.57 (m, 4 H), 7.73 (dd, J = 7.2, 1.1 Hz, 1 H), 7.84 (d, J = 7.9 Hz, 1 H), 8.17 (br d, J = 9.0 Hz, 1 H), 8.22 (d, J = 7.7 Hz, 1 H), 8.40 (s, 1 H) |
| | 1-[5-({4-[(2S)-2-[(8-{3- | |
| | [(dimethylamino)methyl]phenyl}quinazolin- | |
| | 4-yl)amino]propyl]piperazin-1- | |
| | yl}sulfonyl)-2,3-dihydro-1H-indol-1- | |
| | yl]ethan-1-one | |
| 55 | 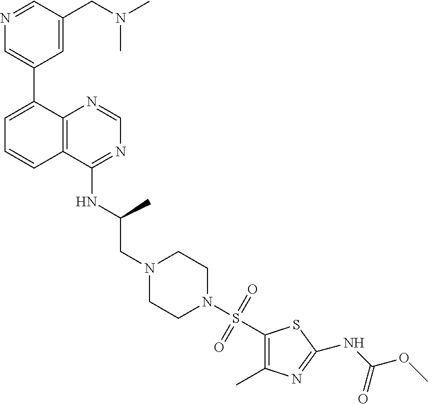 [SMILES: COC(=O)Nc1nc(C)c(S(=O)(=O)N2CCN(C[C@H](C)Nc3ncnc4c(-c5cncc(CN(C)C)c5)cccc34)CC2)s1] | LC-MS (ESI): m/z (M + 1): 640.2 (Method 2) ¹H NMR (400 MHz, DMSO-d₆) δ ppm 1.22 (d, J = 6.6 Hz, 3 H), 2.24 (s, 6 H), 2.38-2.48 (m, 1 H), 2.43 (s, 3 H), 2.53-2.65 (m, 5 H), 2.96 (br s, 4 H), 3.50- 3.67 (m, 2 H), 3.72 (s, 3 H), 4.64 (dt, J = 13.9, 6.9 Hz, 1 H), 7.57 (t, J = 7.8 Hz, 1 H), 7.82 (dd, J = 7.2, 1.1 Hz, 1 H), 7.86- 8.00 (m, 2 H), 8.31 (d, J = 8.3 Hz, 1 H), 8.42 (s, 1 H), 8.48 (d, J = 1.8 Hz, 1 H), 8.70 (d, J = 2.0 Hz, 1 H), 11.00-13.71 (m, 1 H) |
| | methyl N-[5-({4-[(2S)-2-[(8-{5- | |
| | [(dimethylamino)methyl]pyridin-3- | |
| | yl}quinazolin-4- | |
| | yl)amino]propyl]piperazin-1- | |
| | yl}sulfonyl)-4-methyl-1,3-thiazol-2- | |
| | yl]carbamate | |
| 60 | 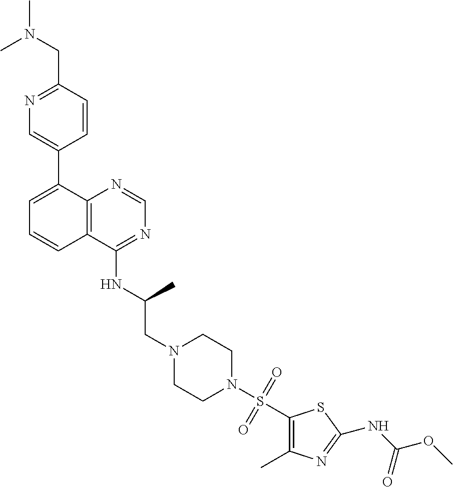 | LC-MS (ESI): m/z (M + 1): 640.2 (Method 2) ¹H NMR (400 MHz, DMSO-d₆) δ ppm 1.22 (d, J = 6.6 Hz, 3 H), 2.26 (s, 6 H), 2.40-2.47 (m, 4 H), 2.58 (br s, 5 H), 2.99 (br s, 4 H), 3.60 (s, 2 H), 3.74 (s, 3 H), 4.61 (dt, J = 13.9, 6.8 Hz, 1 H), 7.50 (d, J = 8.1 Hz, 1 H), 7.57 (t, J = 7.8 Hz, 1 H), 7.83 (d, J = 7.0 Hz, 1 H), 7.93 (d, J = 7.9 Hz, 1 H), 8.03 (dd, J = 8.0, 2.1 Hz, 1 H), 8.30 (d, J = 7.7 Hz, 1 H), 8.44 (s, 1 H), 8.70 (d, J = 2.0 Hz, 1 H), 11.30-12.88 (m, 1 H) |
| | methyl N-[5-({4-[(2S)-2-[(8-{6- | |
| | [(dimethylamino)methyl]pyridin-3- | |
| | yl}quinazolin-4- | |
| | yl)amino]propyl]piperazin-1- | |
| | yl}sulfonyl)-4-methyl-1,3-thiazol-2- | |
| | yl]carbamate | |
| 76 | 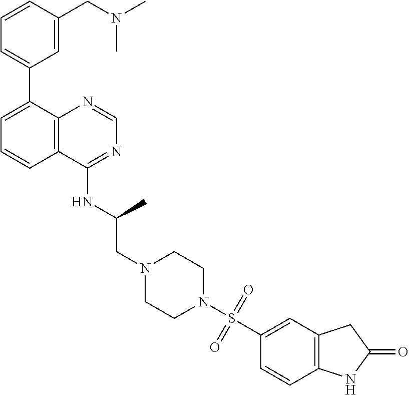 [SMILES: C[C@@H](CN1CCN(S(=O)(=O)c2ccc3c(c2)CC(=O)N3)CC1)Nc1ncnc2c(-c3cccc(CN(C)C)c3)cccc12] | LC-MS (ESI): m/z (M + 1): (Method) ¹H NMR (400 MHz, DMSO-d₆) δ ppm 1.22 (d, J = 6.6 Hz, 3 H), 2.26 (s, 6 H), 2.40-2.47 (m, 4 H), 2.58 (br s, 5 H), 2.99 (br s, 4 H), 3.60 (s, 2 H), 3.74 (s, 3 H), 4.61 (dt, J = 13.9, 6.8 Hz, 1 H), 7.50 (d, J = 8.1 Hz, 1 H), 7.57 (t, J = 7.8 Hz, 1 H), 7.83 (d, J = 7.0 Hz, 1 H), 7.93 (d, J = 7.9 Hz, 1 H), 8.03 (dd, J = 8.0, 2.1 Hz, 1 H), 8.30 (d, J = 7.7 Hz, 1 H), 8.44 (s, 1 H), 8.70 (d, J = 2.0 Hz, 1 H), 11.30-12.88 (m, 1 H) |
| | 5-({4-[(2S)-2-[(8-{3- | |
| | [(dimethylamino)methyl]phenyl}quinazolin- | |
| | 4-yl)amino]propyl]piperazin-1- | |
| | yl}sulfonyl)-2,3-dihydro-1H-indol-2-one | |
| 77 | 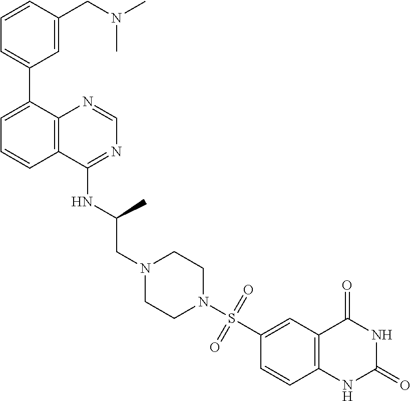 [SMILES: C[C@@H](CN1CCN(S(=O)(=O)c2ccc3[nH]c(=O)[nH]c(=O)c3c2)CC1)Nc1ncnc2c(-c3cccc(CN(C)C)c3)cccc12] | LC-MS (ESI): m/z (M + 1): 629.28 (Method 2) ¹H NMR (400 MHz, DMSO-d₆) δ ppm 1.17 (d, J = 6.4 Hz, 3 H), 2.17 (s, 6 H), 2.39 (dd, J = 12.4, 6.7 Hz, 1 H), 2.52-2.57 (m, 4 H), 2.61 (dd, J = 12.3, 7.3 Hz, 1 H), 2.86 (br s, 4 H), 3.45 (s, 2 H), 4.52-4.64 (m, 1 H), 7.24- 7.32 (m, 2 H), 7.37 (t, J = 7.5 Hz, 1 H), 7.44-7.55 (m, 3 H), 7.72 (dd, J = 7.3, 1.3 Hz, 1 H), 7.83 (d, J = 7.9 Hz, 1 H), 7.87 (dd, J = 8.8, 2.2 Hz, 1 H), 8.08 (d, J = 2.2 Hz, 1 H), 8.22 (dd, J = 8.4, 1.3 Hz, 1 H), 8.39 (s, 1 H), 11.43 (br s, 2 H) |
| | 6-({4-[(2S)-2-[8-{3- | |
| | [(dimethylamino)methyl]phenyl}quinazolin- | |
| | 4-yl)amino]propyl]piperazin-1- | |
| | yl}sulfonyl)-1,2,3,4- | |
| | tetrahydroquinazoline-2,4-dione | |
| 84 | 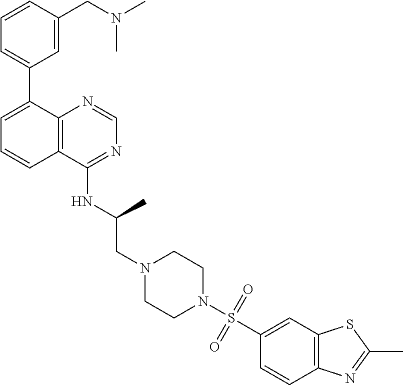 [SMILES: Cc1nc2ccc(S(=O)(=O)N3CCN(C[C@H](C)Nc4ncnc5c(-c6cccc(CN(C)C)c6)cccc45)CC3)cc2s1] | LC-MS (ESI): m/z (M + 1): 616.29 (Method 2) ¹H NMR (400 MHz, DMSO-d₆) δ ppm 1.16 (d, J = 6.6 Hz, 3 H), 2.17 (s, 6 H), 2.39 (br dd, J = 12.4, 6.9 Hz, 1 H), 2.54 (br s, 4 H), 2.56-2.62 (m, 1 H), 2.84 (s, 3 H), 2.90 (br s, 4 H), 3.44 (s, 2 H), 4.57 (dt, J = 14.0, 6.9 Hz, 1 H), 7.27 (d, J = 7.7 Hz, 1 H), 7.37 (t, J = 7.5 Hz, 1 H), 7.42- 7.54 (m, 3 H), 7.70 (dd, J = 7.2, 1.2 Hz, 1 H), 7.76 (dd, J = 8.6, 1.9 Hz, 1 H), 7.82 (d, J = 7.9 Hz, 1 H), 8.08 (d, J = 8.6 Hz, 1 H), 8.20 (dd, J = 8.4, 1.0 Hz, 1 H), 8.39 (s, 1 H), 8.52 (d, J = 1.6 Hz, 1 H) |
| | 8-{3-[(dimethylamino)methyl]phenyl}- | |
| | N-[(2S)-1-{4-[(2-methyl-1,3- | |
| | benzothiazol-6-yl)sulfonyl]piperazin-1- | |
| | yl}propan-2-yl]quinazolin-4-amine | |
| 89 | 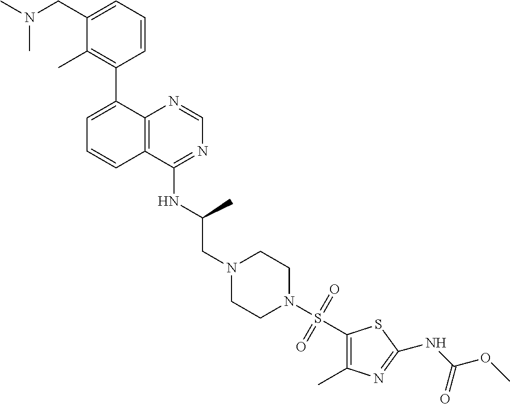 | LC-MS (ESI): m/z (M + 1): 653.3 (Method 2) ¹H NMR (400 MHz, DMSO-d₆) δ ppm 1.21 (dd, J = 6.4, 3.9 Hz, 3 H), 1.94 (s, 3 H), 2.33 (br s, 6 H), 2.38-2.46 (m, 1 H), 2.43 (d, J = 2.9 Hz, 3 H), 2.54-2.65 (m, 5 H), 2.93-3.08 (m, 4 H), 3.31 (br s, 2 H), 3.76 (d, J = 1.5 Hz, 3 H), 4.60 (dq, J = 12.7, 6.4 Hz, 1 H), 7.00-7.37 (m, 3 H), 7.46-7.60 (m, 2 H), 7.89 (br d, J = 6.8 Hz, 1 H), 8.28 (dd, J = 7.8, 1.4 Hz, 1 H), 8.32 (s, 1 H), 11.46-13.10 (m, 1 H) |
| | methyl N-[5-({4-[(2S)-2-[(8-{3- | |
| | [(dimethylamino)methyl]-2- | |
| | methylphenyl}quinazolin-4- | |
| | yl)amino]propyl]piperazin-1- | |
| | yl}sulfonyl)-4-methyl-1,3-thiazol-2- | |
| | yl]carbamate | |
| 90 | 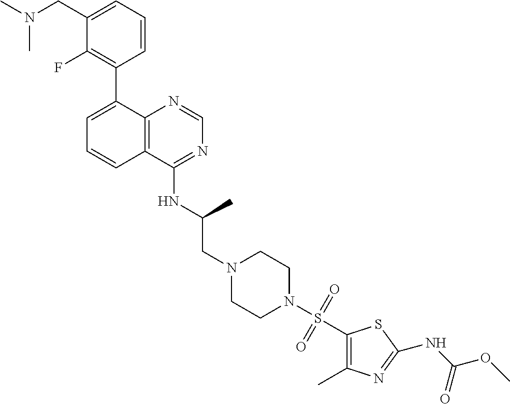 | LC-MS (ESI): m/z (M + 1): 657.3 (Method 2) ¹H NMR (400 MHz, DMSO-d₆) δ ppm 1.21 (d, J = 6.4 Hz, 3 H), 2.20 (s, 6 H), 2.42 (s, 4 H), 2.54- 2.65 (m, 5 H), 2.99 (br s, 4 H), 3.50 (s, 2 H), 3.73 (s, 3 H), 4.53- 4.69 (m, 1 H), 7.17-7.25 (m, 1 H), 7.26-7.32 (m, 1 H), 7.41 (td, J = 7.1, 1.6 Hz, 1 H), 7.54 (t, J = 7.4 Hz, 1 H), 7.70 (d, J = 7.0 Hz, 1 H), 7.90 (d, J = 7.9 Hz, 1 H), 8.31 (d, J = 7.5 Hz, 1 H), 8.36 (s, 1 H), 12.21 (br s, 1 H) |
| | methyl N-[5-({4-[(2S)-2-[(8-{3- | |
| | [(dimethylamino)methyl]-2- | |
| | fluorophenyl}quinazolin-4- | |
| | yl)amino]propyl]piperazin-1- | |
| | yl}sulfonyl)-4-methyl-1,3-thiazol-2- | |
| | yl]carbamate | |
| 91 | 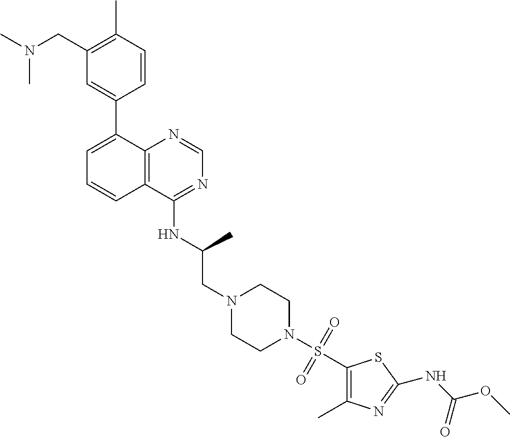 | LC-MS (ESI): m/z (M + 1): 653.3 (Method 2) ¹H NMR (400 MHz, DMSO-d₆) δ ppm 1.21 (d, J = 6.6 Hz, 3 H), 2.22 (s, 6 H), 2.37 (s, 3 H), 2.39- 2.46 (m, 4 H), 2.54-2.65 (m, 5 H), 2.97 (br s, 4 H), 3.46 (br d, J = 3.9 Hz, 2 H), 3.71 (s, 3 H), 4.51-4.72 (m, 1 H), 7.22 (d, J = 7.9 Hz, 1 H), 7.41 (dd, J = 7.7, 1.5 Hz, 1 H), 7.45 (s, 1 H), 7.52 (t, J = 7.8 Hz, 1 H), 7.66-7.75 (m, 1 H), 7.83 (d, J = 8.1 Hz, 1 H), 8.22 (d, J = 8.3 Hz, 1 H), 8.39 (s, 1 H) |
| | methyl N-[5-({4-[(2S)-2-[(8-{3- | |
| | [(dimethylamino)methyl]-4- | |
| | methylphenyl}quinazolin-4- | |
| | yl)amino]propyl]piperazin-1- | |
| | yl}sulfonyl)-4-methyl-1,3-thiazol-2- | |
| | yl]carbamate | |
| 93 | 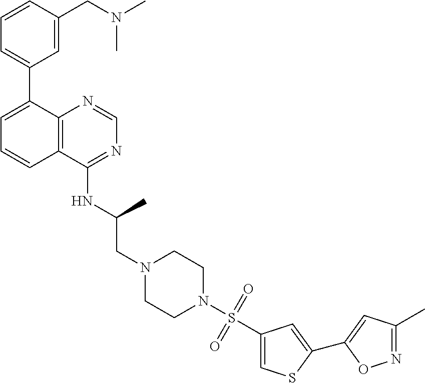 | LC-MS (ESI): m/z (M + 1): 623.3 (Method 1) ¹H NMR (500 MHz, DMSO-d₆) δ ppm 1.20 (d, J = 6.6 Hz, 3 H), 2.24 (br s, 6 H), 2.26 (s, 3 H), 2.42 (dd, J = 12.3, 6.7 Hz, 1 H), 2.56 (br s, 4 H), 2.59-2.65 (m, 1 H), 2.95 (br s, 4 H), 3.52 (br s, 2 H), 4.60 (spt, J = 6.9 Hz, 1 H), 6.92 (s, 1 H), 7.30 (br d, J = 7.5 Hz, 1 H), 7.39 (t, J = 7.5 Hz, 1 H), 7.47-7.54 (m, 3 H), 7.72 (dd, J = 7.1, 1.1 Hz, 1 H), 7.83 (d, J = 1.5 Hz, 1 H), 7.86 (d, J = 8.0 Hz, 1 H), 8.23 (dd, J = 8.4, 1.1 Hz, 1 H), 8.39 (d, J = 1.4 Hz, 1 H), 8.40 (s, 1 H) |
| | 8-{3-[(dimethylamino)methyl]phenyl}- | |
| | N-[(2S)-1-(4-{[5-(3-methyl-1,2-oxazol-5- | |
| | yl)thiophen-3-yl]sulfonyl}piperazin-1- | |
| | yl)propan-2-yl]quinazolin-4-amine | |
| 96 | 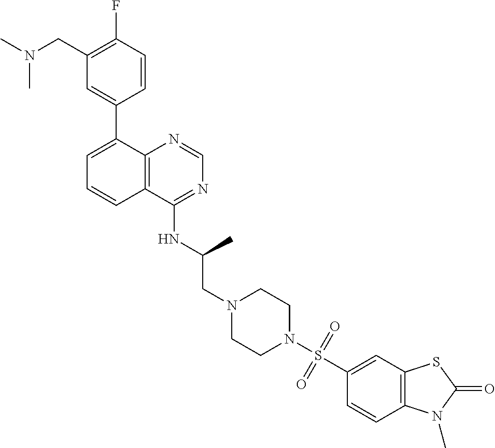 [SMILES: C[C@@H](CN1CCN(S(=O)(=O)c2ccc3c(c2)sc(=O)n3C)CC1)Nc1ncnc2c(-c3ccc(F)c(CN(C)C)c3)cccc12] | LC-MS (ESI): m/z (M + 1): 650.3 (Method 2) ¹H NMR (400 MHz, DMSO-d₆) δ ppm 1.17 (d, J = 6.6 Hz, 3 H), 2.19 (s, 6 H), 2.38 (dd, J = 12.3, 6.6 Hz, 1 H), 2.54 (br s, 4 H), 2.62 (dd, J = 12.4, 7.6 Hz, 1 H), 2.85 (br s, 4 H), 3.41 (s, 3 H), 3.50 (s, 2 H), 4.59 (dt, J = 13.9, 7.2 Hz, 1 H), 7.22 (dd, J = 9.9, 8.3 Hz, 1 H), 7.46-7.60 (m, 4 H), 7.69 (dd, J = 8.6, 2.0 Hz, 1 H), 7.71-7.76 (m, 1 H), 7.85 (d, J = 8.1 Hz, 1 H), 8.12 (d, J = 1.8 Hz, 1 H), 8.22 (d, J = 7.7 Hz, 1 H), 8.40 (s, 1 H) |
| | 6-({4-[(2S)-2-[(8-{3- | |
| | [(dimethylamino)methyl]-4- | |
| | fluorophenyl}quinazolin-4- | |
| | yl)amino]propyl]piperazin-1- | |
| | yl}sulfonyl)-3-methyl-2,3-dihydro-1,3- | |
| | benzothiazol-2-one | |
| 97 | 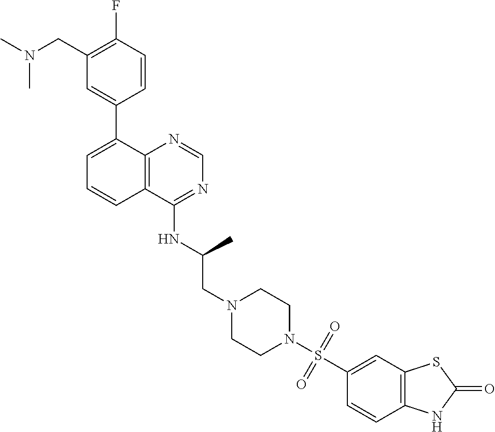 [SMILES: C[C@@H](CN1CCN(S(=O)(=O)c2ccc3[nH]c(=O)sc3c2)CC1)Nc1ncnc2c(-c3ccc(F)c(CN(C)C)c3)cccc12] | LC-MS (ESI): m/z (M + 1): 636.3 (Method 2) ¹H NMR (400 MHz, DMSO-d₆) δ ppm 1.18 (d, J = 6.6 Hz, 3 H), 2.22 (s, 6 H), 2.40 (dd, J = 12.4, 6.7 Hz, 1 H), 2.53 (br s, 4 H), 2.60 (dd, J = 12.4, 7.4 Hz, 1 H), 2.84 (br s, 4 H), 3.54 (s, 2 H), 4.59 (dt, J = 13.8, 6.8 Hz, 1 H), 7.19-7.30 (m, 2 H), 7.48-7.63 (m, 4 H), 7.73 (dd, J = 7.3, 1.3 Hz, 1 H), 7.85 (d, J = 7.9 Hz, 1 H), 7.96 (s, 1 H), 8.22 (dd, J = 8.5, 1.2 Hz, 1 H), 8.40 (s, 1 H) |
| | 6-({4-[(2S)-2-[(8-{3- | |
| | [(dimethylamino)methyl]-4- | |
| | fluorophenyl}quinazolin-4- | |
| | yl)amino]propyl]piperazin-1- | |
| | yl}sulfonyl)-2,3-dihydro-1,3- | |
| | benzothiazol-2-one | |
| 105 | 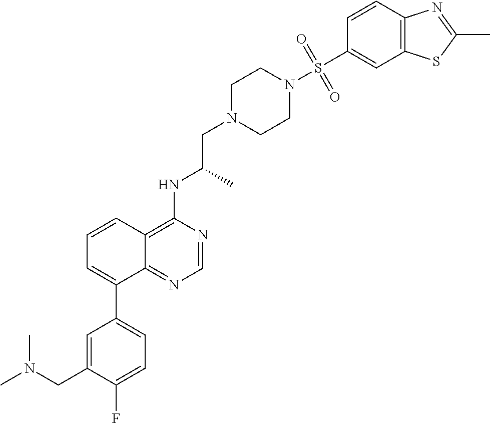 | LC-MS (ESI): m/z (M + 1): 634.3 (Method 2) ¹H NMR (400 MHz, DMSO-d₆) δ ppm 1.16 (d, J = 6.6 Hz, 3 H), 2.19 (s, 6 H), 2.39 (dd, J = 12.4, 6.9 Hz, 1 H), 2.51-2.54 (m, 4 H), 2.56-2.62 (m, 1 H), 2.85 (s, 3 H), 2.90 (br s, 4 H), 3.50 (s, 2 H), 4.44-4.72 (m, 1 H), 7.22 (dd, J = 10.0, 8.3 Hz, 1 H), 7.49 (dd, J = 8.1, 7.5 Hz, 1 H), 7.52-7.61 (m, 2 H), 7.68-7.78 (m, 2 H), 7.83 (d, J = 7.9 Hz, 1 H), 8.08 (d, J = 8.6 Hz, 1 H), 8.20 (dd, J = 8.5, 1.2 Hz, 1 H), 8.39 (s, 1 H), 8.52 (d, J = 1.5 Hz, 1 H) |
| | 8-{3-[(dimethylamino)methyl]-4- | |
| | fluorophenyl}-N-[(2S)-1-{4-[(2-methyl- | |
| | 1,3-benzothiazol-6-yl)sulfonyl]piperazin- | |
| | 1-yl}propan-2-yl]quinazolin-4-amine | |
| 106 | 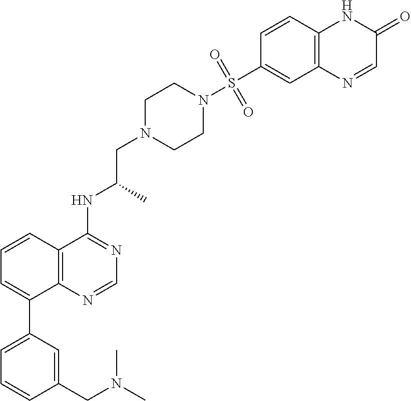 [SMILES: C[C@@H](CN1CCN(S(=O)(=O)c2ccc3[nH]c(=O)cnc3c2)CC1)Nc1ncnc2c(-c3cccc(CN(C)C)c3)cccc12] | LC-MS (ESI): m/z (M + 1): 613.3 (Method 2) ¹H NMR (500 MHz, DMSO-d₆) δ ppm 1.16 (d, J = 6.6 Hz, 3 H), 2.18 (s, 6 H), 2.32-2.45 (m, 1 H), 2.52-2.57 (m, 4 H), 2.60 (dd, J = 12.6, 7.4 Hz, 1 H), 2.78- 2.99 (m, 4 H), 3.45 (s, 2 H), 4.58 (dt, J = 14.1, 6.9 Hz, 1 H), 7.27 (d, J = 7.7 Hz, 1 H), 7.37 (t, J = 7.5 Hz, 1 H), 7.41-7.53 (m, 4 H), 7.71 (dd, J = 7.4, 1.4 Hz, 1 H), 7.77-7.89 (m, 2 H), 7.95- 8.01 (m, 1 H), 8.17-8.26 (m, 2 H), 8.39 (s, 1 H) |
| | 6-({4-[(2S)-2-[(8-{3- | |
| | [(dimethylamino)methyl]phenyl}quinazolin- | |
| | 4- | |
| | yl)amino]propyl]piperazin-1- | |
| | yl}sulfonyl)-1,2- | |
| | dihydroquinoxalin-2-one | |
| 107 | 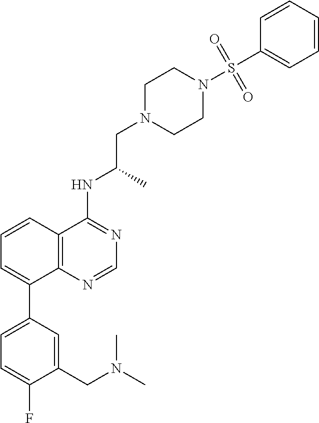 [SMILES: C[C@@H](CN1CCN(S(=O)(=O)c2ccccc2)CC1)Nc1ncnc2c(-c3ccc(F)c(CN(C)C)c3)cccc12] | LC-MS (ESI): m/z (M + 1): 563.2 (Method 2) ¹H NMR (400 MHz, DMSO-d₆) δ ppm 1.17 (d, J = 6.6 Hz, 3 H), 2.19 (s, 6 H), 2.39 (dd, J = 12.3, 6.7 Hz, 1 H), 2.51-2.56 (m, 4 H), 2.59 (dd, J = 12.4, 7.3 Hz, 1 H), 2.84 (br s, 4 H), 3.50 (s, 2 H), 4.59 (dt, J = 14.0, 6.9 Hz, 1 H), 7.23 (dd, J = 9.9, 8.6 Hz, 1 H), 7.46-7.60 (m, 3 H), 7.61- 7.66 (m, 2 H), 7.66-7.78 (m, 4 H), 7.85 (d, J = 7.9 Hz, 1 H), 8.22 (d, J = 7.7 Hz, 1 H), 8.40 (s, 1 H) |
| | N-[(2S)-1-[4-(benzenesulfonyl)piperazin- | |
| | 1-yl]propan-2-yl]-8-{3- | |
| | [(dimethylamino)methyl]-4- | |
| | fluorophenyl}quinazolin-4-amine | |

### Example 39

### methyl N-[5-({4-[(2S)-2-{[8-(pyridin-3-yl)quinazolin-4-yl]amino}propyl]piperazin-1-yl}sulfonyl)-1,3-thiazol-2-yl]carbamate

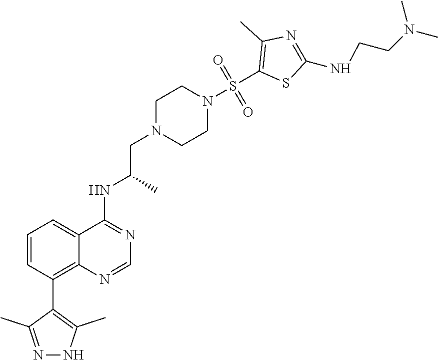

### Step 1: Preparation of tert-butyl 4-[4-[[(2S)-1-[4-[[2-(methoxycarbonylamino)-4-methyl-1,3-thiazol-5-yl]sulfonyl]piperazin-1-yl]propan-2-yl]amino]quinazolin-8-yl]-3,5-dimethylpyrazole-1-carboxylate (Intermediate 11)

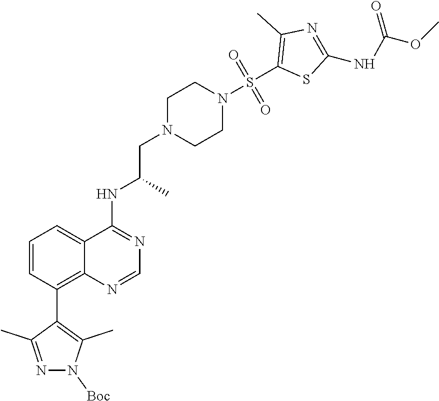

Title compound was prepared following the procedure used for the synthesis of Example 1 starting from methyl N-[5-[4-[(2S)-2-[(8-bromoquinazolin-4-yl)amino]propyl]piperazin-1-yl]sulfonyl-4-methyl-1,3-thiazol-2-yl]carbamate (Intermediate 3, 176 mg, 0.3 mmol) and [3,5-dimethyl-1-[(2-methylpropan-2-yl)oxycarbonyl]pyrazol-4-yl]boronic acid (108 mg, 0.45 mmol) to afford title compound (Intermediate 11, 82 mg, 0.117 mmol, 39% yield)

LC-MS (ESI): m/z (M+1): 700.2 (Method 2)

### Step 2: Preparation of methyl N-[5-[4-[(2S)-2-[[8-(3,5-dimethyl-1H-pyrazol-4-yl)quinazolin-4-yl]amino]propyl]piperazin-1-yl]sulfonyl-4-methyl-1,3-thiazol-2-yl]carbamate hydrochloride (Intermediate 12)

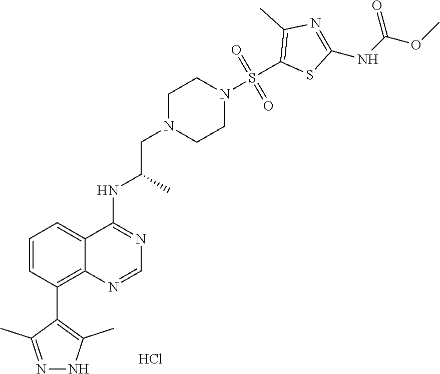

Title compound was prepared following the procedure used for the synthesis of Intermediate 2 starting from tert-butyl 4-[4-[[(2S)-1-[4-[[2-(methoxycarbonylamino)-4-methyl-1,3-thiazol-5-yl]sulfonyl]piperazin-1-yl]propan-2-yl]amino]quinazolin-8-yl]-3,5-dimethylpyrazole-1-carboxylate (Intermediate 11, 82 mg, 0.117 mmol) to afford title compound (87 mg, crude) that was used in the next step without further purification.

LC-MS (ESI): m/z (M+1): 600.1 (Method 2)

### Step 3: Preparation of methyl N-[5-({4-[(2S)-2-{[8-(pyridin-3-yl)quinazolin-4-yl]amino}propyl]piperazin-1-yl}sulfonyl)-1,3-thiazol-2-yl]carbamate (Example 39)

A solution of Intermediate 12 (87.3 mg, 0.150 mmol) and K₂CO₃ (40 mg, 0.29 mmol) in DMF (1 mL) was stirred for 5 min then 2-Chloro-N,N-dimethylethylamine hydrochloride (25 mg, 0.17 mmol) was added and the reaction was stirred at 50° C. rt for 3 h. NaHCO₃ sat. solution was added and the mixture extracted with AcOEt. Organic layer was separated, dried and solvent was removed under reduced pressure. The crude was purified by flash chromatography using a gradient of MeOH in DCM from 0% to 4% affording title compound (20 mg, 0.033 mmol, 22% yield) as a white solid.

LC-MS (ESI): m/z (M+1): 613.2 (Method 2)

¹H NMR (500 MHz, DMSO-d₆) δ ppm 1.22 (d, J=6.6 Hz, 3H), 1.91-2.10 (m, 6H), 2.14 (s, 6H), 2.30 (s, 3H), 2.33-2.47 (m, 3H), 2.51-2.63 (m, 7H), 2.96 (br s, 4H), 4.61 (dt, J=14.2, 7.0 Hz, 1H), 7.49 (dd, J=8.2, 7.4 Hz, 1H), 7.57 (dd, J=7.1, 1.4 Hz, 1H), 7.82 (d, J=8.0 Hz, 1H), 8.20 (dd, J=8.4, 1.2 Hz, 1H), 8.26 (br t, J=5.2 Hz, 1H), 8.38 (s, 1H), 12.19 (br s, 1H)

### Example 47

### methyl N-[5-({4-[(2S)-2-{[8-(pyridin-3-yl)quinazolin-4-yl]amino}propyl]piperazin-1-yl}sulfonyl)-1,3-thiazol-2-yl]carbamate

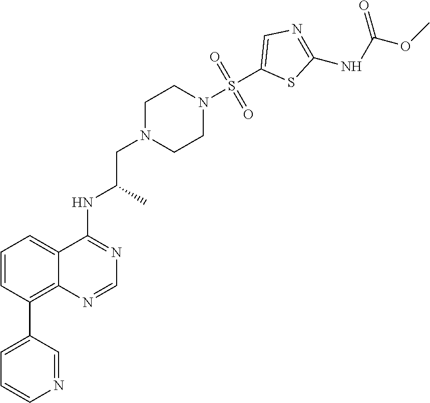 [SMILES: COC(=O)Nc1ncc(S(=O)(=O)N2CCN(C[C@H](C)Nc3ncnc4c(-c5cccnc5)cccc34)CC2)s1]

### Step 1: Preparation of tert-butyl 4-[(2S)-2-[(8-pyridin-3-ylquinazolin-4-yl)amino]propyl]piperazine-1-carboxylate (Intermediate 13)

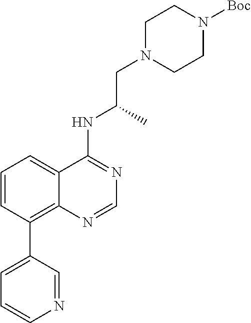 [SMILES: C[C@@H](CN1CCN(C(=O)OC(C)(C)C)CC1)Nc1ncnc2c(-c3cccnc3)cccc12]

Title compound was prepared following the procedure used for the synthesis of Example 1, starting from tert-butyl 4-[(2S)-2-[(8-bromoquinazolin-4-yl)amino]propyl]piperazine-1-carboxylate (Intermediate 1, 1.8 g, 4 mmol) and 3-pyridinylboronic acid (737 mg, 6 mmol) to afford title compound (1.72 g, 3.83 mmol, 96% yield) as a pale orange solid

LC-MS (ESI): m/z (M+1): 449.2 (Method 2)

### Step 2: Preparation of N-[(2S)-1-piperazin-1-ylpropan-2-yl]-8-pyridin-3-ylquinazolin-4-amine hydrochloride (Intermediate 14)

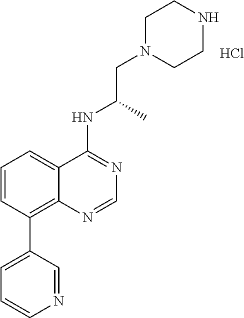 [SMILES: C[C@@H](CN1CCNCC1)Nc1ncnc2c(-c3cccnc3)cccc12 ; Cl]

Title compound was prepared following the procedure used for the synthesis of Intermediate 2 starting from tert-butyl 4-[(2S)-2-[(8-pyridin-3-ylquinazolin-4-yl)amino]propyl]piperazine-1-carboxylate (Intermediate 13, 1.72 g, 3.83 mmol) to afford title compound (2 g, crude) that was used in the next step without further purification.

LC-MS (ESI): m/z (M+1): 349.1 (Method 2)

### Step 3: Preparation of N-[5-[4-[(2S)-2-[(8-pyridin-3-ylquinazolin-4-yl)amino]propyl]piperazin-1-yl]sulfonyl-1,3-thiazol-2-yl]acetamide (Intermediate 15)

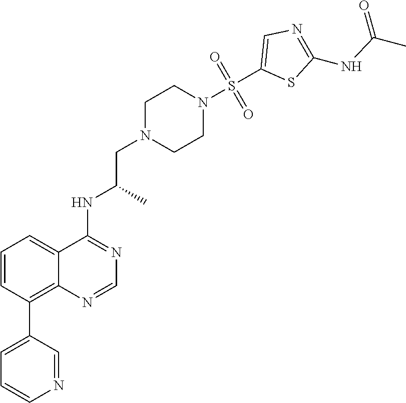 [SMILES: CC(=O)Nc1ncc(S(=O)(=O)N2CCN(C[C@H](C)Nc3ncnc4c(-c5cccnc5)cccc34)CC2)s1]

Title compound was prepared following the procedure used for the synthesis of Intermediate 3, starting from (Intermediate 14, 165 mg, 0.43 mmol) and 2-Acetylamino-thiazole-5-sulfonyl chloride (103 mg, 0.43 mmol) to afford title compound (Intermediate 15, 167 mg, 0.3 mmol, 70% yield).

LC-MS (ESI): m/z (M+1): 553.1 (Method 2)

### Step 4: Preparation of 5-[4-[(2S)-2-[(8-pyridin-3-ylquinazolin-4-yl)amino]propyl]piperazin-1-yl]sulfonyl-1,3-thiazol-2-amine (Intermediate 16)

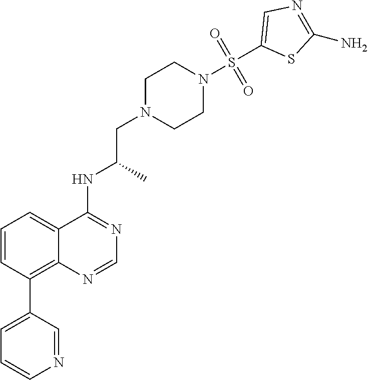 [SMILES: C[C@@H](CN1CCN(S(=O)(=O)c2cnc(N)s2)CC1)Nc1ncnc2c(-c3cccnc3)cccc12]

Compound N-[5-[4-[(2S)-2-[(8-pyridin-3-ylquinazolin-4-yl)amino]propyl]piperazin-1-yl]sulfonyl-1,3-thiazol-2-yl]acetamide (Intermediate 15, 167 mg, 0.3 mmol) was dissolved in a solution of sulfuric acid (1.64 mL, 31 mmol) and Water (1.67 mL) and the mixture was stirred at 40° C. overnight. Ice was added to the reaction mixture followed by a solution of NaOH 2M until pH 6. Then NaHCO₃sat. solution was added dropwise until formation of a white precipitate. The solid product was filtered off and dried under vacuum to afford title compound (Intermediate 16, 166 mg, crude) which was used in the next step without purification.

LC-MS (ESI): m/z (M+1): 511.1 (Method 2)

### Step 5: preparation of methyl N-[5-({4-[(2S)-2-{[8-(pyridin-3-yl)quinazolin-4-yl]amino}propyl]piperazin-1-yl}sulfonyl)-1,3-thiazol-2-yl]carbamate (Example 47)

To a solution of 5-[4-[(2S)-2-[(8-pyridin-3-ylquinazolin-4-yl)amino]propyl]piperazin-1-yl]sulfonyl-1,3-thiazol-2-amine (Intermediate 16, 82 mg, 0.16 mmol) in DCM (3 mL) at 0° C. DMAP (49 mg, 0.4 mmol) was added followed by methyl chloroformate (15 mg, 0.16 mmol). The mixture was stirred at room temperature for 12 h. The mixture was concentrated under reduced pressure and then purified by flash chromatography using a gradient of MeOH in DCM from 0% to 5% affording title compound (36.4 mg, 0.06 mmol, 40% yield).

LC-MS (ESI): m/z (M+1): 569.1 (Method 2)

¹H NMR (400 MHz, DMSO-d₆) δ ppm 1.21 (d, J=6.6 Hz, 3H), 2.44 (dd, J=12.3, 7.0 Hz, 1H), 2.54-2.68 (m, 5H), 2.94 (br s, 4H), 3.76 (s, 3H), 4.56-4.67 (m, 1H), 7.48 (dd, J=7.4, 4.7 Hz, 1H), 7.58 (t, J=7.8 Hz, 1H), 7.83 (dd, J=7.3, 1.1 Hz, 1H), 7.86 (s, 1H), 7.94 (d, J=7.7 Hz, 1H), 8.03 (dt, J=7.9, 2.0 Hz, 1H), 8.31 (d, J=7.3 Hz, 1H), 8.43 (s, 1H), 8.56 (dd, J=4.8, 1.5 Hz, 1H), 8.79 (d, J=1.5 Hz, 1H), 12.47 (br s, 1H)

The Examples in the following table were prepared from commercially available reagents by using methods analogous to Example 47.

| Example | | |
|---|---|---|
| No. | Structure & Name | Analytical data |
| 33 | 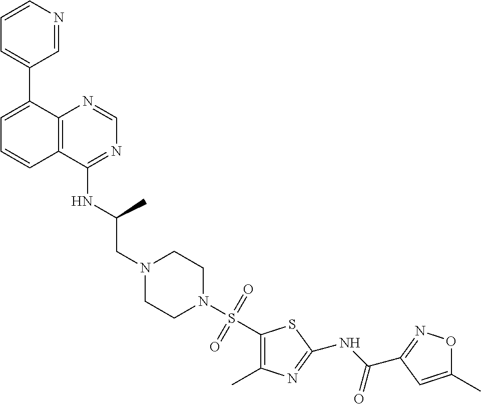 [SMILES: Cc1cc(C(=O)Nc2nc(C)c(S(=O)(=O)N3CCN(C[C@H](C)Nc4ncnc5c(-c6cccnc6)cccc45)CC3)s2)no1] | LC-MS (ESI): m/z (M + 1): 634.1 (Method 2) ¹H NMR (400 MHz, DMSO-d₆) δ ppm 1.21 (d, J = 6.4 Hz, 6 H), 2.38-2.74 (m, 9 H), 3.01 (br s, 7 H), 4.52-4.76 (m, 2 H), 6.61- 6.91 (m, 2 H), 7.47 (dd, J = 7.7, 4.8 Hz, 2 H), 7.57 (t, J = 7.8 Hz, 2 H), 7.82 (d, J = 7.2 Hz, 2 H), 7.94 (d, J = 7.7 Hz, 2 H), 8.02 (dt, J = 7.9, 1.8 Hz, 2 H), 8.31 (d, J = 7.9 Hz, 2 H), 8.43 (s, 2 H), 8.56 (dd, J = 4.7, 1.6 Hz, 2 H), 8.78 (d, J = 1.5 Hz, 2 H), 12.91- 14.03 (m, 1 H) |
| | 5-methyl-N-[4-methyl-5-({4-[(2S)-2-{[8- | |
| | (pyridin-3-yl)quinazolin-4- | |
| | yl]amino}propyl]piperazin-1- | |
| | yl}sulfonyl)-1,3-thiazol-2-yl]-1,2- | |
| | oxazole-3-carboxamide | |
| 49 | 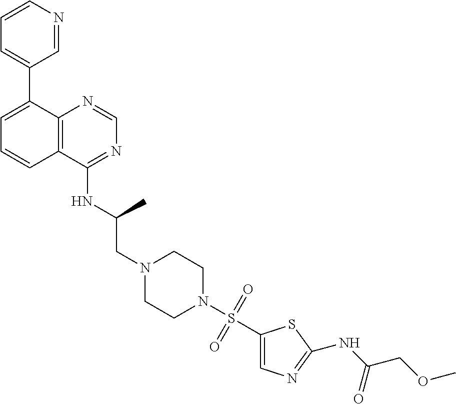 [SMILES: COCC(=O)Nc1ncc(S(=O)(=O)N2CCN(C[C@H](C)Nc3ncnc4c(-c5cccnc5)cccc34)CC2)s1] | LC-MS (ESI): m/z (M + 1): 583.1 (Method) ¹H NMR (400 MHz, DMSO-d₆) δ ppm 1.21 (d, J = 6.6 Hz, 3 H), 2.43 (dd, J = 12.5, 6.8 Hz, 1 H), 2.54-2.70 (m, 5 H), 2.94 (br s, 4 H), 3.34 (s, 3 H), 4.18 (s, 2 H), 4.61 (dt, J = 14.1, 7.0 Hz, 1 H), 7.48 (dd, J = 7.8, 4.9 Hz, 1 H), 7.58 (t, J = 7.8 Hz, 1 H), 7.83 (dd, J = 7.2, 1.1 Hz, 1 H), 7.94 (d, J = 7.9 Hz, 1 H), 7.97 (s, 1 H), 8.03 (dt, J = 7.9, 1.9 Hz, 1 H), 8.31 (d, J = 7.6 Hz, 1 H), 8.43 (s, 1 H), 8.56 (dd, J = 4.8, 1.6 Hz, 1 H), 8.78 (d, J = 1.5 Hz, 1 H), 12.74 (br s, 1 H) |
| | 2-methoxy-N-[5-({4-[(2S)-2-{[8- | |
| | (pyridin-3-yl)quinazolin-4- | |
| | yl]amino}propyl]piperazin-1- | |
| | yl}sulfonyl)-1,3-thiazol-2-yl]acetamide | |
| 75 | 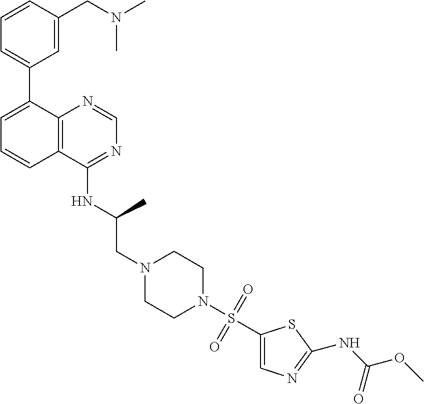 [SMILES: COC(=O)Nc1ncc(S(=O)(=O)N2CCN(C[C@H](C)Nc3ncnc4c(-c5cccc(CN(C)C)c5)cccc34)CC2)s1] | LC-MS (ESI): m/z (M + 1): 625.2 (Method 2) ¹H NMR (400 MHz, DMSO-d₆) δ ppm 1.21 (d, J = 6.6 Hz, 3 H), 2.30 (s, 6 H), 2.43 (br dd, J = 12.4, 6.2 Hz, 1 H), 2.58 (br d, J = 20.0 Hz, 5 H), 2.85 (br s, 4 H), 3.57-3.63 (m, 1 H), 3.67 (s, 3 H), 3.68-3.76 (m, 1 H), 4.65 (quin, J = 7.2 Hz, 1 H), 7.31 (d, J = 7.7 Hz, 1 H), 7.41 (t, J = 7.6 Hz, 1 H), 7.47-7.57 (m, 3 H), 7.72 (dd, J = 7.2, 1.1 Hz, 1 H), 7.79 (s, 1 H), 7.82 (d, J = 8.1 Hz, 1 H), 8.18-8.28 (m, 1 H), 8.38 (s, 1 H) |
| | methyl N-[5-({4-[(2S)-2-[(8-{3- | |
| | [(dimethylamino)methyl]phenyl}quinazolin- | |
| | 4-yl)amino]propyl]piperazin-1- | |
| | yl}sulfonyl)-1,3-thiazol-2-yl]carbamate | |
| 81 | 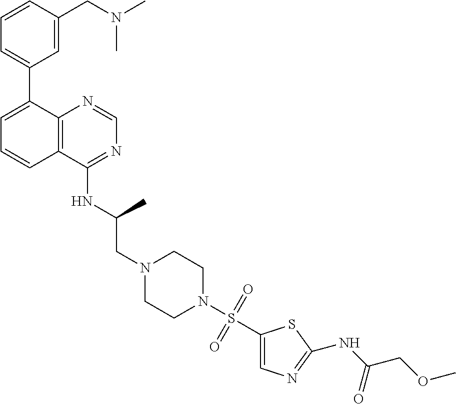 [SMILES: COCC(=O)Nc1ncc(S(=O)(=O)N2CCN(C[C@H](C)Nc3ncnc4c(-c5cccc(CN(C)C)c5)cccc34)CC2)s1] | LC-MS (ESI): m/z (M + 1): 639.2 (Method 2) ¹H NMR (400 MHz, DMSO-d₆) δ ppm 1.20 (d, J = 6.6 Hz, 3 H), 2.26 (s, 6 H), 2.43 (dd, J = 12.5, 6.8 Hz, 1 H), 2.55-2.64 (m, 5 H), 2.90 (br s, 4 H), 3.29 (s, 4 H), 3.51-3.67 (m, 2 H), 4.11 (s, 2 H), 4.56-4.69 (m, 1 H), 7.30 (d, J = 7.5 Hz, 1 H), 7.40 (t, J = 7.3 Hz, 1 H), 7.48-7.55 (m, 3 H), 7.72 (dd, J = 7.3, 1.2 Hz, 1 H), 7.84 (d, J = 8.1 Hz, 1 H), 7.92 (s, 1 H), 8.24 (d, J = 7.9 Hz, 1 H), 8.39 (s, 1 H) |
| | N-[5-({4-[(2S)-2-[(8-{3- | |
| | [(dimethylamino)methyl]phenyl}quinazolin- | |
| | 4-yl)amino]propyl]piperazin-1- | |
| | yl}sulfonyl)-1,3-thiazol-2-yl]-2- | |
| | methoxyacetamide | |
| 86 | 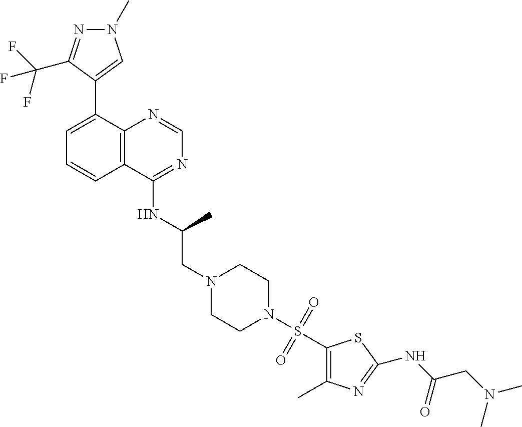 [SMILES: Cc1nc(NC(=O)CN(C)C)sc1S(=O)(=O)N1CCN(C[C@H](C)Nc2ncnc3c(-c4cn(C)nc4C(F)(F)F)cccc23)CC1] | LC-MS (ESI): m/z (M + 1): 681.3 (Method 3) ¹H NMR (400 MHz, DMSO-d₆) δ ppm 1.20 (d, J = 6.6 Hz, 3 H), 2.28 (s, 6 H), 2.38-2.43 (m, 1 H), 2.44 (s, 3 H), 2.54-2.66 (m, 5 H), 2.98 (br s, 5 H), 3.18- 3.45 (m, 2 H), 3.99 (s, 3 H), 4.59 (dt, J = 14.0, 6.8 Hz, 1 H), 7.50 (t, J = 7.8 Hz, 1 H), 7.66 (d, J = 7.2 Hz, 1 H), 7.89 (d, J = 7.7 Hz, 1 H), 8.06 (s, 1 H), 8.26 (dd, J = 8.6, 1.1 Hz, 1 H), 8.40 (s, 1 H), 10.87-13.01 (m, 1 H) |
| | 2-(dimethylamino)-N-[4-methyl-5-({4- | |
| | [(2S)-2-({8-[1-methyl-3- | |
| | (trifluoromethyl)-1H-pyrazol-4- | |
| | yl]quinazolin-4- | |
| | yl}amino)propyl]piperazin-1- | |
| | yl}sulfonyl)-1,3-thiazol-2-yl]acetamide | |

### Example 101

### methyl N-[5-({4-[(2S)-2-[(8-{1-[2-(dimethylamino)ethyl]-3,5-dimethyl-1H-pyrazol-4-yl}quinazolin-4-yl)amino]propyl]piperazin-1-yl}sulfonyl)-4-methyl-1,3-thiazol-2-yl]carbamate

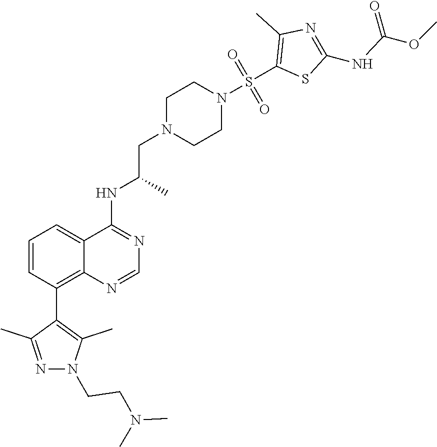

### Step 1: Preparation of tert-butyl 4-[(2S)-2-[[8-(3,5-dimethyl-1H-pyrazol-4-yl)quinazolin-4-yl]amino]propyl]piperazine-1-carboxylate (Intermediate 17)

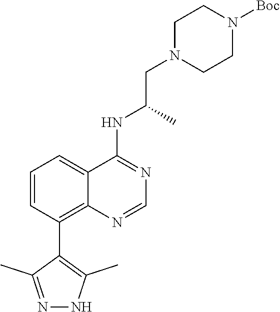 [SMILES: Cc1n[nH]c(C)c1-c1cccc2c(N[C@@H](C)CN3CCN(C(=O)OC(C)(C)C)CC3)ncnc12]

Title compound was prepared following the procedure used for the synthesis of Example 1, starting from tert-butyl 4-[(2S)-2-[(8-bromoquinazolin-4-yl)amino]propyl]piperazine-1-carboxylate (Intermediate 1, 200 mg, 0.4 mmol) and 3,5-Dimethylpyrazole-4-boronic acid pinacol ester (142 mg, 0.6 mmol) to afford title compound (130 mg, 0.28 mmol, 67% yield) as a brown solid.

LC-MS (ESI): m/z (M+1): 466.5 (Method 1)

### Step 2: Preparation of tert-butyl 4-[(2S)-2-[[8-[1-[2-(dimethylamino)ethyl]-3,5-dimethylpyrazol-4-yl]quinazolin-4-yl]amino]propyl]piperazine-1-carboxylate (Intermediate 18)

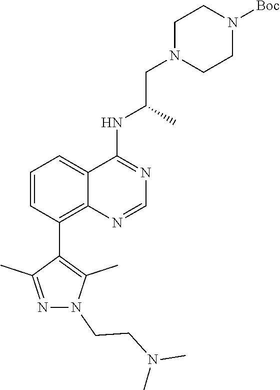 [SMILES: Cc1nn(CCN(C)C)c(C)c1-c1cccc2c(N[C@@H](C)CN3CCN(C(=O)OC(C)(C)C)CC3)ncnc12]

A suspension of tert-butyl 4-[(2S)-2-[[8-(3,5-dimethyl-1H-pyrazol-4-yl)quinazolin-4-yl]amino]propyl]piperazine-1-carboxylate (Intermediate 17, 130 mg, 0.28 mmol) and 2-Chloro-N,N-dimethylethylamine hydrochloride (100 mg, 0.7 mmol) in DMF (3 mL) was cooled down at 0° C. Sodium hydride (33.51 mg, 0.84 mmol) was added and the reaction mixture was stirred at r.t. overnight. The mixture was evaporated to dryness and the residue was taken up with AcOEt and H₂O and extracted 3 times. The organic layers were collected together, dried over Na₂SO₄, filtered and concentrated under reduced pressure to afford title compound (109 mg, 0.2 mmol, 73% yield) that was used in the next step without further purification.

LC-MS (ESI): m/z (M+1): 537.6 (Method 1)

### Step 3: Preparation of 8-[1-[2-(dimethylamino)ethyl]-3,5-dimethylpyrazol-4-yl]-N-[(2S)-1-piperazin-1-ylpropan-2-yl]quinazolin-4-amine (Intermediate 19)

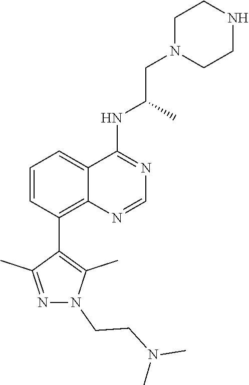 [SMILES: Cc1nn(CCN(C)C)c(C)c1-c1cccc2c(N[C@@H](C)CN3CCNCC3)ncnc12]

Title compound was prepared following the procedure used for the synthesis of Intermediate 2 starting from tert-butyl 4-[(2S)-2-[[8-[1-[2-(dimethylamino)ethyl]-3,5-dimethylpyrazol-4-yl]quinazolin-4-yl]amino]propyl]piperazine-1-carboxylate (Intermediate 18, 109 mg, 0.2 mmol). The crude was purified using a SCX cartridge loaded with MeOH and eluted with NH₃ 2M in MeOH to afford title compound (60 mg, 0.137 mmol, 67% yield) as a brown solid.

LC-MS (ESI): m/z (M+1): 437.6 (Method 2)

### Step 4: Preparation of methyl N-[5-({4-[(2S)-2-[(8-{1-[2-(dimethylamino)ethyl]-3,5-dimethyl-1H-pyrazol-4-yl}quinazolin-4-yl)amino]propyl]piperazin-1-yl}sulfonyl)-4-methyl-1,3-thiazol-2-yl]carbamate (Example 101)

Title compound was prepared following the procedure used for the synthesis of Intermediate 3, starting from 8-[1-[2-(dimethylamino)ethyl]-3,5-dimethylpyrazol-4-yl]-N-[(2S)-1-piperazin-1-ylpropan-2-yl]quinazolin-4-amine (Intermediate 19, 60 mg, 0.137 mmol) and methyl N-[5-(chlorosulfonyl)-4-methyl-1,3-thiazol-2-yl]carbamate (44.6 mg, 0.165 mmol) to afford title compound (32.4 mg, 0.048 mmol, 35% yield) as a white solid.

LC-MS (ESI): m/z (M+1): 671.5 (Method 1)

¹H NMR (400 MHz, DMSO-d₆) δ ppm 1.21 (d, J=6.4 Hz, 3H), 1.95 (s, 3H), 2.05 (s, 3H), 2.22 (s, 6H), 2.35-2.46 (m, 1H), 2.42 (s, 3H), 2.58 (br s, 4H), 2.60-2.66 (m, 3H), 2.99 (br s, 4H), 3.73 (s, 3H), 4.08 (t, J=7.0 Hz, 2H), 4.52-4.70 (m, 1H), 7.44-7.52 (m, 1H), 7.52-7.57 (m, 1H), 7.81 (d, J=7.9 Hz, 1H), 8.18-8.23 (m, 1H), 8.38 (s, 1H)

The Example in the following table was prepared from commercially available reagents by using methods analogous to Example 101.

| Example | | |
|---|---|---|
| No. | Structure & Name | Analytical data |
| 102 | 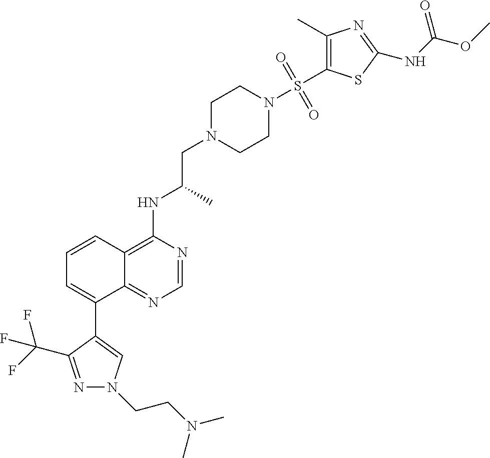 [SMILES: COC(=O)Nc1nc(C)c(S(=O)(=O)N2CCN(C[C@H](C)Nc3ncnc4c(-c5cn(CCN(C)C)nc5C(F)(F)F)cccc34)CC2)s1] | LC-MS (ESI): m/z (M + 1): 711.3 (Method 2) ¹H NMR (400 MHz, DMSO-d₆) δ ppm 1.21 (d, J = 6.6 Hz, 3 H), 2.20 (s, 6 H), 2.39-2.45 (m, 1 H), 2.41 (s, 3 H), 2.57 (br s, 4 H), 2.59-2.65 (m, 1 H), 2.75 (t, J = 6.5 Hz, 2 H), 2.97 (br s, 4 H), 3.72 (s, 3 H), 4.35 (t, J = 6.5 Hz, 2 H), 4.50-4.69 (m, 1 H), 7.50 (t, J = 7.8 Hz, 1 H), 7.67 (d, J = 7.1 Hz, 1 H), 7.89 (d, J = 8.1 Hz, 1 H), 8.12 (s, 1 H), 8.26 (d, J = 7.6 Hz, 1 H), 8.40 (s, 1 H), 12.28 (br s, 1 H) |
| | methyl N-[5-({4-[(2S)-2-[(8-{1-[2- | |
| | (dimethylamino)ethyl]-3- | |
| | (trifluoromethyl)-1H-pyrazol-4- | |
| | yl}quinazolin-4- | |
| | yl)amino]propyl]piperazin-1- | |
| | yl}sulfonyl)-4-methyl-1,3-thiazol-2- | |
| | yl]carbamate | |

Pharmacological Activity of the Compounds of the Invention

In Vitro Assays

The effectiveness of compounds of the present invention as LPA2 antagonist can be determined at the human recombinant LPA2 expressed in CHO cells, using a FLIPR assay in 384 well format.

CHO-hLPA2 cell lines are cultured in a humidified incubator at 5% C₀₂ in DMEM/F-12 (1:1) MIXTURE with 2 mM Glutamax, supplemented with 10% of Foetal Bovine Serum, 1 mM Sodium Pyruvate, 11 mM Hepes and 1× Penicillin/Streptomycin. CHO hLPA2 cells are seeded into black walled clear-bottom 384-well plates (#781091, Greiner Bio-One GmbH) at a density of 7,500 cells per well in 50 μl culture media and grown overnight in a 37° C. humidified CO2-incubator. Serial dilutions (1:3 or 1:4, 11 points CRC) of compounds are performed in 100% DMSO at 200× the final concentration. The compounds are diluted 1:50 prior to the experiment with Assay Buffer (20 mM HEPES, 145 mM NaCl, 5 mM KCl, 5.5 mM glucose, 1 mM MgCl₂ and 2 mM CaCl₂, pH 7.4 containing 0.01% Pluronic F-127) to obtain a solution corresponding to 5-fold the final concentration in the assay (4×, 2% DMSO). The final concentration of DMSO in the assay will be 0.5% in each well. Medium is removed by aspiration and cells are then incubated with 30 μl of a loading solution containing 5 μM of the cytoplasmic Ca2+ indicator Cal-520 AM in Assay Buffer containing 2.5 mM probenecid for 30 min at 37° C. incubator (cell loading). The loaded cell plates are transferred into the FLIPR instrument and calcium responses are monitored during the on-line addition protocols. For testing of compounds, after the cell loading, 10 μl/well of 4× antagonists' solution was added onto the cells. After 30 min pre-incubation (at 37° C.), 10 μl/well of 5× concentrated LPA EC80 was added and Ca2+ mobilization responses was followed during the on-line addition protocol. Intracellular peak fluorescence values subtracted by baseline fluorescence are exported and analysed to determine IC₅₀ values, respectively. The calcium response is expressed as percentage of the maximal inhibition of the EC80 agonist response.

The raw data obtained in unstimulated controls (DMSO, no LPA) are set as “100% inhibition”, while the raw data obtained in negative controls, i.e. in the absence of compounds and stimulating with LPA EC80, are set as “0% inhibition”.

The raw data (peak height expressed as relative fluorescence units) are normalized and transformed into “percent of inhibition”. Curve fitting and pIC₅₀ (−LogIC₅₀) estimations are carried out using a four-parameter logistic model using XLfit Software.

The results for individual compounds are provided below in Table 2 wherein the compounds are classified in term of potency with respect to their inhibitory activity on LPA2 isoform, according to the following classification criterion:

TABLE 2
LPA receptor 2 (LPA2)

| Example No | LPA2 IC50 |
|---|---|
| 19 | + |
| 15 | + |
| 103 | ++ |
| 41 | ++ |
| 94 | ++ |
| 73 | ++ |
| 66 | ++ |
| 100 | ++ |
| 2 | ++ |
| 104 | ++ |
| 52 | ++ |
| 56 | ++ |
| 39 | ++ |
| 99 | ++ |
| 78 | ++ |
| 88 | ++ |
| 49 | ++ |
| 42 | ++ |
| 59 | ++ |
| 95 | ++ |
| 74 | ++ |
| 34 | ++ |
| 65 | +++ |
| 20 | +++ |
| 33 | +++ |
| 43 | +++ |
| 40 | +++ |
| 69 | +++ |
| 83 | +++ |
| 86 | +++ |
| 46 | +++ |
| 72 | +++ |
| 76 | +++ |
| 31 | +++ |
| 58 | +++ |
| 61 | +++ |
| 4 | +++ |
| 47 | +++ |
| 36 | +++ |
| 81 | +++ |
| 67 | +++ |
| 26 | +++ |
| 38 | +++ |
| 85 | +++ |
| 51 | +++ |
| 28 | +++ |
| 8 | +++ |
| 57 | +++ |
| 55 | +++ |
| 84 | +++ |
| 37 | +++ |
| 29 | +++ |
| 96 | +++ |
| 80 | +++ |
| 87 | +++ |
| 11 | +++ |
| 32 | +++ |
| 105 | +++ |
| 77 | +++ |
| 25 | +++ |
| 60 | +++ |
| 53 | +++ |
| 71 | +++ |
| 64 | +++ |
| 27 | +++ |
| 63 | +++ |
| 101 | +++ |
| 9 | +++ |
| 12 | +++ |
| 92 | +++ |
| 13 | +++ |
| 97 | +++ |
| 75 | +++ |
| 6 | +++ |
| 98 | +++ |
| 45 | +++ |
| 5 | +++ |
| 79 | +++ |
| 10 | +++ |
| 18 | +++ |
| 82 | +++ |
| 68 | +++ |
| 50 | +++ |
| 44 | +++ |
| 3 | +++ |
| 23 | +++ |
| 54 | +++ |
| 35 | +++ |
| 48 | +++ |
| 93 | +++ |
| 24 | +++ |
| 102 | +++ |
| 91 | +++ |
| 89 | +++ |
| 30 | +++ |
| 14 | +++ |
| 90 | +++ |
| 21 | +++ |
| 17 | +++ |
| 22 | +++ |
| 1 | +++ |
| 16 | +++ |
| 7 | +++ |

+: LPA2 IC₅₀ less than 1000 nM
++: LPA2 IC₅₀ comprised between about 100 nM and 10 nm
+++: LPA2 IC₅₀ less than about 10 nM.

As it can be appreciated in Table 2, the compounds of the present invention show a good activity as antagonists of LPA2 receptor.

### Comparative Example A

### Methyl (S)-(4-methyl-5-((4-(2-(pyrido[2,3-d]pyrimidin-4 ylamino)propyl)piperazin-1-yl)sulfonyl)thiazol-2-yl)carbamate

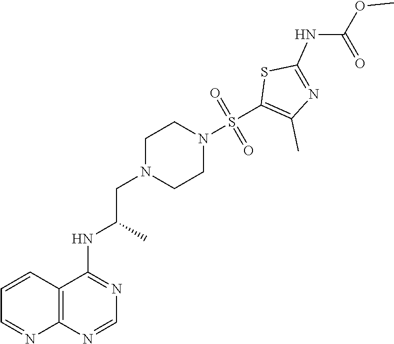 [SMILES: COC(=O)Nc1nc(C)c(S(=O)(=O)N2CCN(C[C@H](C)Nc3ncnc4ncccc34)CC2)s1]

The activity of comparative Example A as has been tested in the in vitro assay for the determination of activity on LPA2 receptor as described above.

Differently from the compounds of formula (I) of the present invention, the comparative Example A shows an IC₅₀ greater than 1 μm, even greater than 3 μm, and thus the compound is inactive on receptor LPA2.

The above results demonstrate that the scaffold of the compounds of formula (I) of the invention comprising a quinazoline moiety linked to the piperazine through an amino-alkyl linker leads unexpectedly to a series of compounds that is active on receptor LPA2.

1. A compound of formula (I) 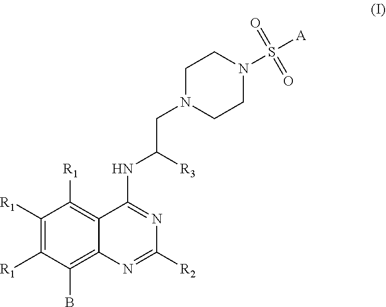
wherein
B is selected from the group consisting of (C₄-C₈) heterocycloalkyl, heteroaryl, (C₃-C₈)cycloalkyl, and aryl wherein each of said cycloalkyl, heterocycloalkyl, heteroaryl and aryl may be optionally substituted by one or more group selected from (C₁-C₄)alkyl, halo, (C₁-C₄)haloalkyl, —(C₁-C₄)alkylene-NR_{A}R_{B}, —NR_{A}R_{B}, —NR_{A}C(O)R₁; —C(O)R₁, —CN and (C₃-C₈)cycloalkyl, or
each of said aryl may be fused to a second saturated or unsaturated ring optionally containing one or more heteroatoms selected from N, O and S to form a bicyclic ring system optionally substituted by one or more group selected from —C(O)R₁, (C₁-C₄)alkyl and oxo;
R₁ is H or (C₁-C₄)alkyl;
R₂ is H or selected from the group consisting of (C₁-C₄)alkyl, (C₁-C₄)haloalkyl, —(C₁-C₄)alkylene-OR₁ and (C₃-C₈)cycloalkyl;
R₃ is H or (C₁-C₄)alkyl;
A is selected from the group consisting of 5-6 membered heteroaryl and aryl wherein each of said heteroaryl and aryl may be optionally substituted by one or more group selected from (C₁-C₄)alkyl, —C(O)R₁, —C(O)OR₁, —C(O)R₁, (C₁-C₄)haloalkyl, halo, —NR_{A}C(O)R₁, —NR_{A}C(O)OR₁, —NR_{A}C(O)—(C₁-C₄)alkylene-OR₁, —NR_{A}C(O)R_{C}, —NR_{A}C(O)NR_{A}R_{B}, —NR_{A}C(O)—(C₁-C₄)alkylene-NR_{A}R_{B}, —N(C₁-C₄)alkylene-NR_{A}R_{B}, aryl and heteroaryl optionally substituted by one or more (C₁-C₄)alkyl and (C₁-C₄)haloalkyl, or
when A is aryl it may be fused to a second saturated or unsaturated ring optionally containing one or more heteroatoms selected from N, O and S to form a bicyclic ring system optionally substituted by one or more group selected from —C(O)R₁, (C₁-C₄)alkyl and oxo;
R_{C} is selected from the group consisting of heteroaryl, aryl, (C₃-C₈) cycloalkyl and (C₄-C₈) heterocycloalkyl wherein said heteroaryl, aryl, heterocycloalkyl and cycloalkyl may be optionally substituted by one or more (C₁-C₄)alkyl and —C(O)OR₁;
R_{A} and R_{B} are at each occurrence independently H or selected from the group consisting of (C₁-C₄)alkyl, (C₃-C₈)cycloalkyl, (C₁-C₆)haloalkyl and halo, or
R_{A} and R_{B} may form together with the nitrogen atom to which they are attached a 4-6 membered saturated heterocyclic ring system optionally containing a further heteroatom selected from N, S and O, said heterocyclic ring system may be optionally substituted by one or more groups selected from (C₁-C₄)alkyl, (C₁-C₄) haloalkyl and halo.

2. The compound of formula (I) according to claim 1, wherein
B is selected from the group consisting of (C₄-C₈) heterocycloalkyl, heteroaryl, (C₃-C₈)cycloalkyl, and aryl wherein each of said heterocycloalkyl, heteroaryl and aryl may be optionally substituted by one or more group selected from (C₁-C₄)alkyl, halo, (C₁-C₄)haloalkyl, —(C₁-C₄)alkylene-NR_{A}R_{B}, —NR_{A}R_{B}, —C(O)R₁, —NR_{A}C(O)R₁, —CN and (C₃-C₈)cycloalkyl, or
each of said aryl may be fused to a saturated ring containing N to form a bicyclic ring system, optionally substituted by one or more (C₁-C₄)alkyl;
R₁ is H or (C₁-C₄)alkyl;
R₂ is H or selected from the group consisting of (C₁-C₄)alkyl, (C₁-C₄)haloalkyl, —(C₁-C₄)alkylene-OR₁ and C₃-C₈)cycloalkyl;
R₃ is (C₁-C₄)alkyl;
A is selected from the group consisting of 5-6 membered heteroaryl and aryl wherein each of said heteroaryl and aryl may be optionally substituted by one or more group selected from (C₁-C₄)alkyl, halo, —NR_{A}C(O)R₁, —NR_{A}C(O)OR₁, —NR_{A}C(O)—(C₁-C₄)alkylene-OR₁, —NR_{A}C(O)R_{C}, —N(C₁-C₄)alkylene-NR_{A}R_{B}, heteroaryl optionally substituted by one or more (C₁-C₄)alkyl; or
when A is aryl it may be fused to a second saturated or partially saturated ring optionally containing one or more heteroatoms selected from N, S and O, to form a bicyclic ring system optionally substituted by one or more group selected from oxo, —C(O)R₁, and (C₁-C₄)alkyl;
R_{C} is heteroaryl optionally substituted by one or more (C₁-C₄)alkyl;
R_{A} and R_{B} are at each occurrence independently H or selected from the group consisting of (C₁-C₄)alkyl, (C₃-C₈)cycloalkyl, (C₁-C₆)haloalkyl and halo, or R_{A} and R_{B} may form together with the nitrogen atom to which they are attached a 4-6 membered saturated heterocyclic ring system optionally containing a further heteroatom selected from N, S and O, said heterocyclic ring system may be optionally substituted by one or more groups selected from (C₁-C₄)alkyl, (C₁-C₄) haloalkyl and halo.

3. The compound of formula (I) according to claim 1 and 2, wherein when B is heteroaryl said heteroaryl is selected from the group consisting of thiazole, pyrazine, isoxazole, pyrazole, pyridine and pyrimidine.

4. The compound of formula (I) according to any claims 1 to 3, wherein A is selected from the group consisting of 5-6 membered heteroaryl and aryl wherein each of said heteroaryl and aryl may be optionally substituted by one or more group selected from (C₁-C₄)alkyl, halo, —NR_{A}C(O)R₁, —NR_{A}C(O)OR₁, —NR_{A}C(O)—(C₁-C₄)alkylene-OR₁, —NR_{A}C(O)R_{C}, —N(C₁-C₄)alkylene-NR_{A}R_{B} and isoxazole optionally substituted by one or more (C₁-C₄)alkyl; or
when A is aryl it may be fused to a second saturated or unsaturated ring optionally containing one or more heteroatoms selected from N, O and S to form a bicyclic ring system optionally substituted by one or more group selected from —C(O)R₁, (C₁-C₄)alkyl and oxo;

5. The compound of formula (I) according to any claims 1 to 4, wherein when A is 5-6 membered heteroaryl said 5-6 membered heteroaryl is selected from the group consisting of thiazole, thiophene and pyridine.

6. The compound of formula (I) according to any claims 1 to 5, wherein when R_{C} is heteroaryl said heteroaryl is isoxazole optionally substituted by one or more (C₁-C₄)alkyl and —C(O)OR₁.

7. The compound of formula (I) according to any claims 1 to 6 selected from at least one of:
methyl N-[5-({4-[(2S)-2-{[8-(2,4-dimethyl-1,3-thiazol-5-yl)quinazolin-4-yl]amino}propyl]piperazin-1-yl}sulfonyl)-4-methyl-1,3-thiazol-2-yl]carbamate,
methyl N-[4-methyl-5-({4-[(2S)-2-{[8-(5-methylpyrazin-2-yl)quinazolin-4-yl]amino}propyl]piperazin-1-yl}sulfonyl)-1,3-thiazol-2-yl]carbamate,
8-(3,5-dimethyl-1,2-oxazol-4-yl)-N-[(2S)-1-(4-{[5-(3-methyl-1,2-oxazol-5-yl)thiophen-2-yl]sulfonyl}piperazin-1-yl)propan-2-yl]quinazolin-4-amine,
N-[(2S)-1-(4-{[5-(3-methyl-1,2-oxazol-5-yl)thiophen-2-yl]sulfonyl}piperazin-1-yl)propan-2-yl]-8-phenylquinazolin-4-amine,
N-[(2S)-1-(4-{[5-(3-methyl-1,2-oxazol-5-yl)thiophen-2-yl]sulfonyl}piperazin-1-yl)propan-2-yl]-8-(1,3,5-trimethyl-1H-pyrazol-4-yl)quinazolin-4-amine,
N-[(2S)-1-(4-{[5-(3-methyl-1,2-oxazol-5-yl)thiophen-2-yl]sulfonyl}piperazin-1-yl)propan-2-yl]-8-(pyridin-3-yl)quinazolin-4-amine,
8-(2,4-dimethyl-1,3-thiazol-5-yl)-N-[(2S)-1-(4-{[5-(3-methyl-1,2-oxazol-5-yl)thiophen-2-yl]sulfonyl}piperazin-1-yl)propan-2-yl]quinazolin-4-amine,
N-[(2S)-1-(4-{[5-(3-methyl-1,2-oxazol-5-yl)thiophen-2-yl]sulfonyl}piperazin-1-yl)propan-2-yl]-8-(pyridin-4-yl)quinazolin-4-amine,
8-(3,5-dimethyl-1,2-oxazol-4-yl)-N-[(2S)-1-(4-{[5-(3,4-dimethyl-1,2-oxazol-5-yl)thiophen-2-yl]sulfonyl}piperazin-1-yl)propan-2-yl]quinazolin-4-amine,
methyl N-[5-({4-[(2S)-2-{[8-(3,5-dimethyl-1,2-oxazol-4-yl)quinazolin-4-yl]amino}propyl]piperazin-1-yl}sulfonyl)-4-methyl-1,3-thiazol-2-yl]carbamate,
N-[(2S)-1-(4-{[5-(3-methyl-1,2-oxazol-5-yl)thiophen-2-yl]sulfonyl}piperazin-1-yl)propan-2-yl]-8-(pyrimidin-5-yl)quinazolin-4-amine,
N-[(2S)-1-(4-{[5-(3-methyl-1,2-oxazol-5-yl)thiophen-2-yl]sulfonyl}piperazin-1-yl)propan-2-yl]-8-(pyridin-2-yl)quinazolin-4-amine,
methyl N-[4-methyl-5-({4-[(2S)-2-{[8-(pyridin-3-yl)quinazolin-4-yl]amino}propyl]piperazin-1-yl}sulfonyl)-1,3-thiazol-2-yl]carbamate,
N-[(2S)-1-(4-{[5-(3-methyl-1,2-oxazol-5-yl)thiophen-2-yl]sulfonyl}piperazin-1-yl)propan-2-yl]-8-[1-methyl-3-(trifluoromethyl)-1H-pyrazol-4-yl]quinazolin-4-amine,
N-[(2S)-1-(4-{[5-(3-methyl-1,2-oxazol-5-yl)thiophen-2-yl]sulfonyl}piperazin-1-yl)propan-2-yl]-8-[2-(trifluoromethyl)-1,3-thiazol-5-yl]quinazolin-4-amine,
N-[(2S)-1-(4-{[5-(3-methyl-1,2-oxazol-5-yl)thiophen-2-yl]sulfonyl}piperazin-1-yl)propan-2-yl]-8-{3-[(pyrrolidin-1-yl)methyl]phenyl}quinazolin-4-amine,
8-{3-[(dimethylamino)methyl]phenyl}-N-[(2S)-1-(4-{[5-(3-methyl-1,2-oxazol-5-yl)thiophen-2-yl]sulfonyl}piperazin-1-yl)propan-2-yl]quinazolin-4-amine,
8-(3-cyclopropyl-1-methyl-1H-pyrazol-4-yl)-N-[(2S)-1-(4-{[5-(3-methyl-1,2-oxazol-5-yl)thiophen-2-yl]sulfonyl}piperazin-1-yl)propan-2-yl]quinazolin-4-amine,
N-[2-(4-{[5-(3-methyl-1,2-oxazol-5-yl)thiophen-2-yl]sulfonyl}piperazin-1-yl)ethyl]-8-(pyridin-3-yl)quinazolin-4-amine,
N-[(2S)-1-(4-{[5-(3-methyl-1,2-oxazol-5-yl)thiophen-2-yl]sulfonyl}piperazin-1-yl)propan-2-yl]-8-(pyrrolidin-1-yl)quinazolin-4-amine,
methyl N-[5-({4-[(2S)-2-[(8-{3-[(dimethylamino)methyl]phenyl}quinazolin-4-yl)amino]propyl]piperazin-1-yl}sulfonyl)-4-methyl-1,3-thiazol-2-yl]carbamate,
methyl N-[4-methyl-5-({4-[(2S)-2-[(8-{3-[(pyrrolidin-1-yl)methyl]phenyl}quinazolin-4-yl)amino]propyl]piperazin-1-yl}sulfonyl)-1,3-thiazol-2-yl]carbamate,
methyl N-[4-methyl-5-({4-[(2S)-2-[(8-{4-[(pyrrolidin-1-yl)methyl]phenyl}quinazolin-4-yl)amino]propyl]piperazin-1-yl}sulfonyl)-1,3-thiazol-2-yl]carbamate,
methyl N-[4-methyl-5-({4-[(2S)-2-[(8-{3-[(4-methylpiperazin-1-yl)methyl]phenyl}quinazolin-4-yl)amino]propyl]piperazin-1-yl}sulfonyl)-1,3-thiazol-2-yl]carbamate,
methyl N-[4-methyl-5-({4-[(2S)-2-[(8-{5-[(pyrrolidin-1-yl)methyl]pyridin-3-yl}quinazolin-4-yl)amino]propyl]piperazin-1-yl}sulfonyl)-1,3-thiazol-2-yl]carbamate,
methyl N-[5-({4-[(2S)-2-({8-[6-(dimethylamino)pyridin-3-yl]quinazolin-4-yl}amino)propyl]piperazin-1-yl}sulfonyl)-4-methyl-1,3-thiazol-2-yl]carbamate,
methyl N-[5-({4-[(2S)-2-{[8-(3,5-dimethyl-1H-pyrazol-4-yl)quinazolin-4-yl]amino}propyl]piperazin-1-yl}sulfonyl)-4-methyl-1,3-thiazol-2-yl]carbamate,
methyl N-[4-methyl-5-({4-[(2S)-2-{[8-(6-methylpyridin-3-yl)quinazolin-4-yl]amino}propyl]piperazin-1-yl}sulfonyl)-1,3-thiazol-2-yl]carbamate,
methyl N-[4-methyl-5-({4-[(2S)-2-{[8-(pyridin-2-yl)quinazolin-4-yl]amino}propyl]piperazin-1-yl}sulfonyl)-1,3-thiazol-2-yl]carbamate,
methyl N-[4-methyl-5-({4-[(2S)-2-({8-[1-methyl-3-(trifluoromethyl)-1H-pyrazol-4-yl]quinazolin-4-yl}amino)propyl]piperazin-1-yl}sulfonyl)-1,3-thiazol-2-yl]carbamate,
N-[(2S)-1-[4-(3,4-dichlorobenzenesulfonyl)piperazin-1-yl]propan-2-yl]-8-(pyridin-3-yl)quinazolin-4-amine,
N-[4-methyl-5-({4-[(2S)-2-{[8-(pyridin-3-yl)quinazolin-4-yl]amino}propyl]piperazin-1-yl}sulfonyl)-1,3-thiazol-2-yl]acetamide,
5-methyl-N-[4-methyl-5-({4-[(2S)-2-{[8-(pyridin-3-yl)quinazolin-4-yl]amino}propyl]piperazin-1-yl}sulfonyl)-1,3-thiazol-2-yl]-1,2-oxazole-3-carboxamide,
methyl N-[4-methyl-5-({4-[(2S)-2-{[8-(2-methylpyrimidin-5-yl)quinazolin-4-yl]amino}propyl]piperazin-1-yl}sulfonyl)-1,3-thiazol-2-yl]carbamate,
methyl N-[4-methyl-5-({4-[(2S)-2-({8-[3-(4-methylpiperazin-1-yl)phenyl]quinazolin-4-yl}amino)propyl]piperazin-1-yl}sulfonyl)-1,3-thiazol-2-yl]carbamate,
methyl N-[4-methyl-5-({4-[(2S)-2-({8-[4-(4-methylpiperazin-1-yl)phenyl]quinazolin-4-yl}amino)propyl]piperazin-1-yl}sulfonyl)-1,3-thiazol-2-yl]carbamate,
N-[5-({4-[(2S)-2-{[8-(pyridin-3-yl)quinazolin-4-yl]amino}propyl]piperazin-1-yl}sulfonyl)-1,3-thiazol-2-yl]acetamide,
6-({4-[(2S)-2-{[8-(pyridin-3-yl)quinazolin-4-yl]amino}propyl]piperazin-1-yl}sulfonyl)-2,3-dihydro-1,3-benzothiazol-2-one,
8-(3,5-dimethyl-1H-pyrazol-4-yl)-N-[(2S)-1-{4-[(2-{[2-(dimethylamino)ethyl]amino}-4-methyl-1,3-thiazol-5-yl)sulfonyl]piperazin-1-yl}propan-2-yl]quinazolin-4-amine,
methyl N-[4-methyl-5-({4-[(2S)-2-{[8-(pyrimidin-5-yl)quinazolin-4-yl]amino}propyl]piperazin-1-yl}sulfonyl)-1,3-thiazol-2-yl]carbamate,
methyl N-[5-({4-[(2S)-2-[(8-cyclopropylquinazolin-4-yl)amino]propyl]piperazin-1-yl}sulfonyl)-4-methyl-1,3-thiazol-2-yl]carbamate,
N-[5-fluoro-2-methyl-4-({4-[(2S)-2-{[8-(pyridin-3-yl)quinazolin-4-yl]amino}propyl]piperazin-1-yl}sulfonyl)phenyl]acetamide,
1-[5-({4-[(2S)-2-{[8-(pyridin-3-yl)quinazolin-4-yl]amino}propyl]piperazin-1-yl}sulfonyl)-2,3-dihydro-1H-indol-1-yl]ethan-1-one,
methyl N-[4-methyl-5-({4-[(2S)-2-({8-[2-(4-methylpiperazin-1-yl)pyridin-4-yl]quinazolin-4-yl}amino)propyl]piperazin-1-yl}sulfonyl)-1,3-thiazol-2-yl]carbamate,
6-({4-[(2S)-2-[(8-{3-[(dimethylamino)methyl]phenyl}quinazolin-4-yl)amino]propyl]piperazin-1-yl}sulfonyl)-2,3-dihydro-1,3-benzothiazol-2-one,
N-[(2S)-1-[4-(3,4-dichlorobenzenesulfonyl)piperazin-1-yl]propan-2-yl]-8-{3-[(dimethylamino)methyl]phenyl}quinazolin-4-amine,
methyl N-[5-({4-[(2S)-2-{[8-(pyridin-3-yl)quinazolin-4-yl]amino}propyl]piperazin-1-yl}sulfonyl)-1,3-thiazol-2-yl]carbamate,
methyl N-[4-methyl-5-({4-[(2S)-2-{[8-(2-methylpyridin-3-yl)quinazolin-4-yl]amino}propyl]piperazin-1-yl}sulfonyl)-1,3-thiazol-2-yl]carbamate,
2-methoxy-N-[5-({4-[(2S)-2-{[8-(pyridin-3-yl)quinazolin-4-yl]amino}propyl]piperazin-1-yl}sulfonyl)-1,3-thiazol-2-yl]acetamide,
methyl N-[5-({4-[(2S)-2-[(8-{3-[(dimethylamino)methyl]-4-fluorophenyl}quinazolin-4-yl)amino]propyl]piperazin-1-yl}sulfonyl)-4-methyl-1,3-thiazol-2-yl]carbamate,
6-({4-[(2S)-2-{[8-(pyridin-3-yl)quinazolin-4-yl]amino}propyl]piperazin-1-yl}sulfonyl)-1,2,3,4-tetrahydroquinazoline-2,4-dione,
N-[(2S)-1-[4-(3,4-difluorobenzenesulfonyl)piperazin-1-yl]propan-2-yl]-8-(pyridin-3-yl)quinazolin-4-amine,
N-[4-({4-[(2S)-2-[(8-{3-[(dimethylamino)methyl]phenyl}quinazolin-4-yl)amino]propyl]piperazin-1-yl}sulfonyl)-5-fluoro-2-methylphenyl]acetamide,
1-[5-({4-[(2S)-2-[(8-{3-[(dimethylamino)methyl]phenyl}quinazolin-4-yl)amino]propyl]piperazin-1-yl}sulfonyl)-2,3-dihydro-1H-indol-1-yl]ethan-1-one,
methyl N-[5-({4-[(2S)-2-[(8-{5-[(dimethylamino)methyl]pyridin-3-yl}quinazolin-4-yl)amino]propyl]piperazin-1-yl}sulfonyl)-4-methyl-1,3-thiazol-2-yl]carbamate,
methyl N-[5-({4-[(2S)-2-{[8-(6-aminopyridin-3-yl)quinazolin-4-yl]amino}propyl]piperazin-1-yl}sulfonyl)-4-methyl-1,3-thiazol-2-yl]carbamate,
methyl N-[5-({4-[(2S)-2-{[8-(2,3-dihydro-1H-isoindol-5-yl)quinazolin-4-yl]amino}propyl]piperazin-1-yl}sulfonyl)-4-methyl-1,3-thiazol-2-yl]carbamate,
methyl N-[5-({4-[(2S)-2-[(8-{1-[2-(dimethylamino)ethyl]-1H-pyrazol-4-yl}quinazolin-4-yl)amino]propyl]piperazin-1-yl}sulfonyl)-4-methyl-1,3-thiazol-2-yl]carbamate,
6-({4-[(2S)-2-{[8-(pyridin-3-yl)quinazolin-4-yl]amino}propyl]piperazin-1-yl}sulfonyl)-1,2-dihydroquinoxalin-2-one,
methyl N-[5-({4-[(2S)-2-[(8-{6-[(dimethylamino)methyl]pyridin-3-yl}quinazolin-4-yl)amino]propyl]piperazin-1-yl}sulfonyl)-4-methyl-1,3-thiazol-2-yl]carbamate,
methyl N-[5-({4-[(2S)-2-{[8-(5-aminopyridin-3-yl)quinazolin-4-yl]amino}propyl]piperazin-1-yl}sulfonyl)-4-methyl-1,3-thiazol-2-yl]carbamate,
methyl N-[5-({4-[(2S)-2-{[8-(1,3-dimethyl-1H-pyrazol-4-yl)quinazolin-4-yl]amino}propyl]piperazin-1-yl}sulfonyl)-4-methyl-1,3-thiazol-2-yl]carbamate,
methyl N-[5-({4-[(2S)-2-{[8-(1,5-dimethyl-1H-pyrazol-4-yl)quinazolin-4-yl]amino}propyl]piperazin-1-yl}sulfonyl)-4-methyl-1,3-thiazol-2-yl]carbamate,
methyl N-[4-methyl-5-({4-[(2S)-2-{[8-(1-methyl-1H-pyrazol-4-yl)quinazolin-4-yl]amino}propyl]piperazin-1-yl}sulfonyl)-1,3-thiazol-2-yl]carbamate,
5-({4-[(2S)-2-{[8-(pyridin-3-yl)quinazolin-4-yl]amino}propyl]piperazin-1-yl}sulfonyl)-2,3-dihydro-1H-indol-2-one,
methyl N-[4-methyl-5-({4-[(2S)-2-({8-[5-(4-methylpiperazin-1-yl)pyridin-3-yl]quinazolin-4-yl}amino)propyl]piperazin-1-yl}sulfonyl)-1,3-thiazol-2-yl]carbamate,
1-[5-({4-[(2S)-2-({8-[1-methyl-3-(trifluoromethyl)-1H-pyrazol-4-yl]quinazolin-4-yl}amino)propyl]piperazin-1-yl}sulfonyl)-2,3-dihydro-1H-indol-1-yl]ethan-1-one,
1-[5-({4-[(2S)-2-{[8-(6-methylpyridin-3-yl)quinazolin-4-yl]amino}propyl]piperazin-1-yl}sulfonyl)-2,3-dihydro-1H-indol-1-yl]ethan-1-one,
methyl N-[4-methyl-5-({4-[(2S)-2-{[8-(6-methylpyridin-2-yl)quinazolin-4-yl]amino}propyl]piperazin-1-yl}sulfonyl)-1,3-thiazol-2-yl]carbamate,
methyl N-[5-({4-[(2S)-2-{[8-(5-formamidopyridin-3-yl)quinazolin-4-yl]amino}propyl]piperazin-1-yl}sulfonyl)-4-methyl-1,3-thiazol-2-yl]carbamate,
methyl N-[5-({4-[(2S)-2-{[8-(2-cyanopyrimidin-4-yl)quinazolin-4-yl]amino}propyl]piperazin-1-yl}sulfonyl)-4-methyl-1,3-thiazol-2-yl]carbamate,
methyl N-[5-({4-[(2S)-2-{[8-(6-chloro-5-fluoropyridin-2-yl)quinazolin-4-yl]amino}propyl]piperazin-1-yl}sulfonyl)-4-methyl-1,3-thiazol-2-yl]carbamate,
methyl N-[5-({4-[(2S)-2-[(8-{3-[(dimethylamino)methyl]phenyl}quinazolin-4-yl)amino]propyl]piperazin-1-yl}sulfonyl)-1,3-thiazol-2-yl]carbamate,
5-({4-[(2S)-2-[(8-{3-[(dimethylamino)methyl]phenyl}quinazolin-4-yl)amino]propyl]piperazin-1-yl}sulfonyl)-2,3-dihydro-1H-indol-2-one,
6-({4-[(2S)-2-[(8-{3-[(dimethylamino)methyl]phenyl}quinazolin-4-yl)amino]propyl]piperazin-1-yl}sulfonyl)-1,2,3,4-tetrahydroquinazoline-2,4-dione,
6-({4-[(2S)-2-{[8-(6-methylpyridin-3-yl)quinazolin-4-yl]amino}propyl]piperazin-1-yl}sulfonyl)-2,3-dihydro-1,3-benzothiazol-2-one,
6-({4-[(2S)-2-({8-[1-methyl-3-(trifluoromethyl)-1H-pyrazol-4-yl]quinazolin-4-yl}amino)propyl]piperazin-1-yl}sulfonyl)-2,3-dihydro-1,3-benzothiazol-2-one,
methyl N-[4-methyl-5-({4-[(2S)-2-{[8-(2-methyl-2,3-dihydro-1H-isoindol-5-yl)quinazolin-4-yl]amino}propyl]piperazin-1-yl}sulfonyl)-1,3-thiazol-2-yl]carbamate,
N-[5-({4-[(2S)-2-[(8-{3-[(dimethylamino)methyl]phenyl}quinazolin-4-yl)amino]propyl]piperazin-1-yl}sulfonyl)-1,3-thiazol-2-yl]-2-methoxyacetamide,
methyl N-[4-methyl-5-({4-[(2S)-2-({8-[3-(trifluoromethyl)-1H-pyrazol-4-yl]quinazolin-4-yl}amino)propyl]piperazin-1-yl}sulfonyl)-1,3-thiazol-2-yl]carbamate,
N-[(2S)-1-{4-[(2-methyl-1,3-benzothiazol-6-yl)sulfonyl]piperazin-1-yl}propan-2-yl]-8-(pyridin-3-yl)quinazolin-4-amine,
8-{3-[(dimethylamino)methyl]phenyl}-N-[(2S)-1-{4-[(2-methyl-1,3-benzothiazol-6-yl)sulfonyl]piperazin-1-yl}propan-2-yl]quinazolin-4-amine,
N-[(2S)-1-(4-{[5-(3-methyl-1,2-oxazol-5-yl)thiophen-2-yl]sulfonyl}piperazin-1-yl)propan-2-yl]-8-(6-methylpyridin-3-yl)quinazolin-4-amine,
2-(dimethylamino)-N-[4-methyl-5-({4-[(2S)-2-({8-[1-methyl-3-(trifluoromethyl)-1H-pyrazol-4-yl]quinazolin-4-yl}amino)propyl]piperazin-1-yl}sulfonyl)-1,3-thiazol-2-yl]acetamide,
methyl N-[5-({4-[(2S)-2-{[8-(5-formylpyridin-3-yl)quinazolin-4-yl]amino}propyl]piperazin-1-yl}sulfonyl)-4-methyl-1,3-thiazol-2-yl]carbamate,
N-[(2S)-1-[4-(benzenesulfonyl)piperazin-1-yl]propan-2-yl]-8-[1-methyl-3-(trifluoromethyl)-1H-pyrazol-4-yl]quinazolin-4-amine,
methyl N-[5-({4-[(2S)-2-[(8-{3-[(dimethylamino)methyl]-2-methylphenyl}quinazolin-4-yl)amino]propyl]piperazin-1-yl}sulfonyl)-4-methyl-1,3-thiazol-2-yl]carbamate,
methyl N-[5-({4-[(2S)-2-[(8-{3-[(dimethylamino)methyl]-2-fluorophenyl}quinazolin-4-yl)amino]propyl]piperazin-1-yl}sulfonyl)-4-methyl-1,3-thiazol-2-yl]carbamate,
methyl N-[5-({4-[(2S)-2-[(8-{3-[(dimethylamino)methyl]-4-methylphenyl}quinazolin-4-yl)amino]propyl]piperazin-1-yl}sulfonyl)-4-methyl-1,3-thiazol-2-yl]carbamate,
methyl N-[5-({4-[(2S)-2-{[8-(2,6-dimethylpyridin-3-yl)quinazolin-4-yl]amino}propyl]piperazin-1-yl}sulfonyl)-4-methyl-1,3-thiazol-2-yl]carbamate,
8-{3-[(dimethylamino)methyl]phenyl}-N-[(2S)-1-(4-{[5-(3-methyl-1,2-oxazol-5-yl)thiophen-3-yl]sulfonyl}piperazin-1-yl)propan-2-yl]quinazolin-4-amine,
3-methyl-6-({4-[(2S)-2-{[8-(pyridin-3-yl)quinazolin-4-yl]amino}propyl]piperazin-1-yl}sulfonyl)-2,3-dihydro-1,3-benzothiazol-2-one,
methyl N-[4-methyl-5-({4-[(2S)-2-{[8-(6-methylpyrimidin-4-yl)quinazolin-4-yl]amino}propyl]piperazin-1-yl}sulfonyl)-1,3-thiazol-2-yl]carbamate,
6-({4-[(2S)-2-[(8-{3-[(dimethylamino)methyl]-4-fluorophenyl}quinazolin-4-yl)amino]propyl]piperazin-1-yl}sulfonyl)-3-methyl-2,3-dihydro-1,3-benzothiazol-2-one,
6-({4-[(2S)-2-[(8-{3-[(dimethylamino)methyl]-4-fluorophenyl}quinazolin-4-yl)amino]propyl]piperazin-1-yl}sulfonyl)-2,3-dihydro-1,3-benzothiazol-2-one,
6-({4-[(2S)-2-{[8-(2,4-dimethyl-1,3-thiazol-5-yl)quinazolin-4-yl]amino}propyl]piperazin-1-yl}sulfonyl)-2,3-dihydro-1,3-benzothiazol-2-one,
N-[(2S)-1-{4-[(2-methyl-1,3-benzothiazol-6-yl)sulfonyl]piperazin-1-yl}propan-2-yl]-8-(6-methylpyridin-3-yl)quinazolin-4-amine,
3-methyl-6-({4-[(2S)-2-{[8-(6-methylpyridin-3-yl)quinazolin-4-yl]amino}propyl]piperazin-1-yl}sulfonyl)-2,3-dihydro-1,3-benzothiazol-2-one,
methyl N-[5-({4-[(2S)-2-[(8-{1-[2-(dimethylamino)ethyl]-3,5-dimethyl-1H-pyrazol-4-yl}quinazolin-4-yl)amino]propyl]piperazin-1-yl}sulfonyl)-4-methyl-1,3-thiazol-2-yl]carbamate,
methyl N-[5-({4-[(2S)-2-[(8-{1-[2-(dimethylamino)ethyl]-3-(trifluoromethyl)-1H-pyrazol-4-yl}quinazolin-4-yl)amino]propyl]piperazin-1-yl}sulfonyl)-4-methyl-1,3-thiazol-2-yl]carbamate,
8-[1-methyl-3-(trifluoromethyl)-1H-pyrazol-4-yl]-N-[(2S)-1-[4-(pyridine-2-sulfonyl)piperazin-1-yl]propan-2-yl]quinazolin-4-amine,
8-[1-methyl-3-(trifluoromethyl)-1H-pyrazol-4-yl]-N-[(2S)-1-[4-(pyridine-3-sulfonyl)piperazin-1-yl]propan-2-yl]quinazolin-4-amine,
8-{3-[(dimethylamino)methyl]-4-fluorophenyl}-N-[(2S)-1-{4-[(2-methyl-1,3-benzothiazol-6-yl)sulfonyl]piperazin-1-yl}propan-2-yl]quinazolin-4-amine.

8. A pharmaceutical composition comprising a compound of formula (I) according to any one of claims 1 to 7, in admixture with one or more pharmaceutically acceptable carrier or excipient.

9. The pharmaceutical composition according to claim 8 for oral administration.

10. A compound of formula (I) according to any one of claims 1 to 7 or a pharmaceutical composition according to claims 8 and 9 for use as a medicament.

11. A compound of formula (I) or a pharmaceutical composition for use according to claim 10 in treating disease, disorder, or condition associated with dysregulation of lysophosphatidic acid receptor 2 (LPA2).

12. A compound of formula (I) or a pharmaceutical composition for use according to claims 10 and 11 in the prevention and/or treatment of fibrosis and/or diseases, disorders, or conditions that involve fibrosis.

13. A compound of formula (I) or a pharmaceutical composition for use according to claim 12 in the prevention and/or treatment of fibrosis including pulmonary fibrosis, idiopathic pulmonary fibrosis (IPF), hepatic fibrosis, renal fibrosis, ocular fibrosis, cardiac fibrosis, arterial fibrosis and systemic sclerosis.

14. A compound of formula (I) or a pharmaceutical composition for use according to claim 13 in the prevention and/or treatment idiopathic pulmonary fibrosis (IPF).
